(19) United States
(12) Patent Application Publication
(10) Pub. No.: US 2017/0088600 A1
(43) Pub. Date: Mar. 30, 2017

(54) NOVEL CRYSTAL STRUCTURE AND LIGAND BINDING SITES OF TRAIL RECEPTOR

(71) Applicant: La Jolla Institute for Allergy and Immunology, La Jolla, CA (US)

(72) Inventors: Christopher Benedict, Carlsbad, CA (US); Dirk Zajonc, San Diego, CA (US); Ivana Nemcovicova, Bratislava (SK); Shilpi Verma, Encinitas, CA (US)

(21) Appl. No.: 15/151,391

(22) Filed: May 10, 2016

Related U.S. Application Data
(63) Continuation of application No. 14/069,030, filed on Oct. 31, 2013, now abandoned.
(60) Provisional application No. 61/721,368, filed on Nov. 1, 2012.

(51) Int. Cl. C07K 14/705; G01N 33/68

(52) U.S. Cl. CPC C07K 14/70578; A61K 47/48276; G01N 33/6863; G01N 2333/525; G01N 2500/04; G01N 2510/00

(57) ABSTRACT

A composition comprising a TRAIL-R2 receptor or fragment thereof bound to a ligand in crystalline form is presently provided along with novel binding sites and binding agents of a TRAIL receptor. Also provided are methods of designing a compound, protein or peptide and identifying a binding agent that interacts with a TRAIL receptor. The present invention further provides methods of modulating binding of a TRAIL receptor to a ligand, the methods comprising contacting the TRAIL receptor with a binding agent, ligand, or an agonist or antagonist thereof, that interacts with a novel binding site described herein.

### RELATED APPLICATIONS

This application claims the benefit of priority to provisional application Ser. No. 61/721,368, filed Nov. 1, 2011, which is expressly incorporated herein by reference in its entirety.

### FIELD OF THE INVENTION

The present invention provides a novel composition of TRAIL-R2 receptor or fragment thereof bound to a ligand in crystalline form and novel ligand binding sites and binding agents of TRAIL receptors. Also provided are methods of designing a compound, protein or peptide or identifying a binding agent and methods of modulating binding of a TRAIL receptor to a ligand comprising use of the novel composition or novel ligand binding sites described herein.

### BACKGROUND OF INVENTION

The immune system has evolved to protect against the many pathogens that are encountered throughout the lifetime of an individual. In turn, the selective pressure that is exerted by the immune system has shaped pathogen evolution. This co-evolutionary relationship between host and pathogen is particularly clear for viruses that establish persistent infections, such as human herpesviruses (HHV)^{1,2}. Human cytomegalovirus (HCMV, a β-herpesvirus, HHV-5), is a large double-stranded DNA virus that causes a lifelong, persistent/latent infection in ˜50 80% of the US population, varying with age, geography and socioeconomic status. While HCMV infection is largely asymptomatic in healthy persons, it can induce serious disease in those with naïve or compromised immunity, and the high incidence of congenital infection has spurred a strong initiative for vaccine development³. Primary clinical isolates carry at least 19 additional genes within the UL/b′ genomic region (UL133-151 locus) that have been lost in several commonly used HCMV strains that have been passaged extensively in tissue culture^{4,5}, with several of them targeting signaling by the TNFR superfamily (e.g. UL144 and UL138)⁶.

The interaction between TNF ligands and their respective TNFRs controls pleiotropic biological responses, including cell differentiation, proliferation and apoptosis⁷. Both TNF ligands and TNFRs are expressed on T cells and, as such, play important roles in T cell co-stimulation. In addition, TNF superfamily members are crucial in controlling herpesvirus infection by initiating the direct killing of infected cells and by enhancing immune responses^{8,9}. For instance, TRAIL death receptor (TRAIL-DR) regulation of apoptosis is critical for maintaining immune homeostasis during HHV infection. The herpesviruses, however, can block apoptosis, likely facilitating their ability to establish lifelong infection ^{10,11}. Using specific genetic mutants of HCMV UL141 has been recently identified to restrict expression of TRAIL-DR (TRAIL-R1/DR4 and TRAIL-R2/DR5)¹². It has been shown that cells infected with an HCMVΔUL141 mutant are more susceptible to killing by TRAIL, and that UL141 is both necessary and sufficient to inhibit expression of both the TRAIL receptors¹².

HCMV UL141 is also necessary and sufficient to inhibit cell surface expression of CD155 (PVR, poliovirus receptor; nectin-like molecule 5), a ligand for the NK cell activating receptor DNAM-1 (CD226). DNAM-1 also binds a second ligand, CD112 (nec-2, PRR-2, poliovirusreceptor-related protein 2), and UL141 is required, but not sufficient, to target CD112 for proteasome-mediated degradation. As a consequence, both activating ligands for DNAM-1 are removed from the surface of HCMV infected cells, and NK cell killing of those cells is markedly inhibited¹³⁻¹⁶. In addition, it has been recently shown that UL141 inhibition of TRAIL DR also contributes to inhibit TRAIL-mediated NK cell killing¹².

### SUMMARY OF THE INVENTION

Despite inducing a strong host immune response, HCMV persists for life in a latent form, which can be rapidly reactivated in the absence of host immunity, highlighting the dynamic relationship between the host and this virus. Characterizing the structural and molecular basis of the interactions that occur between specific HCMV proteins and the host molecules they target is crucial for understanding of viral persistence, and will ultimately facilitate vaccine and antiviral drug development.

The present inventors have discovered the structural and biochemical basis of a novel ligand binding site of a TRAIL receptor.

The present inventors have discovered a novel, non-canonical interaction between UL141 and TRAIL-R2, an interaction that has evolved to inhibit cell death mediated by TRAIL signaling and mute host defenses. Previously, TRAIL (TNFSF10) was the only known ligand for the four TRAIL receptors (TNFRSF10a-d). Remarkably, UL141 displays no structural homology to TNF superfamily ligands, and instead utilizes its Ig-domain to bind with high affinity to the TRAIL death receptor. Without being limited to any particular theory, the UL141 protein encoded by low-passage isolates of human cytomegalovirus (HCMV) may mimic a host molecule that binds the TRAIL death receptors (DR). Thus in one aspect, there is presently provided a composition comprising a TRAIL-R2 receptor or fragment thereof bound to a ligand in crystalline form. In particular embodiments, the ligand is bound to an amino acid sequence of TRAIL-R2 receptor that comprises, consists of or consists essentially of amino acid residues 58-184 of TRAIL-R2 receptor or a subsequence, portion, homologue, variant or derivative thereof. In certain embodiments, the ligand is bound to an amino acid sequence of TRAIL-R2 receptor that comprises, consists of or consists essentially of amino acid residues 58-212 of TRAIL-R2 receptor or a subsequence, portion, homologue, variant or derivative thereof.

In particular embodiments of the composition of the present invention, the crystalline form has unit cell parameters of a=67.74 Å, b=97.01 Å and c=140.94 Å or a=67.71 Å, b=97.67 Å, c=141.31 Å. In further embodiments, the composition comprises the relative structural coordinates set forth in FIG. 23 wherein the resolution is 2.1 Angstrom. In still further embodiments, the composition comprises a structure set forth in FIG. 1 or FIG. 3.

In certain embodiments of the presently described composition, the TRAIL-R2 receptor has one or more binding patches in contact with the ligand, the binding patches comprising, consisting of or consisting essentially of: amino acid residues E78 and D109 of a TRAIL-R2 receptor; amino acid residue D148 of a TRAIL-R2 receptor; amino acid residues V167, V179 and W173 of a TRAIL-R2 receptor; amino acid residues Y103, N134 and R133 of a TRAIL-R2 receptor; amino acid residues L110, L114 and F112 of a TRAIL-R2 receptor; and amino acid residues E151 and E147 of a TRAIL-R2 receptor; or a subsequence, portion, homologue, variant or derivative thereof.

In particular embodiments of the composition of the present invention, the ligand is UL141.

In another aspect, there is provided an isolated or purified ligand binding site of a TRAIL-R2 receptor, or a subsequence, portion, homologue, variant or derivative thereof. In different embodiments, the isolated or purified ligand binding site or subsequence, portion, homologue, variant or derivative thereof comprises a structure set forth in any one of FIG. 1, 2, 3, 4 or 6.

In certain embodiments of the present inventions, the isolated or purified ligand binding site or subsequence, portion, homologue, variant or derivative thereof comprises, consists of or consists essentially of amino acid residues 58-184 of TRAIL-R2 receptor or a subsequence, portion, homologue, variant or derivative thereof. In particular embodiments, the isolated or purified ligand binding site or subsequence, portion, homologue, variant or derivative thereof of comprises, consists of or consists essentially of amino acid residues 58-212 of TRAIL-R2 receptor or a subsequence, portion, homologue, variant or derivative thereof.

In certain embodiments, the isolated or purified ligand binding site or subsequence, portion, homologue, variant or derivative thereof comprises, consists of or consists essentially of structural coordinates set forth in FIG. 23, or a subsequence, portion, homologue, variant or derivative thereof. In still further embodiments, the isolated or purified ligand binding site or subsequence, portion, homologue, variant or derivative thereof comprises, consists of or consists essentially of one or more of: amino acid residues E78 and D109 of a TRAIL-R2 receptor; amino acid residue D148 of a TRAIL-R2 receptor; amino acid residues V167, V179 and W173 of a TRAIL-R2 receptor; amino acid residues Y103, N134 and R133 of a TRAIL-R2 receptor; amino acid residues L110, L114 and F112 of a TRAIL-R2 receptor; and amino acid residues E151 and E147 of a TRAIL-R2 receptor; or a subsequence, portion, homologue, variant or derivative thereof.

In another aspect, there is presently provided a method of designing a compound, protein or peptide that interacts with a TRAIL-R2 receptor, the method comprising: use of the composition of the present invention to design the compound, protein or peptide, wherein the compound, protein or peptide interacts with a ligand binding site of the TRAIL-R2 receptor, the ligand binding site comprising, consisting of or consisting essentially of amino acid residues 58-184 of the TRAIL-R2 receptor or a subsequence, portion, homologue, variant or derivative thereof; producing or synthesizing the compound, protein or peptide; contacting the compound, protein or peptide with the TRAIL-R2 receptor; and detecting interaction of the compound, protein or peptide with the ligand binding site comprising, consisting of or consisting essentially of amino acid residues 58-184 of the TRAIL-R2 receptor or a subsequence, portion, homologue, variant or derivative thereof.

In a further aspect, there is presently provided a method of identifying a binding agent that interacts with at least one amino acid of a TRAIL-R2 receptor ligand binding site, the method comprising providing a test agent; contacting the test agent with a protein or peptide comprising, consisting of or consisting essentially of amino acid residues 58-184 of the TRAIL-R2 receptor or subsequence, portion, homologue, variant or derivative thereof; and detecting interaction of the test agent with the protein or peptide comprising, consisting of or consisting essentially of amino acid residues 58-184 of TRAIL-R2 receptor or subsequence, portion, homologue, variant or derivative thereof. In particular embodiments, the method comprises contacting the test agent with a protein or peptide comprising, consisting of or consisting essentially of amino acid residues 58-212 of the TRAIL-R2 receptor or subsequence, portion, homologue, variant or derivative thereof; and detecting interaction of the test agent with the protein or peptide comprising, consisting of or consisting essentially of amino acid residues 58-212 of TRAIL-R2 receptor or subsequence, portion, homologue, variant or derivative thereof. In certain embodiments, the method comprises identifying a binding agent that interacts with one or more binding patches of the TRAIL-R2 receptor, the binding patches comprising, consisting of or consisting essentially of: amino acid residues E78 and D109 of a TRAIL-R2 receptor; amino acid residue D148 of the TRAIL-R2 receptor; amino acid residues V167, V179 and W173 of a TRAIL-R2 receptor; amino acid residues Y103, N134 and R133 of aTRAIL-R2 receptor; amino acid residues L110, L114 and F112 of a TRAIL-R2 receptor; and amino acid residues E151 and E147 of the TRAIL-R2 receptor; or a subsequence, portion, homologue, variant or derivative thereof; the method comprising: providing a test agent; contacting the test agent with a protein or peptide comprising, consisting of or consisting essentially of one or more the binding patches of the TRAIL receptor; and detecting interaction of the test agent with the protein or peptide comprising, consisting of or consisting essentially of one or more of the binding patches of the TRAIL receptor.

In particular embodiments of the presently provided method of identifying a binding agent, the binding agent modulates binding of a TRAIL-R2 receptor to a ligand that interacts with at least one amino acid of a TRAIL receptor ligand binding site. In different embodiments, the binding agent is a TRAIL-R2 receptor agonist, a TRAIL-R2 receptor antagonist, an antibody, an inhibitory nucleic acid or a ligand mimetic.

In yet a further aspect, there is presently provided a binding agent that interacts with a TRAIL-R2 receptor, the binding agent interacting with at least one amino acid of a ligand binding site of the TRAIL-R2 receptor, the ligand binding site comprising, consisting of or consisting essentially of amino acid residues 58-184 of TRAIL-R2 receptor or a subsequence, portion, homologue, variant or derivative thereof. In certain embodiments, the ligand binding site comprises, consists of or consists essentially of amino acid residues 58-212 of TRAIL-R2 receptor or a subsequence, portion, homologue, variant or derivative thereof. In particular embodiments, the binding agent of the present invention interacts with one or more binding patches of the TRAIL-R2 receptor, the binding patches comprising, consisting of or consisting essentially of: amino acid residue D148 of a TRAIL-R2 receptor; amino acid residues V167, V179 and W173 of a TRAIL-R2 receptor; amino acid residues Y103, N134 and R133 of a TRAIL-R2 receptor; amino acid residues L110, L114 and F112 of aTRAIL-R2 receptor; and amino acid residues E151 and E147 of a TRAIL-R2 receptor; or a subsequence, portion, homologue, variant or derivative thereof.

In particular embodiments of the present invention, the binding agent modulates binding of the TRAIL-R2 receptor to a ligand that interacts with at least one amino acid of the TRAIL-R2 receptor ligand binding site. In certain embodiments, the binding agent modulates TRAIL-R2 receptor activity. In different embodiments, the binding agent is a TRAIL receptor agonist, a TRAIL receptor antagonist, an antibody, an inhibitory nucleic acid or a ligand mimetic.

In particular embodiments of the present invention, the binding agent modulates an immune response, an anti-inflammatory response, cell proliferation or an apoptotic response mediated by the TRAIL-R2 receptor or a ligand thereof.

In another aspect of the present invention, there is provided a method for modulating binding of a TRAIL-R2 receptor to a ligand, the method comprising contacting the TRAIL-R2 receptor with a binding agent or ligand, or an agonist or antagonist thereof, that interacts with at least one amino acid of a ligand binding site of the TRAIL-R2 receptor, the ligand binding site comprising, consisting of or consisting essentially of amino acid residues 58-184 of TRAIL-R2 receptor or a subsequence, portion, homologue, variant or derivative thereof. In particular embodiments, the ligand binding site comprises, consists of or consists essentially of amino acid residues 58-212 of TRAIL-R2 receptor or a subsequence, portion, homologue, variant or derivative thereof.

In certain embodiments of the presently provided method for modulating binding of a TRAIL-R2 receptor to a ligand, the method comprises modulating the activity of a TRAIL-R2 receptor. In different embodiments, the method comprises decreasing, reducing, inhibiting, suppressing, disrupting, eliciting, stimulating, inducing, promoting, increasing or enhancing TRAIL-R2 receptor activity. In certain embodiments, the method comprises modulating an immune response, an anti-inflammatory response, cell proliferation or an apoptotic response mediated by the TRAIL-R2 receptor or a ligand thereof.

Other aspects and features of the present invention will become apparent to those of ordinary skill in the art upon review of the following description of specific embodiments of the invention in conjunction with the accompanying figures.

### DESCRIPTION OF DRAWINGS

The patent or application file contains at least one drawing executed in color. Copies of this patent or patent application publication with color drawing(s) will be provided by the Office upon request and payment of the necessary fee.

In the figures, which illustrate, by way of example only, embodiments of the present invention:

FIG. 1: Crystal structure of the UL141-TRAIL-R2 complex. (a) Heterotetrameric structure of the UL141 dimer (rainbow cartoon) in complex with two TRAILR2 monomers (grey surface). Six distinct binding patches between UL141 and TRAIL-R2 are indicated with dotted circles. (b) 2-D topology of the UL141 subunit with rainbow colors from N-terminus (blue) to C-terminus (red). The N-terminal domain of UL141 exhibits a V-type immunoglobulin superfamily fold containing ten β-strands (a-g) and two short α-helices Y and Z. The C-terminal domain contains three β-strands (1-3) and α-helix X. Disulfide bonds (C84-C234 and C68-C143) are indicated as pink lines. Potential N-linked asparagines are drawn as pink sticks and glycans are shown in grey for one UL141 monomer (chain A) at position N132 and N147, while N117 is occupied in the other monomer (chain B). Disordered loops 168-174,199-207 and 217-226 are indicated as dotted lines. (c) The cartoon representation of TRAIL-R2 structure colored by CRDs (Cysteine Reach Domains); CRD-1 in blue (residues 78-94), CRD-2 in green (residues 95-137) and CRD-3 in red (residues 138-178). Disulfide bonds are depicted in yellow as a ball-and-stick representation. The β1β2 loop of CRD-3 (residues 143-157) and that of CRD-2 (residues 104-115) that make the key contacts with the ligands as well as other important loops (β5β6 of CRD-2, β2β3 of CRD-3, N-term loop) and the CXC motif are highlighted by circle or arrows.

FIG. 2: Comparison of the UL141 and TRAIL binding footprints on TRAIL-R2. TRAIL-R2 is shown as a grey molecular surface in three different orientations: left (a), front (b), and right (c). The binding interface between the UL141 and TRAIL-R2 (c) is divided into six binding patches, with patches 3 and 5 being similar to that of the TRAIL-TRAIL-R2 complex (a). TRAIL contact residues on TRAIL-R2 in cyan (a-b), UL141 contact residues on TRAIL-R2 in yellow (b-c), while the overlapping residues are green (b).

FIG. 3: Comparison of TRAIL-R2 structures. The TRAIL-R2 structure derived from the complex with UL141 (in cyan) is superimposed with all available crystal structures found in PDB database. Three TRAIL-R2/TRAIL structures: 1D4V (grey), 1DU3 (green) and 1D0G (light purple) and two TRAIL-R2 Fab structures: YSd1 Fab (1Z3A, red) and BdF1 Fab (2H9G, yellow). (a) Structures superimpose very well with the exception of the β1β2 loop of CRD-3 (Patch 3). Representative 2FO-FC electron density map contoured at 1σ, showing the key residues of receptor β1β2 loop (cyan) interacting with UL141 residues (orange) in patch 3 (b) and patch 3U (c). The well-defined electron density indicates, that the β1β2 loop of CRD-3 is well ordered upon UL141 binding.

FIG. 4: Comparison of receptor-ligand interaction between UL141 and TRAIL with TRAIL-R2 and mutational binding data. (a) Detailed interaction is shown for the six binding patches of the UL141-TRAIL-R2 complex; 1-2 (yellow), 3 (green), 3U (light-green), 4 (pink), 5 (red), 6 (orange), as well as for the patches 3, 3T, 4 and 5 of the TRAIL-TRAIL-R2 complex (same coloring scheme). Interaction residues are labeled and drawn as orange (UL141), salmon (TRAIL) and cyan (TRAIL-R2) sticks with atoms colored as follows: nitrogen, blue, oxygen, red; and sulfur, yellow. Hydrogen bonds, salt bridges and hydrophobic contacts (distance<4.0 Å) are shown as dashed black line. The name of interacting loop, helix or strand is listed around each box as well as the specificity of particular contact patch. (b) Open book view of UL141-TRAIL-R2 complex with their molecular surfaces outlined in grey. All binding patches (fingerprints on both molecules) follow the same color-code as above including residues selected for alanine scanning mutagenesis. (c) Relative effect on alanine mutagenesis of TRAIL-R2 on UL141 (middle column) and TRAIL (right column) binding, as analyzed by SPR (FIG. 5 and Table 3). Mutated residues are listed (left column). Mutation that do not affect receptor binding are labeled ‘YES’ while ‘NO’ indicates binding is abrogated. X-fold reduction in binding (compared to wild-type) is quantitated by numbered arrows.

FIG. 5: Binding of TRAIL-R2 mutants to TRAIL and UL141. Surface plasmon resonance study to assess the binding contribution of individual TRAIL-R2 residues to both viral UL141 and endogenous TRAIL. A Sensorgram for each kinetics experiment is shown in colored boxes (colored by binding patch as in FIG. 4). The specific alanine mutation on TRAIL-R2 as well as the calculated binding constant KD (nM) are indicated for each panel. Mutations that fully abrogate binding are indicated as n.d. (binding not detected).

FIG. 6: UL141 surface accessibility for receptor binding. Structure of UL141 (orange cartoon) in complex with TRAIL-R2 (cyan cartoon) shown in three views. All three potential N-linked glycosylation sites (Asn117, Asn132 and Asn147) where modeled with a five-sugar Man2GluNac2Fuc glycans, shown in dark grey ball-and-stick). Area A and B indicate available and accessible protein binding sites on UL141, while other available areas are expected to be mostly covered with glycans in the fully glycosylated protein. Location of potential protein-protein binding sites for unbound UL141 were calculated using ProMate (http://bioinfo.weizmann.ac.il/promate). For simplicity, only one UL141 subunit is shown here in molecular surface colored from blue reflecting the lowest probability assigned, to red, assigned to the highest probability. The highest probability areas that reflect possible binding sites in UL141; excluding those binding sites 1-6 of TRAIL-R2 (shown in dotted line here); and are not shielded by glycans, are areas A and B.

FIG. 7: TRAIL is constitutively expressed by immature liver NK cells. Total liver mononuclear cells were isolated from C57BL/6 (B6) wild-type and TRAIL-KO mice and analyzed by flow cytometry. NK cells were identified as NK1.1⁺CD3⁻, and were further analyzed for expression of DX5 and CD11b to identify both mature (DX5^{hi}CD11b^{hi}) and immature)(DX5^{lo}CD11b^{lo}) cells. It has been previously reported that TRAIL is constitutively expressed by immature NK cells in the mouse liver, but not in the spleen⁵⁸. TRAIL expression in immature NK cells was detected in WT mice, but not in TRAIL-KO mice, verifying the specificity of the anti-mTRAIL antibody (clone N2B2, rat IgG2a) and the genetically deficient mice used in these experiments.

FIG. 8. TRAIL expression is not detectable on the surface of splenocytes from naïve or poly I:C treated mice. Spleens were harvested from either naive or poly I:C treated (100 ug injected in vivo ˜18 hours prior to harvest) WT or TRAIL-KO B6 mice prior to analysis by flow cytometry. Shown is the analysis of TRAIL expression by either macrophages, dendritic, NK or T cells based on the expression of known cellular markers. These analyses were done in parallel with \ NK cells isolated from livers to assure detection methodologies were working. As depicted in the multicolored histograms, no differences in binding of N2B2 was observed to cells isolated from WT or TRAIL-KO mice, indicating TRAIL cell surface expression is not detectable under these experimental conditions in WT mice.

FIG. 9. Recombinant mouse and human TRAIL-R2:Fc proteins bind equivalently to mouse TRAIL. TRAIL-R2:Fc proteins from both mouse and human were incubated with 293T cells transiently transfected with a plasmid vector expressing mouse (m) TRAIL. These Fc proteins consist of the extracellular domains of TRAIL-R2 from the respective species, fused to the human IgG V_{H} constant domain. Fc proteins were added at the indicated concentrations, and binding was detected with an anti-IgG RPE conjugated antibody. Also shown is binding of anti-mTRAIL to the same cells (clone N2B2). Results indicate that both the mouse and human TRAIL-R2:Fc proteins are functional and both bind with roughly equivalent affinity to mTRAIL.

FIG. 10. Myeloid and NK cells express a novel, surface-associated molecule (‘ligand X’) that binds TRAIL-R2. The same spleen cells from WT or TRAIL-KO mice analyzed in FIG. 2 (no poly I:C injection) were incubated with either mouse or human TRAIL-R2:Fc protein, human IgG or mLTβR:Fc and binding was detected by flow cytometry as in FIG. 3. The results show that both mouse and human TRAIL-R2 binds strongly to the surface of dendritic cells, macrophages and, to a lesser extent, NK cells isolated from both WT and TRAIL-KO mice. As binding of TRAIL-R2:Fc to cells from TRAIL KO mice was at least as robust as binding to cells from WT mice, this indicates that a novel ligand(s) is expressed by these cell types, referred to herein as a novel ligand of TRAIL-R2 receptor, with the understanding that “novel ligand” could represent binding of TRAIL-R2 to one or more proteins. No binding of TRAIL-R2 was observed to T cells under these conditions, highlighting the binding specificity of TRAIL-R2 for the novel ligand in the other cell types. Importantly, this does not rule out that T cells may express the novel ligand under different conditions.

FIG. 11. Cellular recognition of ‘danger signals’ enhances expression of the novel ligand of TRAIL-R2 receptor. Expression of TNFRs and CD28-family proteins are often regulated upon cellular activation. To test whether this was true for the novel ligand of TRAIL-R2 receptor, splenocytes from naïve or poly I:C injected TRAIL-KO mice were analyzed for binding of mTRAIL-R2:Fc. Poly I:C cells enhanced cell surface expression of the novel ligand in dendritic, macrophage and NK cells. Identical results were observed for binding of hTRAIL-R2:Fc, consistent with results shown in FIG. 4.

FIG. 12: Size-exclusion elution profiles. Profiles of UL141 dimer (a), TRAIL-R2 monomer (b), TRAIL-R1-Fc dimer (c), and TRAIL-R2-Fc dimer (d). The purified proteins elute as 64, 17, 111, and 125 kDa mono-disperse peaks, respectively.

FIG. 13: Sequence alignment of various TNF ligands with UL141 (a) and TNFSFR (b). Residues that are conserved throughout the TNF family are shaded in blue according percentage identity (dark blue for identical residue). Residues that form a particular binding patch in the UL141-TRAIL-R2 structure are boxed using the colors of FIG. 4. (a) TNF ligands: TNFSF1/TNFβ/LTα (1-205), TNFSF2/TNFα (1-233), TNFSF6/FasL/CD96L (1-281), TNFSF10/TRAIL/Apo2L (1-281) and TNFSF11/RANKL/TRANCE/OpgL (1-317). (b) TNF receptors: TNFRSF10A/TRAIL-R1/DR4 (1-468), TNFRSF10B/TRAIL-R2/DR5 (1-440), TNFRSF10C/TRAIL-R3/DcR1 (1-259), TNFRSF10D/TRAIL-R4/DcR2 (1-386), TNFRSF11A/RANK (1-616), TNFRSF11B/OPG/OCIF (1-401), TNFRSF1A/TNFR1 (1-455), TNFRSF1B/TNFR2 (1-461) and TNFRSF6/Fas/APT1 (1-335).

FIG. 14. Purification of UL141-TRAIL-R2 Fc-fusion protein complex. Purification of seleno-methionine (SeMet) labeled UL141-TRAIL-R2 protein complex from Spodoptera Frugiperda (Sf9) insect cells. (a) Affinity chromatography by His-tag capturing Ni-NTA agarose column (Hi-TRAP 1 ml column, GE Healthcare) performed by linear step gradient of Imidazole. (b) Anion exchange chromatography (Mono Q 1 ml column, GE Healthcare) performed by gradient of sodium chloride. (c) High affinity chromatography by human Ig-binding (Fc-capturing) column (Protein A 1 ml column, GE Healthcare) running with Thrombin cleaved sample. (d) Size exclusion chromatography (Superdex S200 10/300 column, GE Healthcare) elution profile of SeMet-UL141-TR2 protein complex. Shaded areas represent SeMet-UL141-TR2-containing fractions.

FIG. 15. SDS-PAGE of the UL141-TRAIL-R2 Fc-fusion protein complex. Gradient 4-20% SDS-PAGE of freshly purified samples of UL141-TR2 Fc-fusion protein complex. (R) stands for reduced and (NR) for non-reduced sample condition. Lanes 3 and 4 are samples treated by one unit (1U) of Thrombin (Thr) per mg of protein.

FIG. 16. HCMV restriction of TRAIL DR cell surface expression requires UL141. A, B) NHDF were infected with the AD169 or FIX strains of HCMV or various deletion mutants at an MOI of ˜2, and cell surface levels of TRAIL-R1 and -R2 were analysed 72 hours later by flow cytometry. Black histograms, mock infected; grey histograms, HCMV infected; dotted (A) or shaded (B) histograms, isotype control.

FIG. 17. TRAIL-R2 expression in HCMV-infected cells. Human fibroblasts (HFF) were infected (72 h, MOI=20) with HCMV Merlin (Mer) or MerlinAUL141 (MerA141) and analyzed for TRAIL-R2 expression by A) flow cytometry and B) western blot. IgG(−), isotype control antibody staining.

FIG. 18. UL141 is sufficient to inhibit cell surface expression of TRAIL DR. A) NHDF cells or B) 293T cells co-transfected with UL141 and a GFP expressing plasmid were analyzed for cell surface expression of the indicated proteins by flow cytometry 48 h later. Black histogram, GFP(−) cells; grey histogram, GFP(+) cells; grey shaded histogram: isotype control. Human fibroblasts (HF-CAR) were infected (48 h, MOI=3) with RAd-CTRL or RAd-UL141 and analyzed for TRAIL-R2 expression by C) flow cytometry and D) western blot.

FIG. 19. UL141 binds directly to TRAIL DR. Sensorgrams of UL141 binding to TRAIL-R1 (left) and -R2 (right). Each curve (top) represents the binding response of UL141 to both DR at a different concentration (0.78-5004, left and 0.016-1 μM, right). The corresponding residual statistics representing the deviation from the fitted data to the actual response values is shown below. The KD of 2.304 and 6 nM were determined for UL141ecto binding to TRAIL-R1:Fc and TRAIL-R2:Fc, respectively, immobilized on the chip.

FIG. 20. UL141 restricts expression of TRAIL DR to the endoplasmic reticulum. Human fibroblasts were co-infected for 48 h with adenovirus vectors expressing TRAILR2-ΔDeathDomain-GFP (TR2-GFP), TR2-RFP, CD155-cherry or MICA-GFP, as indicated. A proportion of cells were also co-infected with adenovirus vector expressing UL141 (panels F-J and P-T). Slides were counter stained with WGA-AF350 to visualize the outline of the cells.

FIG. 21. UL141 inhibits TRAIL-mediated apoptosis. A) Human fibroblasts (HFCAR) were infected with RAd-UL141 or RAd-CTRL (48 h, MOI=3), incubated with TRAIL or TNFα as indicated and analyzed for caspase 3/7 activation (n=4, error bars represent standard deviation). B) NHDF cells were either mock infected or infected with the indicated HCMV viruses at an MOI of ˜2. 48 h later, 50 or 100 ng/ml of purified hTRAIL+5 μg/ml cycloheximide (CHX) was added for an additional 48 hours before assessing cell viability. In all cases, % live cells were calculated by normalizing TRAIL+CHX treated cells to cultures treated with CHX only. In (A), a Student's T test was used for statistical analysis, and the 8 h time point in TNFα-treated cells has a p value of 0.048. In (B), statistical analysis was performed using the one-way ANOVA (both groups are p<0.0001, ***) and displayed are Tukey's multiple comparison post-test results.

FIG. 22. UL141 blockade of both TRAIL DR and CD155 contributes to NK cell inhibition. A) RAd control or UL141 transduced A549 cells were analyzed for expression of TRAIL-R2, CD155 and CD112 at the time of NK cell addition by flow cytometry. B) Western blot of RAd transduced A549 cells. C) Expression of TRAIL by IFNα activated (blue) or unactivated (red) human NK cells assessed by flow cytometry. D) IFNα activated NK cells were purified from human peripheral blood and added to A549 lung epithelial cells transduced with either control adenovirus vector (Rad-Cntrl) or Rad-UL141 (E:T of 2). 10 μg/ml of blocking αDNAM-1 antibody or blocking soluble TRAIL-R2 were added to cultures where indicated (+), and control mIgG or sCD30 were added as controls to the other cultures. Apoptosis of A549 cells was assessed 4 hours later. Shown are two representative experiments of more than 6 performed.

FIG. 23: Structural Coordinates of the TRAIL-R2/UL141 crystal structure

### DETAILED DESCRIPTION

In accordance with the present invention, there is provided a novel ligand binding site of a TRAIL receptor, or a subsequence, portion, homologue, variant or derivative thereof. In different embodiments, the novel binding site may be present on a TRAIL-R1, TRAIL-R2, TRAIL-R3 or TRAIL-R4 receptor. In a particular embodiment of the present invention there is provided a novel ligand binding site of a TRAIL-R2 receptor or a subsequence, portion, homologue, variant or derivative thereof.

The present inventors have solved the crystal structure of a TRAIL-R2 receptor bound to a ligand (UL141) and have discovered a novel ligand binding site of a TRAIL-R2 receptor as well as novel ligand binding patches within the novel ligand binding site.

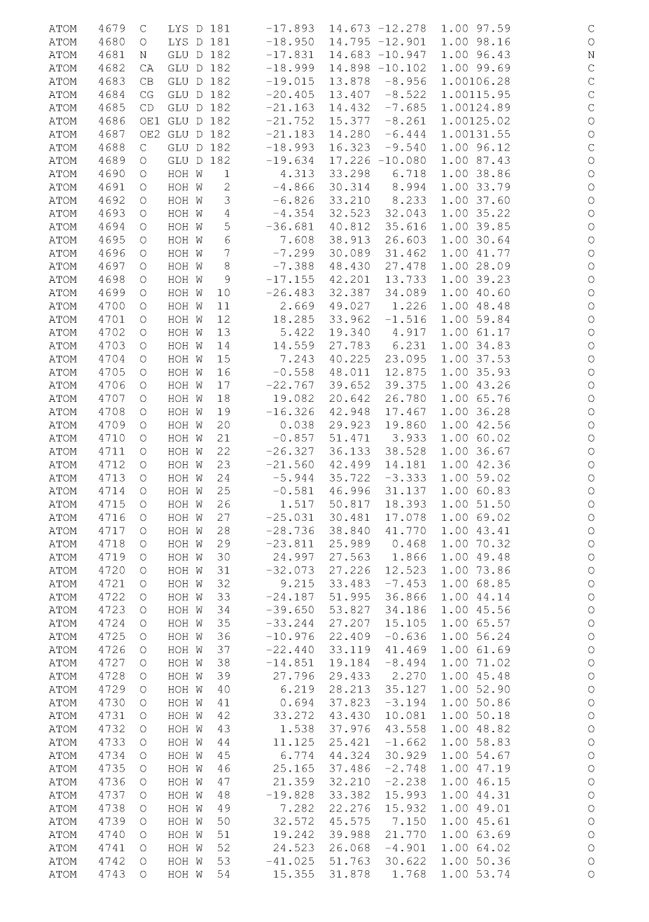

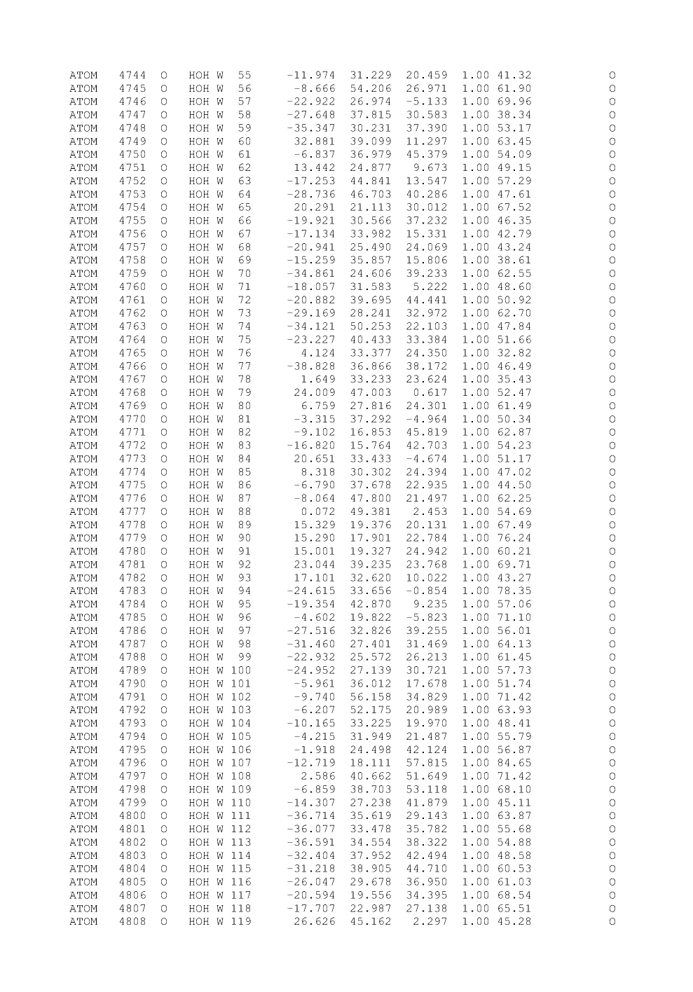

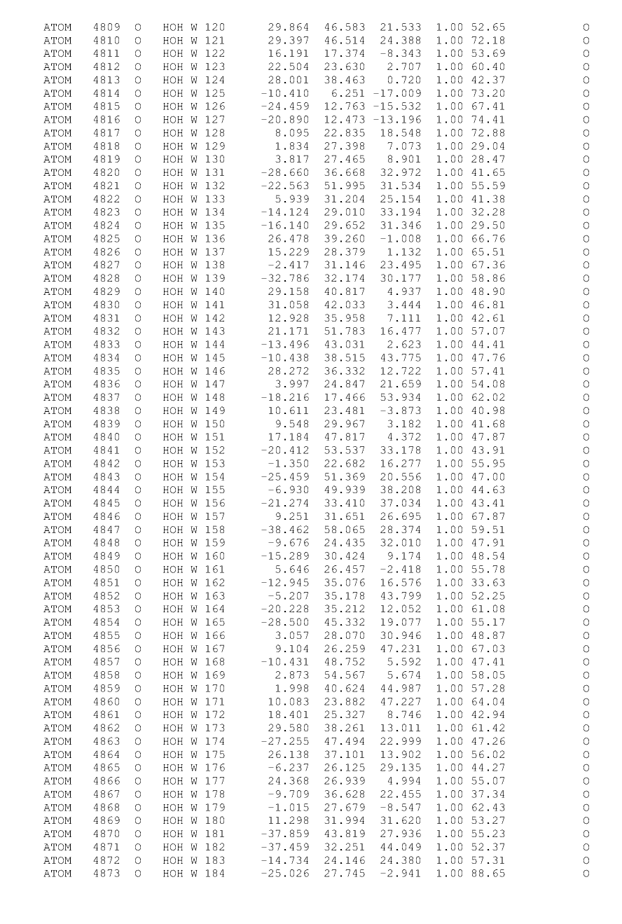

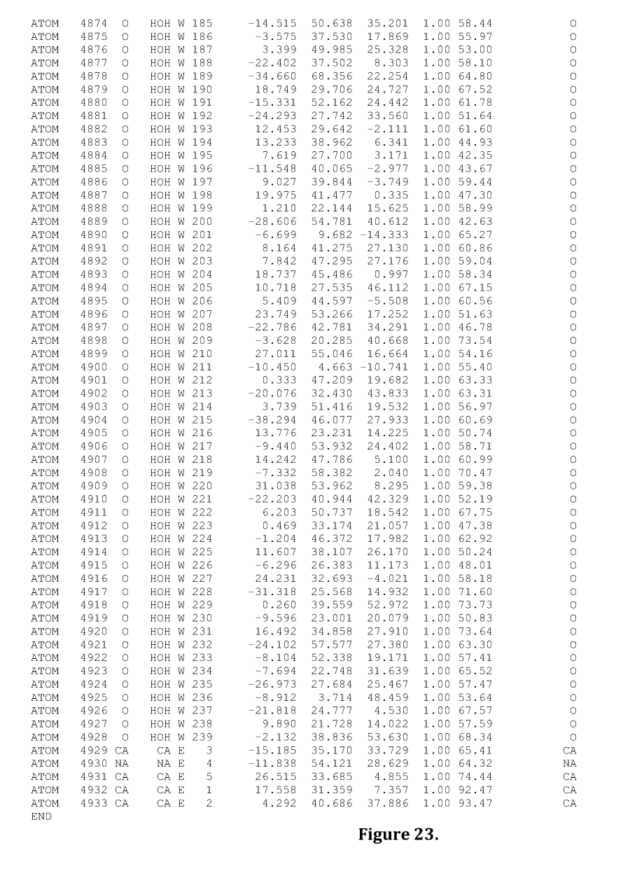

Thus, there is presently provided a novel ligand binding site of a TRAIL-R2 receptor or a subsequence, portion, homologue, variant or derivative thereof, comprising relative structural coordinates set forth in FIG. 23, wherein resolution is 2.1 Angstrom.

In particular embodiments of the present invention, the ligand binding site, or subsequence, portion, homologue, variant or derivative thereof, described herein comprises, consists or consists essentially of amino acid residues 58-184 of TRAIL-R2 receptor or a subsequence, portion, homologue, variant or derivative thereof. In certain embodiments

In particular embodiments, the ligand binding site, or subsequence, portion, homologue, variant or derivative thereof, comprises one or more novel binding patches. In different embodiments, the binding patches may comprise, consist of or consist essentially of one or more of amino acid residues E78 and D109 of TRAIL-R2 receptor; amino acid residue D148 of TRAIL-R2 receptor; amino acid residues V167, V179 and W173 of TRAIL-R2 receptor; amino acid residues Y103, N134 and R133 of TRAIL-R2 receptor; amino acid residues L110, L114 and F112 of TRAIL-R2 receptor and amino acid residues E151 and E147 of TRAIL-R2 receptor, or a subsequence, portion, homologue, variant or derivative thereof.

As used herein, the terms “bind” or “binding” refers to any interaction between two molecules, whether direct or indirect or whether functional or physical. Thus, the term binding may refer to a physical interaction at the molecular level or functional interaction that need not require physical interaction or binding. A ligand or binding agent that binds a TRAIL receptor may partially or completely inhibit, decrease or reduce a physical interaction or a functional interaction between a TRAIL receptor and another ligand or binding agent Inhibition of binding can be due to steric hindrance, occupation, blocking or modification or alteration of the site of physical or functional interaction, or alteration of a modification or another factor that participates in binding between the TRAIL receptor and a ligand or binding agent.

Binding and interaction as used herein includes both cis and trans binding or interaction. As used herein, a “cis” interaction or binding refers to interaction/binding of two entities (e.g., proteins) expressed on the same cell. A “trans” interaction or binding refers to interaction/binding between proteins expressed on distinct cells (i.e., two different cells). Such cis and trans interactions between two entities can involve a direct interaction/binding. Alternatively, such cis and trans interactions between two entities can also be mediated by an intermediary molecule and need not involve direct interaction/binding between the two entities. For example, a “trans” interaction between two cells can occur when a ligand of a TRAIL receptor expressed on a cell binds to a TRAIL receptor expressed on a different cell or when a soluble ligand of a TRAIL receptor binds to a TRAIL receptor on one cell to link the TRAIL receptor to another molecule expressed on a different cell. Thus, a novel ligand of a TRAIL receptor as described herein can function as an intermediary that mediates the interaction/binding of a TRAIL receptor to other molecules on the same cell or on another cell. A novel ligand of a TRAIL receptor as described herein may also function to aggregate cells or molecules on different cells.

As used herein, a “ligand binding site” is a portion of one or more proteins or peptides that binds a ligand. Binding sites can vary in size, for example, from one amino acid up to a polypeptide that is one amino acid less than the entire length of the full-length protein containing the ligand binding site. As will be understood by a person of skill in the art, a ligand binding site may comprise contiguous amino acids or may comprise two or more non-contiguous amino acids of a TRAIL receptor, the amino acids of a ligand binding sited being separated by one or more amino acid residues. In certain embodiments, a ligand binding site may comprise contiguous and non-contiguous amino acids. In particular embodiments, the ligand binding site of the present invention comprises one or more continguous or non-contiguous amino acids of amino acid residues 58 to 212 of a TRAIL-R2 receptor or a subsequence, portion, homologue, variant or derivative thereof. In certain embodiments, the ligand binding site comprises one or more amino acids of amino acid residues 58 to 184 of a TRAIL-R2 receptor or a subsequence, portion, homologue, variant or derivative thereof. In certain embodiments, the ligand binding site comprises one or more amino acids of amino acid residues 185 to 212 of a TRAIL-R2 receptor or a subsequence, portion, homologue, variant or derivative thereof. Thus in different embodiments, the ligand binding site comprises one or more amino acids of amino acid residues 58 to 185, 58 to 186, 58 to 187, 58 to 188, 58 to 189, 58 to 190, 58 to 191, 58 to 192, 58 to 193, 58 to 194, 58 to 195, 58 to 196, 58 to 197, 58 to 198, 58 to 199, 58 to 200, 58 to 201, 58 to 202, 58 to 203, 58 to 204, 58 to 205, 58 to 206, 58 to 207, 58 to 208, 58 to 209, 58 to 210, 58 to 211 or 58 to 212 of TRAIL-R2 receptor or a subsequence, portion, homologue, variant or derivative thereof. In certain embodiments, the ligand binding site comprises one or more amino acids of amino acid residues 58 to 184 and one or more amino acids of amino acid residues 185 to 212 of a TRAIL-R2 receptor or a subsequence, portion, homologue, variant or derivative thereof.

As used herein, a “binding patch” refers to one or more amino acids of a ligand binding site that bind one or more amino acids of a ligand. Thus a binding patch of a TRAIL receptor refers to one or more amino acids within a ligand binding site of the TRAIL receptor that bind one or more amino acids of a ligand. As will be understood by a person of skill in the art, a binding patch may comprise contiguous amino acids or may comprise two or more non-contiguous amino acids of a TRAIL receptor binding site, the amino acids of a binding patch being separated by one or more amino acid residues. In certain embodiments, a ligand binding site may comprise contiguous and non-contiguous amino acids. In particular embodiments, a binding patch comprises one or more amino acids of amino acid residues 58-212 of a TRAIL-R2 receptor or a subsequence, portion, homologue, variant or derivative thereof. In further embodiments, a binding patch comprises one or more amino acids of amino acid residues 58-184 of a TRAIL-R2 receptor or a subsequence, portion, homologue, variant or derivative thereof. In certain embodiments, a binding patch comprises one or more amino acids of amino acid residues 185-212 of a TRAIL-R2 receptor or a subsequence, portion, homologue, variant or derivative thereof.

In particular embodiments, the present invention provides a novel ligand binding patch of a TRAIL-R2 receptor comprising, consisting of or consisting essentially of one or more of amino acid residues E78 and D109 of TRAIL-R2 receptor; amino acid residue D148 of TRAIL-R2 receptor; amino acid residues V167, V179 and W173 of TRAIL-R2 receptor; amino acid residues Y103, N134 and R133 of TRAIL-R2 receptor; amino acid residues L110, L114 and F112 of TRAIL-R2 receptor; and amino acid residues E151 and E147 of TRAIL-R2 receptor, or subsequence, portion, homologue, variant or derivative thereof.

Provided in the present invention are homologues of the novel ligand binding site of a TRAIL receptor described herein. In particular embodiments of the present invention, there is provided homologues of the novel ligand binding site of a TRAIL-R2 receptor described herein. In certain embodiments, there is provided a novel ligand binding site of a TRAIL-R1 receptor, TRAIL-R3 receptor or TRAIL-R4 receptor that is homologous to the novel ligand binding site of a TRAIL-R2 receptor described herein. In particular embodiments, there is presently provided a novel ligand binding site of a TRAIL-R1 receptor, TRAIL-R3 receptor or TRAIL-R4 receptor comprising, consisting of or consisting essentially of an amino acids sequence that is homologous to amino acids residues 58-184 of a TRAIL-R2 receptor, or a subsequence, portion, homologue, variant or derivative thereof. In particular embodiments, there is presently provided a novel ligand binding site of a TRAIL-R1 receptor, TRAIL-R3 receptor or TRAIL-R4 receptor comprising, consisting of or consisting essentially of an amino acids sequence that is homologous to amino acids residues 185-212 of a TRAIL-R2 receptor, or a subsequence, portion, homologue, variant or derivative thereof. In certain embodiments of the present invention, a novel ligand binding site of a TRAIL-R1 receptor, TRAIL-R3 receptor or TRAIL-R4 receptor described herein may comprise, consist of or consist essentially of amino acids that are homologous to one or more of amino acid residues E78 and D109 of TRAIL-R2 receptor; amino acid residue D148 of TRAIL-R2 receptor; amino acid residues V167, V179 and W173 of TRAIL-R2 receptor; amino acid residues Y103, N134 and R133 of TRAIL-R2 receptor; amino acid residues L110, L114 and F112 of TRAIL-R2 receptor; and amino acid residues E151 and E147 of TRAIL-R2 receptor.

HCMV is a β-herpesvirus with a dsDNA genome of ˜230 kB, and like all herpesviruses, establishes a lifelong, persistent/latent infection of its host. To accomplish this, HCMV encodes a multitude of ‘immune modulatory’ proteins that target immune pathways and thwart host defenses⁵⁴. The present inventors had previously identified several viral proteins that target signaling by members of the TNF-TNFR superfamily. A specific example of this is the HCMV UL144 protein⁵⁵. The present inventors characterized UL144 as an orthologue of HVEM (TNFRSF14) that specifically binds to BTLA. BTLA is an inhibitory receptor that encodes an Ig-domain and restricts activation of T cells (and potentially other immune cells), similar to the CD28-related inhibitory receptors CTLA-4 and PD-1. Strikingly, UL144 is a much more potent inhibitor of T cell proliferation than HVEM⁵⁶, likely due to refinement of UL144 function resulting from extensive co-evolution between HCMV and its host. Consequently, UL144 only binds to BTLA, and not to LIGHT (homologous to LT, shows inducible expression, competes with HSV glycoprotein D for HVEM, a receptor expressed by T lymphocytes) or CD160, whereas HVEM binds all 3 of these molecules⁵⁷.

The UL144-BTLA-HVEM signaling network serves as a primary example where a HCMV protein has ‘taught’ about alternate/novel TNFR ligands. Consequently, without being limited to any particular theory, it appears that UL141 is also a structural and/or functional mimic of a novel ligand for TRAIL-R2. Support for this is provided by the several examples of seemingly disparate results that have been observed in mice genetically deficient for either TRAIL or TRAIL-R2, which was also the case for HVEM and LIGHT deficient mice before BTLA was identified to bind HVEM. Further, the present invention provides characterization of the UL141-TRAIL-R2 binding complex by X-ray crystallographic methods which reveals that UL141 uses an Ig-like domain to bind to TRAIL-R2, paralleling how BTLA utilizes an Ig-domain to bind to UL144 and HVEM. In addition, the present inventors have also discovered through structural, biochemical and mutagenesis analysis of the UL141-TRAILR2 complex that UL141 binds to TRAIL-R2 in a fashion distinct from TRAIL, indicating it is not a TRAIL mimic.

Thus novel ligands of a TRAIL receptor, or subsequence, portion, homologue, variant or derivative thereof, of the present invention may bind the novel ligand binding sites of a TRAIL receptor described herein. Thus, in different embodiments, a novel ligand of a TRAIL receptor, or subsequence, portion, homologue, variant or derivative thereof, may bind one or more of a novel ligand binding site of TRAIL-R1, a novel ligand binding site of TRAIL-R2, a novel ligand binding site of TRAIL-R3 and a novel ligand binding site of TRAIL-R4, described herein. The novel ligand may bind one or more TRAIL receptors. In different embodiments, the novel ligand may bind two or more TRAIL receptors substantially contemporaneously or sequentially.

In particular embodiments of the present invention, a novel ligand may bind a protein or peptide comprising, consisting of or consisting essentially of an amino acid sequence of residues 58-184, an amino acid sequence of residues 185-212 or an amino acid sequence of 58 to 212 of a TRAIL-R2 receptor or a subsequence, portion, homologue, variant or derivative thereof. In certain aspects, the novel ligand may bind a novel ligand binding site of a TRAIL-R2 receptor as described herein. In specific embodiments, the novel ligand binds to one or more binding patches of the TRAIL-R2 receptor, the binding patches comprising, consisting of or consisting essentially of amino acid residues E78 and D109 of the TRAIL-R2 receptor; amino acid residue D148 of the TRAIL-R2 receptor; amino acid residues V167, V179 and W173 of the TRAIL-R2 receptor; amino acid residues Y103, N134 and R133 of the TRAIL-R2 receptor; amino acid residues L110, L114 and F112 of the TRAIL-R2 receptor; and amino acid residues E151 and E147 of the TRAIL-R2 receptor.

In some embodiments, the novel ligand of a TRAIL receptor of the present invention comprises a purified natural protein or peptide or a subsequence, portion, homologue, variant or derivative thereof. In certain embodiments, the novel ligand may modulate the activity of the TRAIL receptor, including but not limited to decreasing, reducing, inhibiting, suppressing, disrupting, eliciting, stimulating, inducing, promoting, increasing or enhancing activity of a TRAIL receptor or a ligand thereof. In particular embodiments, the novel ligand decreases, reduces, inhibits, suppresses, disrupts, elicits, stimulates, induces, promotes, increases or enhances an immune response, an anti-inflammatory response, cell proliferation or an apoptotic response mediated by the TRAIL receptor or a ligand thereof.

In certain embodiments of the present invention, the novel ligand of a TRAIL receptor may be homologous in whole or in part to UL141.

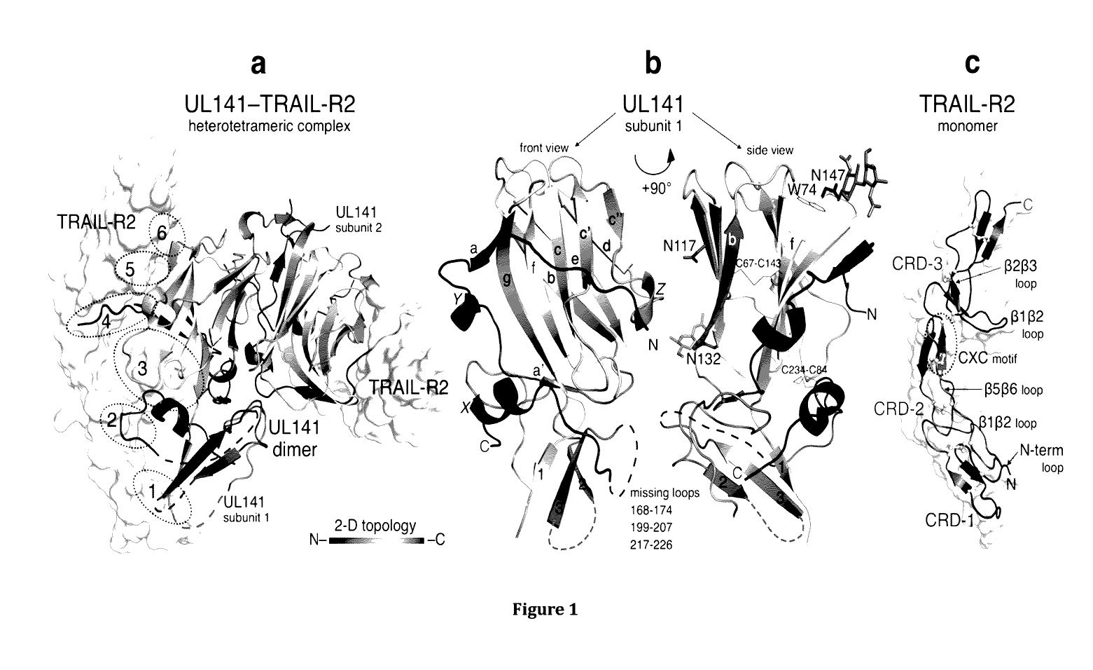

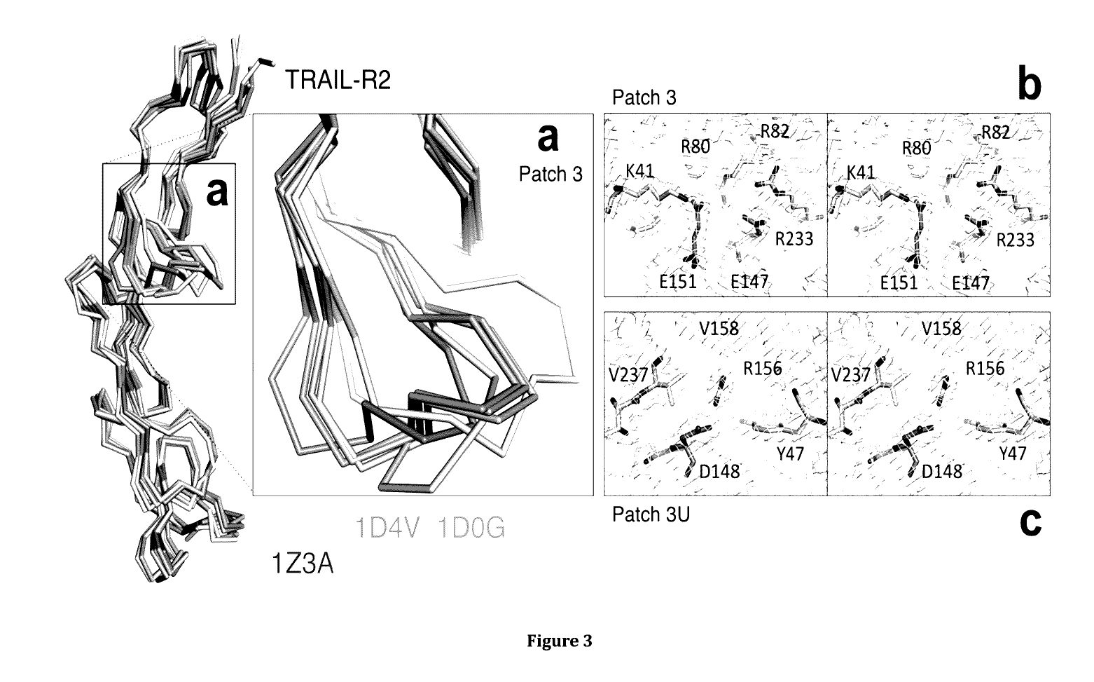

As disclosed herein, presently provided novel ligand binding site of a TRAIL receptor or novel ligand of a TRAIL receptor, or subsequence, portion, homologue, variant or derivative thereof, include those having all or at least partial sequence identity to one or more exemplary novel ligand binding sites of a TRAIL receptor or novel ligand of a TRAIL receptor, or a subsequence, portion, homologue, variant or derivative thereof (e.g., the novel ligand binding site of TRAIL-R2 receptor set forth in FIGS. 1 and 3) The percent identity of such novel ligand binding sites of a TRAIL receptor or novel ligands of a TRAIL receptor, can be as little as 60%, or can be greater (e.g., 65%, 70%, 75%, 80%, 85%, 90%, 95%, 96%, 97%, 98%, 99%, etc.). The percent identity can extend over the entire ligand binding site or entire sequence length of the novel ligand or a portion of the ligand binding site or sequence of the novel ligand. In particular aspects, the portion of the ligand binding site or ligand sharing the percent identity is 2, 3, 4, 5 or more contiguous amino acids, e.g., 6, 7, 8, 9, 10, 11, 12, 13, 14, 15, 16, 17, 18, 19, 20, etc. contiguous amino acids. In additional particular aspects, the length of the ligand binding site or ligand sharing the percent identity is 20 or more contiguous amino acids, e.g., 20, 21, 22, 23, 24, 25, 26, 27, 28, 29, 30, 31, 32, 33, 34, 35, etc. contiguous amino acids. In further particular aspects, the length of the ligand binding site or ligand sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet further particular aspects, the length of the ligand binding site or ligand sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The term “identity” and grammatical variations thereof, mean that two or more referenced entities are the same. Thus, where ligand binding sites or ligands, or subsequences, portions or modifications thereof are identical, they have the same amino acid sequence. The identity can be over a defined area (region or domain) of the sequence. As will be understood by a person of skill in the art, areas, regions, amino acids, domains, ligands or binding sites that are “homologous” or “homologues” mean that a portion of two or more referenced entities share homology.

The extent of identity between two ligand binding sites or ligands can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

In accordance with the invention, modified, derivative and variant forms of the presently described novel ligand binding sites of a TRAIL receptor or novel ligands of a TRAIL receptor, or subsequences or portions thereof are provided. Such forms, referred to as “modifications” or “variants” and grammatical variations thereof, are a novel ligand binding site of a TRAIL receptor or a novel ligand of a TRAIL receptor, or subsequence or portion thereof, that deviates from a reference sequence, such as the sequences for TRAIL receptors provided herein. Such modifications may have greater or less activity or function than a reference novel ligand binding site of a TRAIL receptor or novel ligand of a TRAIL receptor, or subsequence or portion thereof, such as the ability to decrease, reduce, inhibit, suppress, disrupt, elicit, stimulate, induce, promote, increase or enhance TRAIL receptor activity including but not limited to an immune response, an anti-inflammatory response, cell proliferation or an apoptotic response mediated by the TRAIL receptor or a ligand thereof. Thus, novel ligand binding sites of a TRAIL receptor or novel ligands of a TRAIL receptor, or subsequences or portions thereof include a ligand binding site of a TRAIL receptor or a novel ligand of a TRAIL receptor having substantially the same, greater or less relative activity or function as a reference novel ligand binding site of a TRAIL receptor or a novel ligand of a TRAIL receptor, in vitro or in vivo.

Non-limiting examples of modifications include one or more amino acid substitutions (e.g., 1, 2, 3, 4, 5, 6, 7, 8, 9, 10, 1, 12, 13, 14, 15, 16, 17, 18, 19, 20, 20-25, 25-30, 30-50, 50-100, or more residues), additions and insertions (e.g., 1, 2, 3, 4, 5, 6, 7, 8, 9, 10, 1, 12, 13, 14, 15, 16, 17, 18, 19, 20, 20-25, 25-30, 30-50, 50-100, or more residues) and deletions (e.g., 1, 2, 3, 4, 5, 6, 7, 8, 9, 10, 1, 12, 13, 14, 15, 16, 17, 18, 19, 20, 20-25, 25-30, 30-50, 50-100) of a reference novel ligand binding site of a TRAIL receptor or novel ligand of a TRAIL receptor, or subsequence or portion thereof.

Specific non-limiting examples of substitutions include conservative and non-conservative amino acid substitutions. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge, or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

An addition can be the covalent or non-covalent attachment of any type of molecule to the sequence. Specific examples of additions include glycosylation, acetylation, phosphorylation, amidation, formylation, ubiquitination, and derivatization by protecting/blocking groups and any of numerous chemical modifications. Additional specific non-limiting examples of an addition are one or more additional amino acid residues. Accordingly, a novel ligand binding site of a TRAIL receptor or a novel ligand of a TRAIL receptor, or a subsequence, portion, homologue, variant or derivative thereof, as described herein can be a part of or contained within a larger molecule, such as another protein or peptide sequence, such as a fusion or chimera with a different novel ligand binding site of a TRAIL receptor or novel ligand of a TRAIL receptor, or a subsequence, portion, homologue, variant or derivative thereof, or a non-novel ligand binding site of a TRAIL receptor or a novel ligand of a TRAIL receptor, or a subsequence, portion, homologue, variant or derivative thereof. In particular embodiments, an addition is a fusion (chimeric) sequence, an amino acid sequence having one or more molecules not normally present in a reference native (wild type) sequence covalently attached to the sequence.

The term “chimeric” and grammatical variations thereof, when used in reference to a sequence, means that the sequence contains one or more portions that are derived from, obtained or isolated from, or based upon other physical or chemical entities. For example, a chimera of two or more different proteins may have one part a novel ligand binding site of a TRAIL receptor or a novel ligand of a TRAIL receptor, or a subsequence, portion, homologue, variant or derivative thereof, and a second part of the chimera may be from a different novel ligand binding site of a TRAIL receptor or novel ligand of a TRAIL receptor, or a may not be from a novel ligand binding site of a TRAIL receptor or a novel ligand of a TRAIL receptor.

Another particular example of a modified sequence having an amino acid addition is one in which a second heterologous sequence, i.e., heterologous functional domain is attached (covalent or non-covalent binding) that confers a distinct or complementary function. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), and radioisotope. Thus, in other embodiments, there is presently provided a novel ligand binding site of a TRAIL receptor or a novel ligand of a TRAIL receptor, or a subsequence, portion, homologue, variant or derivative thereof, wherein the heterologous functional domain confers a distinct function, on the novel ligand binding site of a TRAIL receptor or novel ligand of a TRAIL receptor, or a subsequence, portion, homologue, variant or derivative thereof. Such constructs containing a novel ligand binding site of a TRAIL receptor or a novel ligand of a TRAIL receptor, or a subsequence, portion, homologue, variant or derivative thereof, and a heterologous domain are also referred to as chimeras.

Linkers, such as amino acid or peptidomimetic sequences may be inserted between the sequence and the addition (e.g., heterologous functional domain) so that the two entities maintain, at least in part, a distinct function or activity. Linkers may have one or more properties that include a flexible conformation, an inability to form an ordered secondary structure or a hydrophobic or charged character, which could promote or interact with either domain. Amino acids typically found in flexible protein regions include Gly, Asn and Ser. Other near neutral amino acids, such as Thr and Ala, may also be used in the linker sequence. The length of the linker sequence may vary without significantly affecting a function or activity of the fusion protein (see, e.g., U.S. Pat. No. 6,087,329). Linkers further include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further non-limiting examples of additions are detectable labels. Thus, in another embodiment, the invention provides a novel ligand binding site of a TRAIL receptor or a novel ligand of a TRAIL receptor, or a subsequence, portion, homologue, variant or derivative thereof, that is detectably labeled. Specific examples of detectable labels include fluorophores, chromophores, radioactive isotopes (e.g., S³⁵, P³², I¹²⁵), electron-dense reagents, enzymes, ligands and receptors. Enzymes are typically detected by their activity. For example, horseradish peroxidase is usually detected by its ability to convert a substrate such as 3,3-′,5,5-′-tetramethylbenzidine (TMB) to a blue pigment, which can be quantified.

Another non-limiting example of an addition is an insertion of an amino acid within any novel ligand binding site of a TRAIL receptor or a novel ligand of a TRAIL receptor, or a subsequence, portion, homologue, variant or derivative thereof. In particular embodiments, an insertion is of one or more amino acid residues inserted into the amino acid sequence of a novel ligand binding site of a TRAIL receptor or a novel ligand of a TRAIL receptor, or a subsequence, portion, homologue, variant or derivative thereof,

Modified and variant novel ligand binding sites of a TRAIL receptor or novel ligands of a TRAIL receptor, or a subsequence, portion, homologue, variant or derivative thereof, also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond. Novel ligand binding sites of a TRAIL receptor or novel ligands of a TRAIL receptor, or a subsequence, portion, homologue, variant or derivative thereof, may be modified in vitro or in vivo, e.g., post-translationally modified to include, for example, sugar residues, phosphate groups, ubiquitin, fatty acids, lipids, etc.

Specific non-limiting examples of a novel ligand binding site of a TRAIL receptor or a novel ligand of a TRAIL receptor, or a subsequence, portion, homologue, variant or derivative thereof, include an amino acid sequence comprising at least one amino acid deletion from a full length amino acid sequence of a novel ligand binding site of a TRAIL receptor or a novel ligand of a TRAIL receptor, or a homologue, variant or derivative thereof. In particular embodiments, a protein subsequence or portion is from about 2 to 127 amino acids in length, provided that said subsequence or portion is at least one amino acid less in length than the full-length sequence of the novel ligand binding site of a TRAIL receptor or novel ligand of a TRAIL receptor, or a homologue, variant or derivative thereof. In additional particular embodiments, a protein subsequence or portion is from about 2 to 5, 5 to 10, 10 to 15, 15 to 20, 20 to 25, 25 to 50, 50 to 100, 100 to 125 or 125 to 127 amino acids in length, provided that said subsequence or portion is at least one amino acid less in length than the full-length sequence of a novel ligand binding site of a TRAIL receptor or novel ligand of a TRAIL receptor, or a homologue, variant or derivative thereof.

Novel ligand binding sites of a TRAIL receptor or novel ligands of a TRAIL receptor, or a subsequence, portion, homologue, variant or derivative thereof, can be produced by any of a variety of standard protein purification or recombinant expression techniques. For example, a novel ligand binding site of a TRAIL receptor or a novel ligand of a TRAIL receptor, or a subsequence, portion, homologue, variant or derivative thereof, can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

A novel ligand binding site of a TRAIL receptor or a novel ligand of a TRAIL receptor, or a subsequence, portion, homologue, variant or derivative thereof, can be made using recombinant DNA technology via cell expression or in vitro translation. Polypeptide sequences including modified forms can also be produced by chemical synthesis using methods known in the art, for example, an automated peptide synthesis apparatus (see, e.g., Applied Biosystems, Foster City, Calif.).

The invention provides isolated and/or purified novel ligand binding sites of a TRAIL receptor or novel ligands of a TRAIL receptor, or a subsequence, portion, homologue, variant or derivative thereof, including, comprising or consisting of amino acid sequence of a TRAIL-R1 receptor, a TRAIL-R2 receptor, a TRAIL-R3 receptor, a TRAIL-R4 receptor, a ligand of a TRAIL-R1 receptor, a ligand of a TRAIL-R2 receptor, a ligand of a TRAIL-R3 receptor or a ligand of a TRAIL-R4 receptor or a subsequence, portion, homologue, variant or derivative thereof.

In particular embodiments, the present invention provides an isolated and/or purified novel ligand binding site of a TRAIL-R2 receptor, or a subsequence, portion, homologue, variant or derivative thereof. In other embodiments, the present invention provides an isolated and/or purified novel ligand of a TRAIL-R2 receptor.

In certain embodiments, the present invention provides an isolated and/or purified novel ligand binding site of a TRAIL-R2 receptor, or a subsequence, portion, homologue, variant or derivative thereof, that includes, comprises, consists of or consists essentially of amino acid residues 58-184 of TRAIL-R2 receptor or a subsequence, portion, homologue, variant or derivative thereof. In certain embodiments, the present invention provides an isolated and/or purified novel ligand binding site of a TRAIL-R2 receptor, or a subsequence, portion, homologue, variant or derivative thereof, that includes, comprises, consists of or consists essentially of amino acid residues 185-212 of TRAIL-R2 receptor or a subsequence, portion, homologue, variant or derivative thereof. In particular embodiments, the present invention provides an isolated and/or purified novel ligand binding site of a TRAIL-R2 receptor, or a subsequence, portion, homologue, variant or derivative thereof, that includes, comprises, consists of or consists essentially of amino acid residues 58-212 of TRAIL-R2 receptor or a subsequence, portion, homologue, variant or derivative thereof. In other embodiments, the present invention provides an isolated and/or purified novel ligand binding site of a TRAIL-R2 receptor, or a subsequence, portion, homologue, variant or derivative thereof, that includes, comprises, consist of or consists essentially of structural coordinates set forth in FIG. 23, or a subsequence, portion, homologue, variant or derivative thereof. In some embodiments, the present invention provides an isolated and/or purified novel ligand binding site of a TRAIL-R2 receptor, or a subsequence, portion, homologue, variant or derivative thereof, that includes, comprises, consist of or consists essentially of amino acid residues E78 and D109 of the TRAIL-R2 receptor; amino acid residue D148 of the TRAIL-R2 receptor; amino acid residues V167, V179 and W173 of the TRAIL-R2 receptor; amino acid residues Y103, N134 and R133 of the TRAIL-R2 receptor; amino acid residues L110, L114 and F112 of the TRAIL-R2 receptor; and/or amino acid residues E151 and E147 of the TRAIL-R2 receptor, or a subsequence, portion, homologue, variant or derivative thereof.

The term “isolated,” when used as a modifier of a composition (e.g., novel ligand binding sites of a TRAIL receptor or novel ligands of a TRAIL receptor, or a subsequence, portion, homologue, variant or derivative thereof, nucleic acids encoding same, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., novel ligand binding sites of a TRAIL receptor or novel ligands of a TRAIL receptor, or a subsequence, portion, homologue, variant or derivative thereof) can also be “substantially pure” or “purified” when free of most or all of the materials with which it typically associates with in nature. Thus, an isolated novel ligand binding site of a TRAIL receptor or a novel ligand of a TRAIL receptor, or a subsequence, portion, homologue, variant or derivative thereof, that also is substantially pure or purified does not include polypeptides or polynucleotides present among millions of other sequences, such as peptides of an peptide library or nucleic acids in a genomic or cDNA library, for example.

A “substantially pure” or “purified” composition can be combined with one or more other molecules. Thus, “substantially pure” or “purified” does not exclude combinations of compositions, such as combinations of novel ligand binding sites of a TRAIL receptor or novel ligands of a TRAIL receptor, or a subsequence, portion, homologue, variant or derivative thereof, and other proteins, peptides, agents, drugs or therapies.

In accordance with the present invention, there are also provided methods of identifying a binding agent that will interact with the novel ligand binding sites of a TRAIL receptor described herein and binding agents that interact with the novel ligand binding site of a TRAIL receptor described herein.

As used herein, a “binding agent” includes agents that decrease, reduce, inhibit, suppress or disrupt binding of a TRAIL receptor to another ligand or binding agent. Agents also include agents that increase, enhance, stimulate, or promote binding of a TRAIL receptor to another ligand or binding agent. Furthermore, agents include antagonists and agonists of TRAIL receptor function or activity, i.e., agents that decrease, reduce, inhibit, suppress or disrupt a function or activity of a TRAIL receptor; or increase, enhance, stimulate, or promote a function or activity of a TRAIL receptor. In certain embodiments agents include antagonists and agonists of a TRAIL receptor ligand function or activity, i.e., agents that decrease, reduce, inhibit, suppress or disrupt a function or activity of a TRAIL receptor ligand; or increase, enhance, stimulate, or promote a function or activity of a TRAIL receptor ligand.

Non-limiting particular examples of agents include amino acid sequences, such as antibodies, proteins, peptides, and polypeptides, including fusion polypeptides and chimeric polypeptides. Non-limiting examples of agents also include nucleic acid sequences or polynucleotides/polynucleotides, including inhibitory nucleic acids. Further non-limiting examples of agents include ligand mimetics and small molecules. In certain embodiments, binding agents may bind to TRAIL receptor or a ligand of TRAIL receptor thereby modulating (altering or affecting) binding between the TRAIL receptor and a ligand of the TRAIL receptor, and in turn modulating TRAIL receptor activity.

In one aspect, the present invention provides methods of identifying a binding agent that will interact with a novel TRAIL receptor ligand binding site described herein. In certain embodiments, the present methods comprise providing a test agent; contacting the test agent with a protein or peptide comprising, consisting of or consisting essentially of amino acid residues 58-184 of the TRAIL-R2 receptor, or subsequence, portion, homologue, variant or derivative thereof; and detecting interaction of the test agent with the protein or peptide comprising, consisting of or consisting essentially of amino acid residues 58-184 of TRAIL-R2 receptor or subsequence, portion, homologue, variant or derivative thereof. In certain embodiments, the method comprises contacting the test agent with a protein or peptide comprising, consisting of or consisting essentially of amino acid residues 58-212 of the TRAIL-R2 receptor, or subsequence, portion, homologue, variant or derivative thereof; and detecting interaction of the test agent with the protein or peptide comprising, consisting of or consisting essentially of amino acid residues 58-212 of TRAIL-R2 receptor or subsequence, portion, homologue, variant or derivative thereof. In other embodiments, the method comprises contacting the test agent with a protein or peptide comprising, consisting of or consisting essentially of amino acid residues 185-212 of the TRAIL-R2 receptor, or subsequence, portion, homologue, variant or derivative thereof; and detecting interaction of the test agent with the protein or peptide comprising, consisting of or consisting essentially of amino acid residues 185-212 of TRAIL-R2 receptor or subsequence, portion, homologue, variant or derivative thereof. In specific embodiments, the methods comprise identifying a binding agent that interacts with one or more binding patches of the TRAIL-R2 receptor, the method comprising providing a test agent; contacting the test agent with a protein or peptide comprising, consisting of or consisting essentially of one or more the binding patches of the TRAIL-R2 receptor: detecting interaction of the test agent with the protein or peptide comprising, consisting of or consisting essentially of one or more of the binding patches of the TRAIL-R2 receptor, or a subsequence, portion, homologue, variant or derivative thereof. In certain embodiments of the methods described herein, the binding patches comprise, consist of or consist essentially of amino acid residues E78 and D109 of the TRAIL-R2 receptor; amino acid residue D148 of the TRAIL-R2 receptor; amino acid residues V167, V179 and W173 of the TRAIL-R2 receptor; amino acid residues Y103, N134 and R133 of the TRAIL-R2 receptor; amino acid residues L110, L114 and F112 of the TRAIL-R2 receptor; and/or amino acid residues E151 and E147 of the TRAIL-R2 receptor; or a subsequence, portion, homologue, variant or derivative thereof. In different embodiments of the present methods, the protein or peptide comprising, consisting of or consisting essentially of one or more of the binding patches of the TRAIL-R2 receptor, or a subsequence, portion, homologue, variant or derivative thereof, is a protein or peptide comprising ligand bindings sites or amino acid sequences of a TRAIL-R1 receptor, TRAIL-R3 receptor or TRAIL-R4 receptor that are homologous to ligand bindings sites or amino acid sequences of a TRAIL-R2 receptor, or a subsequence, portion, variant or derivative thereof.

The present invention provides cell-free (e.g., in solution, in solid phase) and cell-based (e.g., in vitro or in vivo) methods of identifying a binding agent that will interact with a novel ligand binding site of a TRAIL receptor and methods of identifying a binding agent that will modulate TRAIL receptor activity. The methods can be performed in solution, in solid phase, in silica, in silico, in vitro, in a cell, and in vivo.

As used herein, the term “modulate,” means an alteration or effect on the term modified. For example, the term modulate can be used in various contexts to refer to an alteration or effect of an activity, a function, or expression of a polypeptide, gene or signaling pathway, or a physiological condition or response of an organism. In certain embodiments of the present invention, modulating involves decreasing, reducing, inhibiting, suppressing or disrupting binding of a TRAIL receptor to another ligand or binding agent or function or activity of a TRAIL receptor or ligand thereof. In other embodiments of the present invention, modulating involves increasing, enhancing, stimulating, or promoting binding of a TRAIL receptor to another ligand or binding agent or function or activity of a TRAIL receptor or ligand thereof. Thus, where the term “modulate” is used to modify the term “binding of a TRAIL receptor to a ligand” this means that binding of the TRAIL receptor to a ligand is altered or affected (e.g., decreased, reduced, inhibited, suppressed, limited, controlled, prevented, stimulated, increased or enhanced etc.).

Also provided by the present invention are methods comprising the binding agents or novel ligands of a TRAIL receptor, or a subsequence, portion, homologue, variant or derivative thereof, described herein, or an agonist or antagonist thereof. In one aspect, there is presently provided a method for modulating the activity of a TRAIL receptor, the method comprising contacting the TRAIL receptor with a binding agent or novel ligand of a TRAIL receptor, or a subsequence, portion, homologue, variant or derivative thereof, described herein, or an agonist or antagonist thereof. Methods and uses of the present invention can be performed in vivo, such as in a subject, in vitro, ex vivo, in a cell, in solution, in solid phase, in silico or in silica. In different embodiments of the present methods, the TRAIL receptor is expressed on a cell present in vivo or in vitro.

In different embodiments of the present methods, the activity of a TRAIL receptor may be modulated to decrease, reduce, inhibit, suppress, disrupt, elicit, stimulate, induce, promote, increase or enhance TRAIL receptor activity. In particular embodiments, the TRAIL receptor activity modulated is an immune response, an anti-inflammatory response, cell proliferation or an apoptotic response mediated by the TRAIL receptor or a ligand thereof.

The present invention also provides methods of treatment comprising administration to a subject of a binding agent or novel ligands of a TRAIL receptor, or a subsequence, portion, homologue, variant or derivative thereof, described herein, or an agonist or antagonist thereof.

In certain embodiments, the present methods comprise for treating an undesirable or aberrant immune response, immune disorder, inflammatory response, inflammation or an autoimmune response, disorder or disease in a subject.

In various aspects of the presently provided methods, a subject has or has had an adverse symptom of an undesirable or aberrant immune response, immune disorder, inflammatory response, inflammation or an autoimmune response, disorder or disease.

In additional various aspects of methods and uses of the invention, a subject is in need of treatment for an undesirable or aberrant immune response, immune disorder, inflammatory response, inflammation or an autoimmune response, disorder or disease.

In further various aspects of methods and uses of the invention, a subject is at risk of an undesirable or aberrant immune response, immune disorder, inflammatory response, inflammation or an autoimmune response, disorder or disease.

As used herein, an “undesirable immune response” or “aberrant immune response” refers to any immune response, activity or function that is greater or less than desired or physiologically normal, acute or chronic. An undesirable immune response, function or activity can be a normal response, function or activity. However, such responses are generally characterized as an undesirable or aberrant increased or inappropriate response, activity or function of the immune system. Thus, normal immune responses so long as they are undesirable, even if not considered aberrant, are included within the meaning of these terms. An undesirable immune response, function or activity can also be an abnormal response, function or activity. An abnormal (aberrant) immune response, function or activity deviates from normal.

One non-limiting example of an undesirable or aberrant immune response is where the immune response is hyper-responsive, such as in the case of an autoimmune disorder or disease. Another example of an undesirable or aberrant immune response is where an immune response leads to acute or chronic inflammatory response or inflammation in any tissue or organ.

Undesirable or aberrant immune responses, inflammatory responses, or inflammation are characterized by many different physiological adverse symptoms or complications, which can be humoral, cell-mediated or a combination thereof. Responses, disorders and diseases that can be treated in accordance with the invention include, but are not limited to, those that either directly or indirectly lead to or cause cell or tissue/organ damage in a subject. At the whole body, regional or local level, an immune response, inflammatory response, or inflammation can be characterized by swelling, pain, headache, fever, nausea, skeletal joint stiffness or lack of mobility, rash, redness or other discoloration. At the cellular level, an immune response, inflammatory response, or inflammation can be characterized by one or more of T cell activation and/or differentiation, cell infiltration of the region, production of antibodies, production of cytokines, lymphokines, chemokines, interferons and interleukins, cell growth and maturation factors (e.g., proliferation and differentiation factors), cell proliferation, accumulation or migration and cell, tissue or organ damage. Thus, methods and uses of the invention include treatment of and an ameliorative effect upon any such physiological symptoms or cellular or biological responses characteristic of immune responses, inflammatory response, or inflammation.

Autoimmune responses, disorders and diseases are generally characterized as an undesirable or aberrant response, activity or function of the immune system characterized by increased or undesirable humoral or cell-mediated immune responsiveness or memory, or decreased or insufficient tolerance to self-antigens. Autoimmune responses, disorders and diseases that may be treated in accordance with the invention include but are not limited to responses, disorders and diseases that cause cell or tissue/organ damage in the subject. The terms “immune disorder” and “immune disease” mean, an immune function or activity which is characterized by different physiological symptoms or abnormalities, depending upon the disorder or disease.

In particular embodiments, a method or use decreases, reduces, inhibits, suppresses, limits or controls an undesirable or aberrant immune response, disorder or disease, inflammatory response, disorder or disease, or inflammation, in a subject. In additional particular embodiments, a method or use decreases, reduces, inhibits, suppresses, limits or controls an autoimmune response, disorder or disease in a subject.

In further particular embodiments, a method or use decreases, reduces, inhibits, suppresses, limits or controls an adverse symptom of the undesirable or aberrant immune response, disorder or disease, inflammatory response, disorder or disease, inflammation, or an autoimmune response, disorder or disease.

In additional particular embodiments, methods and uses according to the invention can result in a reduction in occurrence, frequency, severity, progression, or duration of a symptom of the condition (e.g., undesirable or aberrant immune response, disorder or disease, inflammatory response, disorder or disease, inflammation, or an autoimmune response, disorder or disease). For example, methods of the invention can protect against or decrease, reduce, inhibit, suppress, limit or control progression, severity, frequency, duration or probability of an adverse symptom of the undesirable or aberrant undesirable or aberrant immune response, disorder or disease, inflammatory response, disorder or disease, inflammation, or an autoimmune response, disorder or disease.

Examples of adverse symptoms of an undesirable or aberrant immune response, disorder or disease, inflammatory response, disorder or disease, inflammation, or an autoimmune response, disorder or disease include swelling, pain, rash, discoloration, headache, fever, nausea, diarrhea, bloat, lethargy, skeletal joint stiffness, reduced muscle or limb mobility or of the subject, paralysis, a sensory impairment, such as vision or tissue or cell damage. Examples of adverse symptoms occur in particular tissues, or organs, or regions or areas of the body, such as in skin, epidermal or mucosal tissue, gut, gastrointestinal, bowel, genito-urinary tract, pancreas, thymus, lung, liver, kidney, muscle, central or peripheral nerves, spleen, skin, a skeletal joint (e.g., knee, ankle, hip, shoulder, wrist, finger, toe, or elbow), blood or lymphatic vessel, or a cardio-pulmonary tissue or organ. Additional examples of adverse symptoms of an autoimmune response, disorder or disease include cell production, survival, proliferation, activation or differentiation, and/or production of auto-antibodies, or pro-inflammatory cytokines or chemokines (e.g., TNF-alpha, IL-6, etc.).

Specific non-limiting examples of aberrant or undesirable immune responses, disorders and diseases, inflammatory responses, disorders and diseases, inflammation, autoimmune responses, disorders and diseases, treatable in accordance with the invention include: comprises rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus (IDDM, type I diabetes), insulin-resistant diabetes mellitus (type II diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (yc) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), or hyper IgE syndrome.

In certain embodiments, the methods of treatment provided herein comprise treating a microbial infection in a subject, including but not limited to a bacterial infection or a viral infection, or an adverse symptom thereof. In other embodiments, there are provided methods of treating a tumor or cancer in a subject, or an adverse symptom thereof. In still further embodiments, there are provided methods of treating a vascular disease in a subject, including but not limited to pulmonary arterial hypertension, or an adverse symptom thereof.

In different embodiments, the methods of treatment presently provided comprise decreasing, reducing, inhibiting, suppressing, disrupting, eliciting, stimulating, inducing, promoting, increasing or enhancing TRAIL receptor activity. In particular embodiments, the TRAIL receptor activity modulated is an immune response, an anti-inflammatory response, cell proliferation or an apoptotic response mediated by the TRAIL receptor or a ligand thereof.

“Treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently.

Binding agents and novel TRAIL receptor ligands or a subsequence, portion, homologue, variant or derivative thereof of the present invention, can be administered in a sufficient or effective amount to a subject in need thereof. An “effective amount” or “sufficient amount” refers to an amount that provides, or is predicted to provide, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Invention agents and ligands, or a subsequence, portion, homologue, variant or derivative thereof, and agents and ligands, or subsequence, portion, homologue, variant or derivative thereof, of the methods described herein can be in various physical forms therein, such as a liquid or solid form. Invention agents, and agents of the methods described herein, can include any amount or dose of the agent, and the agent. In particular embodiments, an agent is in a concentration range of about 10 μg/ml to 100 mg/ml, or in a range of about 100 μg/ml to 1,000 mg/ml, or at a concentration of about 1 mg/ml. In further particular embodiments, an agent is in an amount of 10-1,000 milligrams, or an amount of 10-100 milligrams.

The doses of an “effective amount” or “sufficient amount” for treatment (e.g., to ameliorate or to provide a therapeutic benefit or improvement) typically are effective to ameliorate a response, disorder or disease, or one, multiple or all adverse symptoms, consequences or complications of the response, disorder or disease, one or more adverse symptoms, disorders, illnesses, pathologies, diseases, or complications, for example, caused by or associated with an aberrant immune response to a measurable extent, although decreasing, reducing, inhibiting, suppressing, limiting or controlling progression or worsening of the aberrant immune response is a satisfactory outcome.

An effective amount or a sufficient amount can but need not be provided in a single dose or administration, may require multiple doses or administrations, and, can but need not be, administered alone or in combination with another composition (e.g., agent), treatment, protocol or therapeutic regimen. For example, the amount may be proportionally increased as indicated by the need of the subject, type, status and severity of the response, disorder, or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use or frequency or amount of another treatment, therapeutic regimen or protocol.

In certain embodiments of the methods of the present invention, binding agents or novel TRAIL receptor ligands, or subsequences, portions, homologues, variants or derivatives thereof, described herein are administered in combination with another TRAIL receptor binding agent. In particular embodiments of the present methods, binding agents or novel TRAIL receptor ligands, or subsequences, portions, homologues, variants or derivatives thereof, described herein are administered in combination with an agonist of the TRAIL receptor ligand. In certain embodiments, the other TRAIL receptor binding agent binds a different binding site on the TRAIL receptor ligand then the binding agents or novel TRAIL receptor ligands, or subsequences, portions, homologues, variants or derivatives thereof, presently described. In particular embodiments of the present methods, binding agents or novel TRAIL receptor ligands, or subsequences, portions, homologues, variants or derivatives thereof, described herein are administered in combination with an agonist of the TRAIL receptor ligand. In one embodiment, binding agents or novel TRAIL receptor ligands, or subsequences, portions, homologues, variants or derivatives thereof, described herein are administered in combination with an TRAIL receptor agonist that binds the same binding site of the TRAIL receptor as TRAIL. In still a further embodiment, the present methods comprise a bispecific molecule, including but not limited to a bispecific antibody, that binds both the novel ligand binding site of a TRAIL receptor described herein and an alternative ligand binding site of the same TRAIL receptor. In particular embodiments, the bispecific molecule inhibits binding of a ligand to the novel ligand binding site of a TRAIL receptor described herein and agonizes TRAIL receptor activity by binding an alternative ligand binding site of the TRAIL receptor.

An effective amount or a sufficient amount need not be effective in each and every subject treated, prophylactically or therapeutically, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

As used herein the term “subject” refers to animals, typically mammalian animals, such as humans, non human primates (e.g., apes, gibbons, chimpanzees, orangutans, macaques), domestic animals (e.g., dogs and cats), farm animals (e.g., horses, cows, goats, sheep, pigs) and experimental animals (e.g., mouse, rat, rabbit, guinea pig). Subjects include animal disease models, for example, animal models of an aberrant immune response, disorder or disease for in vivo analysis of an agent of the invention.

Binding agents and novel TRAIL receptor ligands, or subsequences, portions, homologues, variants or derivatives thereof, described herein can be administered to a subject and methods may be practiced prior to, substantially contemporaneously with, or within about 1-60 minutes, hours (e.g., within 1, 2, 3, 4, 5, 6, 8, 12, 24 hours), or days of a symptom or onset of an aberrant immune response, disorder or disease.

A binding agent or novel TRAIL receptor ligand, or subsequences, portions, homologues, variants or derivatives thereof, of the present invention can be administered and methods presently provided can be practiced via systemic, regional or local delivery or administration, by any route. For example, a binding agent, novel TRAIL receptor ligand or composition thereof may be administered systemically, regionally or locally, via injection, infusion, orally (e.g., ingestion or inhalation), topically, intravenously, intraarterially, intramuscularly, intraperitoneally, intradermally, subcutaneously, intracavity, intracranially, transdermally (topical), parenterally, e.g. transmucosally or intrarectally (enema) catheter, optically.

In certain aspects of the present invention there is presently provided pharmaceutical compositions comprising a binding agent or novel TRAIL receptor ligand or subsequences, portions, homologues, variants or derivatives thereof, described herein, or an agonist or antagonist thereof, and a pharmaceutically acceptable carrier. Pharmaceutically acceptable carriers such as saline will be known to a person of skill in the art. Binding agents, novel TRAIL receptor ligands, or subsequences, portions, homologues, variants or derivatives thereof, and methods of the present invention may comprise pharmaceutical formulations that can be administered via a (micro)encapsulated delivery system or packaged into an implant for administration.

As used herein, the term “pharmaceutically acceptable” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation, and can be contained in a tablet (coated or uncoated), capsule (hard or soft), microbead, emulsion, powder, granule, crystal, suspension, syrup or elixir.

The present invention also provides the a novel composition comprising TRAIL receptor bound to a ligand in crystalline form and a novel crystallized complex of a TRAIL receptor and a ligand thereof. In certain embodiments, the TRAIL receptor is TRAIl-R2. In certain embodiments, the novel composition or crystallized complex comprises the relative structural coordinates set forth in FIG. 23. In particular embodiments, the novel composition comprising TRAIL-R2 receptor bound to a ligand in crystalline form or novel crystallized complex of a TRAIL-R2 receptor and a ligand thereof comprises a structure set forth in FIG. 1 or FIG. 3. In particular embodiments, the ligand is bound to an amino acid sequence of the TRAIL-R2 receptor that comprises, consists of or consists essentially of amino acid residues 58-184 of TRAIL-R2 receptor or a subsequence, portion, homologue, variant or derivative thereof. In different embodiments the amino acid sequence of the TRAIL-R2 receptor that binds the ligand comprises, consists of or consists essentially of one or more of amino acid residues E78 and D109 of the TRAIL-R2 receptor; amino acid residue D148 of the TRAIL-R2 receptor; amino acid residues V167, V179 and W173 of the TRAIL-R2 receptor; amino acid residues Y103, N134 and R133 of the TRAIL-R2 receptor; amino acid residues L110, L114 and F112 of the TRAIL-R2 receptor; and amino acid residues E151 and E147 of the TRAIL-R2 receptor.

In certain embodiments, the novel composition comprising TRAIL-R2 receptor bound to a ligand in crystalline form or novel crystallized complex of a TRAIL-R2 receptor and a ligand thereof has unit cell parameters of a=67.74 Å, b=97.01 Å and c=140.94 Å or a=67.71 Å, b=97.67 Å, c=141.31 Å.

In another aspect of the present invention, there is provided a crystallized complex of a novel ligand binding site of a TRAIL receptor described herein. In particular embodiments, the crystallized complex of a novel ligand binding site of a TRAIL receptor comprises a novel ligand binding site of a TRAIL-R2 receptor. In certain embodiments, the crystallized complex of a novel ligand binding site of a TRAIL receptor comprises relative structural coordinates set forth in FIG. 23. In particular embodiments, the crystallized complex of a novel ligand binding site of a TRAIL receptor comprises a structure set forth in FIG. 1 or FIG. 3. In particular embodiments, the crystallized complex of a novel ligand binding site of a TRAIL receptor comprises an amino acid sequence of the TRAIL-R2 receptor that comprises, consists of or consists essentially of amino acid residues 58-184 of TRAIL-R2 receptor or a subsequence, portion, homologue, variant or derivative thereof. In different embodiments the amino acid sequence of the TRAIL-R2 receptor comprises, consists of or consists essentially of one or more of amino acid residues E78 and D109 of the TRAIL-R2 receptor; amino acid residue D148 of the TRAIL-R2 receptor; amino acid residues V167, V179 and W173 of the TRAIL-R2 receptor; amino acid residues Y103, N134 and R133 of the TRAIL-R2 receptor; amino acid residues L110, L114 and F112 of the TRAIL-R2 receptor; and amino acid residues E151 and E147 of the TRAIL-R2 receptor, or a subsequence, portion, homologue, variant or derivative thereof.

Also provided are methods of use of the novel composition comprising a TRAIL receptor bound to a ligand in crystalline form or the novel crystallized complex of a TRAIL receptor and a ligand thereof described herein for designing a compound, protein or peptide that interacts with the TRAIL receptor.

As will be understood by a person of skill in the art, in particular embodiments, the method of use of a novel composition comprising a TRAIL receptor bound to a ligand in crystalline form or a novel crystallized complex of a TRAIL receptor and a ligand thereof described herein for designing a compound, protein or peptide that interacts with the TRAIL receptor is computer implemented. Thus in particular embodiments, a computer system is used to represent a novel composition comprising a TRAIL receptor bound to a ligand in crystalline form or a novel crystallized complex of a TRAIL receptor and a ligand thereof described herein to design a compound, protein or peptide that interacts with the TRAIL receptor, the computer system including data storage means including data corresponding to the coordinates of the novel composition comprising a TRAIL receptor bound to a ligand in crystalline form or a novel crystallized complex of a TRAIL receptor and a ligand thereof described herein. In particular embodiments, the coordinates comprise the coordinates set forth in FIG. 23.

In certain embodiments, the computer system is arranged to provide a representation of a three-dimensional structure of the novel composition comprising a TRAIL receptor bound to a ligand in crystalline form or the novel crystallized complex of a TRAIL receptor and a ligand thereof described herein. The computer system may include a display for displaying a representation of the three-dimensional structure of the novel composition comprising a TRAIL receptor bound to a ligand in crystalline form or a novel crystallized complex of a TRAIL receptor and a ligand thereof described herein.

All publications and patent applications cited in this specification are herein incorporated by reference as if each individual publication or patent application were specifically and individually incorporated herein by reference in their entirety. In case of conflict, the specification, including definitions, will control. The citation of any publication is not to be construed as an admission that the invention is not entitled to antedate such publication by virtue of prior invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, the singular forms “a”, “and,” and “the” include plural referents unless the context clearly indicates otherwise. Thus, for example, reference to “an agent” such as an “antibody” or an “inhibitory nucleic acid” or a “small molecule” includes a plurality of such agents, and reference to “an activity or function” can include reference to one or more activities or functions, and so forth.

As used in this specification and the appended claims, the terms “comprise,” “comprising,” “comprises” and grammatical variations of these terms are intended in the non-limiting inclusive sense, that is, to include the particular recited elements or components without excluding any other element or component.

Concentrations used herein, when given in terms of percentages, include weight/weight (w/w), weight/volume (w/v) and volume/volume (v/v) percentages.

As used herein, numerical values are often presented in a range format throughout this document. The use of a range format is merely for convenience and brevity and should not be construed as an inflexible limitation on the scope of the invention. Accordingly, the use of a range expressly includes all possible subranges and all individual numerical values within that range. Furthermore, all numerical values or numerical ranges include integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-5,000 fold includes 1, 2, 3, 4, 5, 6, 7, 8, 9, 10, 11, 12, 13, 14, 15, 16, 17, 18, 19, 20, etc., . . . . 5,000 fold, etc., as well as 1.1, 1.2, 1.3, 1.4, 1.5, fold, etc., 2.1, 2.2, 2.3, 2.4, 2.5, fold, etc., and any numerical range within such a ranges, such as 1-2, 3-5, 5-10, 10-50, 50-100, 100-500, 100-1000, 500-1000, 1000-2000, 1000-5000, etc.

As also used herein a series of range formats are used throughout this document. The use of a series of ranges includes combinations of the upper and lower ranges to provide a range. This construction applies regardless of the breadth of the range and in all contexts throughout this patent document. Thus, for example, reference to a series of ranges such as 5 to 10, 10 to 20, 20 to 30, 30, to 50, 50 to 100, 100 to 150, 150 to 200, 200 to 300, or 300 to 400, 400-500, 500-600, or 600-705, includes all combinations of the different ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-200, 50 to 200, 50 to 300, 50, to 400, 50 to 500, 100 to 300, 100 to 400, 100 to 500, 100 to 600, 200-400, 200-500, 200 to 600, 200 to 700, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

A number of embodiments of the invention have been described. Nevertheless, it will be understood that various modifications may be made without departing from the spirit and scope of the invention. Accordingly, the following examples, which include data demonstrating a physiological interaction of TRAIL-R2 receptor and UL141 are intended to illustrate but not limit the scope of invention described in the claims.

### Examples

### Materials and Methods

Design of Expression Constructs—U1141, TRAIL-R2

The mature ectodomains of UL141 (amino acids (aa) 30-279 and 30-217, HCMV FIX strain) and TRAIL-R2 (DR5; aa 58-184) were PCR amplified and cloned downstream of the gp67 signal sequence into the baculovirus transfer vector pAcGP67A (BD biosciences) upstream of the Fc domain of human IgG1 (pAc-gp67A-MCS-Thr-Fc; for use in Biacore). A thrombin protease cleavage site (LVPRGS) was also introduced between the individual ectodomain and the Fc fusion protein. In parallel, both UL141 ectodomain constructs were also cloned in pAcGP67A containing only a C-terminal poly-histidine tag. The UL141 constructs were amplified by polymerase chain reaction (PCR) using human cytomegalovirus (HCMV) cDNA as a template. For amplification of TRAIL-R2, human full-length cDNA was used as a PCR template. A nested PCR protocol with two pairs of primers was used to generate the constructs (Table 4). One pair of primers (hcmvUL141/30for/BamHI and hcmvUL141/217rev/His/EcoRI) generated a DNA product coding for residues 30-217 of UL141 followed by C-terminal polyhistidine-tag, where forward primer introduced a BamHI restriction site and the reverse primer an EcoRI restriction site with preceding stop codon. Similarly, the second pair of primers (hcmvUL141/30for/BamHI and hcmvUL141/279rev/His/EcoRI) generated a DNA fragment coding for residues 30-279 of UL141. The Fc-fusion expression constructs were generated by amplifying corresponding DNA genes and further ligated into the C-terminal Fc-fusion protein containing baculovirus transfer vector (pAc-gp67A-MCS-Thr-Fc). The following pairs of primers were used in PCR: TRAIL-R2 58-184 gene (huTRAIL-R2-Fc/58for/EcoRI and huTRAIL-R2-Fc/184rev/PstI), UL141 37-247 gene (hcmvUL141-Fc/37for/EcoRI and hcmvUL141-Fc/247rev/PstI) and UL141 37-273 gene (hcmvUL141-Fc/37for/EcoRI and hcmvUL141-Fc/273rev/PstI). The identity and correct sequence of all PCR-amplified constructs was confirmed by sequencing.

Preparation of Recombinant Baculoviruses

The baculovirus transfer vector pAcGP67A containing the UL141 or TRAIL-R2 expression construct was amplified in bacteria (E. coli DH5α) and maintained under sterile conditions. To increase transfection efficiency, transfection was performed in serum-free media (HyClone SFXlnsect Cell Culture Media, Thermo Scientific) without any antibiotics using Cellfectin reagent (Invitrogen) according to manufacturer's instructions. The transfection complex was formed as follows: 2 μg of recombinant DNA (UL141 or TRAIL-R2 in transfer vector)+0.1 μg of BaculoGold DNA (Invitrogen)+10 μl of Cellfectin Reagent were filled up to 1 ml with media. As a negative control, 20 μl of Cellfectin+1 ml media was mixed. The transfection mixture was vigorously vortexed for 30 sec and incubated at RT for 15 min in the dark. 2×10⁶ healthy-dividing Spodoptera frugiperda (SF)9 cells were seeded in T-25 (25 cm²) flasks. Culture media was removed and transfection mixture was added drop-wise. Transfection plates were then incubated at RT for 4 hours while rocking back-and-forth every 30 min in dark. After 4 hours, the transfection mixture was replaced with 5 ml fresh media containing antibiotics (mixture of 50 U/ml of penicillin and 50 μg/ml of streptomycin) and plates were incubated at 28° C. for 7 days. For the initial screening for positive recombinant UL141 or TRAIL-R2 virus the dilution virus pool method was applied. Positive recombinant virus was selected and then amplified as follows. Cell supernatant containing recombinant virus was collected (1000×g for 10 min) and used for first round of virus amplification. 300 μl of virus with a multiplicity of infection below 1 (MOI<l) was used to infect 2×10⁶ cells in T-25 flask and the flask was then incubated at 28° C. After 5 days, the second virus amplification was performed in T-175 flask to infect 14×10⁶ cells with volume of 1.5 ml of collected virus from the first amplification (MOI<l) in 50 ml of media and incubated for additional 5 days at 28° C. Virus titer was determined by end-point dilution assay (EPDA). Prior to expression, the high titer virus stock was prepared in several T-175 flasks by infection at MOI=1 of 14×10⁶ cells in total 50 ml volume of media and incubated for 6 days at 28° C. Each flask was then directly used for infection of 2500×10⁶ cells in total 1 L volume of media (MOI between 3 to 5) and incubated for 72 to 84 h at 28° C. as a suspension culture (at 138 rpm).

Expression of Seleno-Methionine Labeled UL141-TRAIL-R2-Fc Complex

Recombinant virus stock containing both UL141 and TRAIL-R2-Fc virus particles was prepared similar to the individual virus stocks (see above). To achieve equal protein synthesis via baculovirus mediated co-expression in Sf9, we prepared the transfection mixture under the following condition: 2 μg of UL141 recombinant DNA+2 μg of TRAIL-R2 Fc-fusion recombinant DNA (both in separate transfer vectors)+0.5 μg of BaculoGold DNA (Invitrogen)+20 μl of Cellfectin Reagent, filled up to 1 ml with media and as a control, 20 μl of Cellfectin+1 ml media was mixed. The first round of virus amplification was done by infecting the Sf9 cells, which were previously adapted for vital growth in ESF-921 protein-free media (Expression systems, Inc.), with heterologous virus from a 7-day transfection at 28° C. Similarly, the second virus amplification and the high titer virus stocks were prepared in several T-175 flasks by infection at MOI=1 of 14×10⁶ cells in total 50 ml volume of ESF-921 media and incubated for 6 days at 28° C. Each flask was then directly used for infection of 3500×106 cells in total 1 L volume of ESF-921 methionine-rich media (MOI between 3 to 5) and incubated for 16 hours at 28° C. as a suspension culture (at 138 rpm). To achieve depletion of methionine from intracellular pools, cells were collected at 300 g for 15 min at RT and resuspended in ESF-921 methionine-free media with antibiotics (50 μg/mL gentamycin). Subsequently, seleno-methionine (50 mg/L) was added, to the suspension culture at 28° C. The critical point of seleno-methionine addition is within the first 16-20 hours following viral infection, as the protein expressing begins at that time. Expression of seleno-methionine labeled UL141-TRAIL-R2 Fc-fusion protein complex was continued for 48-96 hours post-infection (total time of expression 3.5 days at 28° C.). The culture media containing the seleno-methionine labeled protein complex was separated from cells by centrifugation (1000 g for 10 min) and debris was removed by additional centrifugation at 5500 g for 10 min at 4° C.

Expression of UL141 Fc and CD155 Fc- and TRAIL-R1 Fc-Fusion Proteins

Fc-fusion proteins were produced in baculovirus mediated insect cell expression system as well as in mammalian 293T cells. For 293T cells, DNA was prepared using the Endofree Plasmid Maxi kit (Qiagen, Valencia, Calif., USA) and maintained under the sterile condition. The confluent 293T cells were passaged in T-175 flasks in D10 media and incubated at 37° C. with 5% CO2. As a detaching component, 0.05% trypsin-EDTA solution was used to further maintain the cells. 293T cells were transfected by standard calcium phosphate transfection method and subsequently maintained for 72 h. The transfection mixture containing 100 μl of 2.5 M CaCl2, 22 μg DNA filled up to 1 ml with sterile water (calculation for one T-175 plate) was bubbled into 1 ml of 2×HeBS buffer (containing phosphate) and drop-wise transferred to seeded 293T cell in T-175 flask containing 25 ml D10 medium. After one day of transfection the media was changed to 30 ml CellGro media containing antibiotics and L-glutamine. After 48 hours of expression, the media was changed to fresh and supernatant was collected for harvesting, while rest of the cells in fresh media continues for next 24 hours expression. UL141 Fc protein was purified from cell culture supernatant using a HiTrap Protein A HP column (Amersham Biosciences, Piscataway, N.J., USA), while CD155-Fc and TRAIL-R1 Fc were used directly from culture supernatant for SPR studies (see below).

HEK293T Cell Culture

HEK293T cells were grown in Dulbeccos's modified medium (DMEM) supplemented with 10% (v/v) fetal calf serum (FCS), 2 mM L-glutamine and 100 units/ml of penicillin, 100 μg/ml of streptomycin (all together are components of D10 medium). Transfected 293T cells were further maintained in CellGro serum-free, protein-free media (CellGro, Mediatech).

Western Blots

Fc-fusion and His-tagged proteins were run on SDS gradient polyacrylamide gels and transferred to nitrocellulose membranes. Blots were probed with anti human IgG-HRP conjugate for UL141-Fc, CD155-Fc and TRAIL-R2-Fc (BioRad), or with mouse anti-penta-His conjugate and anti-mouse IgG HRP conjugate antibodies (Sigma).

Purification of UL141-TRAIL-R2 Complex (SeMet-Labeled and Native)

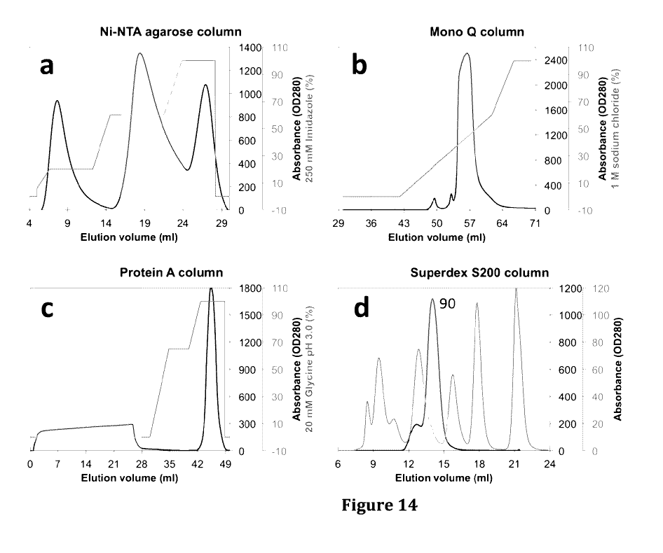

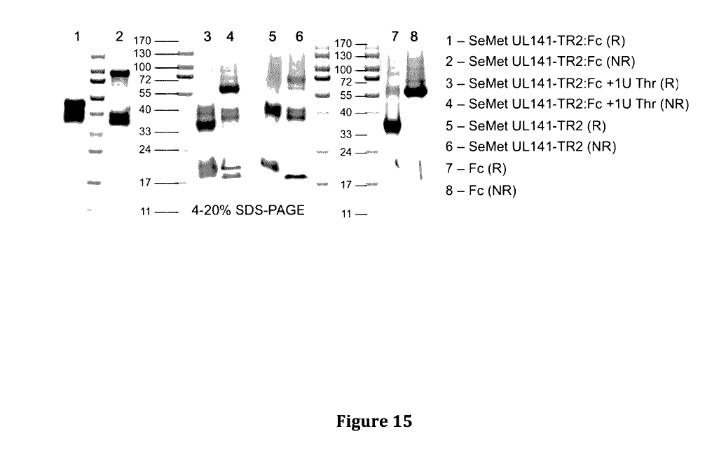

The extracellular domains of TRAIL-R2 and UL141 were cloned into transfer vector pAcGP67A engineered with C-terminal Fc-fusion tag in case of TRAIL-R2 and His-tag for UL141 construct. The proteins were co-expressed via the baculovirus expression system as a non-covalent protein complex, while a cleavage site for thrombin protease was introduced between TRAIL-R2 and the Fc portion of human IgG1. After three days of expression in insect cell media at 28° C., Sf9 cells and debris was removed from the protein containing culture supernatant by centrifugation. The supernatant was concentrated to 500 ml while the buffer was exchanged against 1×PBS by tangential flow-through filtration using 10 kDa molecular weight cut-off membranes (Millipore filtration device, Pelicon-2). Briefly, the UL141-TRAIL-R2-Fc complex was purified by affinity chromatography using Protein A (HiTrap Protein A), followed by Ni2+-affinity chromatography using HisTrap (both GE Healthcare), to purify the protein complex, rather than the individual components (FIGS. 14 and 15). Next, the UL141-TRAIL-R2-Fc containing fractions were pooled and dialysed at 4° C. against 10 mM TRIS pH 8.0 buffer for subsequent purification by anion-exchange chromatography using MonoQ (GE Healthcare) and a 0-1 M sodium chloride gradient (FIG. 14b). The UL141-TRAIL-R2 complex was further released from the Fc fusion tag by thrombin (Sigma) digestion at RT for 2 h, using 1U of thrombin per mg of protein complex. Free Fc protein as well as uncleaved complex was further removed by affinity chromatography using Protein A resin (FIG. 14c). During final purification by size exclusion chromatography (SEC) using Superdex 5200 (GE Healthcare), the UL141-TRAIL-R2 complex eluted as a 90 kDa peak consistent with one UL141 dimer binding two TRAIL-R2 monomers. The protein complex migrated as two major bands (38 and 19 kDa) on both reducing and non-reducing SDS gels (FIG. 15).

Crystallization of UL141-TRAIL-R2 Protein Complex (Native and SeMet-Labeled)

The UL141-TRAIL-R2 containing fractions for both native and selenomethionine labeled protein were pooled and concentrated to final concentrations of 7.3 mg/ml (native) and 8.3 mg/ml (labeled) in 50 mM HEPES, 150 mM NaCl, pH7.5. Initial crystallization trials were carried out by robotic crystallization (Phoenix, Art Robbins Instruments) using the sitting drop vapor diffusion method at room temperature as well as 4° C. Over 700 conditions were screened using several different commercial crystallization screens (Wizard I, II, III; PEG-ion 1, 2; JSCG I-IV and Core; Hampton Research Additive Screen) to find several initial crystallization hits for UL141-TRAIL-R2 native and one condition for labeled complex. Three-dimensional native and derivative crystals of the UL141-TRAIL-R2 protein complex were grown at 22° C. in the presence of high pH buffer (CHES 9.5 and bicine 9.0, respectively) and 20 (w/v) polyethylene glycol (8000 and 6000, respectively). The derivative condition also includes 0.2 M calcium chloride and 5-10% glycerol as an additive. These crystals were further optimized by macro- and micro-seeding techniques, as well as by crystallization under oil to improve diffraction quality. Crystallization under oil and crystallization with glycerol were the most successful optimization. A 5-8 μl drop containing a 1:1 mixture of Silicon and Paraffin Oil (Hampton Research), also known as Al's Oil, was placed as sitting drop. Next, the protein and precipitant (see above) was mixed 1:1 and pipetted under the oil. Reservoir was filled up with 1 ml of precipitant solution. Crystals were grown slowly over several days to maximal dimensions of approximately 1000×30×40 μm.

Data Collection and Processing—UL141-TRAIL-R2 Native and Derivative Data

Crystals were cryo-protected in well solution containing 25% glycerol and then flash-cooled in liquid nitrogen for data collection at 100 K. X-ray diffraction data were collected from the six best diffracting crystals at the Stanford Synchrotron Radiation Lightsource (SSRL) beamline 7-1 after testing crystals by excitation scan at Se-K edge for Se incorporation. The wavelength used for data collection was at the peak of Se f″ (0.9795 Å, 12667 keV). The inverse-beam mode of data collection was used with 7 sec exposure time (the crystal was rotated 180° every 10 frames to measure Friedel mates). To better resolve the reflections corresponding to the long axis, the crystals were aligned in the loop with the long axis roughly parallel to the rotational spindle axis. In addition, a long sample-to-detector distance (300 mm) and an oscillation of 0.5° were used to reduce overlaps. The strategy function in iMosflm ⁴⁴ was used to reduce overlap as well as to maximize data completeness. Five of the six diffraction data sets were selected for analysis. The parameter for collection of the native UL141-TRAIL-R2 dataset at direct beam mode are as follows: crystal-to-detector distance (350 mm), exposure time for 10 sec and oscillation increment was 1°. Both data were indexed and integrated by iMosflm. The crystals of SeMet UL141-TRAIL-R2 belong to space group P212121, with unit-cell parameters a=67.74 Å, b=97.01 Å and c=140.94 Å and native UL141-TRAIL-R2 with a=67.71 Å, b=97.67 Å, c=141.31 Å.

Multi-Crystal Data Reduction—UL141-TRAIL-R2 Derivative

Each single-crystal data set was indexed and integrated by iMosflm ⁴⁴. The CCP4 program (Collaborative Computational Project, Number 4) SCALA 45 was used for data scaling and merging with secondary beam correction and rotational restraints for scale and B factors. The ‘anomalous’ option in SCALA was turned on to allow the separation of Friedel mates in the merged data. For scaling, Friedel mates were not treated separately. A multicrystal dataset was produced by merging the five individual anomalous datasets in SCALA. Different sets of multi-crystal data were generated, including and excluding data from crystal C6. The C6 data proved to have appreciably stronger anomalous signal than the others. Data collection statistics for the native data, the C6 data as well as multi-crystal merged data are presented in Table 2. The strategy for multi-crystal data reduction was adapted from a prior strategy⁴⁶.

Substructure Determination and Phasing—UL141-TRAIL-R2

Selenium-substructure determinations were performed with the SHELXD program package⁴⁷. A resolution cutoff at 4.5 Å and an Emin cutoff at 1.4 were initially used to find Se substructures with SHELXD. Trials were made for each data set and for various merged data sets. For each case, 500 attempts were made to find the expected 20 Se sites. For those single-crystal and multicrystal data sets that did not yield successful Se-substructure determinations using SHELXD, Se substructures were obtained by running Phaser ⁴⁸ in its MR-SAD mode with phases from the model (PDB coordinates for TRAIL-R2 (1D4V) and our incomplete homology model of UL141-Igdomain, data not shown). The model was only used for Se-substructure determination and was excluded from the subsequent SAD phasing. For all cases, initial SAD phases were calculated by Phaser. These initial phases were subjected to automatic density modification with solvent flattening and histogram matching as implemented in the CCP4 program DM and DM-Multi ⁴⁹. An estimated solvent content of 51% was used for the density modification procedure. Map correlation coefficients (map CCs) and mean phase errors were calculated to compare the resulting experimental phases with model phases. In order to improve density, Uniqueify was used to generate Free R value and FFT to generate anomalous density map. Automated model builder ARP/wARP generated polyalanine model and Buccaneer (all of CCP4 package) together with AutoRickshaw structure solving module⁵⁰ were used to extend this model by searching for TRAIL-R2 (from PDB 1D4V). The first interpretable model of UL141-TRAIL-R2 dimer was then rebuilt into CA-weighted 2Fo-Fc and Fo-Fc difference electron density maps using the program COOT ⁵¹. Final steps included the TLS procedure in REFMAC5 ⁵² with three TLS domains (residues 80-180 of TRAIL-R2, 34-165 and 176-198 of UL141). The UL141-TRAIL-R2 structure was refined to 2.1 Å with a final Rfree of 27.4%. The quality of the model was examined with the program Molprobity⁵³.

Surface Plasmon Resonance

After purification, the proteins were concentrated with an Amicon Centrifugal Filter Unit (Millipore, Ultracell-30K or 10K) and the buffer was exchanged against 10 mM HEPES pH 7.4, 150 mM sodium chloride and 3 mM EDTA (as Biacore running buffer). The proteins were diluted in Biacore running buffer containing 0.005% Tween 20 to appropriate concentration prior to loading. An anti-human Fc capture antibody was immobilized on a CM5 sensor chip (GE Healthcare) by amine coupling. Approximately 500-1000 response units (RU) of TRAIL-R2-Fc, TRAIL-R1-Fc, UL141-Fc and CD155-Fc were captured on sensor chip. TRAIL-R1 Fc, CD155 Fc and TRAIL-R2 Fc mutant proteins were captured on the sensor chip directly from the filtered culture supernatant. The serial dilutions of UL141 protein (0-0.5 μM), TRAIL-R2 receptor (0-1 μM), UL141-TRAIL-R2 protein complex (0-10 μM) were prepared in running buffer. The analytes were then injected in duplicates for 5 to 10 min association, while dissociation was conducted over 30 min. After each cycle, the chip was regenerated with a 30 sec injection of 2M MgCl₂ at 15 μl/min and freshly coated with ligand (Fc-fusion protein). Experiments were carried out at 18° C. with a flow rate of 10 to 30 μl/min and performed in several repeats, each time with a different stock preparation (except for the experiment with TRAIL-R1, this was performed only once). As a negative control for unspecific binding, human LTβR-Fc (Lymphotoxin β receptor from TNFR family) was immobilized on the first flow-channel (it is know that UL141, TRAIL-R2, -R1 nor CD155 do not bind to LTβR). Kinetic parameters were calculated after subtracting the response to the negative control (LTβR-Fc) and next the buffer only control as a background, using a simple Langmuir 1:1 model in the BIA evaluation software version 4.1

Glycan Modeling

Three potential N-linked glycosylation sites were identified in the UL141 ectodomain. All of the possible asparagine residues (Asn117 in chain A, Asn132 in chain B, and Asn147 in both chains) carry one or two NAG (N-acetylglucosamine) residues that are clearly defined by electron density. While extra density is present also at the Asn117 (in chain B) and as well as Asn132 (in chain A), this density is not well defined, and no NAG was build in this location in crystal structure, but these sites were incorporated in modeling as they are occupied in adjacent UL141 subunit. Energy-minimized PDB coordinates were used for basic mannose containing N-linked carbohydrates (GlcNAG2-Man2) to visualize the surface accessibility on UL141.

Generation of Human TRAIL-R2-Fc Mutants

Human TRAIL-R2 Fc-fusion mutants (Table 5) were generated by site-directed mutagenesis using Quick Change II Multi-site Mutagenesis Kit (Stratagene, La Jolla, Calif., USA). Single mutations were incorporated using the Quick Change II Site-Directed Mutagenesis Kit (Stratagene, Agilent Technologies). Mutated constructs were purified with the Qiagen Miniprep Kit (Qiagen) and the presence of the mutation confirmed by sequencing. All mutants of human TRAIL-R2-Fc were expressed in Sf9 insect cells and the culture supernatant was used for SPR studies.

Cells and Virus.

Neonatal human dermal fibroblasts (NHDF) were obtained from Clonetics (San Diego, Calif.), immortalized human foreskin fibroblasts (HFF) are described (McSharry et al., 2001), and 293T cells were from the ATCC (CRL-11268). All cells were cultured in Dulbecco's modified Eagles medium (DMEM) supplemented with 10% fetal bovine serum, Pen/Strep and L-glutamine (Gibco). Insulin and bFGF (Sigma Aldrich) were added to NHDF media. Cells were verified mycoplasma negative. AD169 was acquired from the ATCC (VR-538, used p2-5), and Toledo was a kind gift from S. Starr (Philadelphia, used p12-15). Mutagenesis of FIX was performed as described (Hahn et al., 2002). MerΔUL141 generation is described (Prod'homme et al., 2010). HCMV virus was generated by BAC transfection into fibroblasts as described (Hahn et al., 2002; Stanton et al., 2010).

RNA Isolation and Analysis.

Total RNA was isolated from HCMV infected cells using TRIzol (Roche) followed by an RNeasy mini kit (Qiagen, Hilden, Germany). cDNA generation and real-time qPCR analysis is described (Schneider et al., 2008). For RACE analysis, a 5′/3′ RACE kit kit was used (Roche), and primers were UL141-5′ CCGGCGACGTG GTCTCATAA, UL141-3′ATCGCGGCAT TTTTGGGATT. The amplified products were purified by agarose gel and sequenced.

Flow Cytometry.

HCMV FIX infected six well dishes of NDHF or HFF were detached with diluted trypsin, washed in PBS and resuspended in PBS+2% FCS. Cells were incubated with 1° antibody for 20-30 min on ice, followed by anti-mouse IgG1 biotin (BD) and Streptavidin-APC (Pharmingen) if needed, and fixed with 1% paraformaldehyde. Anti-TRAIL-R1 and -R2 (HS101 and HS201, Alexis), anti-MHCI (W6/32, eBioscience) and anti CD155-PE (Biolegend) used at 5 μg/ml. Samples were acquired using a BD LSRII or FACScalibur flow cytometer and analyzed using FlowJo software (Tree Star). For Merlin infections, essentially the same methods were used, with secondary detection using anti-mouseAF647 (Molecular Probes, A-21238). Data was analysed with Accuri/CFlowPlus. UL141 transfected 293T and NHDF cells were analysed similarly, as were adenovirus transduced HFF and purified human NK cells.

Cell Death Assays.

MTT cell viability assays in HCMV infected NHDF (Benedict et al., 2001) and caspase 3/7 activation assays (Skaletskaya et al., 2001a) were performed essentially as described.

Plasmids, Adenovirus, Proteins and Transfections.

Plasmid vectors for expressing Fc-fusion proteins are described (PCR3-Fc)(Schneider, 2000). Adenovirus vectors expressing UL141 are described (Tomasec et al., 2005). Generation of TRAILR2ΔDD.GFP, TRAILR2ΔDD.RFP, CD155.RFP and MICA.GFP recombinant adenoviruses is described (Stanton et al., 2008), with modifications. Fc-fusion proteins used in ELISA and SPR were purified by protein A affinity from transfected 293T cell supernatants, except for TRAIL-R1:Fc (R&D Systems). For SPR studies, cell supernatants from SF9 cells transduced with baculovirus expressing His-tagged UL141ecto was collected after 3 at 27.5° C. (MOI=3), and purified using Ni₂₊-affinity chromatography followed by cation exchange chromatography using MonoS (GE Healthcare) and gelfiltration (Superdex 5200, GE Healthcare), by FPLC.

Six well dishes of 293T were transfected with 2 ug of UL141 plasmid as described (Cheung et al., 2005). One μg of UL141 plasmid was co-transfected into NHDF with 0.5 μg of the provided control GFP plasmid according to manufacturer's instructions (AMAXA).

Surface Plasmon Resonance Studies

SF9 cell purified UL141 was exchanged to Biacore running buffer, TRAIL DR Fc fusion proteins and hLTbR:Fc (neg control) were immobilized on an on an anti-human Fc capture chip, and binding was analyzed using a Biacore 3000 (GE Healthcare) essentially as described (Wang et al., 2010).

Western Blots

Cells were dissolved in NuPAGE LDS sample buffer (Invitrogen), proteins resolved on NuPAGE Novex 10% Bis-Tris gels (Invitrogen), transferred to nitrocellulose membrane (Hybond-C, GE) and membranes then treated with Antibody Extender reagent (Pierce) all according to manufacturers' instructions. The following antibodies were used to probe the membranes: TRAILR2 (R&D, AF631), CD155 (SDI) (Aoki, J B C, 1994:8431), UL141 (Tomasec et al., 2005), actin (Sigma A-2066), secondary antibodies were anti-mouse-HRP (BioRad 170-6516), anti-rabbit-HRP (BioRad 170-6515), anti-goat-HRP (SantaCruz sc-2056).

Immunofluorescence

Human fibroblasts (NPi) (Tomasec et al., 2000) were coinfected with relevant adenovirus vectors, 48 h after infection cells were fixed with 4% paraformaldehyde, stained with WGA-AF350 (Molecular Probes W11263) and imaged on Leica DMIRBE microscope with Improvision Openlab software.

NK Killing Assays

Purification of ‘bulk’ NK cells from IFNα activated human PBMC cultures has been described (Tomasec et al., 2005).

Statistical Analysis

Unless otherwise indicated, statistical significance was analysed by the Students T test, and data represent the mean±SEM.

Results

TRAIL death receptors (DRs) belong to the tumor necrosis factor receptor superfamily (TNFRSF), and can both promote apoptosis and regulate antiviral immunity. The UL141 protein of human cytomegalovirus inhibits host defenses by blocking cell surface expression of TRAIL DRs and CD155, a nectin-like Ig-fold molecule. Herein the present inventors discovered that UL141 utilizes at least two distinct binding sites to selectively engage TRAIL DRs or CD155. Binding studies revealed high affinity interaction of UL141 with both TRAIL-R2 and CD155 and low affinity with TRAIL-R1. The crystal structure of UL141 bound to TRAIL-R2 at 2.1 Å resolution revealed that UL141 forms a head-to-tail homodimer, through use of its Ig-domain, to bind two TRAIL-R2 monomers. While UL141 partially mimics the binding of TRAIL to TRAIL-R2, it utilizes its Ig-domain to facilitate non-canonical death receptor interactions, an emerging theme for the TNFRs.

### Example 1: Binding of UL141 to TRAIL Death Receptors and CD155

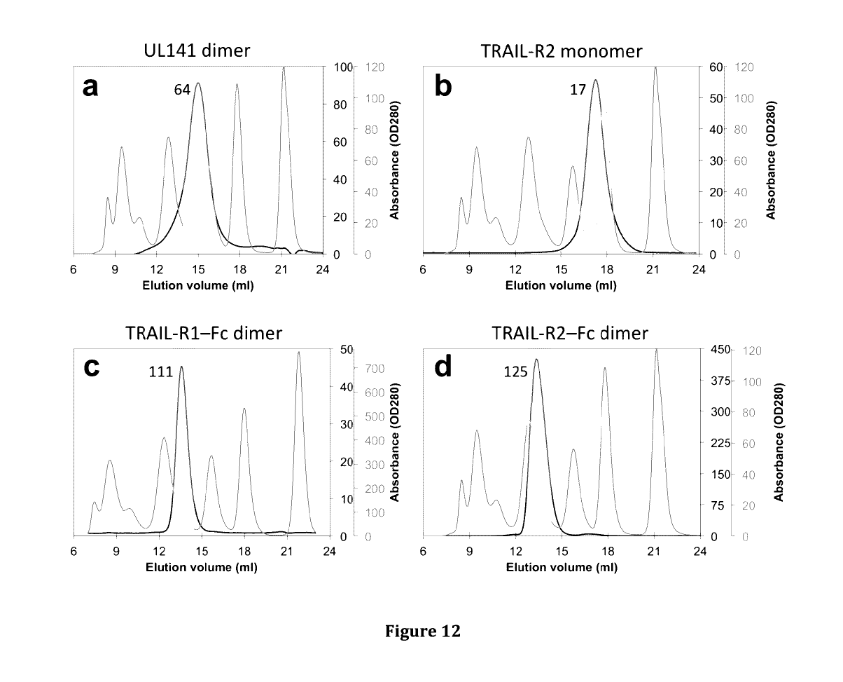

Recently, the UL141 protein of HCMV has been shown to be both necessary and sufficient to inhibit cell surface expression of the TRAIL death receptors and that UL141 can bind directly to the ectodomain of TRAIL-R1 and TRAIL-R2 ¹². This discovery revealed an unexpected pleiotropic role of UL141 in regulating host immunity, as previously this HCMV protein was known to only target the nectin-related molecules CD155 and CD112. TRAIL, is highly expressed by activated immune effector cells and can mediated apoptosis ¹⁷⁻¹⁹, and UL141 restriction of TRAIL DR expression likely contributes to its role as a potent NK cell inhibitor ¹⁶. As UL141 is sufficient to restrict expression of both CD155 and the TRAIL DRs, the inventors set out to determine the relative binding affinities of UL141 for these host cell proteins. All binding partners were produced as Fc fusion proteins, with an engineered protease cleavage site allowing for the release of the individual ectodomains. The monovalent binding interactions were then analyzed by Surface Plasmon Resonance (SPR), while the Fc fusion proteins were immobilized on the sensor chip. Recombinant UL141 bound directly to TRAIL-R2-Fc and CD155-Fc (with high affinity (KD of 6 nM and 2 nM, respectively) (Table 1). Interestingly, an approximately 4-fold higher KD was found when UL141-Fc was immobilized on the chip and monomeric TRAIL-R2 was used as the analyte (without Fc), with even more pronounced differences seen in the association and dissociation rate constants (Table 1). When UL141 was used as the analyte, the dissociation from TRAILR2-Fc was 100-fold slower, suggesting UL141 is not a monomer in solution. This increased avidity is in agreement with size exclusion chromatography results showing UL141 is a noncovalently associated dimer in solution, while recombinant TRAIL-R2 is a monomer (FIG. 12). Interestingly, the binding kinetics of UL141 to either TRAIL-R2 or CD155 differed significantly (Table 1). UL141 bound to CD155 with a 14-fold faster association rate (kₐ, kₒₙ), while dissociation was 5-times faster (k_{d}, k_{off}), resulting in a nearly 3-fold higher equilibrium binding affinity (KD). The 5-times slower dissociation rate of the UL141-TRAIL-R2 complex indicates that this complex is more stable in solution than UL141-CD155. The observed kinetic differences indicated that UL141 uses either distinct binding sites to bind TRAIL-R2 and CD155, or suggested different binding mechanisms (e.g. induced fit versus lock and-key). To test these hypotheses, the high-affinity UL141-TRAIL-R2 complex was pre-formed, and binding to CD155-Fc was assessed by SPR (Table 1). The binding kinetics of the UL141-TRAIL-R2 complex to CD155-Fc showed no difference from that of soluble UL141, strongly suggesting that the UL141 binding sites for TRAIL-R2 and CD155 are largely distinct. Although, TRAIL-R1 is highly homologous in primary sequence to TRAIL-R2, the binding affinity of UL141 for this DR was found to be ˜400-fold reduced (KD=2.304) when compared to TRAIL-R2 (KD=6 nM) 12. The binding kinetics revealed a 2-fold slower association rate (ka, kon), while dissociation was almost 200-times faster (kd, koff) when compared to UL141 binding to TRAILR2-Fc (Table 1). The rapid dissociation from TRAIL-R1 suggests that UL141 has a less optimized binding surface for TRAIL-R1, and instead has evolved to preferentially target TRAILR2.

### Example 2: UL141-TRAIL-R2 Complex Structure Determination

The complex of HCMV UL141 (residues 30-279) bound to human TRAIL-R2 (residues 58-184, both numberings start from the initial codon) was crystallized and the structure determined by single anomalous dispersion (SAD) to a resolution of 2.1 Å, using experimental phases derived from selenomethionine labeled protein expressed in Sf9 insect cells (see Methods herein) (Table 2). With the exception of several mobile loops of UL141, the entire N-terminal Ig-like β-sandwich domain and the cysteine rich domain (CRD) region of TRAIL-R2 (starting at residue 75-182) are well ordered in the final crystal structure and could be refined to a crystallographic R factor of 22.3% and an R_{free} of 27.4%. The molecules form together a heterotetrameric complex, where one UL141 dimer binds two TRAIL-R2 monomers through non-crystallographic two-fold symmetry (FIG. 1a).

### Example 3: Structure of UL141

UL141 has no sequence similarity to any other known cellular protein. For SPR studies, the UL141 ectodomain was expressed as a thrombin-cleavable Fc fusion protein in Spodoptera frugiperda (Sf9) insect cells using the baculovirus mediated expression system. However, recombinant UL141 purified by this method gradually lost its ability to bind to TRAIL-R2 within 3 days, suggesting it was unstable in solution. As an attempt to attain stable and homogeneously glycosylated protein for structural studies, UL141 was co-expressed with TRAIL-R2 in Sf9 cells. The resulting complex was co-purified, and was found to be stable in solution for several weeks and amenable to crystallization. In agreement with biochemical analysis using size exclusion chromatography, UL141 forms a non-covalent homodimer (FIG. 12 and FIG. 1a). Structural analysis revealed that UL141 interacts in a head-to-tail fashion to form a well-packed dimer, connected via several hydrogen bonds and salt bridges. The UL141 ectodomain exhibits an N-terminal immunoglobulin (Ig)-like domain (residues 54-160), followed by an additional β-sandwich domain (residues 161-279) (FIG. 1a,b). The presence of ten β-strands, arranged in two antiparallel β-sheets (formed by β-strands a, a′, g, f, c, c′, c″ and β-strands d, e, b, respectively) and a tryptophan residue (W74) packed over a central disulfide bond linking β-strands b and f clearly classifies it as a variable (V-type) Ig-like domain. In contrast to classical V-type Ig domains, however, UL141 also has an additional C-terminal β-strand domain (amino acids 161-251), formed by a three-stranded antiparallel β-sheet (β-strands 1, 2 and 3) and a short α-helix at the C-terminus (X, residues 234-241). The N-terminal domain also features an additional ‘one-turn’ α-helix (Y, residues 46-51) that separates the β-strand a from a′. The second disulphide bond of UL141 (C84-C234) connects α-helix X with the bottom of the N-terminal β sandwich domain holding these two UL141 domains together.

### Example 4: Structure of the TRAIL-R2 Human Death Receptor

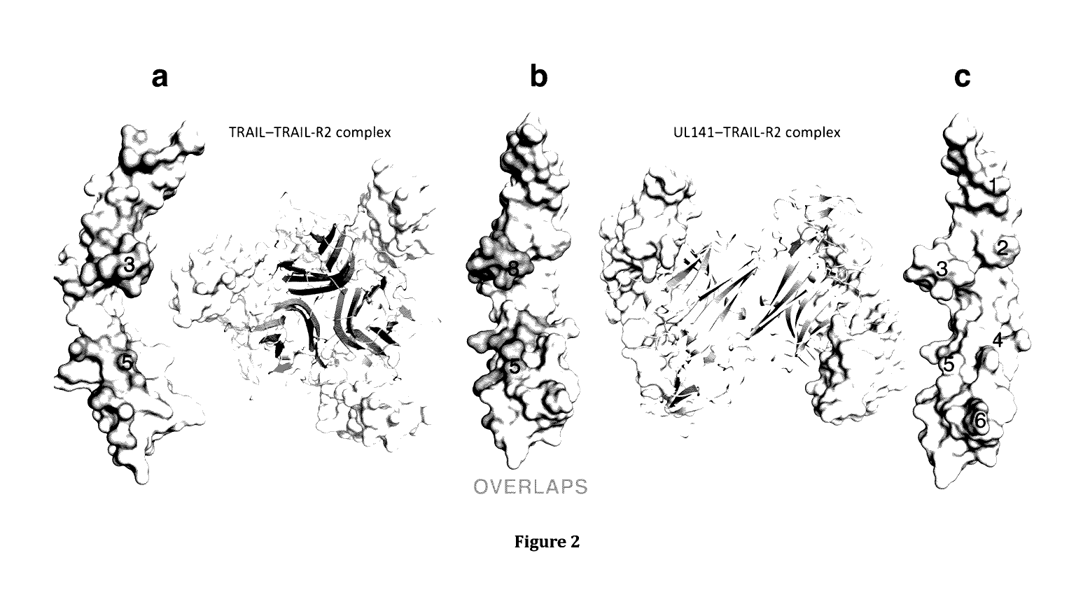

The structure of TRAIL-R2 bound to its homotrimeric cellular ligand TRAIL has been reported previously (²⁰ PDB: 1DU3; ²¹ PDB: 1D4V; ²² PDB: 1DOG). Each monomer of the trimeric TRAIL, binds to one TRAIL-R2 molecule, thereby leading to the trimerization and clustering of TRAIL-R2 on the cell surface, the hallmark oligomerization state thought to initiate signaling by TNFRs (FIG. 2a).

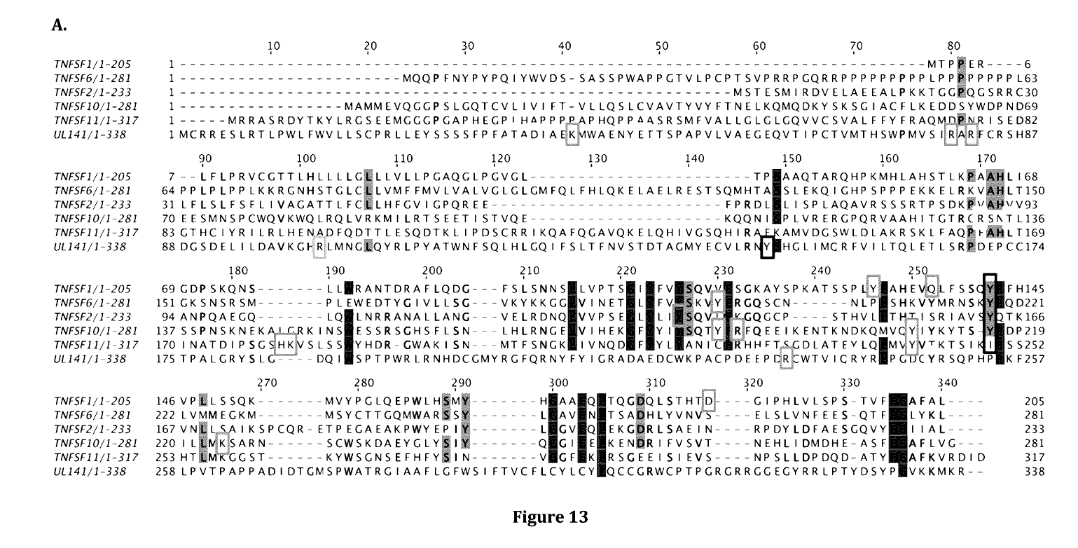

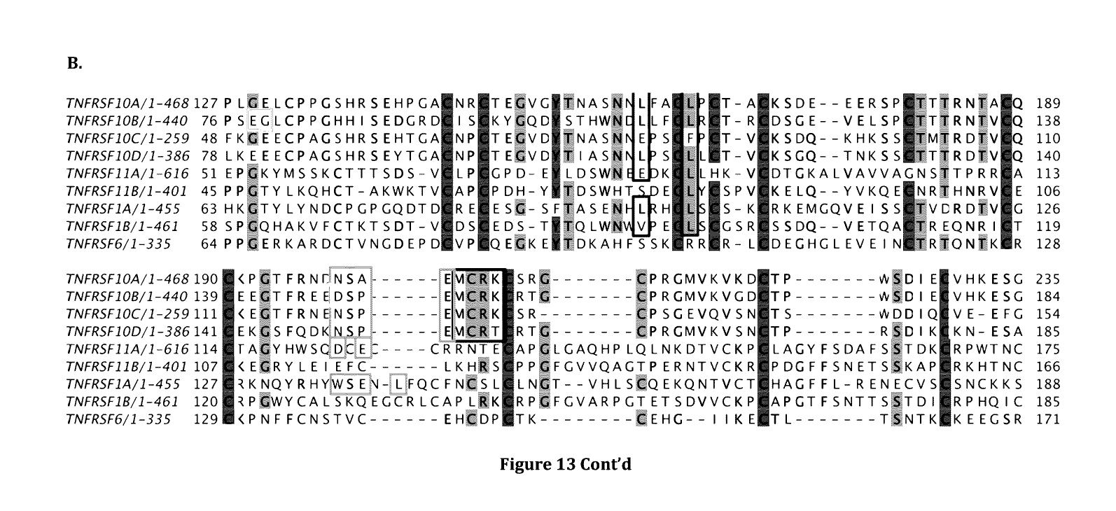

TRAIL-R2 is a monomer in solution (FIG. 12) with structurally conserved features of other members of the TNFR superfamily. It adopts an elongated structure composed of three extracellular pseudorepeats, or CRD's (Cysteine-Rich Domain), characterized by a cysteine knot topology 23,24. CRD-1-3 span a length of 70 Å, and CRD-2 and CRD-3 form the major ligand-binding region in the UL141-TRAIL-R2 complex (FIG. 1a,c). TRAIL-R2 starts with an N-terminal cap containing a cysteine knot with a single non-canonical disulfide bond. This N-terminal cap, which forms an incomplete cysteine repeat, corresponds to the C-terminal half of the first repeat (CRD-1) of other TNFRSFs, while CRD-2 and -3 of TRAIL-R2 correspond to the central two repeats of other TNFRSFs which forms the binding interface for LTα-TNFR-1, TNFα-TNFR-2, RANKL-RANK as well as TRAIL-TRAIL-R2 complex. These two ligand-binding repeats in all TNFRSF molecules are joined by a CXC motif (CQC in all the TRAIL receptors, CGC in TNFR-1, CTC in TNFR-2 and CAC in RANK) (FIG. 13), which acts as a flexible articulation point in all these receptors.

### Example 5: UL141-TRAIL-R2 Complex Architecture

In contrast to TRAIL binding to TRAIL-R2, which leads to head-to-head trimerization of the receptor, UL141 binding to TRAIL-R2 results in head-to-tail dimeric arrangement of TRAIL-R2 (FIGS. 1 and 2). The high affinity interaction between UL141 and TRAIL-R2 correlates well with the extensive buried surface area (1401.33 Å2), which is concentrated in three binding regions (FIG. 2). Furthermore, mutational analysis indicates that those binding regions can be further divided into six distinct binding patches (FIGS. 1 and 2). The binding contact region comprises patch 6, the central region patches 4 and 5, while the upper binding region combines patches 1, 2, 3 (FIG. 2). Structural comparison with the TRAIL-TRAIL-R2 complex reveals that patches 3-5 on TRAIL-R2 partially overlap with the binding site for the endogenous ligand TRAIL, while patches 1, 2, 6, and part of patch 3 are unique to the binding of UL141.

Two structures of TRAIL-R2 in complex with bound Fabs have also been determined (²⁵ PDB: 2H9G; ²⁶ PDB: 1ZA3), allowing the comparison of the TRAIL-R2 structure determined from multiple receptor-ligand complexes. Superimposition of all five TRAIL-R2 structures revealed structural changes within TRAIL-R2 that likely result from binding to distinct ligands (FIG. 3). This structural change is located in the central binding region of TRAIL-R2 (CRD-3 β1β2 loop 143-157, part of patch 3). This β1β2 loop is well conserved among all previously published TRAIL-TRAIL-R2 complexes (grey, green, light purple), but adopts different orientations upon binding of distinct antibodies (red, yellow) or UL141 (cyan).

### Example 6: TRAIL-R2 Binding Site Analysis

Based on the contact residues identified in the UL141-TRAIL-R2 complex, alanine-scanning mutagenesis of TRAIL-R2 was then performed followed by SPR analysis to assess the relative binding requirements for TRAIL and UL141 (FIG. 2). Notably, all six binding patches contain residues that contribute to the binding of UL141 and/or TRAIL. In the following example sections, it is reported how this mutagenesis analysis has revealed that UL141 has evolved to bind uniquely to this TRAIL DR:

### Example 7: UL141 Interacts with TRAIL-R2 in a Unique Fashion

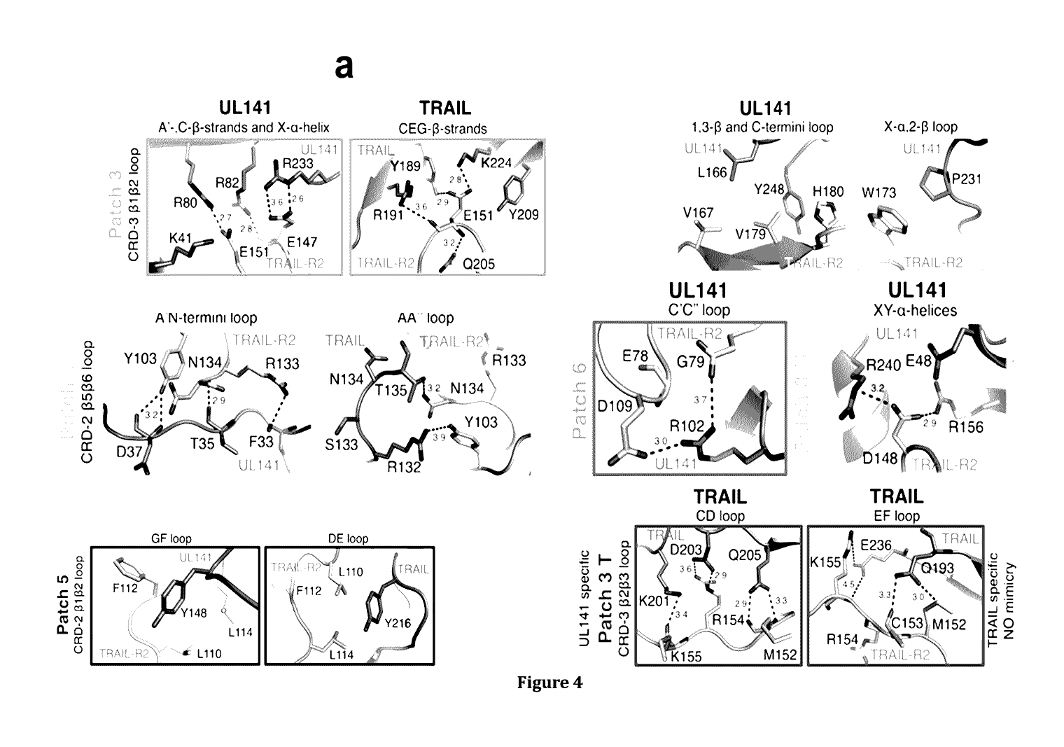

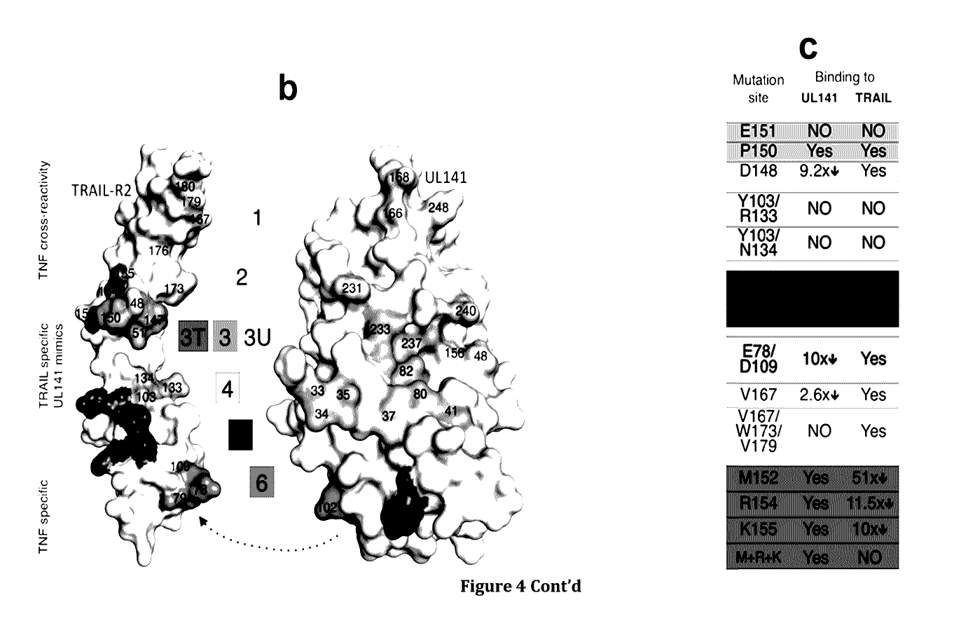

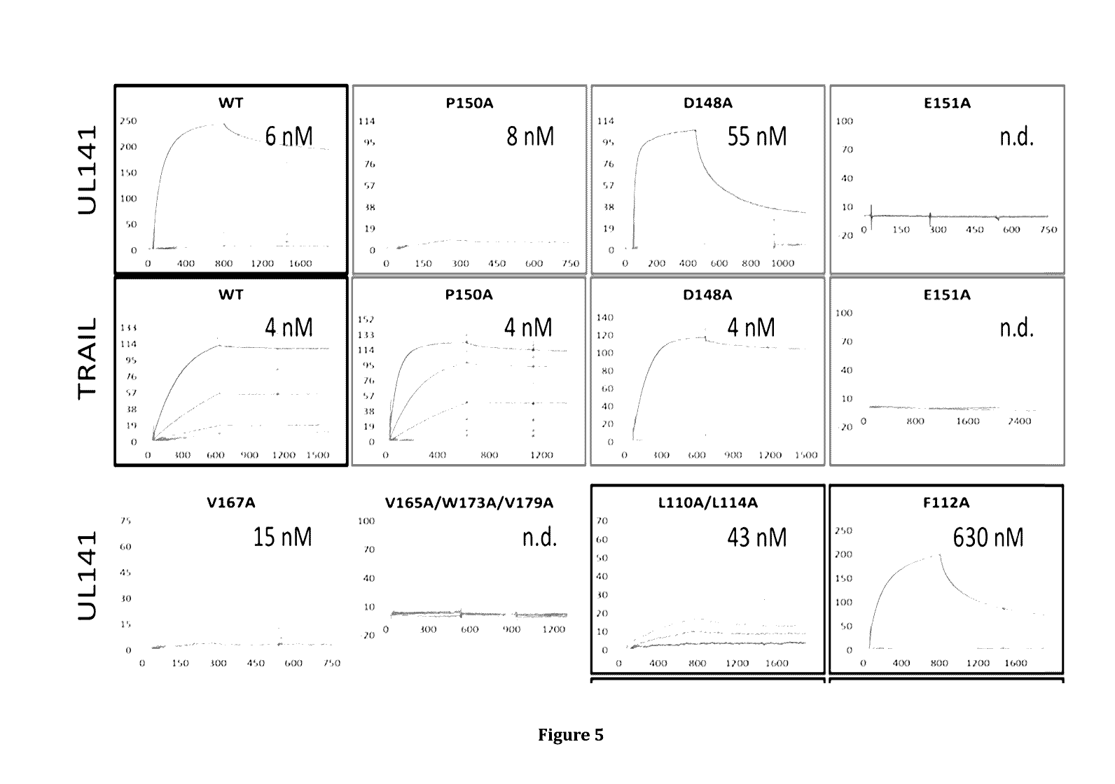

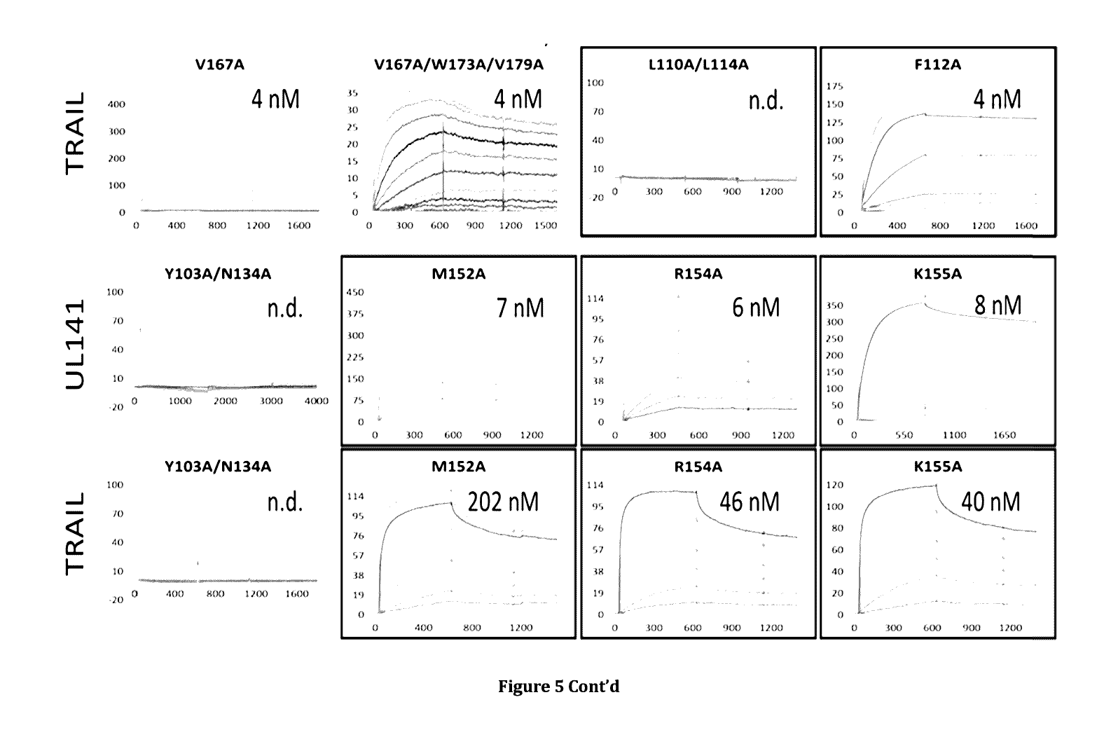

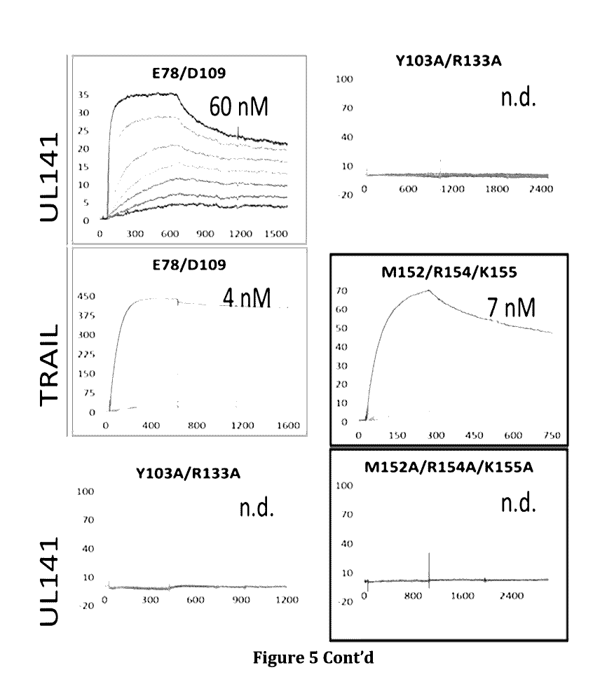

In the UL141-TRAIL-R2 complex, unique contacts are formed involving E78 and D109 of TRAIL-R2 that form two salt-bridges with R102 of UL141 (FIG. 4, Patch 6). TRAIL-R2 mutation D109A together with E78A reduced UL141 binding affinity 10-fold, while having no effect on TRAIL-binding (FIG. 5). In addition, D148 of TRAIL-R2 receptor (FIG. 3c) forms two salt-bridges with R240 and R156 (helices X and Y, respectively) of UL141 (FIG. 4, Patch 3U). The mutation D148A on TRAIL-R2 lead to a 10-fold reduced binding affinity for UL141 (KD=55 nM, FIGS. 4c and 5, Table 3). In addition, the C-terminal loop of TRAIL-R2 (V167, V179 and W173, FIG. 4, Patch 1-2) is slightly pulled toward UL141 compared to those of other TRAIL-R2 structures, as it forms several contacts with UL141 (L166, Y248 and P231, FIG. 4, Patch 1-2). The interactions within this binding region 1 (patches 1-2) are hydrophobic, in contrast to the centrally located patches 2-4, which are dominated by electrostatic interactions. Among the hydrophobic interface residues, the TRAIL-R2 V167A mutant exhibits a 2.5-fold reduced binding affinity to UL141 (KD=15 nM), while the triple mutation (V167A-W173A V179A) abolishes binding to UL141 completely. Strikingly, all these mutations in binding region 1 are unique to UL141, having no effect on TRAIL binding (FIGS. 4c and 5, Table 3)

### Example 8: UL141 Mimics Some TRAIL-Specific Contacts

The central binding interface of the UL141-TRAIL-R2 complex is structurally similar to other TNF-TNFR complexes (FIG. 4, Patch 4), and involves residues 33-37 of UL141 that correspond to TRAIL residues 131-135 (A′N-termini loop connecting strand a′ with the N-terminus of UL141; called AA″ loop in TNF ligands). This binding loop forms several specific polar interactions with CRD-2 β1β2 and β5β6 loop of TRAIL-R2, displaying well-ordered electron density. Y103 forms a hydrogen bond with D37 in UL141 while the same Y103 forms a polar interaction with the guanidino group of R132 in TRAIL. N134 of TRAIL-R2 interacts with T35 and T135 of UL141 and TRAIL, respectively. R133 forms a hydrogen bond with the main chain oxygen of UL141 while it forms no contact with TRAIL. These three interacting residues (Y103, R133 and N134) are also conserved in their nature in the other three TRAIL receptors, and this is shown by sequence alignments of several TNFRSF members (FIG. 13b). However, the AA″ loop in RANKL folds toward the top third of the molecule and is positioned above the β2β3 loop of the RANK receptor, whereas the same loop in LTα is very short and does not make any interaction with TNFRSF1A 23,27. Our mutagenesis data confirmed that these interactions (in patch 4, pink) are crucial for TRAIL binding and mimicked by UL141, as alanine mutations in this region of TRAIL-R2 completely abolished binding to both UL141 and TRAIL (FIG. 4c, pink). Moreover, deleting the AA″ loop in TRAIL completely abolishes its biological activity′. The combined structural and mutational data suggest that contact patch 4 is specific and crucial for TRAIL ligand binding and that viral UL141 mimics this structural motif to specifically engage this TRAIL DR.

### Example 9: UL141 Mimics a Hydrophobic Binding Motif Utilized by TNF-Family Ligands

In addition to UL141 mimicking the electrostatic interaction of TRAIL with TRAIL-R2 through the use of binding patch 4, UL141 also mimics a ‘TNF-specific’ hydrophobic binding motif located within binding patch 5 in the central region of CRD-2 (FIG. 4, Patch 5). This patch on TRAIL-R2 is formed by the hydrophobic residues of the β1β2 loop of CRD-2 (L110, L114 and F112) that cluster around Y148 of the GF loop of UL141 (connecting β-strands g and f). Similar interactions are formed by TRAIL, which utilizes Y216 to interact with L110 and L114 but not F112 of TRAILR2. These contacts are also conserved within other TNF-TNFR complexes and include Y108 in LTα, and 1248 in RANKL (called DE loop in TNF ligands) (FIG. 13). The aromatic interaction formed between Y148 of UL141 and F112 of TRAIL-R2 is critical for maintaining a stable complex, as the F112A mutation of TRAIL-R2 results in a 100-fold decrease in binding affinity (630 nM). In contrast, the F112A mutation does not affect TRAIL binding. However, double mutation of L110A and L114A abolished binding to TRAIL completely, while only a 7-fold decrease in binding affinity was observed for UL141 (43 nM). Therefore, the aromatic interaction involving F112 of TRAIL-R2 (which does not form a contact with TRAIL) dominates this binding interface with UL141, while TRAIL binding depends strongly on the hydrophobic interaction with both L110 and L114 of TRAIL-R2. In addition, both leucines are conserved or substituted with similar amino acids in other TNF-TNFR complexes (L110/L114 in TRAIL-TRAIL-R2, L67/L67 in LTα-TNFR1) (FIG. 13b). Moreover, Y216 in TRAIL has been identified by alanine scanning mutagenesis as a critical residue for bioactivity and receptor binding ²² and sequence comparison indicates its conservation in many of the TNF superfamily ligands including TRAIL, RANKL, TNFα, LTα and FasL (FIG. 13a). The importance of this tyrosine has also been shown by others, where mutation in TRAIL, RANKL, TNFα, LTα and FasL abolished receptor binding ^{21,28-31.} In summary, patch 5 involves strong hydrophobic features important for the stability of complexes throughout the TNF/TNFR superfamily. Without being limited to any particular theory, it appears that UL141 may have evolved to mimic this interaction in order to modulate this TRAIL DR.

### Example 10: Control of Cross-Reactivity Between TNF Superfamily Members

Patch 3 of TRAIL-R2 forms the most intensive interaction in the central to upper binding region with UL141. The contacts are maintained by CRD-3 β1β2 loop of TRAIL-R2, which interacts with a positively charged cluster of UL141 residues centered around K41, R80 and R82 of strands a and c, as well as R233 of helix X (FIG. 3b). Consequently, a positively charged pocket is formed by UL141 that engages the negatively charged glutamic acid residues of TRAIL-R2 (E151 and E147) through several salt bridges (FIG. 4, Patch 3, green). Sequence alignment reveals conservation of this region in TNFRSF10A-C, which covers all four TRAIL receptors, whereas the contacting residues of the cognate TNF ligands are spread across the entire sequence (FIG. 13).

In contrast to the UL141 interacting residues of patch 5, no residues within patch 3 are conserved in the other three TNF-TNFR complexes (FIG. 13), highlighting the complexity of the ligand-receptor binding in the superfamily. Moreover, mutagenesis in the participating β1β2 loop of the receptor was performed and it was found that mutation E151A had the most dramatic effect on both UL141 and TRAIL binding (no binding in SPR with up to 1 μM ligand). Therefore, the electrostatic network contained within patch 3 contributes to the binding specificity and stability and likely controls cross-reactivity among the different TNF superfamily members as well as ligand recognition.

### Example 11: TRAIL Specific Contacts within TRAIL-R2 not Mimicked by UL141

Patch 3T is adjacent to patch 3, but exclusively contacts TRAIL (FIG. 4, Patch 3T). It is maintained mostly by hydrogen-bond interactions within a range of 2.8-3.6 Å. Two separate TRAIL monomers from the homotrimer (CD loop in first subunit and EF loop of the second subunit) contact the CRD-3 β2β3 loop of TRAIL-R2. This patch was first identified in TRAIL-TRAIL-R2 complex as a major binding area ^{20,22} and it was reported that the CD and EF loops are disordered in the unbound TRAIL structure, while becoming ordered upon binding to TRAILR2. Alanine scanning of the TRAIL-R2 residues contained within patch 3T confirmed no effect on UL141 binding, while drastically reducing or abolishing TRAIL binding (FIG. 4c). The TRAIL Q205A mutant had previously been reported to have a 700-fold reduced binding affinity for TRAIL-R2 ²² and the present inventors have further extended this mutational analysis by looking at the TRAIL-R2 interface. Alanine scanning of residues M152, R154 and K155 abolished binding to TRAIL ectodomain completely, while having no effect on UL141 binding. While M152 bridges both TRAIL subunits, the adjacent K155 and R154 of TRAIL-R2 form most contacts with D203 and K201 of the opposing TRAIL subunit. The TRAIL-R2 M152A mutant reduced binding affinity to TRAIL by ˜50-fold (202 nM), suggesting a potential contribution of the sulfur atom in binding, while R154A (KD=46 nM) and K155A (KD=40 nM) mutants resulted in ˜10-fold weaker TRAIL binding. None of these TRAIL-R2 mutations affected UL141 binding. Without being limited to any particular theory, the receptors residues interacting in this patch (CRD-3 β1β2 loop) with the ligand may have an important role in controlling the specificity and cross-reactivity among the different TNF superfamily members, and therefore in ligand recognition, as these residues were not conserved in TNF ligand sequences (FIG. 13)

### Example 12: Accessible Surface for Receptor Binding on UL141

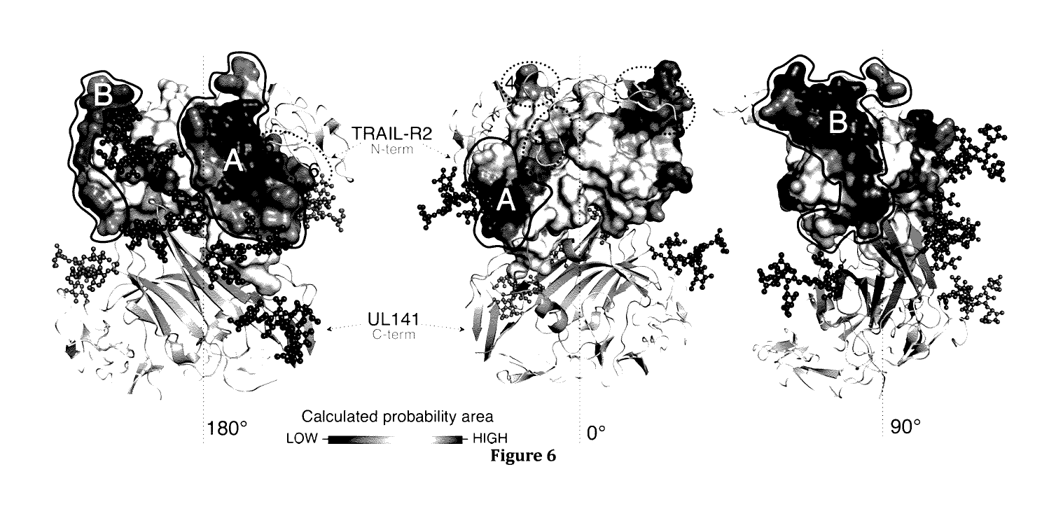

The UL141-TRAIL-R2 complex was not deglycosylated prior to crystallization, and all three putative N-glycosylation sites of UL141 display well-ordered electron density for N-linked carbohydrates (Asn117 and Asn147 of first subunit and Asn132 and Asn147 of the second subunit contained ordered carbohydrates). Modeling experiments predict that native, high-mannose glycosylation would not shield much of the UL141 surface from solvent, leaving ample space for binding to other ligands, such as CD155, assuming that complex glycans would project further outward into solvent (FIG. 6). Importantly, the experimental data indicate that UL141 can simultaneously bind to both TRAIL-R2 and CD155 (see Table 1), indicating the binding of multiple cellular proteins by a single UL141 dimer may have physiological relevance. Only the top of the (a, g, f, c, c′, c″)-β-sheet, as well as the front side of the C-terminal domain are expected to be largely covered with sugar in the fully glycosylated protein. For example, the solvent-exposed face of the (a, g, f, c, c′, c″)-β-sheet, the back face of C-terminal (1, 2, 3)-β-sheet domain and all three α-helices X, Y, and Z are devoid of glycans and available for other potential interactions. In addition, the present inventors have calculated predictions for the location of potential protein-protein binding sites for unbound UL141 using the ProMate server (http://bioinfo.weizmann.ac.il/promate) (FIG. 6). Interestingly, the highest binding area in UL141 is located on the back of the C-terminal domain, including surface exposed inward-facing of (c″, c′, c)-β-strands of the N-terminal domain and two α-helices (X and Z). The highest probability was also calculated for the actual TRAIL-R2 binding sites on UL141, thereby validating the approach. This analysis reveals that UL141 has two additional and separate binding sites that are suitable for protein binding (assessed using ProMate). Together with the present competition binding data indicating that UL141 does not compete with TRAIL-R2 for CD155 binding and without being limited to any particular theory, it appears that UL141 may use one of those two distinct surface-exposed binding sites within its two domains to bind to other proteins, such as the Ig superfamily member CD155. Recently, the crystal structure of another V-set Ig molecule TIGIT, bound to CD155³² has been determined. As UL141 recapitulates some of the structural features of TIGIT that are necessary for CD155 binding, superimposition of UL141 on TIGIT indicates that the potential binding site for CD155 on UL141 is indeed distinct from that of TRAIL-R2 and falls into the highest probability area A calculated by ProMate (FIG. 6, A).

### Example 13: Novel Ligand for TRAIL-R2

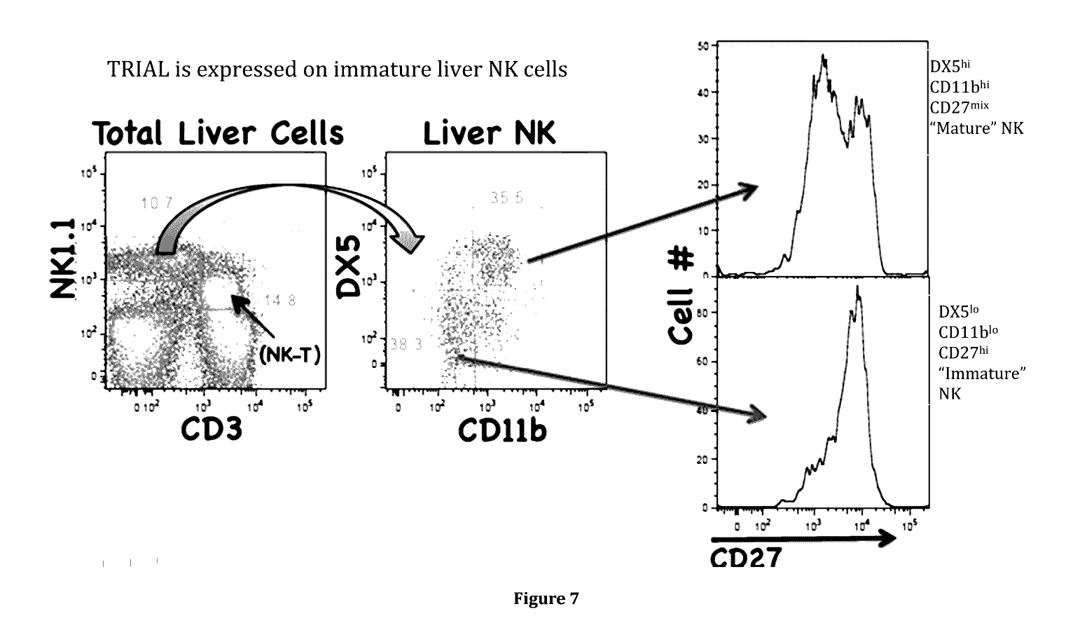

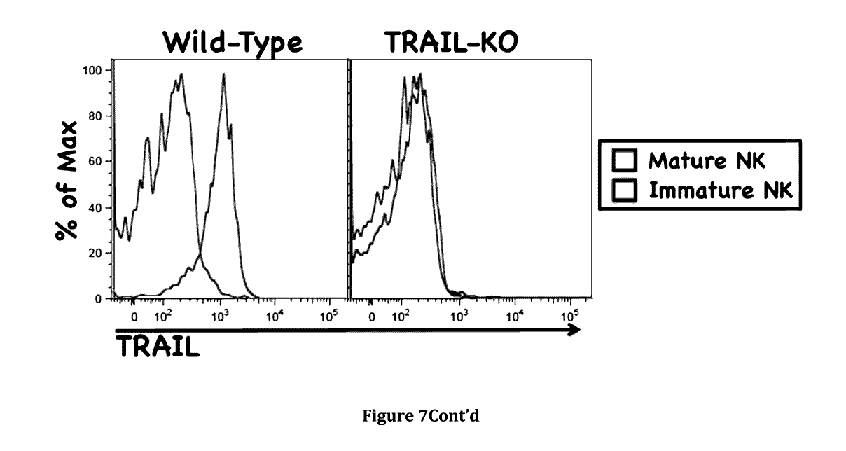

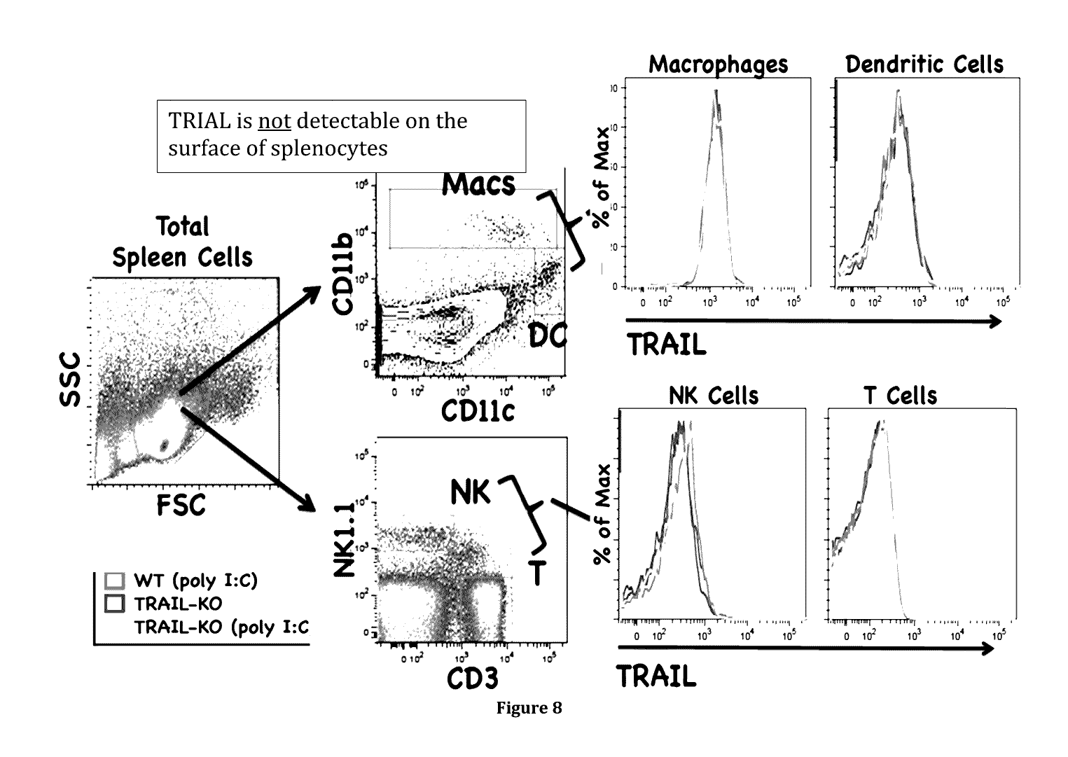

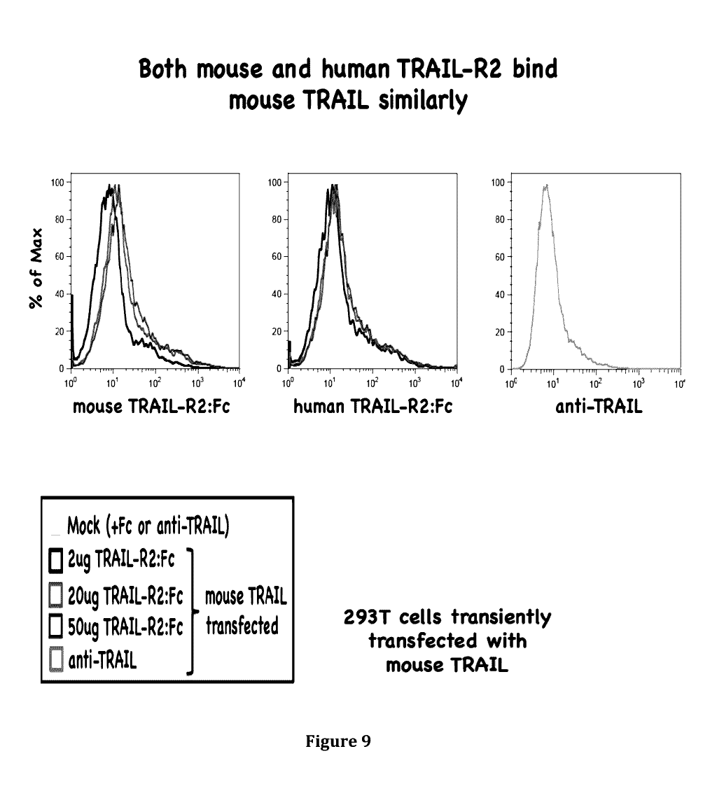

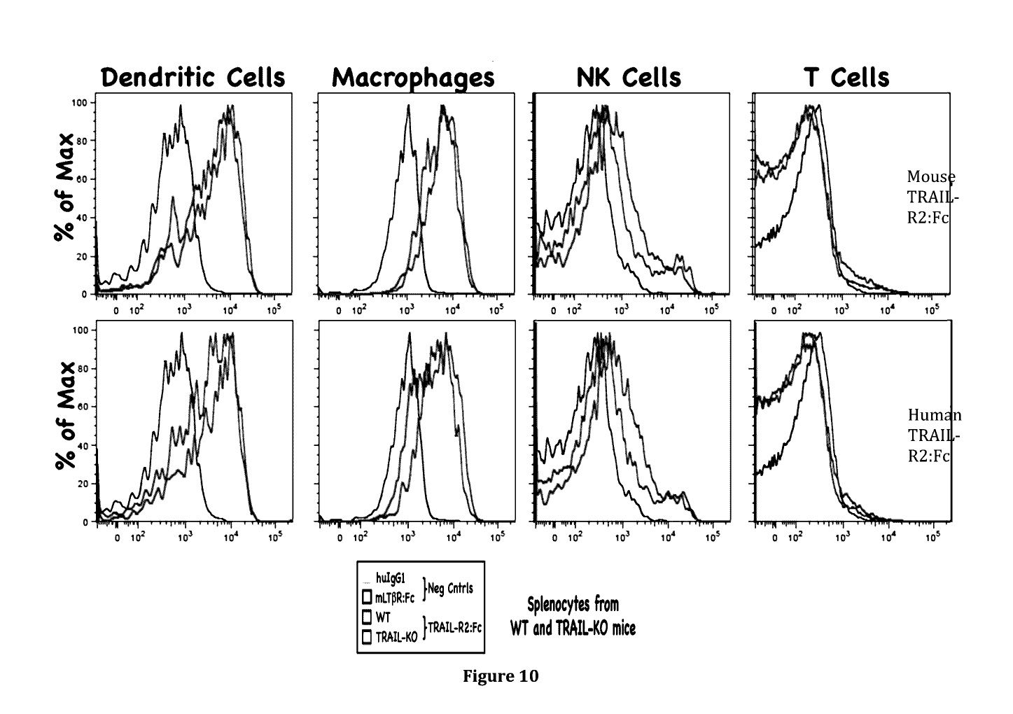

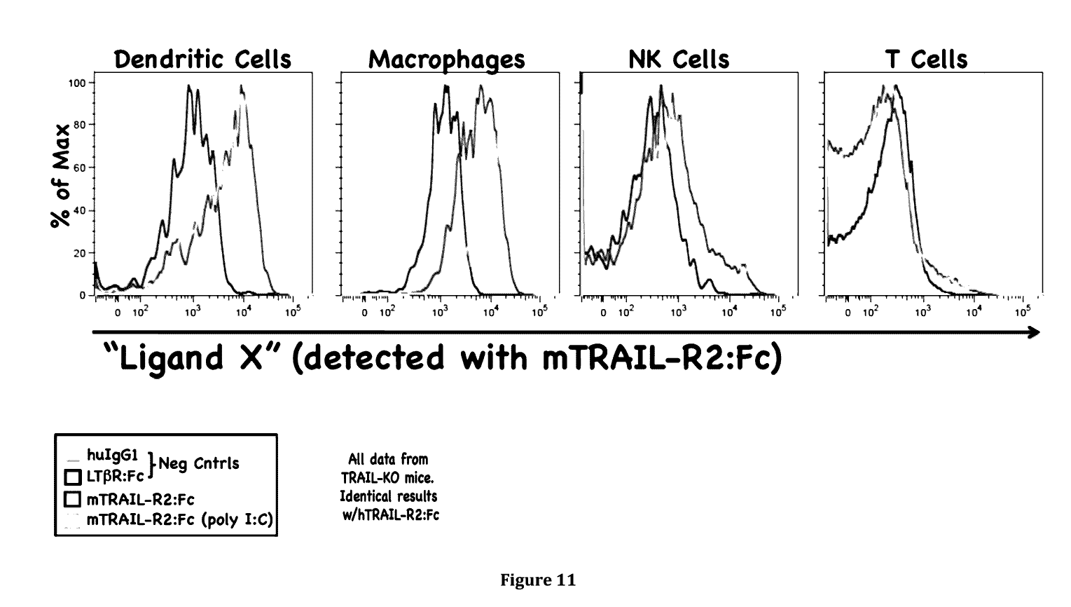

In view of the preceding data, the present inventors set out to further confirm the existence of an additional binding partner(s) for TRAIL-R. The inventors took advantage of mice that are genetically deficient for TRAIL (TRAIL-KO), allowing them to rule out any possible contribution of the known ligand for TRAIL-R2 in the experiments. As result of this approach, definitive data showing that additional binding partner(s) for TRAIL-R2 exist was obtained (FIG. 7-11)

### Example 14: Discussion of Results

Human cytomegalovirus encodes several genes tightly linked to UL141 in the UL/b′ region that modulate host immune responses mediated by TNF-family proteins. These include UL138, which has recently been shown to promote the expression of TNFR-1, and UL144, a partial mimic of HVEM (herpesvirus entry mediator) that exclusively binds the inhibitory receptor BTLA (B- and T-lymphocyte attenuator) 33. Although it is common for herpesvirus immune modulatory proteins to have evolved to target a specific protein, or a family of host proteins, targeting diverse proteins that contain unique folds is rare. The present invention now adds the modulation of the TRAIL DRs to arsenal of UL141 immune modulatory activity, in addition to its previously known role in restricting CD155 and CD112 expression. TRAIL DRs and CD155 belong to two structurally distinct families, the classical TNF receptor superfamily and the nectin-like Ig superfamily, respectively. While the only currently known natural ligand, TRAIL, belongs to the TNF superfamily, the structural analysis provided herein shows that UL141 assumes an Ig-like fold and shows no structural homology to TRAIL. However, UL141 does mimic key TRAIL binding motifs of TRAIL to TRAIL-R2, even though the Ig-fold of UL141 is entirely different from the homotrimeric fold of TRAIL and other TNF ligands. Since UL141 and the Ig-like poliovirus receptors share no primary sequence homology, without being limited to any particular theory, it appears that UL141 may have evolved independently, mimicking the central binding motif of TRAIL in addition to an as-yet unidentified binding motif to engage CD155.

The present structural and biochemical data further reveal that the TRAIL-R2 binding site on UL141 only partially overlaps with that of the endogenous ligand TRAIL, and appears to be entirely distinct from that which interacts with CD155 (FIG. 2 and Table 1). Due to the strong sequence similarity of the TRAIL DRs, one might propose that TRAIL-R1 is likely to bind UL141 similarly to TRAIL-R2. However, the binding affinity of TRAIL-R1 is ˜400-fold reduced compared to that of TRAIL-R2, similar to the large differences in binding affinity that have been observed for TRAIL binding to its two death receptors ³⁴. In lack of a TRAIL-R1 crystal structure, the sequence-similarities between TRAIL-R1 and -R2 and based on the sequence conservation between TNFR-fold proteins were analyzed, and the results suggest that UL141 uses the same surfaces to interact with TRAIL-R1 and TRAIL-R2 receptor.

Viral glycoprotein UL141 is now known to be required for restricting the cell surface expression of four cellular proteins, including TRAIL-R1, TRAIL-R2, CD155 and CD112. As CD155 was the first identified target of UL141¹⁶, the increased sensitivity of cells infected with a HCMVΔUL141 mutant to NK-killing was initially ascribed solely to inhibiting DNAM-1/CD226 activation, which also binds CD112 ¹³. However, NK cells also express high levels of TRAIL when activated by interferons during viral infection ³⁵, and it is a likely possibility that the potent NK inhibition by UL141 is due to its dual role in modulating multiple effector pathways such as DNAM-1/CD226 NK cell activation as well as TRAIL-mediated killing 12.

Viral manipulation of the immune response is typically achieved by virulence factors, which often imitate the function of a host protein by mimicking its key structural features ^{36,37}. One possibility is that a virus first hijacks a host gene(s), and then further evolves/selects those genes for specific functions to target host immune signaling pathways ³⁸. In this case, virulence factors and host proteins would be derived from the same origin, and differences in the structure and/or function of the viral ortholog would arise by divergent evolution. However, structural mimics can also be generated through convergent evolution ³⁶. Although differing in evolutionary origin and three-dimensional structure ³⁹, in this case virulence factors evolve to mimic key structural features of cellular proteins. Examples of the latter strategy, which can only be revealed through structural analysis, are fewer than those that can be identified by primary sequence similarity ^{37,40,41}. While UL141 does not display any sequence homology to other proteins in the database, a DALI search (http://ekhidna.biocenter.helsinki.fi/dali_server), identified significant structural conservation with Ig-domain proteins, including T cell receptors, MHC molecules and immunoglobulins (500 proteins with a Z-score of 8.1-9.6 and RMSD 2.4-4.7 Å). The structural conservation is limited to the Ig-domain of UL141, while no homology found in the C-terminal domain (residues 160-246), indicating this domain adopts a unique structural fold. Interestingly, while the top hit of the DALI search corresponds to a variable TCR chain (Z=9.6, RMSD=3.1 over 50% TCR chain sequence), the second hit was the HCMV protein UL16, an immunoevasin that subverts NKG2D-mediated immune responses by retaining a select group of NKG2D ligands inside the cell³⁶. UL16 aligns with 85% of its structure to the Ig-domain of UL141 (Z=9.4, RMSD=3.7). However, while the top two structural homologs of UL141 (TCR and UL16) both bind to Ig-domain MHC-like molecules, UL141 has also evolved to target the TNFRs, illustrating the functional versatility of the Ig-fold.

The structural and binding data presented herein is the first for a viral glycoprotein that directly binds to both a TNFR and an Ig-domain protein. Currently, the only other known example of a TNFR binding to an Ig molecule is HVEM-BTLA, which was recently structurally characterized ³³. HVEM also binds the TNF-family ligand LIGHT. HVEM-BTLA interaction can lead to both inhibition of immune cells through BTLA signaling and activation through HVEM, while LIGHT binding to HVEM is thought to exclusively mediate co-stimulatory signals ⁴². Notably, UL144, the HVEM ortholog encoded by HCMV, has evolved to only bind BTLA and not LIGHT, and this has resulted in it being an extremely potent inhibitor of T cell activation ⁴³. Consequently, both UL144 and UL141 have evolved to target non-canonical interactions of TNFRs with Ig-domain proteins. The structural analysis provided herein has revealed that HCMV has evolved the pleiotropic UL141 as a potent inhibitor of at least two different immune effector pathways, the TRAIL DRs and nectin-like NK cell activating ligands. The present invention provides new insights into the structural basis of the evolutionary dynamic that exists between persistent viruses and host defenses, exemplified by the promiscuous targeting of immune effector pathways by UL141.

### Example 15: Low-Passage HCMV Strains Inhibit Cell Surface Expression of the TRAIL Death Receptors

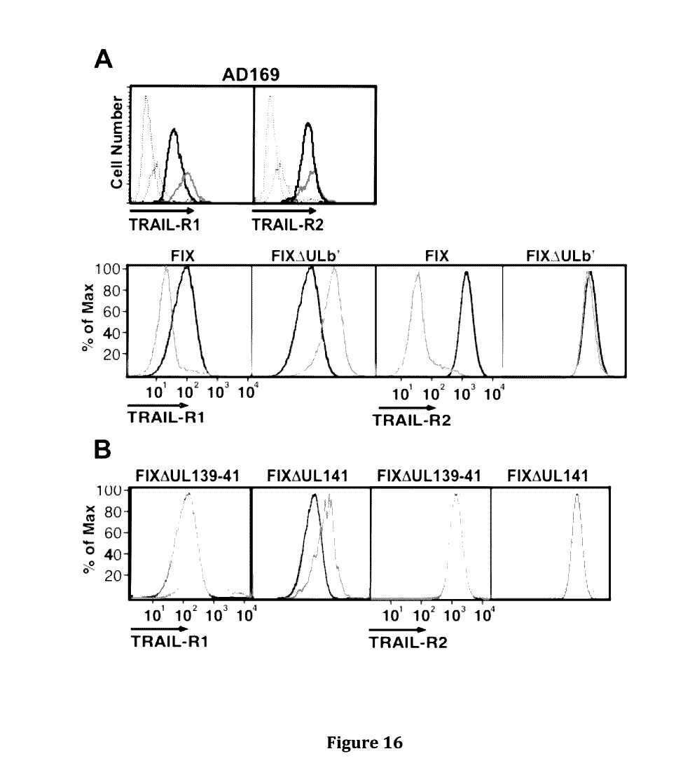

HCMV is known to inhibit signaling by DRs belonging to the TNFR superfamily (e.g. TNFR-1 and Fas) (Baillie et al., 2003; Jarvis et al., 2006; McCormick et al., 2003; Skaletskaya et al., 2001b). However, HCMV isolates that have been passaged extensively in cultured fibroblasts (e.g. AD169 strain) can differentially alter TNFR expression due to the loss of specific immune modulatory proteins (Le et al., 2011; Montag et al., 2011). Consequently, to address whether infection with HCMV would target the TRAIL DRs, fibroblasts infected with distinct viral strains were analysed for their cell surface expression. The high-passage laboratory strain AD169 was used for infection (variant ATCC), as well as the FIX strain of HCMV (originally VR1814) which has been subjected to limited in vitro passage, and whose genome is available as an infectious clone in a bacterial artificial chromosome (BAC) (Hahn et al., 2002; Murphy et al., 2003). In contrast to AD169, FIX induced dramatic downregulation of both TRAIL DRs from the cell surface (FIG. 16A). The function was ascribed to a de novo FIX-encoded gene product, as inhibition of DR expression was ablated by UV-irradiation of input virus (data not shown). Low passage HCMV strains therefore encode a function that downregulates cell surface expression of TRAIL-R1 and TRAIL-R2 that has been lost from the laboratory strain AD169.

### Example 16: UL141 is Implicated in the Inhibition of TRAIL-R2 Expression

In addition to other defects, strain AD169 has suffered a spontaneous 15 kb deletion from the right hand end of the UL region (UL/b′) during passage in vitro (Cha et al., 1996). Consequently, a HCMV mutant generated in the FIX-BAC deleted in the majority of the UL/b′ sequence (FIXAUL/b′(Hahn et al., 2004)) was utilized to test for the ability to restrict TRAIL DR expression. The UL/b′ region contains≧21 genes that are dispensable for viral replication in fibroblasts (Gatherer et al., 2011). FIXAUL/b′ could not restrict cell surface expression of either TRAIL DR, indicating that an HCMV gene contained within this region was required for their inhibition (FIG. 16A). Notably, cell surface expression of TRAIL-R1 was significantly increased after FIXAUL/b′ infection, and this was consistent with enhanced mRNA expression levels seen for this DR in HCMV infected fibroblasts.

Screening through the UL/b′ region, using a panel of pre-existing FIX BAC deletion mutants (Hahn et al., 2004), ruled out UL128, UL129, UL130, UL131a, UL132, UL148ad, C-orf23, C-orf25, C-orf26 in regulating the TRAIL DRs (data not shown). A FIXΔ139-141 mutant was then constructed, and when tested this mutant was incapable of inhibiting TRAIL DR expression, with TRAIL-R1 being commensurately upregulated on the cell surface similar to that seen with FIXAUL/b′ (FIG. 16B). The UL139, UL140 and UL141 genes were then individually disrupted, and revealed that an HCMV FIX mutant lacking an intact UL141 orf was incapable of downregulating cell surface expression of the TRAIL DR (FIG. 16B). Taken together, these results show that UL141 is required to restrict cell surface expression of TRAIL-R1 and -R2 in HCMV infected cells.

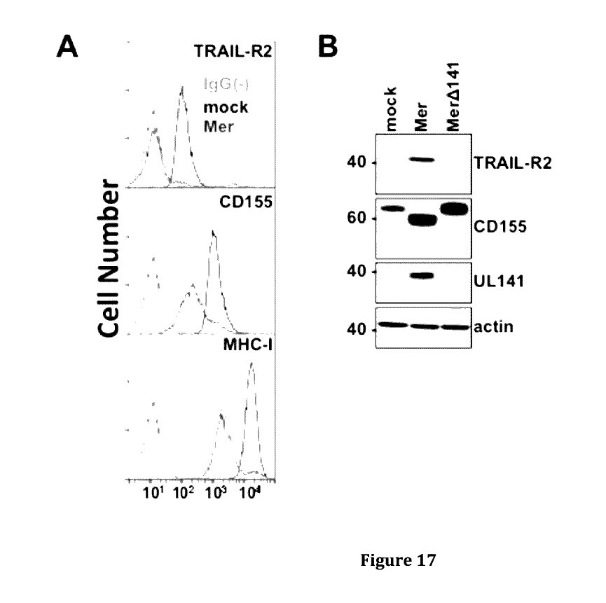

A high level of sequence variation is present in HCMV clinical isolates and cultured strains, although it is not evenly distributed throughout the genome, and this variability has been shown to impact immune evasion functions (Prod'homme et al., 2012). To further examine and confirm the role of UL141 in regulating TRAIL-R1 and TRAIL-R2 expression, the gene was also specifically deleted from HCMV strain Merlin using BAC technology. Consistent with previous findings, UL141 was required for downregulation of CD155 by strain Merlin, but not for inhibition of MHC-I (FIG. 17A). Whereas deletion of UL141 from the FIX strain resulted in restoration of TRAIL-R2 level to those seen in uninfected cells (FIG. 16B), in the Merlin strain restoration was never complete, albeit expression levels returned to levels>90% that of mock. Consequently, while UL141 in both strains clearly targets TRAIL-R2, a difference may exist in the overall regulation of this DR by FIX and Merlin that is UL141-independent, and this is currently being explored.

### Example 17: Expression Kinetics of UL141

Suppression of TRAIL-R2 cell surface expression by HCMV could be detected as early as 24 h post infection, yet became more marked as the infection progressed through 48 and 72 hours. In strain FIX, UL141 is encoded by a single abundant transcript initiated 213 bases upstream of the start codon and extending to 39 bases downstream of the stop codon, compatible with recent transcriptional mapping data for UL141 in strain Merlin (Gatherer et al., 2011). Consistent with the kinetics of TRAIL-R2 downregulation, strain FIX UL141 was found expressed as an early-late gene product, increasing in abundance dramatically throughout the viral replication cycle

### Example 18: Fate of TRAIL-R2 in HCMV Infected Cells

The inventors have previously shown that UL141 restricts the cell surface expression of CD155 and CD112, two NK cell activating ligands belonging to the nectin/nectin-like family of proteins. Notably, the mechanisms by which UL141 modulates these two host cell proteins are quite distinct. UL141 sequesters CD155 in the endoplasmic reticulum (ER) of HCMV-infected cells (Tomasec et al., 2005), yet promotes the proteasomedependent degradation of CD112 (Prod'homme et al., 2010). To gain insight into what mechanism(s)-of-action may be utilized by UL141 to target TRAIL-R2, strain Merlin infected fibroblasts were analyzed by western blot. Notably, fibroblasts infected with Merlin showed demonstrably higher total cellular levels of TRAIL-R2 when compared to uninfected cells, or cells infected with MerΔUL141 (FIG. 17B). A similar pattern of restriction of cell surface expression, but enhanced total cellular expression, of TRAILR2 was also observed in both epithelial cells and glioblastoma cells infected with HCMV (data not shown). In total, these data indicate that while UL141 functions to inhibit cell surface expression of TRAIL-R2 in HCMV-infected cells, it appears to promote the accumulation of this DR in an intracellular compartment.

### Example 19: UL141 is Sufficient to Restrict TRAIL Death Receptor Expression

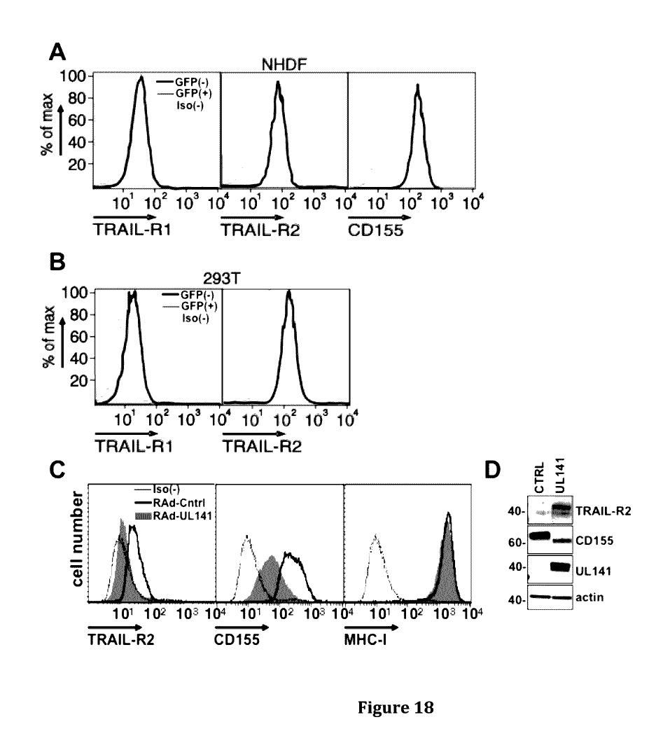

Studies with HCMV deletion mutants clearly demonstrated that UL141 was required to provide efficient downregulation of the TRAIL DRs at the cell surface by both the FIX and Merlin strains. UL141 alone is sufficient to restrict CD155 cell surface expression (Tomasec et al., 2005), but additional HCMV-encoded functions were needed to target CD112 (Prod'homme et al., 2010). It was therefore sought to determine whether UL141 was able to target TRAIL DRs when expressed in isolation. A UL141 expression plasmid was transfected into both primary fibroblasts and 293T cells, and significant downregulation of TRAIL-R1 and -R2 was observed, proving that UL141 alone is sufficient to suppress TRAIL DR expression (FIGS. 18A and B). Inhibition of TRAIL DRs was also observed when a UL141-GFP fusion protein was stably transfected into 293T cells, with an enhanced accumulation of intracellular TRAIL-R2, as observed in HCMV-infected cells. In addition, transduction of human fibroblasts with a recombinant adenovirus encoding UL141 also promoted a reduction of TRAIL DR cell surface levels, combined with enhanced intracellular retention, indicative of UL141 protecting TRAIL-R2 from proteolytic degradation (FIGS. 18C and D). Together these experiments demonstrated that UL141 can inhibit cell surface expression and promote intracellular accumulation of the TRAIL DR without the assistance of any additional HCMV-encoded function.

### Example 20: UL141 Interacts Directly with the Human TRAIL Death Receptors

UL141 is a type I transmembrane glycoprotein with a short C-terminal cytoplasmic domain, and structural algorithms predict it contains an immunoglobulin-like fold in its ectodomain (Tomasec et al., 2005). To determine whether UL141 targets the TRAIL DRs by directly binding to them, the UL141 ectodomain (UL141ecto) was expressed and purified as well as a fusion protein of the ectodomain with the Fc region of human IgG1 (UL141:Fc). The binding assay demonstrated an interaction between UL141:Fc and both TRAIL-R1:Fc and TRAIL-R2:Fc. While UL141:Fc binds to the surface of human fibroblasts and 293T cells (data not shown), this result was not informative as CD155 is also expressed at high levels on most human cells. In contrast, UL141:Fc was incapable of binding to mouse NIH-3T3 fibroblasts (which express mTRAIL-R2), but transfection of hTRAIL-R2 into NIH 3T3 cells promoted strong binding of UL141:Fc, formally showing that UL141 can interact with cell-surface-expressed hTRAIL-R2. Consistent with this result, binding was not observed between mouse TRAIL-R2:Fc and UL141:Fc in an ELISA-based assay (data not shown). Taken together, both of these approaches show that UL141 binds directly to both of the human TRAIL DRs.

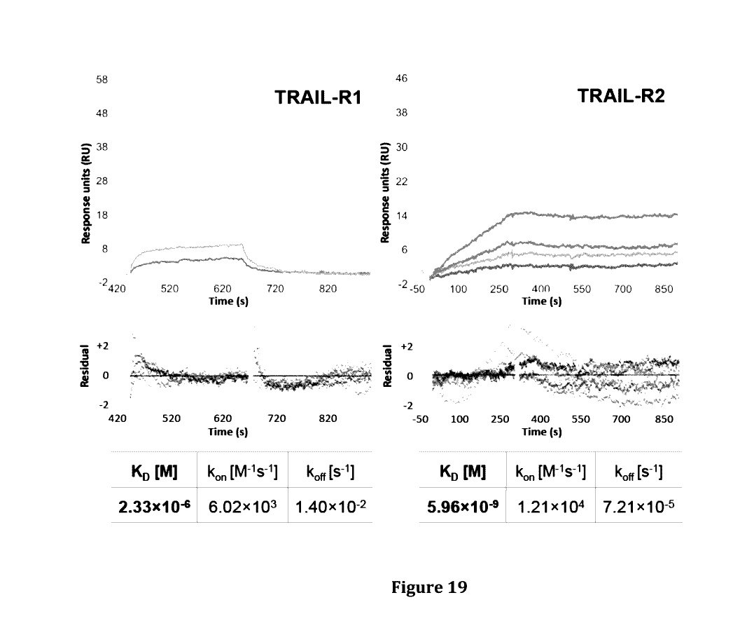

In order to determine the binding kinetics/affinity of UL141 for the TRAIL DRs, surface plasmon resonance analysis of UL141ecto binding to TRAIL-R1 and -R2:Fc proteins was conducted (FIG. 19). UL141 was found to bind to TRAIL-R2 with a KD of 6 nM, an affinity very close to that of TRAIL (2 nM, (Truneh et al., 2000)). In contrast, UL141 bound to TRAIL-R1 with a dramatically lower affinity (KD=2.3 μM), with differences in both the association and dissociation kinetics being observed. The UL141 binding kinetics to TRAIL-R1 revealed only a 2-fold slower association rate (kₒₙ=6.0×10³M⁻¹s⁻¹), while dissociation was almost 200-times faster (k_{off}=1.4 Å˜10⁻²s⁻¹), when compared to UL141 binding to TRAIL-R2-Fc (kₒₙ=1.2 Å˜10⁴M⁻¹s⁻¹, k_{off}=7.2 Å˜10⁻⁵s⁻¹, FIG. 19). Taken together, these results prove that the ectodomain of UL141 binds directly to the ectodomains of both human TRAIL DRs, and displays a significantly lower affinity for TRAIL-R1, mimicking what has been observed for TRAIL binding to its two cognate DRs (Truneh et al., 2000).

The fact that UL141 interacts directly with the TRAIL DRs is interesting, as UL141 shows no primary-sequence or predicted structural homology to any TNF-family ligands (Bodmer et al., 2002). This raised the possibility that UL141 might interact with additional members of the TNFR superfamily. To test this, UL141:Fc protein was used to stain 293T cells transfected with all the known TNFRs, and both positive and negative controls were included to verify that the TNFRs were expressed and functionally capable of binding their cognate TNF-family ligands (Bossen et al., 2006). In this assay format, binding of UL141:Fc (˜5 μg/ml) was only detected to 293T cells transfected with TRAIL-R2, which strongly bound the HCMV protein. Notably, binding of UL141:Fc under these conditions was not detected to 293T cells transfected with TRAIL-R1, most likely due to the relatively low binding affinity for this DR compared to TRAIL-R2, as demonstrated by our SPR analysis. Consequently, these data indicate that TRAIL-R2 appears to be the only member of the TNFR superfamily that is a specific, high affinity target for UL141.

### Example 21: Intracellular Retention of TRAIL-R2 in the Presence of UL141

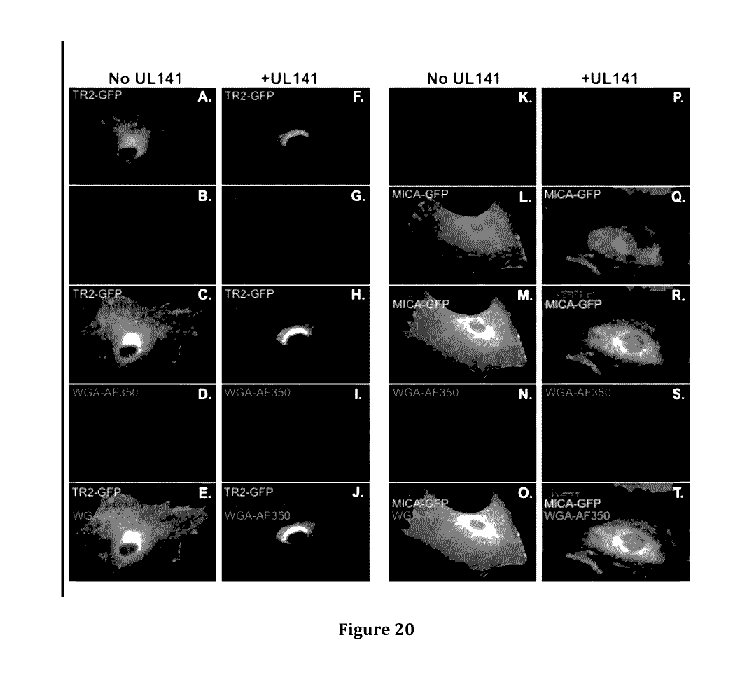

Expression of TRAIL DRs is localized in large part to intracellular membrane compartments in lung and melanoma-derived cell lines, with a minority of the total protein localized to the plasma membrane at steady state (Leithner et al., 2009; Zhang et al., 2000). Virtually nothing is known regarding the mechanisms that regulate the trafficking of TRAIL DRs through various cellular compartments, although ER stress has been shown to upregulate TRAIL-R2 cell surface levels and sensitize them to TRAIL induced killing (Chen et al., 2007). To examine whether UL141 alters the subcellular localization of TRAIL-R2, fibroblasts were transduced with adenoviral recombinants (RAd) encoding UL141 and TRAIL-R2 constructs fused to C-terminal GFP or RFP tags and lacking an intact death domain (averting apoptosis mediated by overexpression of full-length TRAIL-R2) (e.g. RAd-TRAILR2.GFP)(FIG. 20). For comparison, cells were also transduced with RAd-CD155.RFP or RAd-MICA.GFP. TRAILR2.GFP and TRAILR2.RFP were expressed throughout a variety of intracellular membrane compartments, on the cell surface, and co-localized with endosomal markers (FIGS. 20A, 20K). In contrast, when this DR and UL141 were co-expressed, TRAIL-R2 was restricted in large part to the ER, (FIGS. 20F and P and not shown). This pattern of intracellular compartmentalization was similar to that observed in cells transduced with RAd-CD155.RFP and UL141 (FIGS. 20G and H). The interaction between TRAIL-R2 and UL141 was specific, as UL141 did not alter trafficking/localization of MICA.GFP (FIGS. 20P and Q), which is known to be downregulated from the cell surface through the action of HCMV UL142 (Ashiru et al., 2009; Chalupny et al., 2006). Taken together, these data support biochemical analyses showing that UL141 redirects and/or restricts TRAIL DR expression to an intracellular membrane compartment(s).

### Example 22: UL141 Functions Non-Redundantly to Restrict TRAIL-Mediated Killing

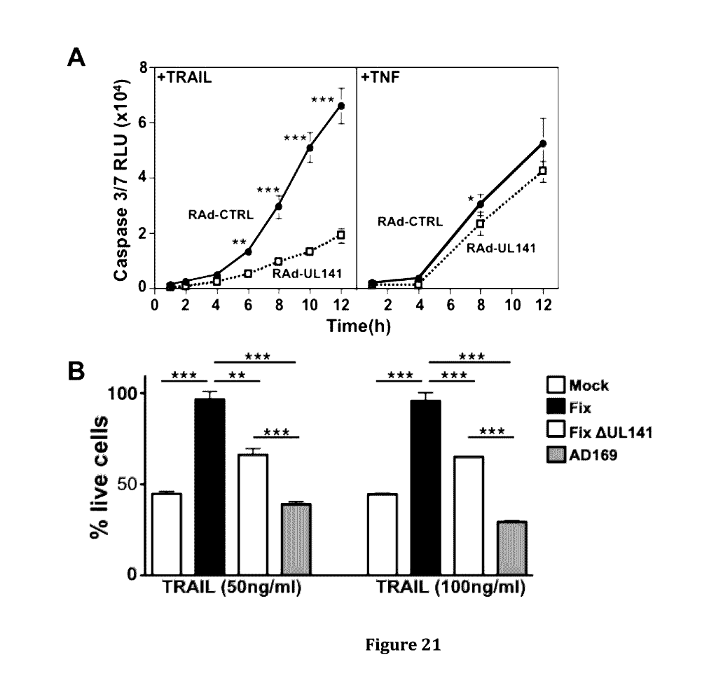

The present inventors sought to investigate whether the intracellular sequestration of TRAIL-R2 by UL141 desensitized cells to TRAIL-mediated apoptosis. To test this, human fibroblasts transduced with UL141 were treated with soluble TRAIL (FIG. 21A). UL141-expressing cells showed dramatically reduced activation of Caspase-3/7, proving that UL141 can desensitize cells to apoptosis mediated by the TRAIL DR. This effect was specific, as the sensitivity of UL141-expressing cells to TNF-mediated apoptosis was not overtly altered (FIG. 21A).

Next, the effect that UL141 restriction of TRAIL DR cell surface expression had on altering the sensitivity of HCMV infected cells to TRAIL killing was analyzed (FIG. 21B). Fibroblasts infected with FIX were completely protected from TRAIL-mediated killing. In contrast, FIXΔUL141 infected cells were significantly more sensitive to TRAIL-induced apoptosis, which was notable, as other potentially redundant mechanisms targeting DR signaling are still operable in this mutant virus (e.g. UL36-mediated inhibition of caspase-8 activation (Skaletskaya et al., 2001a). To further explore this issue, the sensitivity of cells infected with HCMV strain AD169 to TRAIL killing was analyzed, as this strain encodes a non-functional UL36 (Skaletskaya et al., 2001a) in addition to lacking UL141. Notably, strain AD169-infected fibroblasts were even more sensitive to TRAIL killing than those infected with FIXΔUL141, consistent with both UL36 and UL141 contributing to the inhibition of TRAIL DR signaling. Taken together, these studies demonstrate that UL141 restriction of TRAIL DR cell surface expression provides non-redundant protection against TRAIL-mediated apoptosis in HCMV infected cells.

### Example 23: UL141 Inhibition of TRAIL DRs Contributes to NK Cell Inhibition

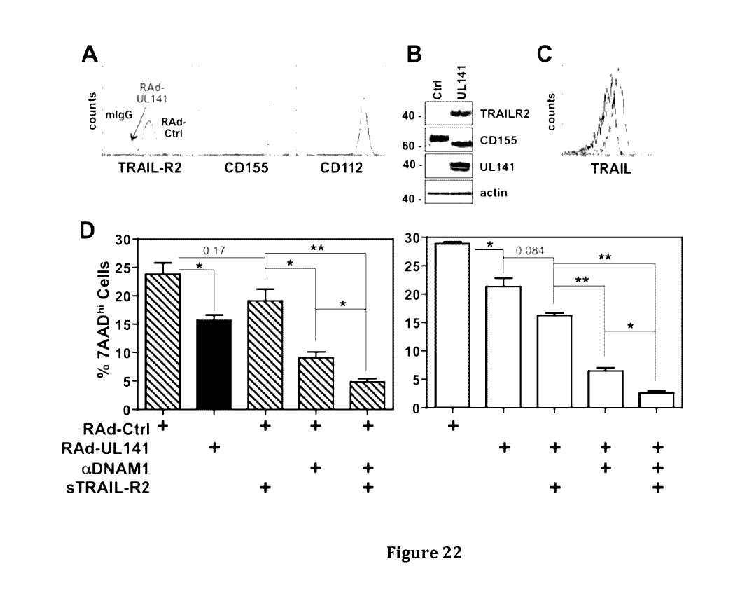

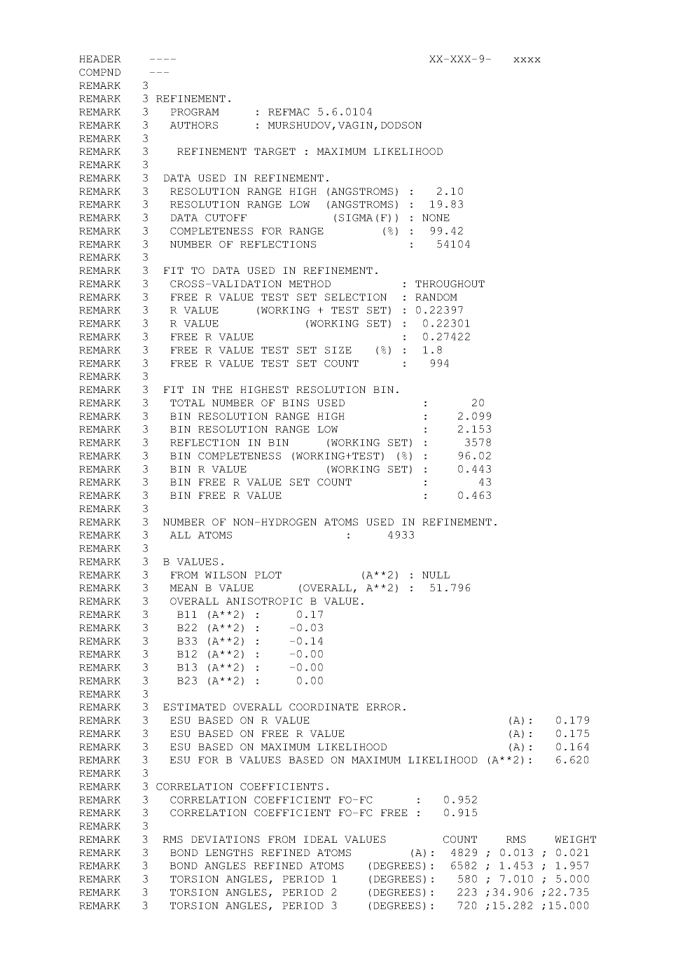

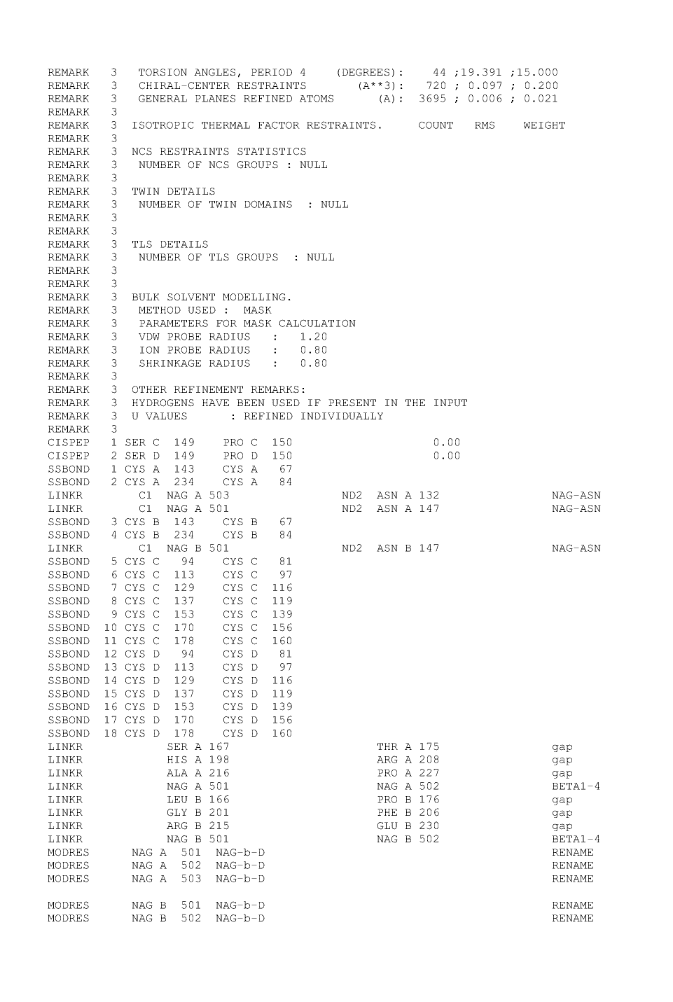

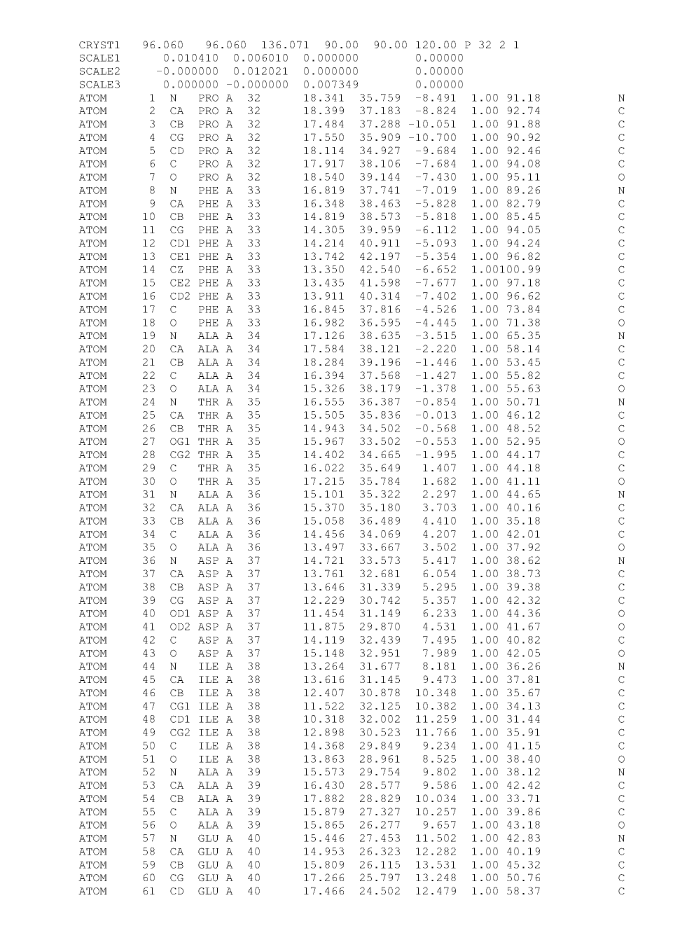

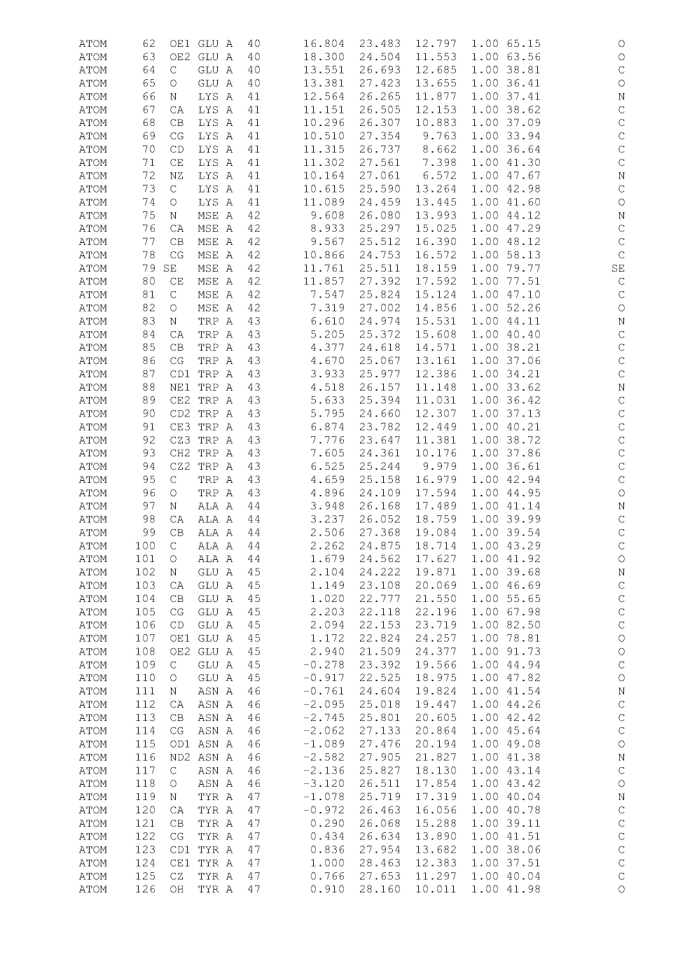

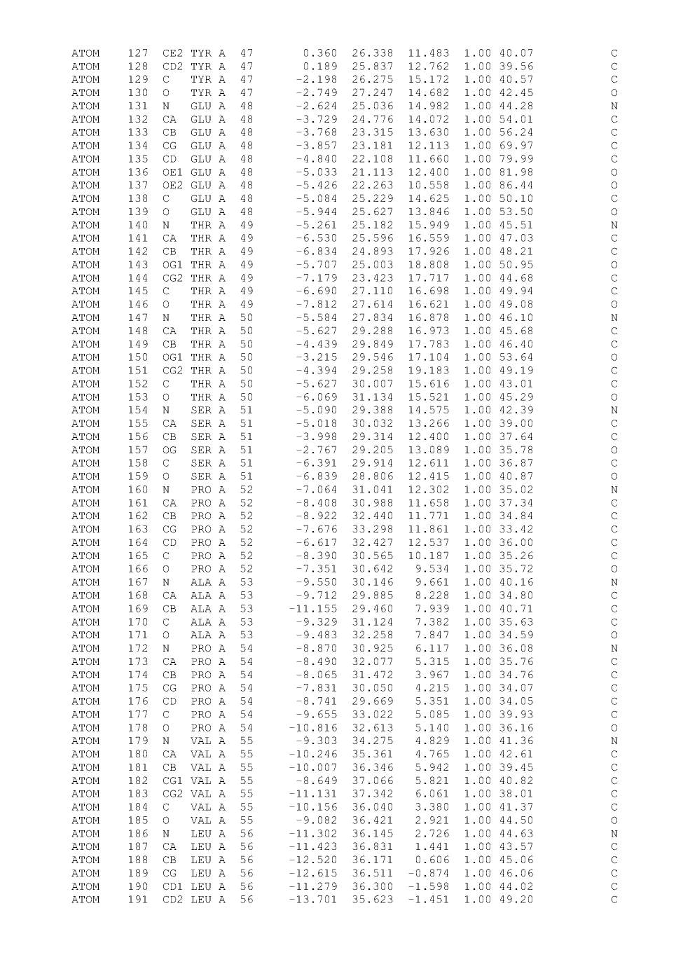

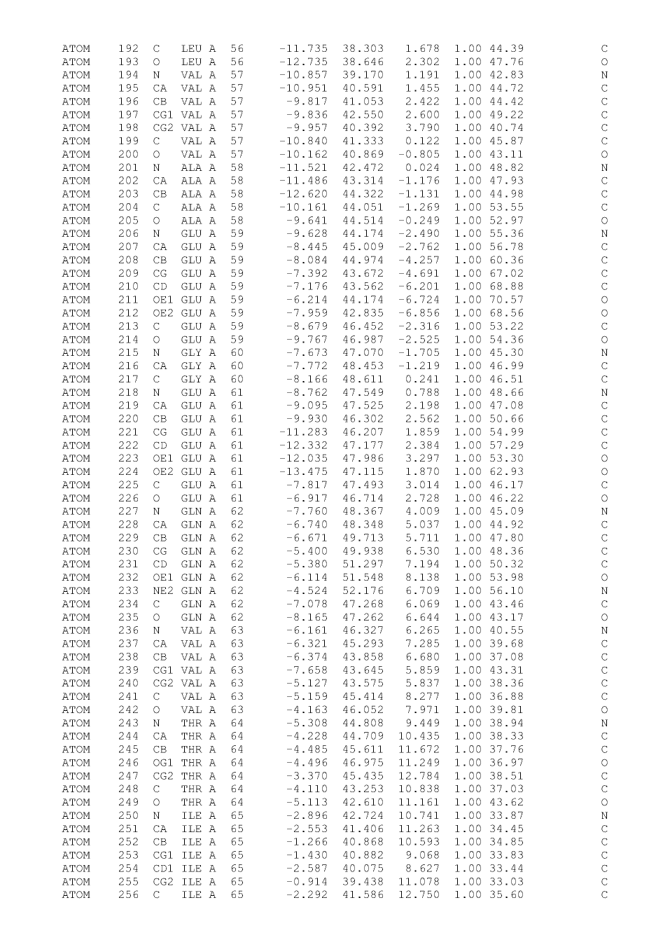

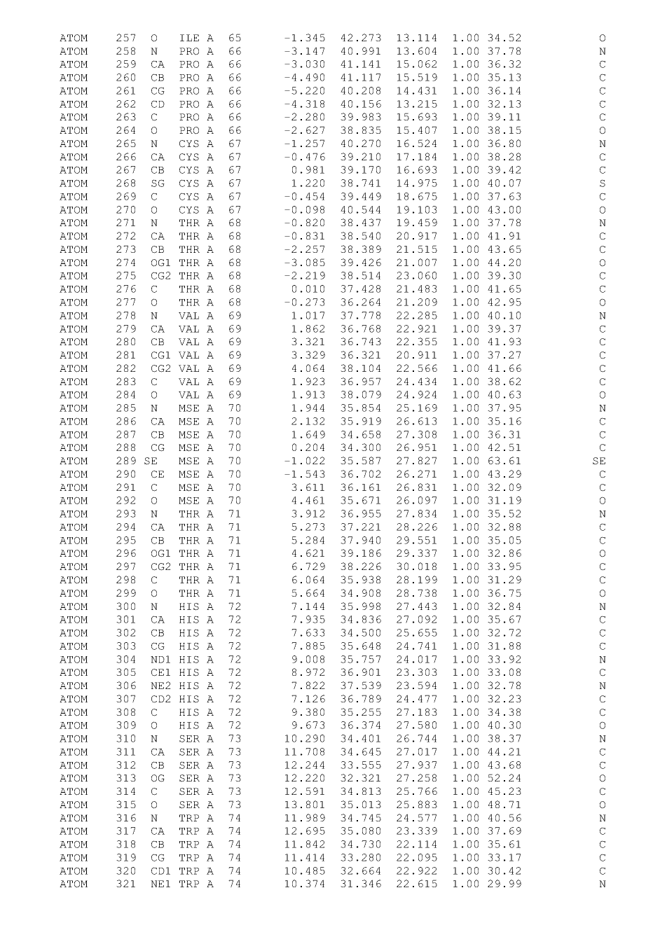

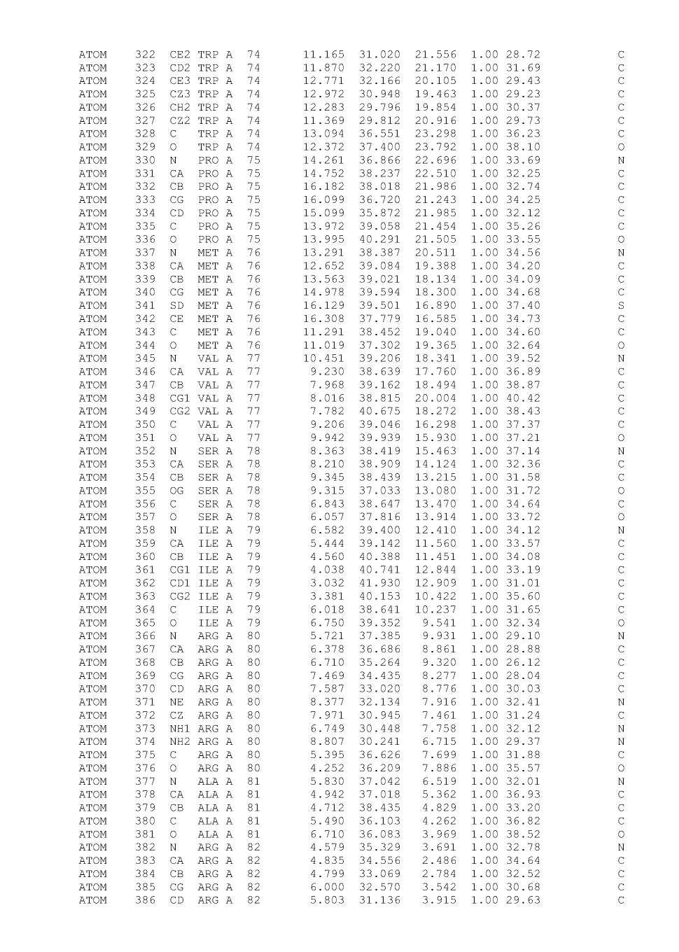

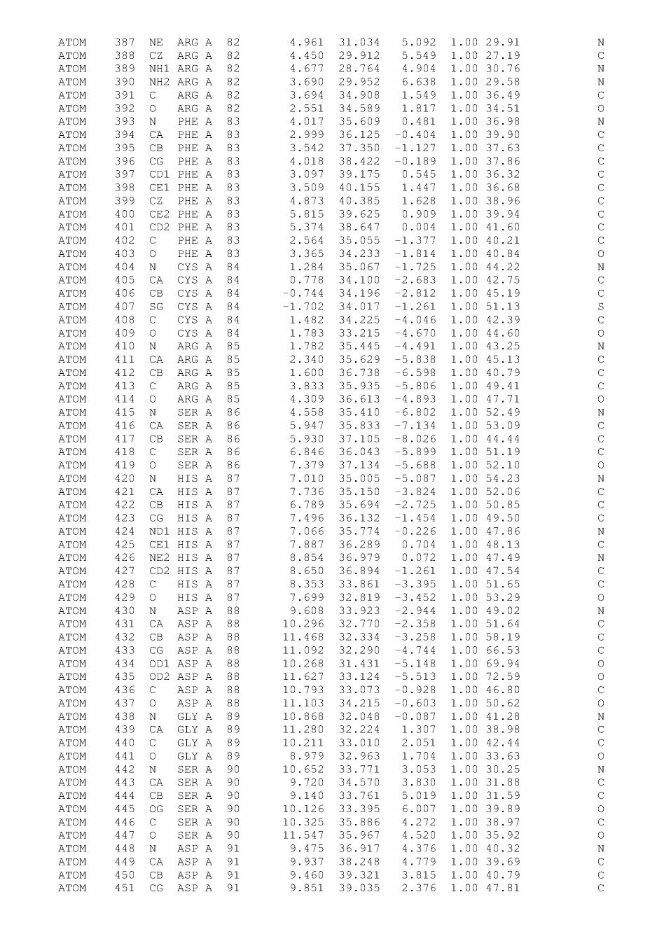

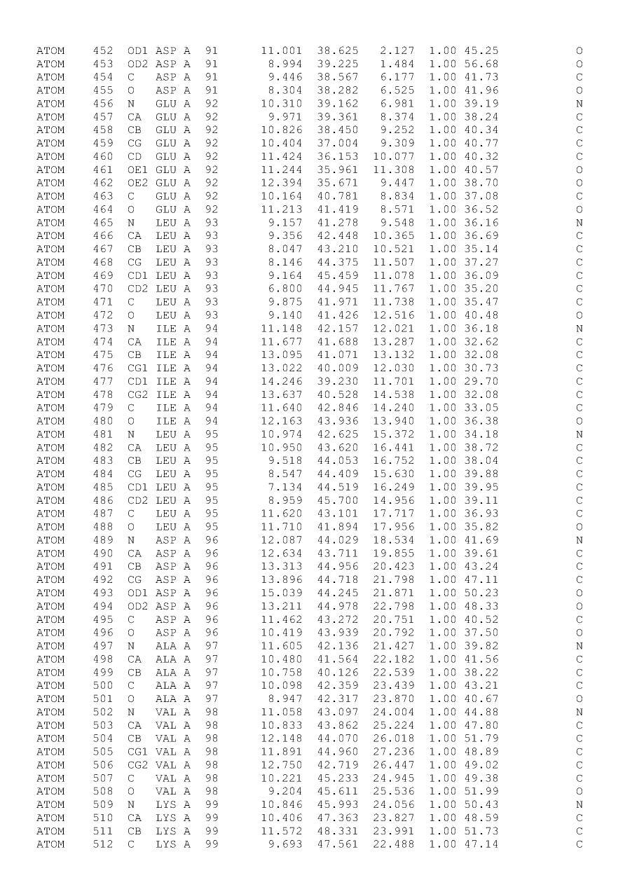

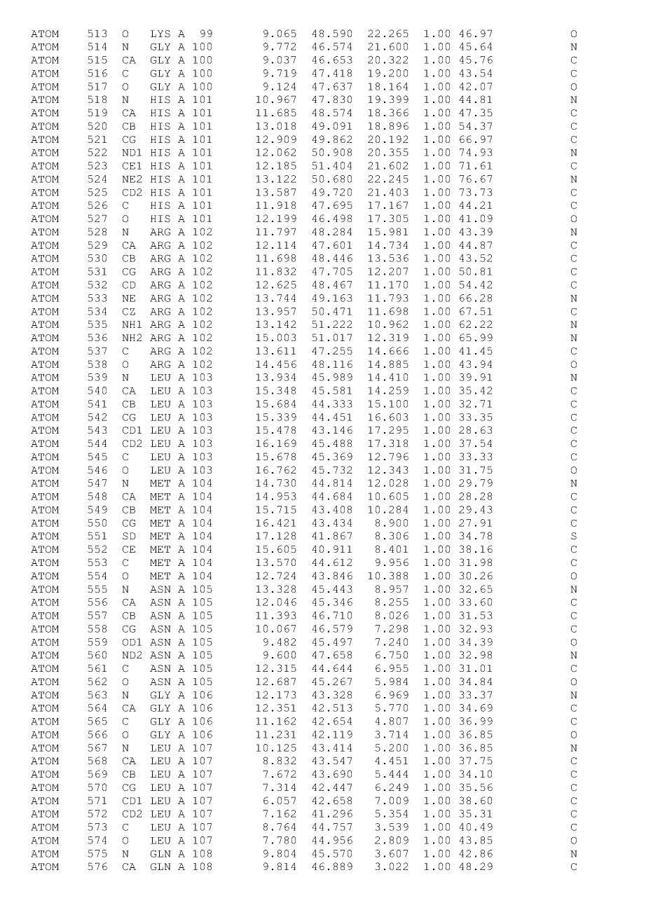

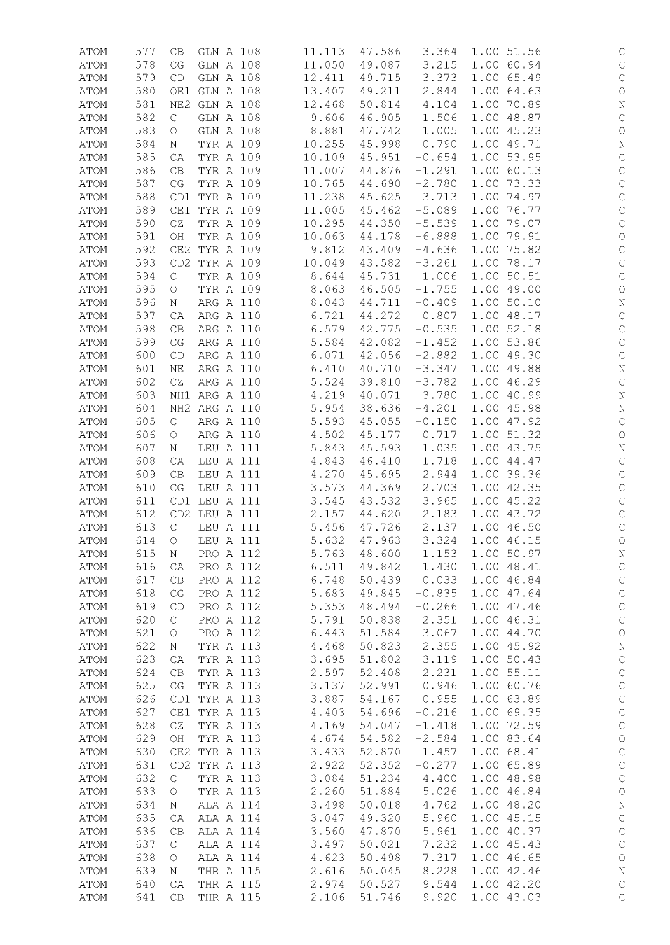

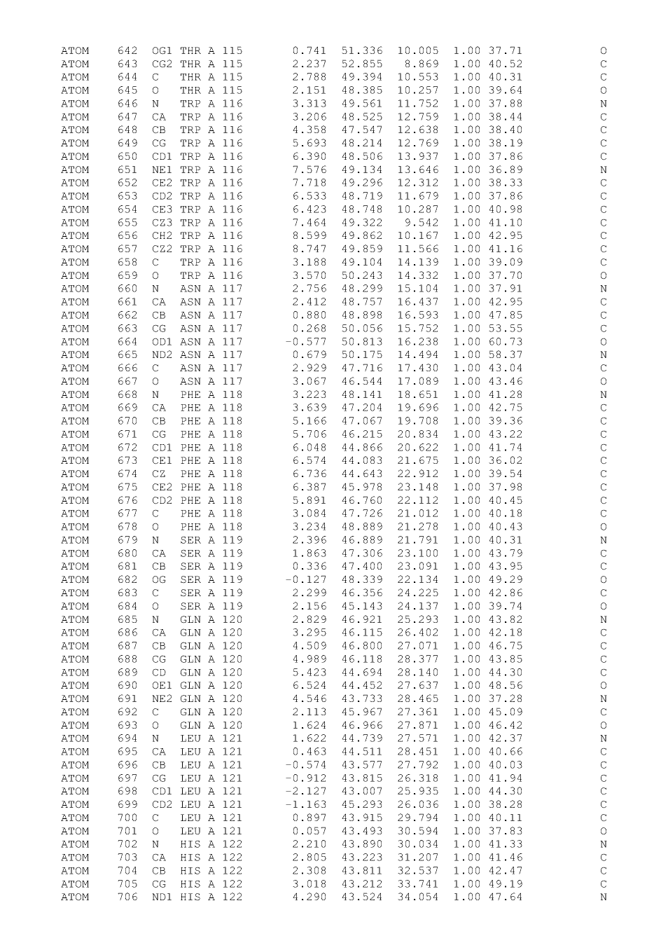

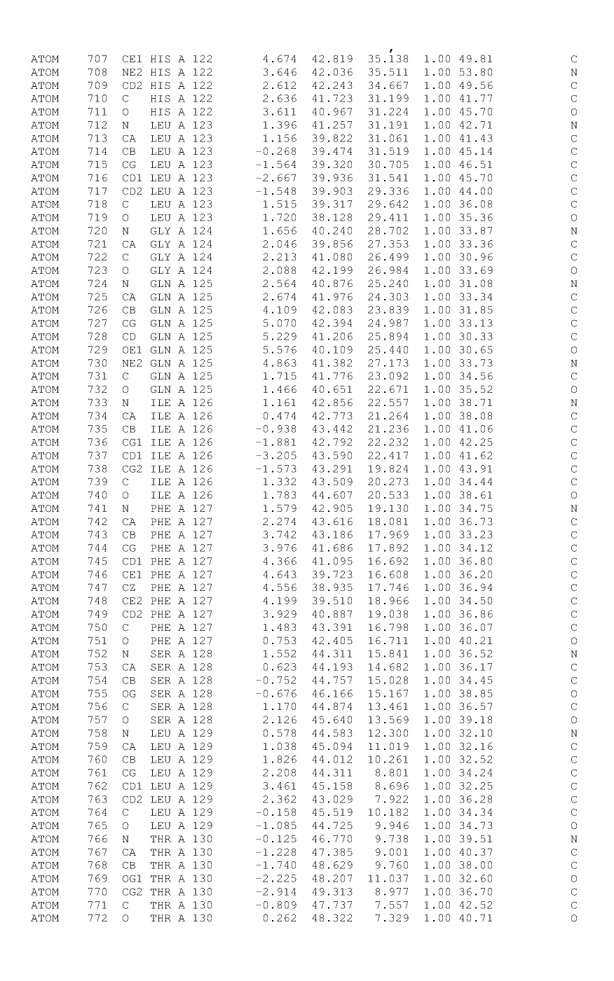

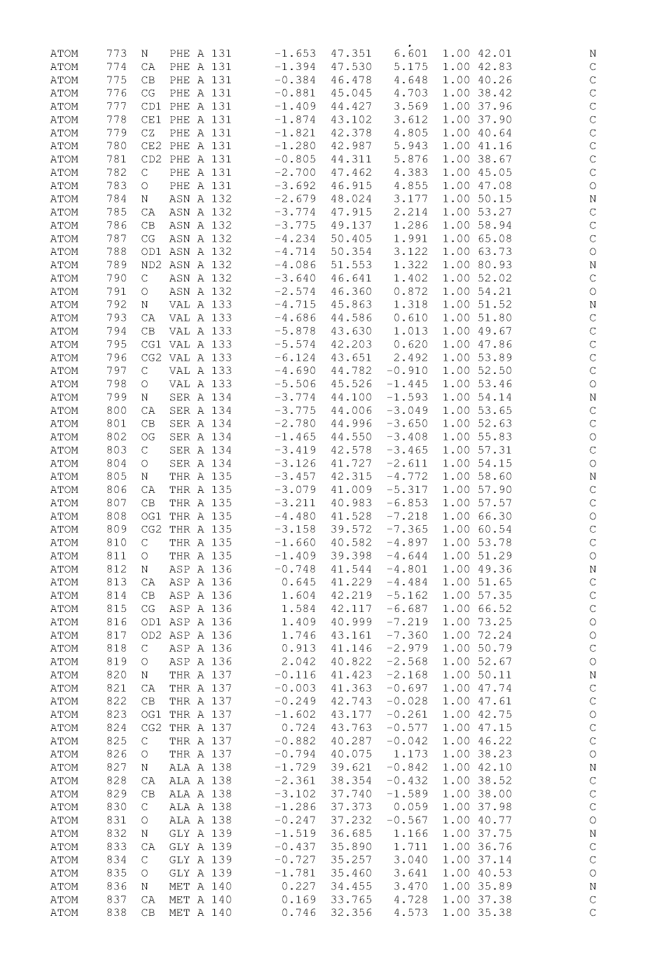

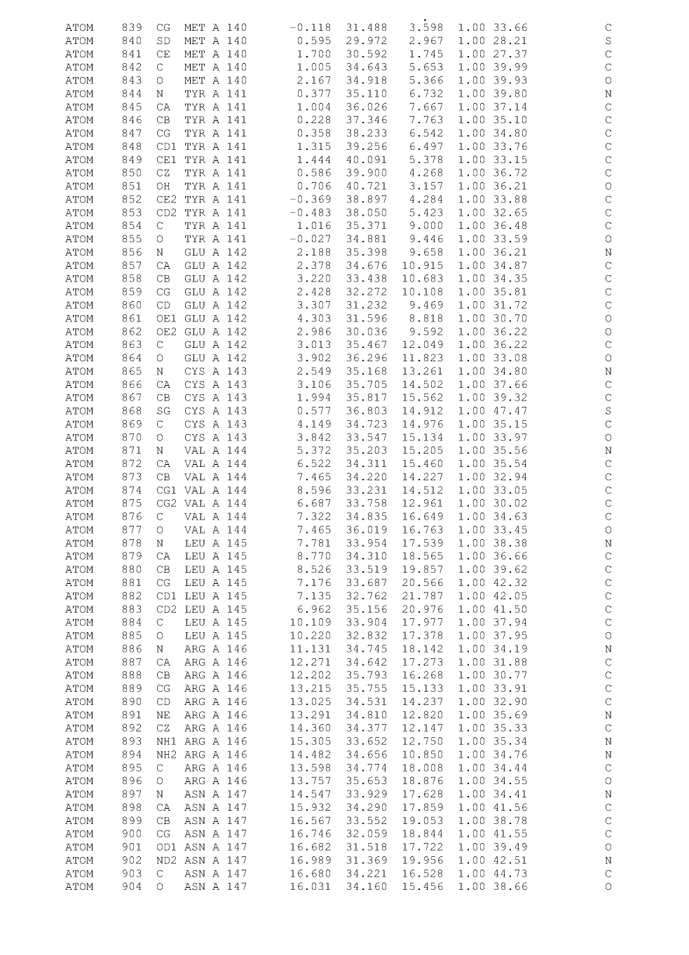

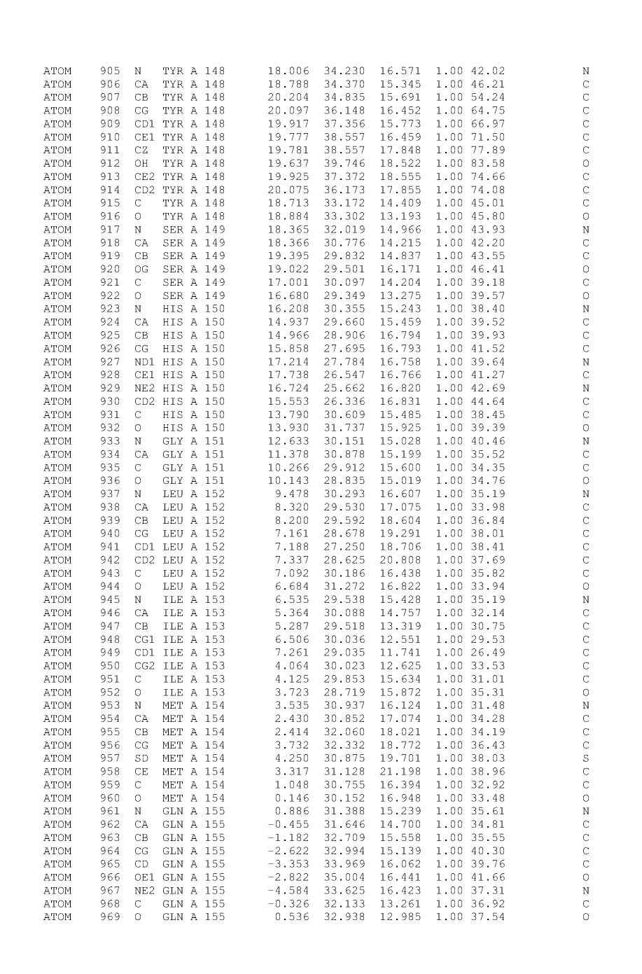

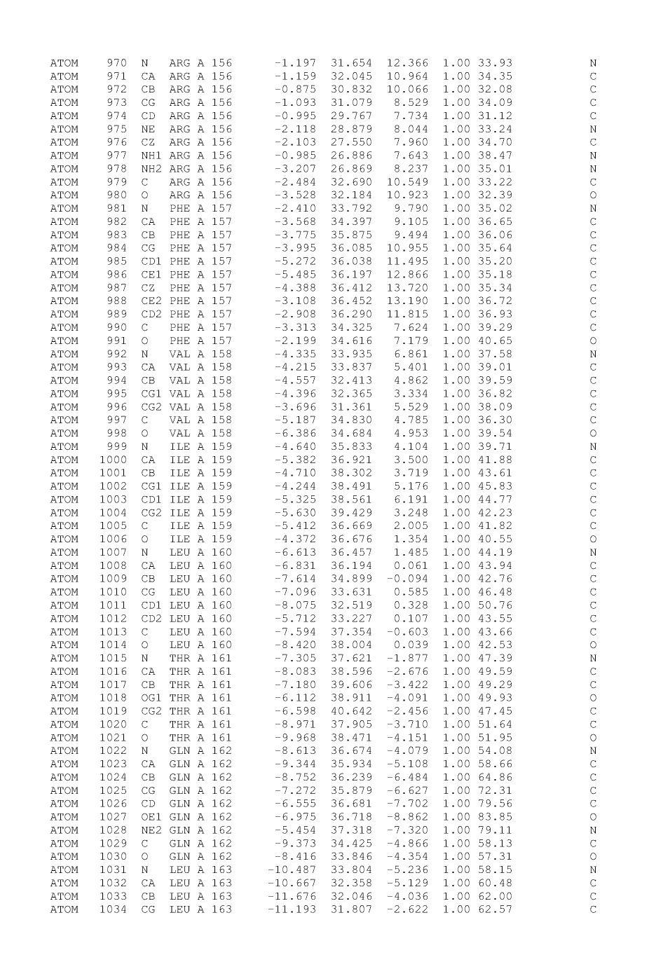

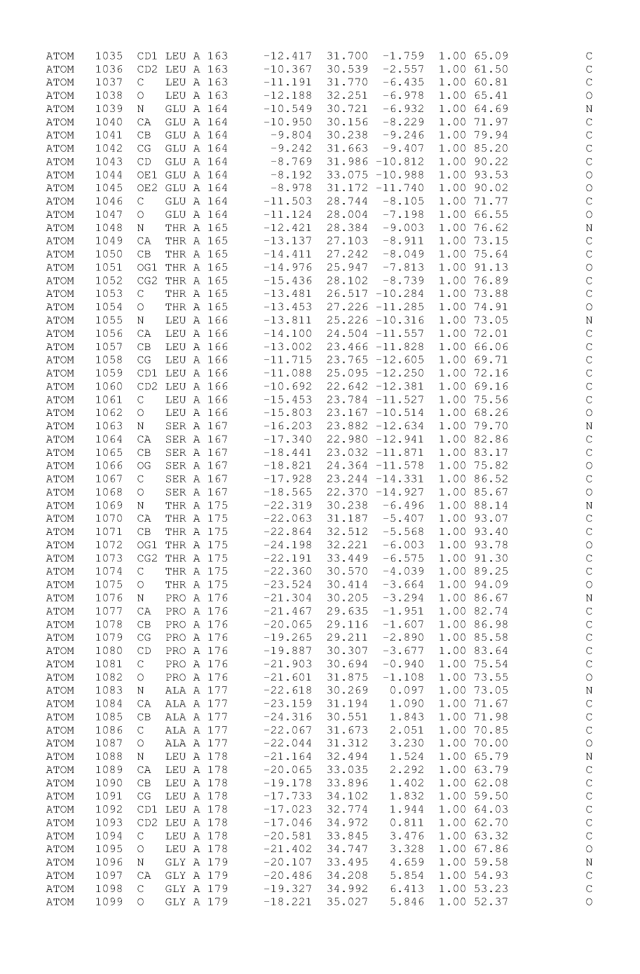

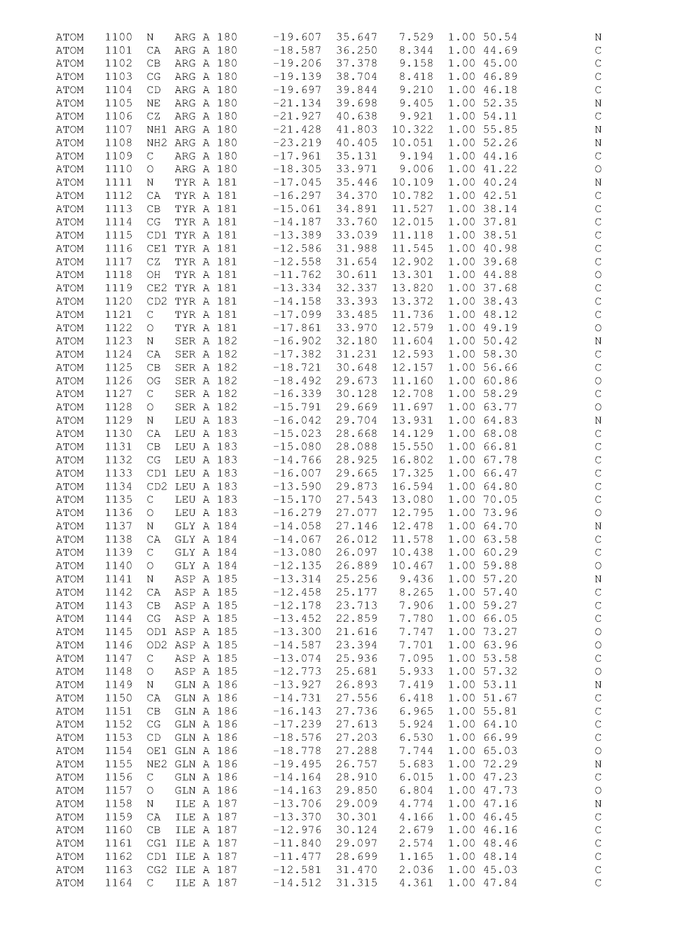

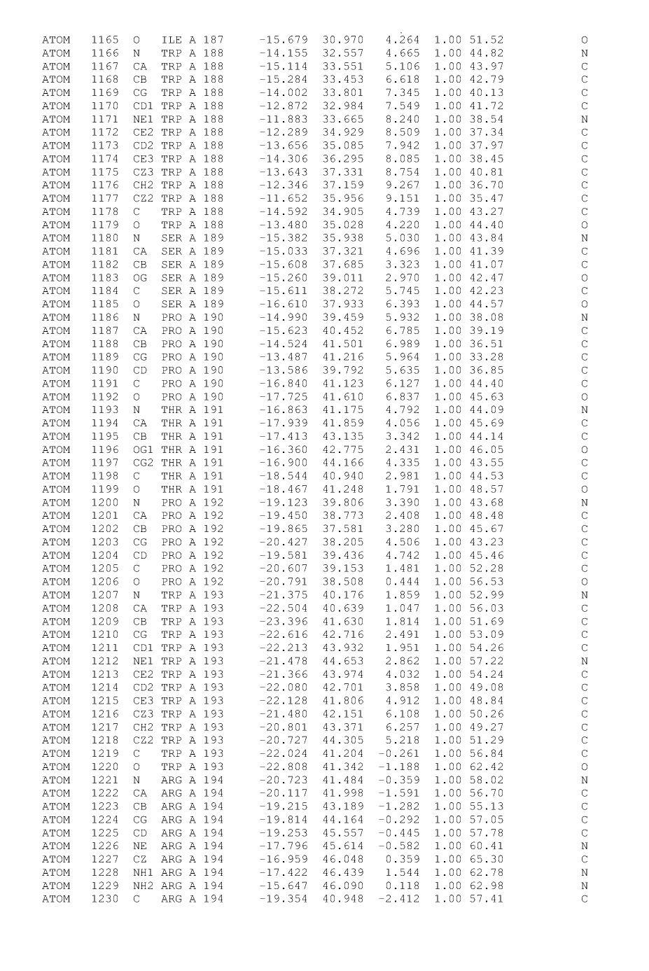

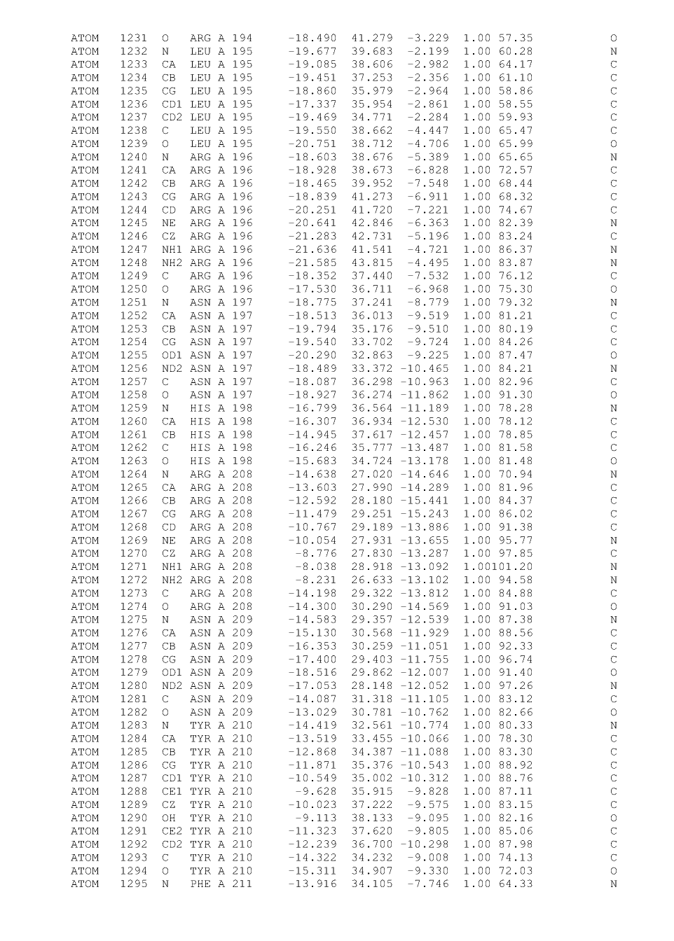

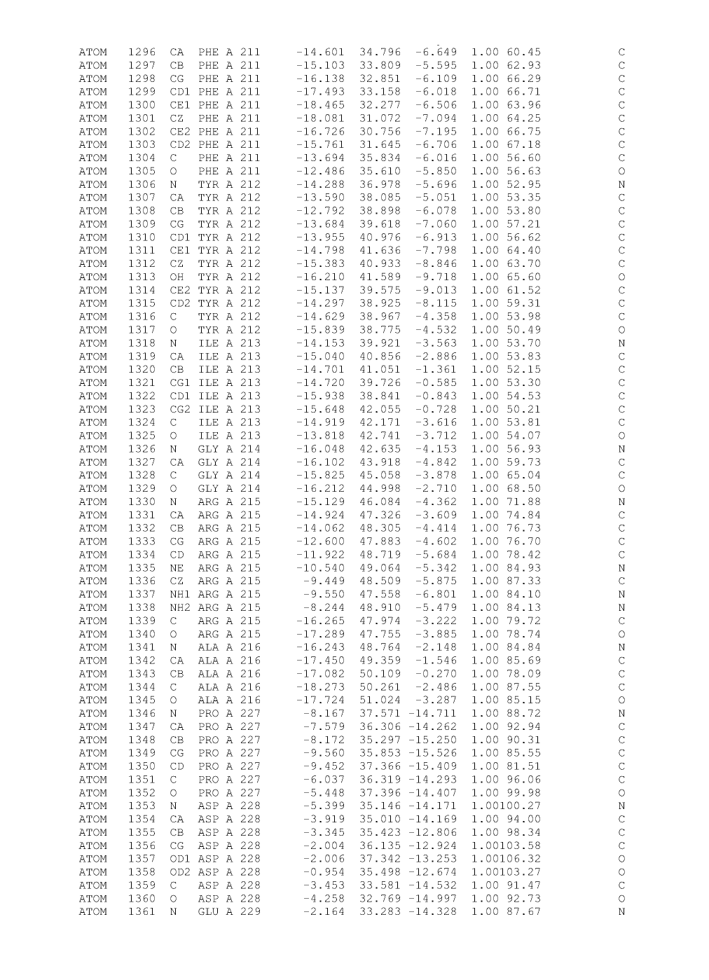

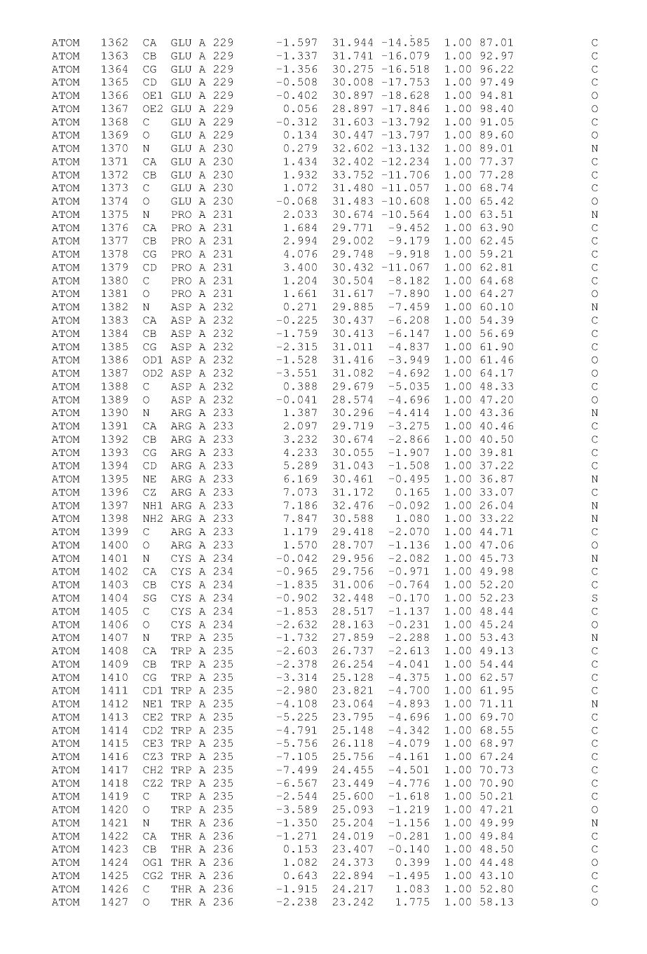

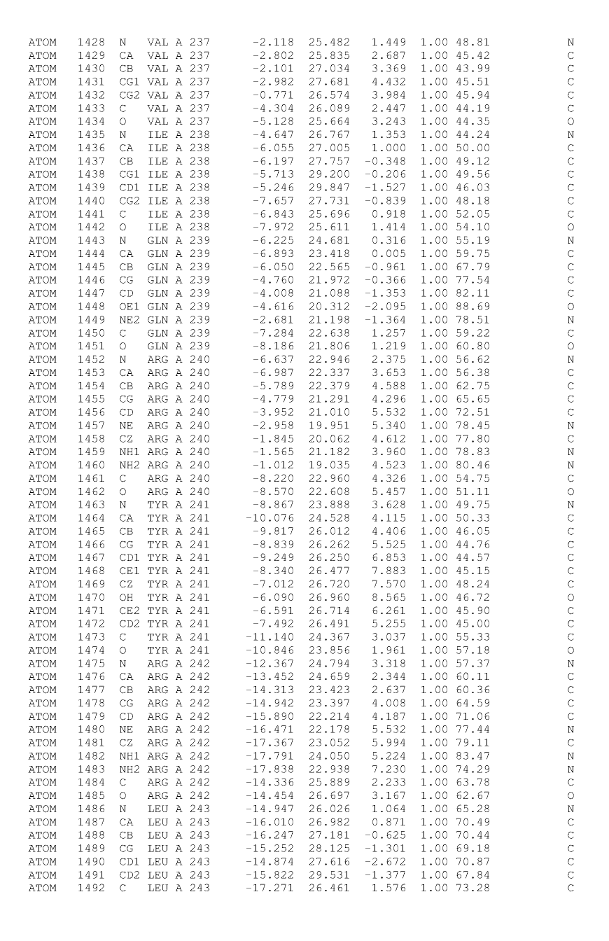

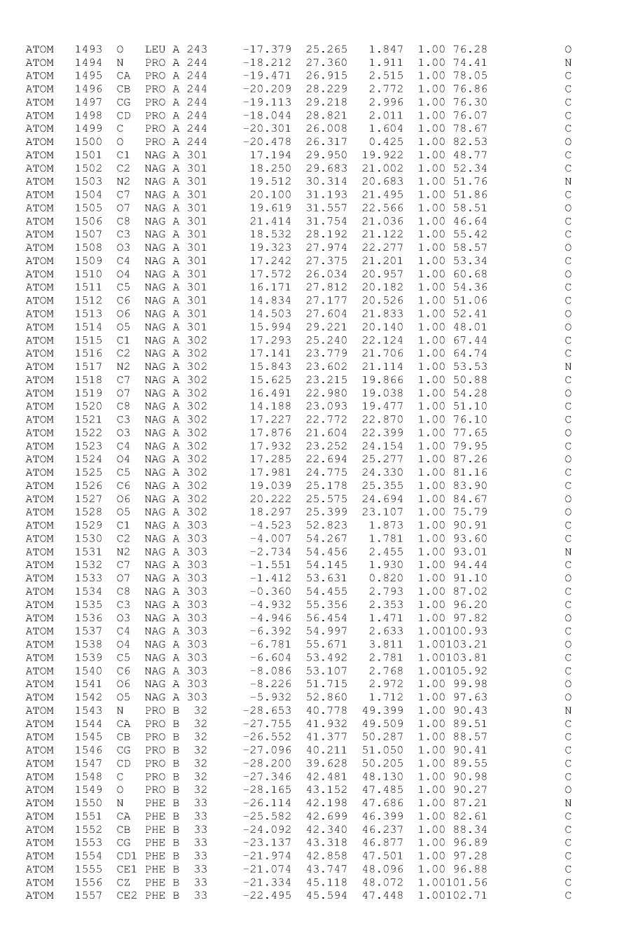

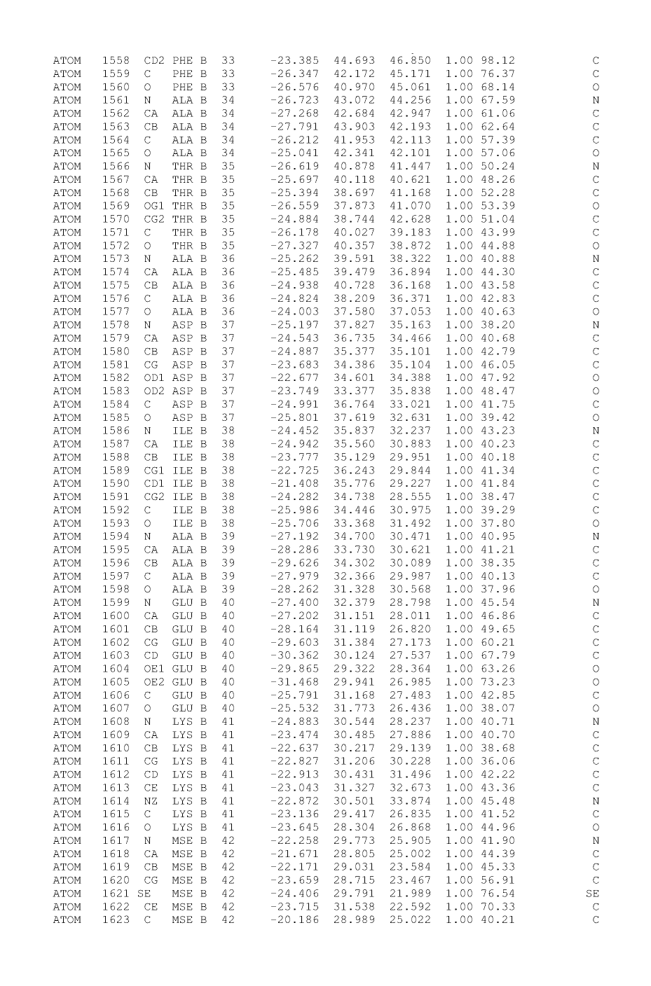

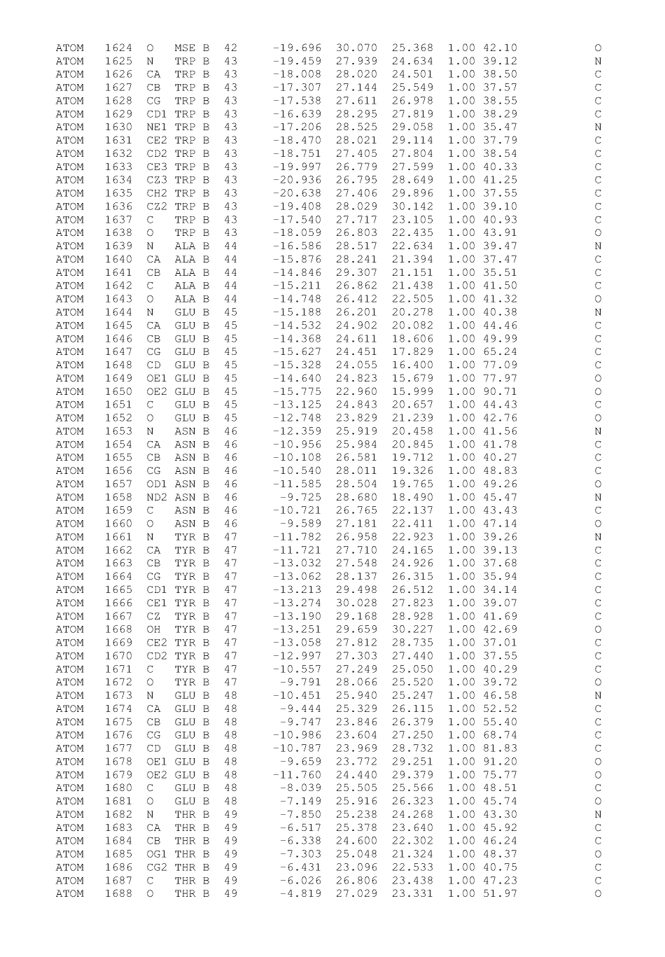

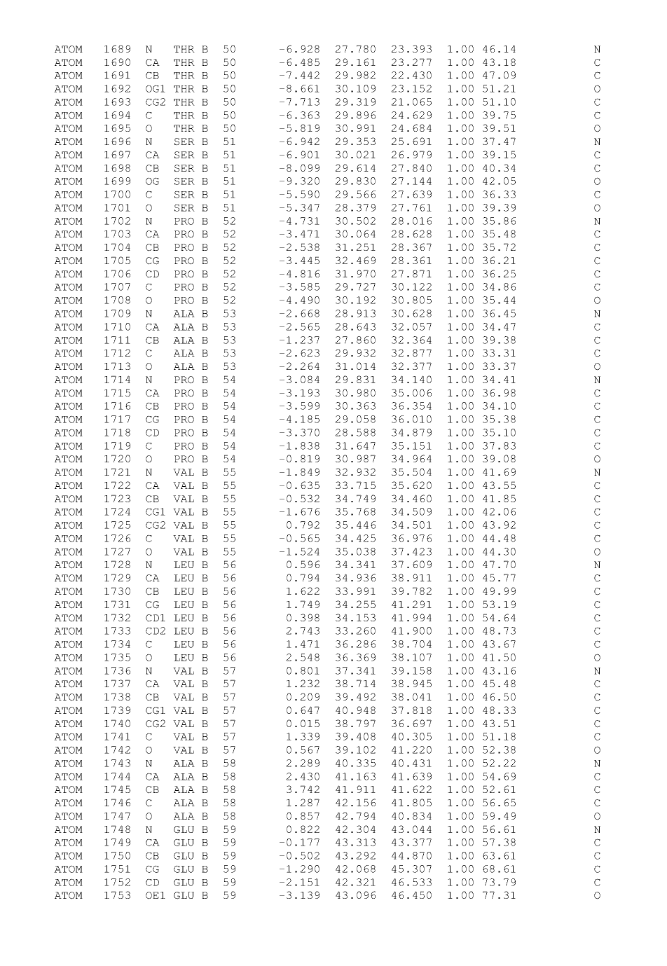

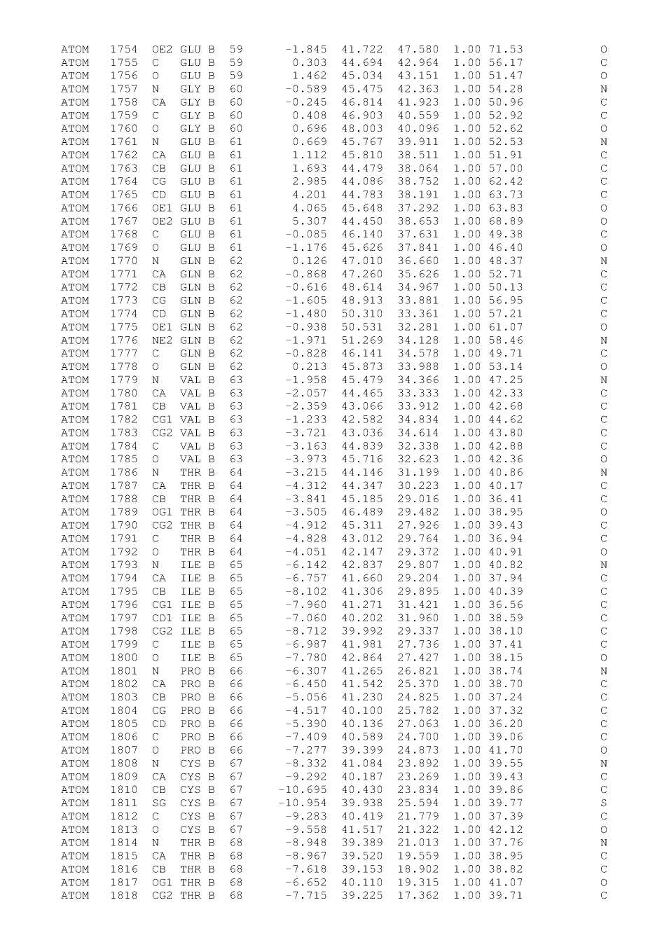

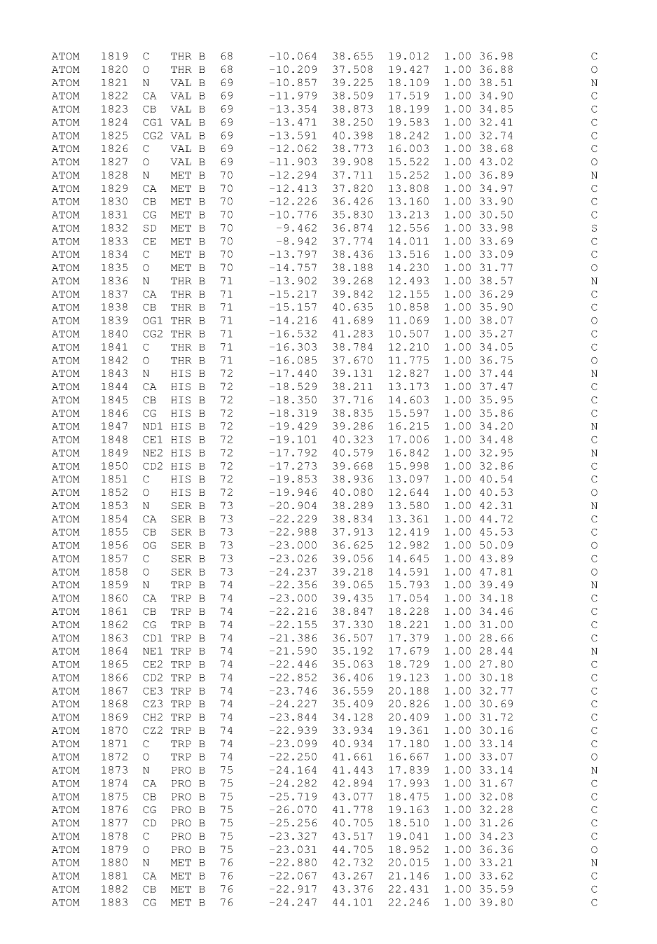

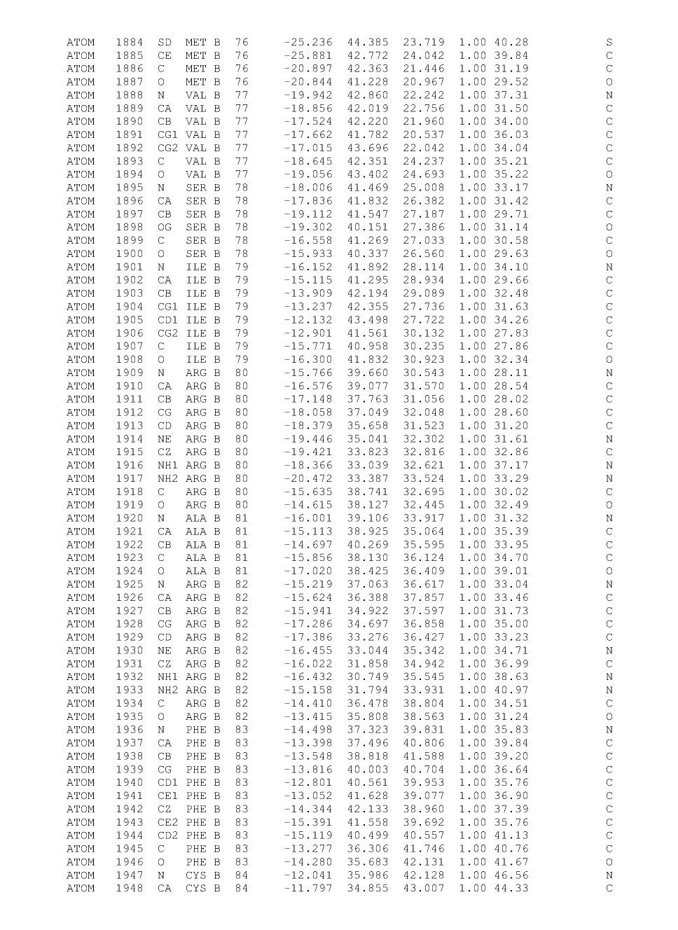

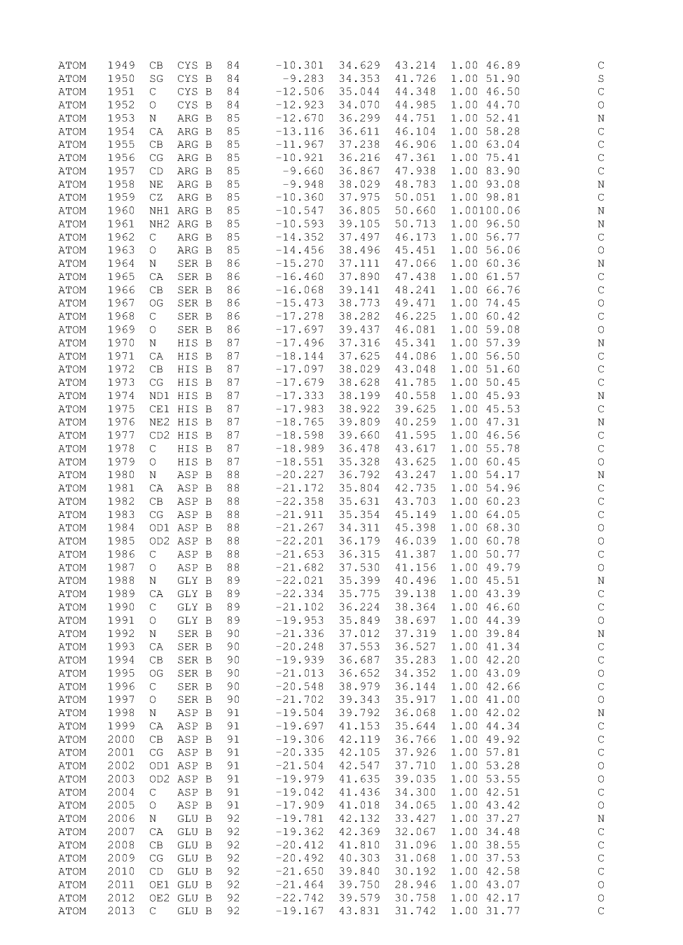

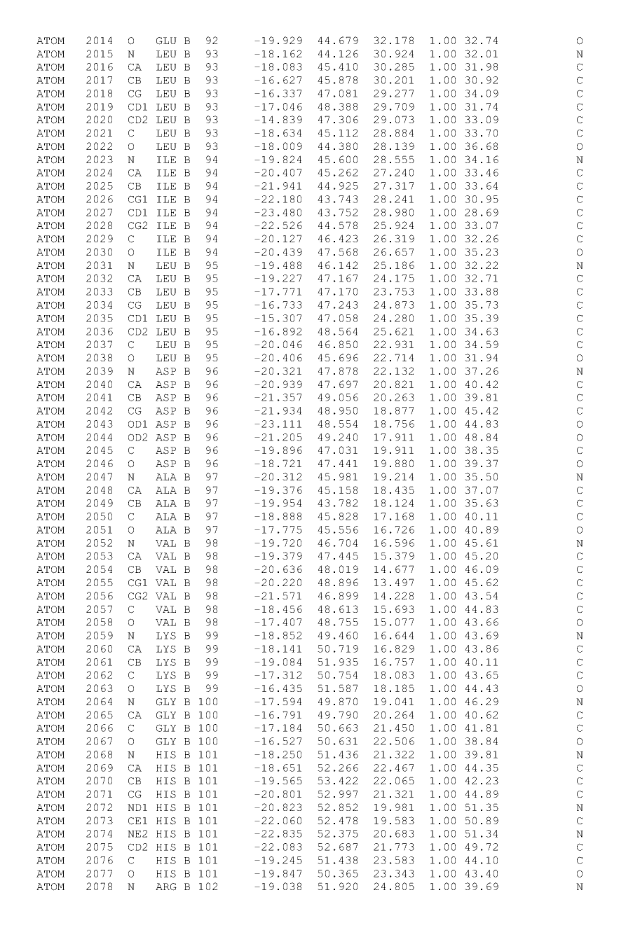

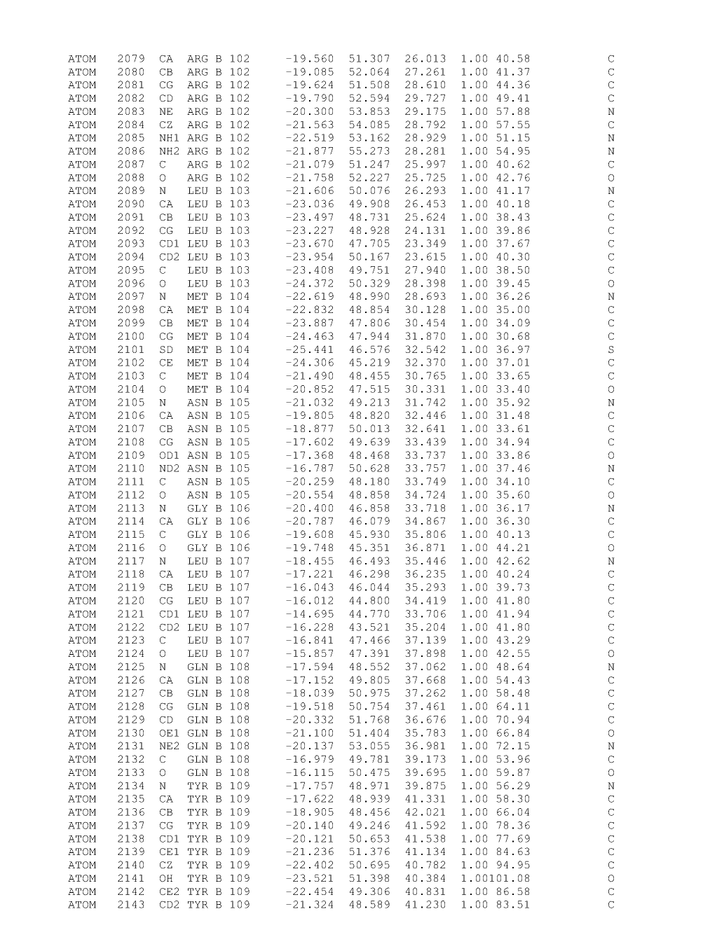

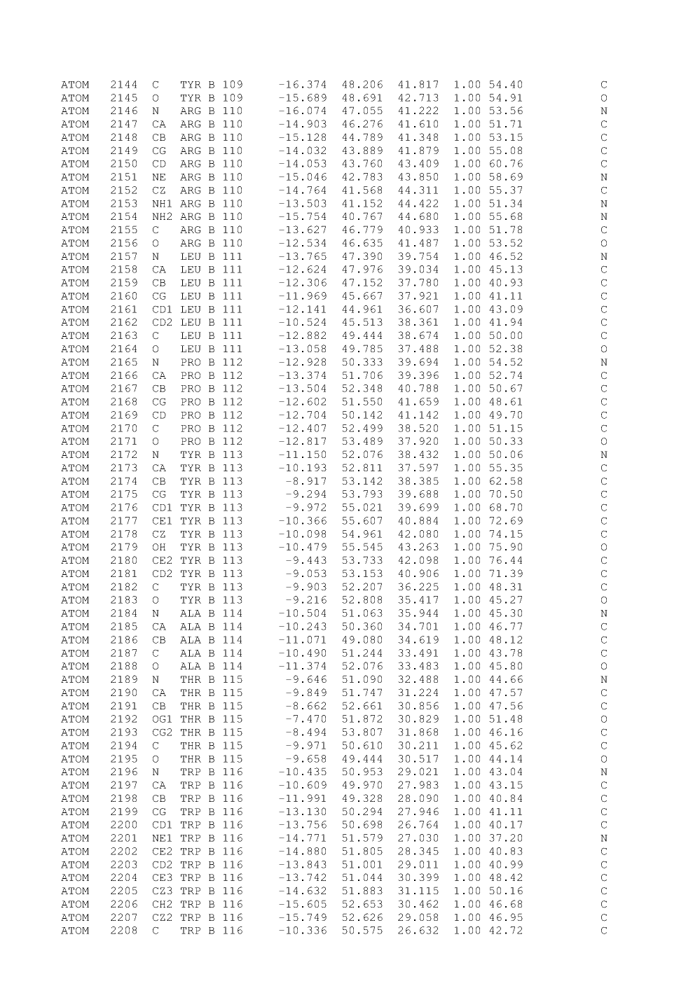

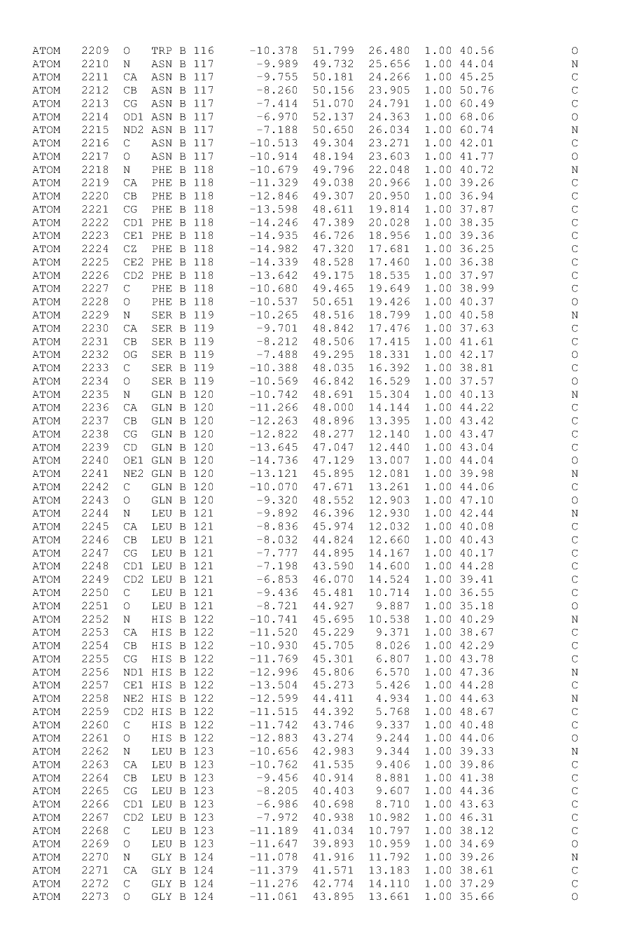

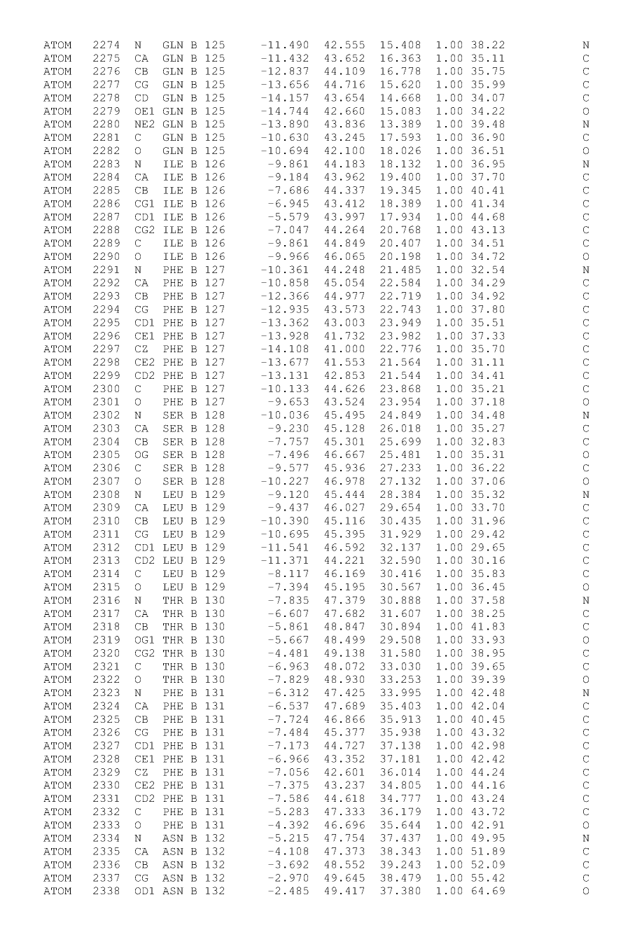

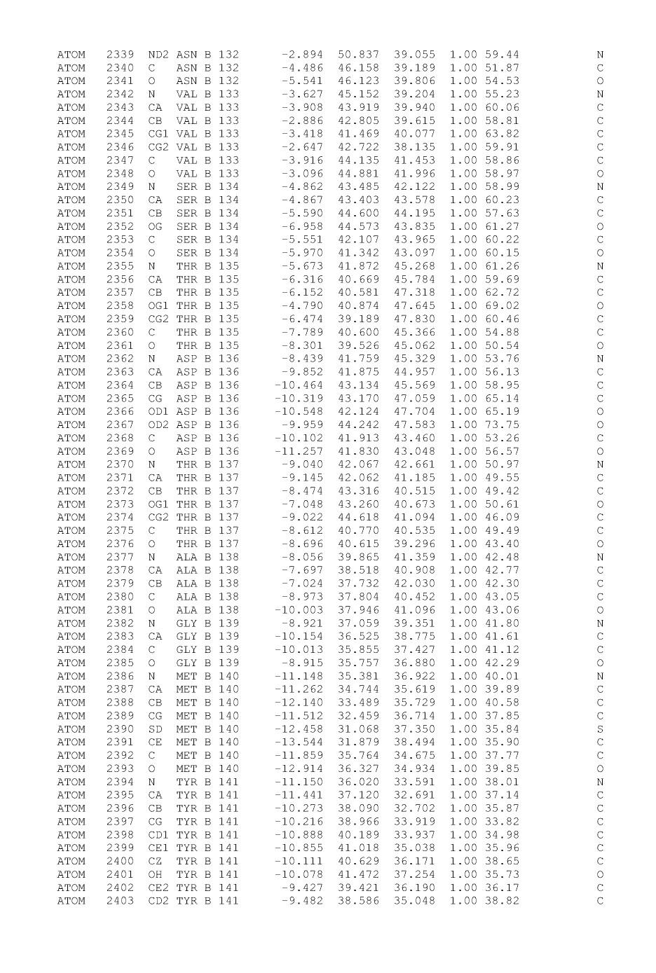

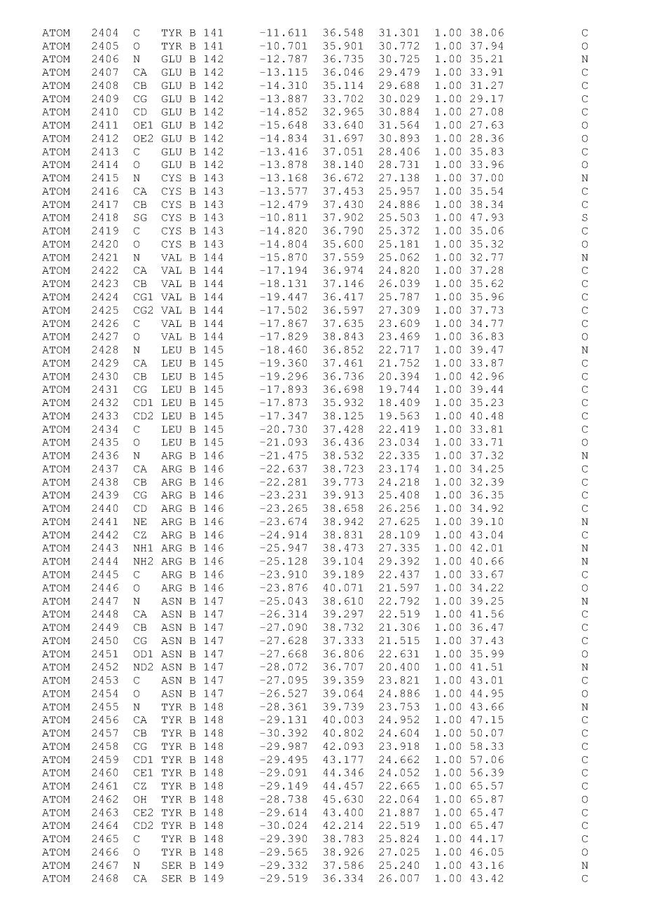

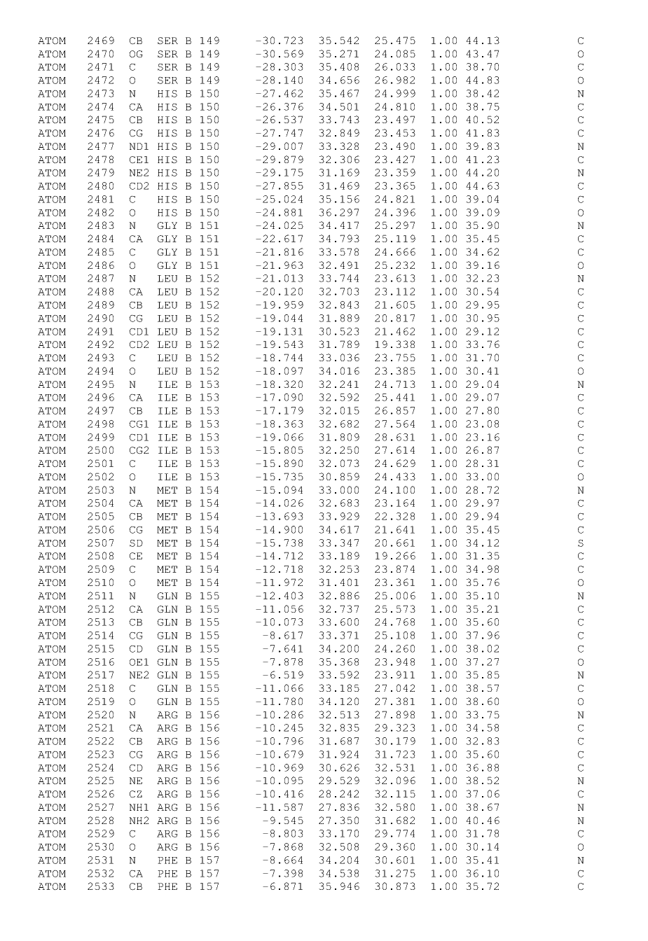

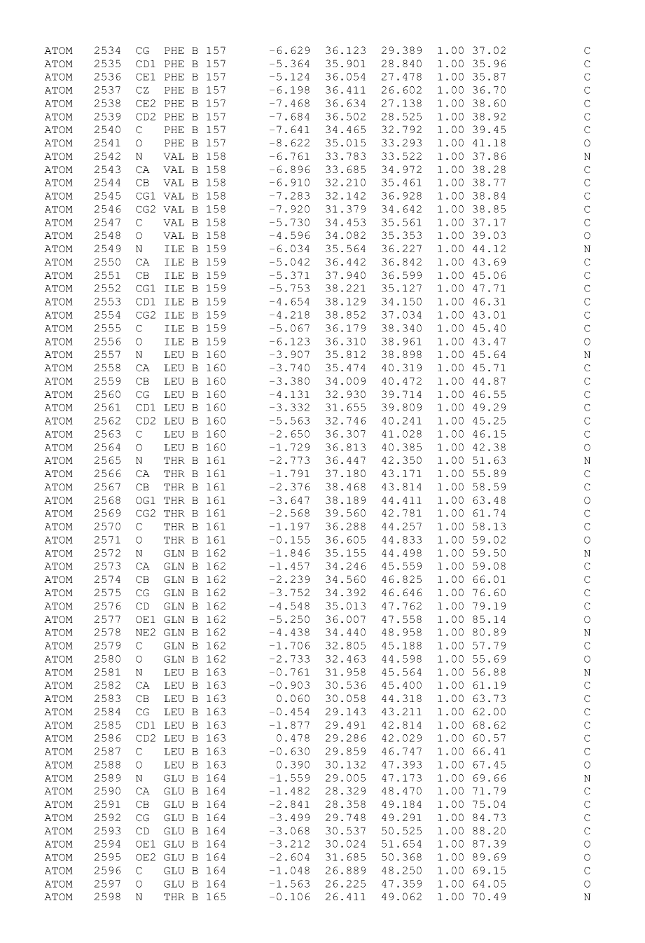

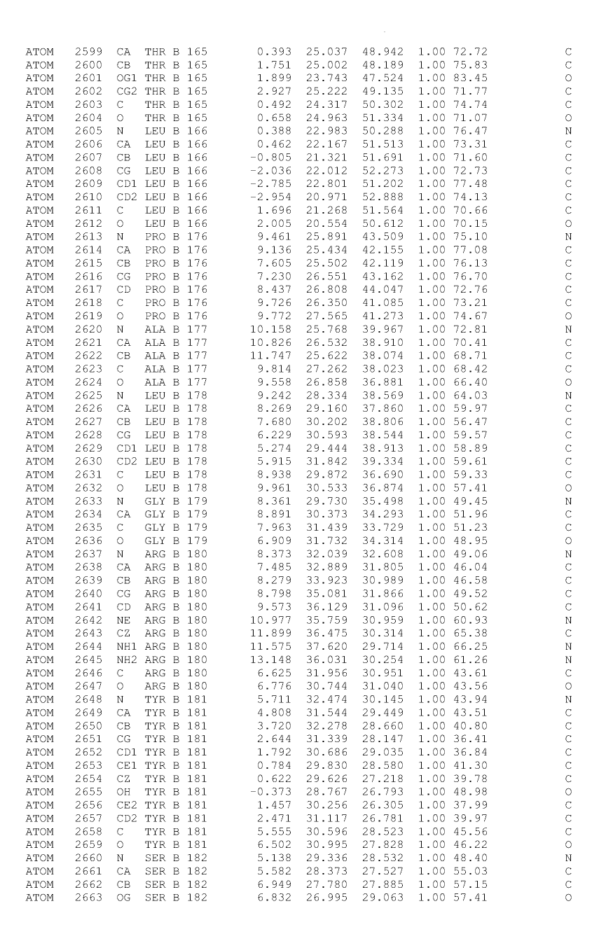

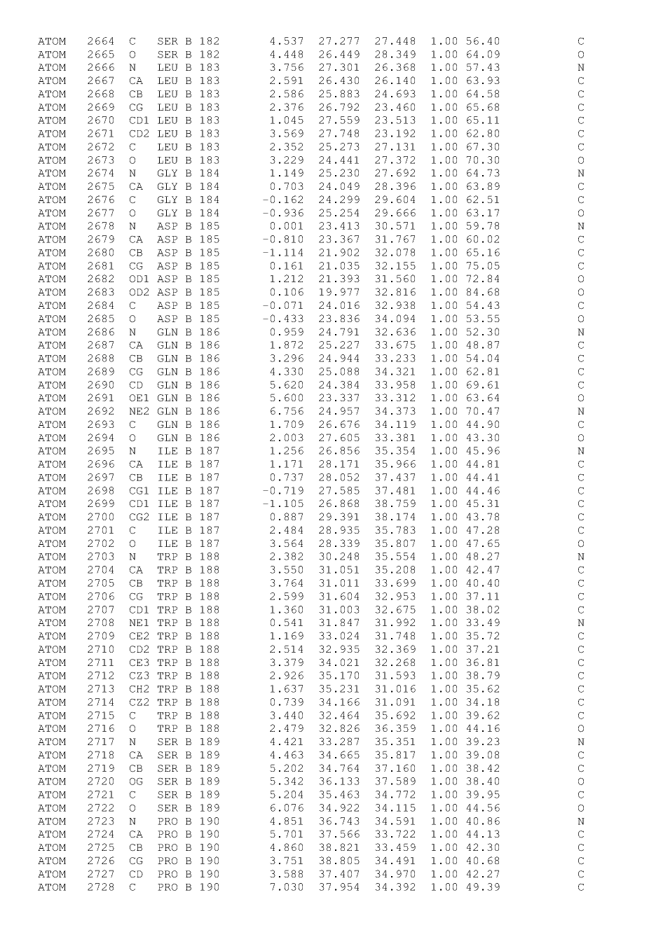

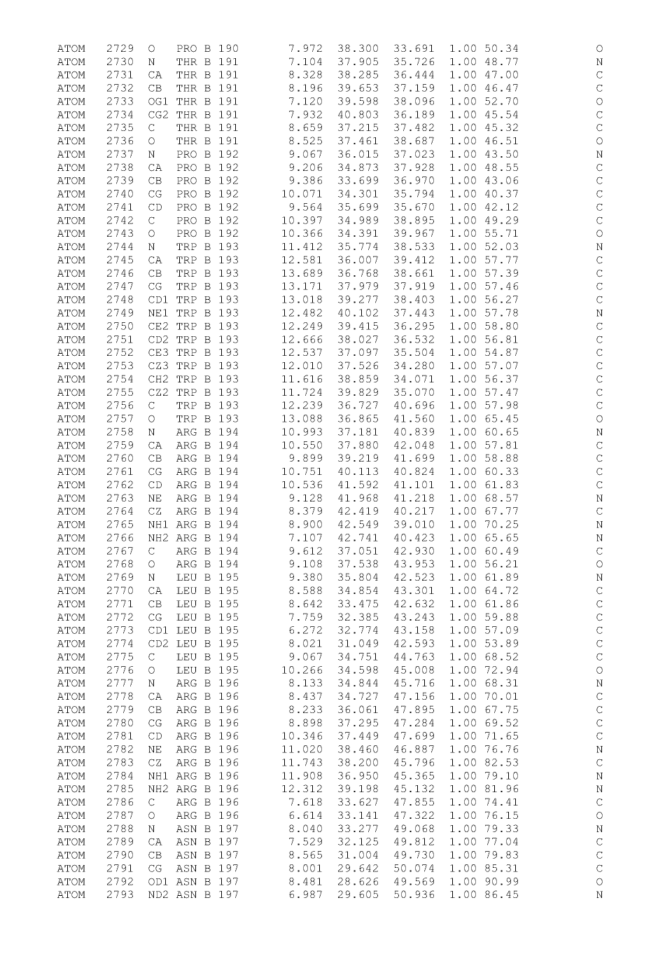

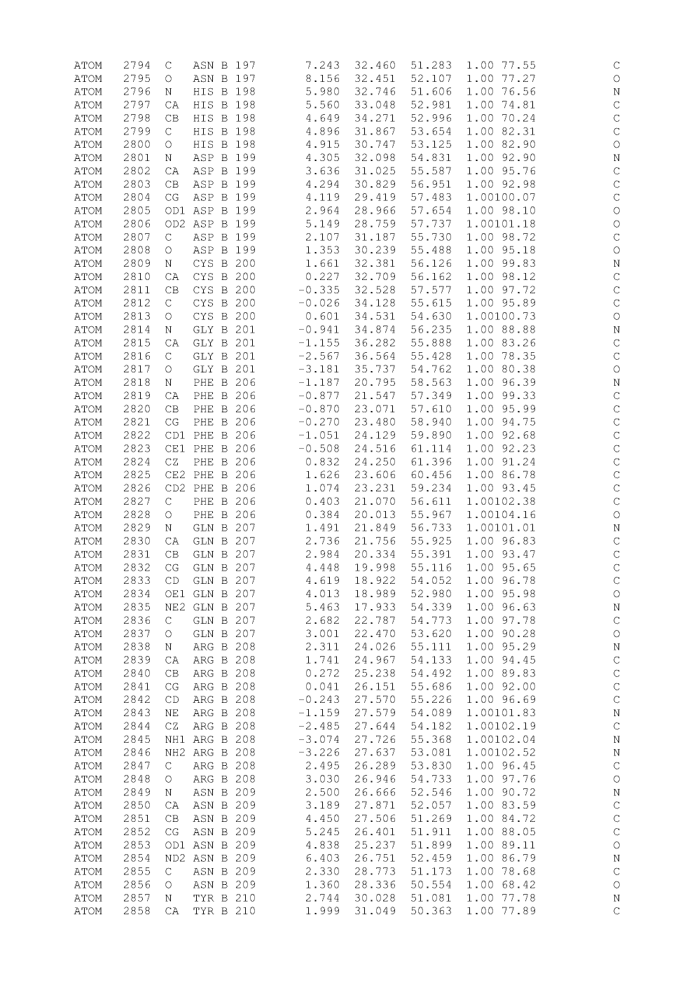

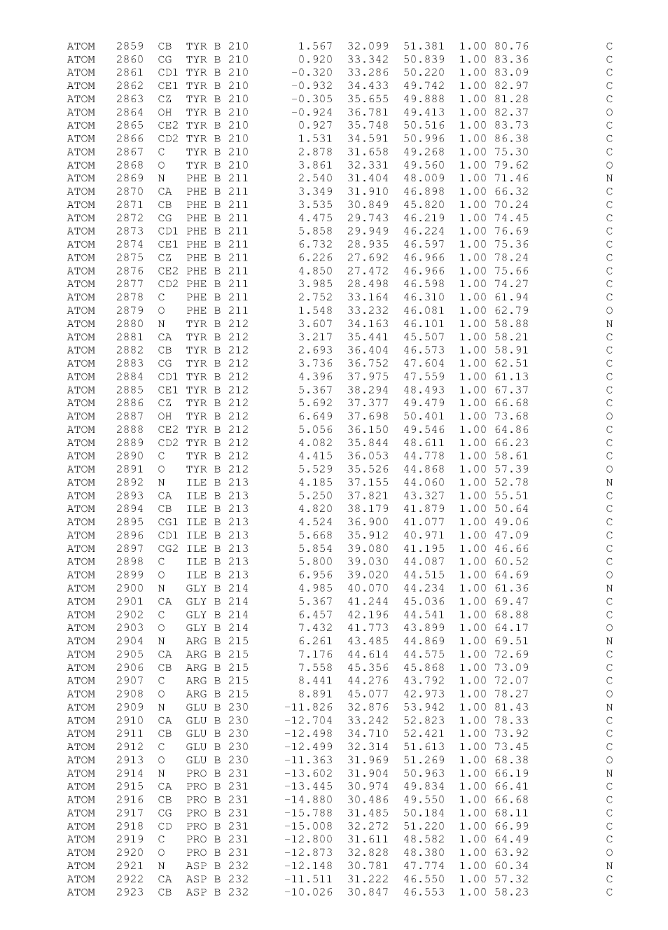

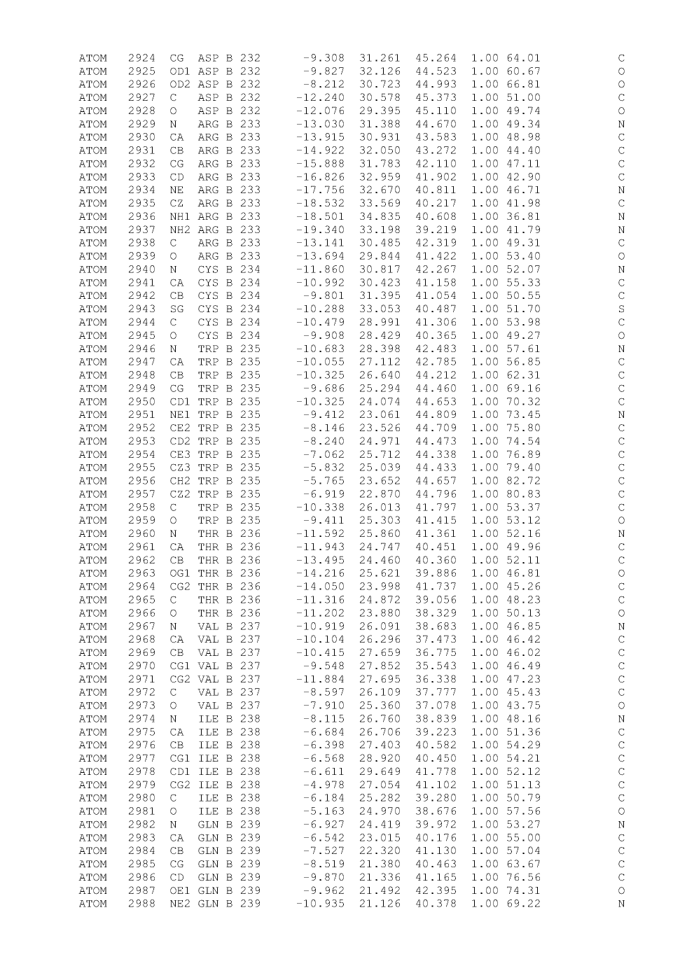

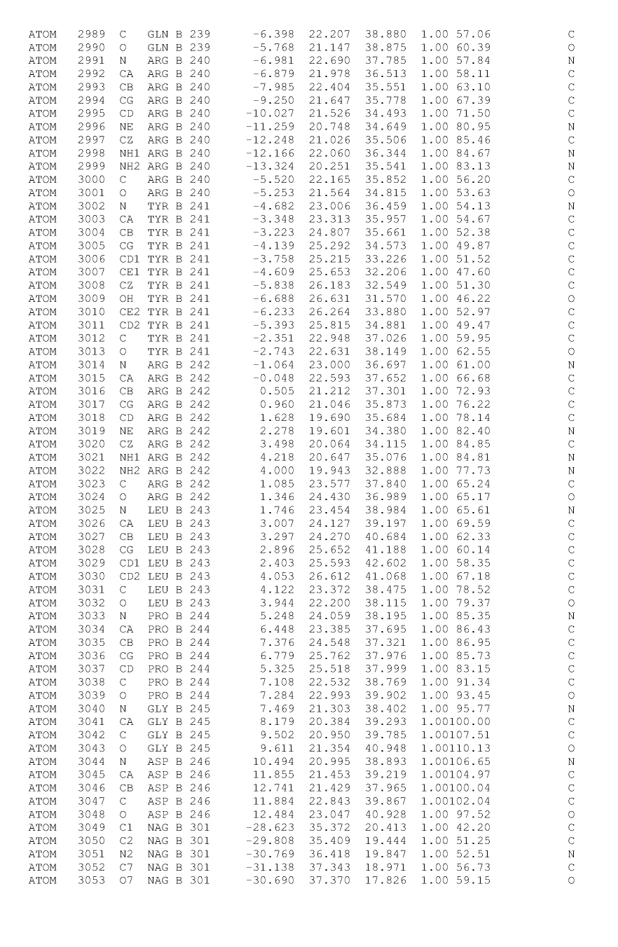

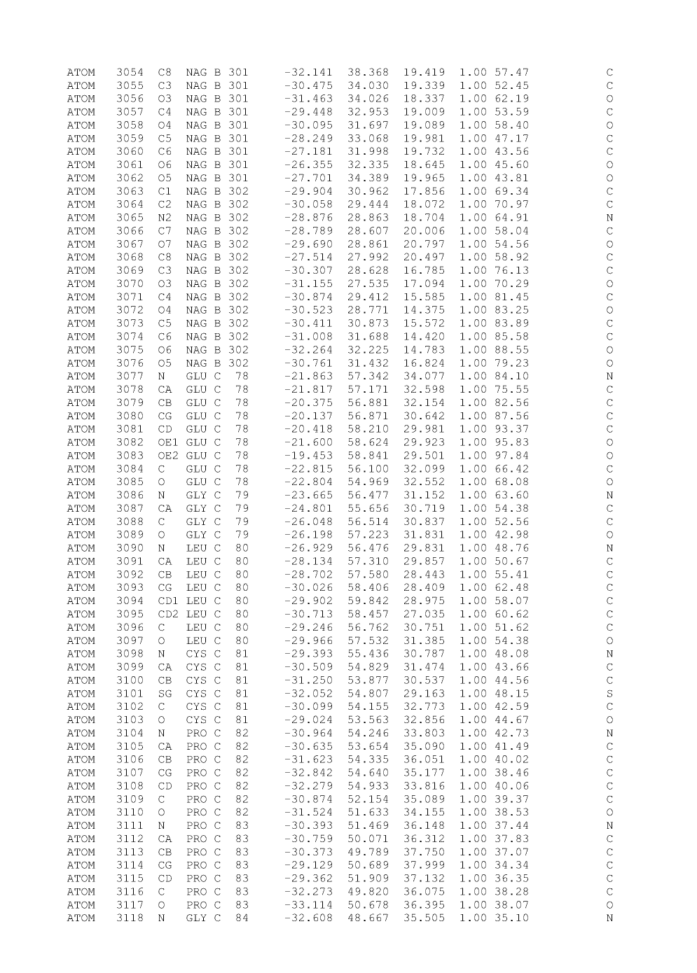

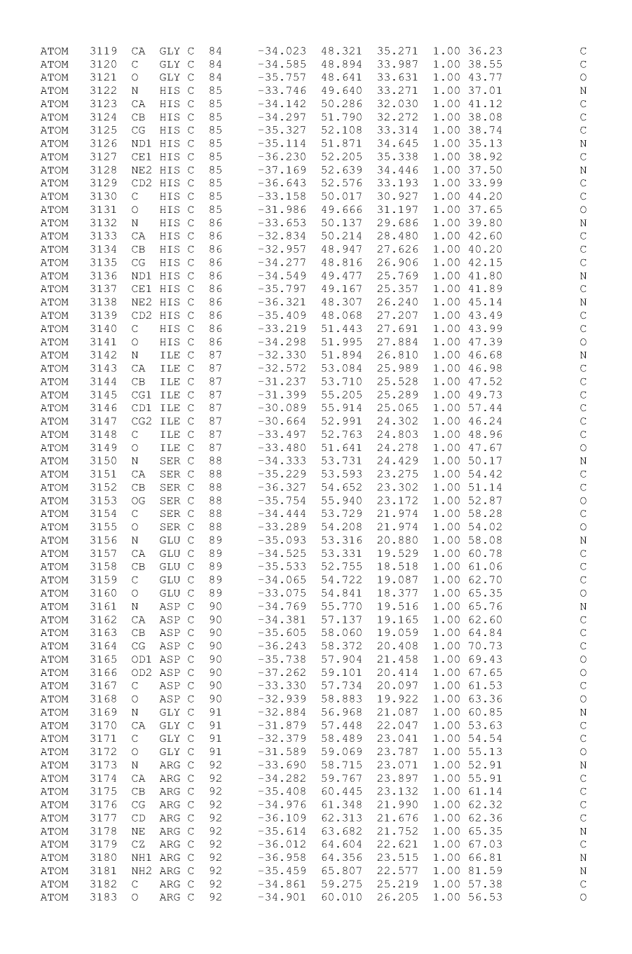

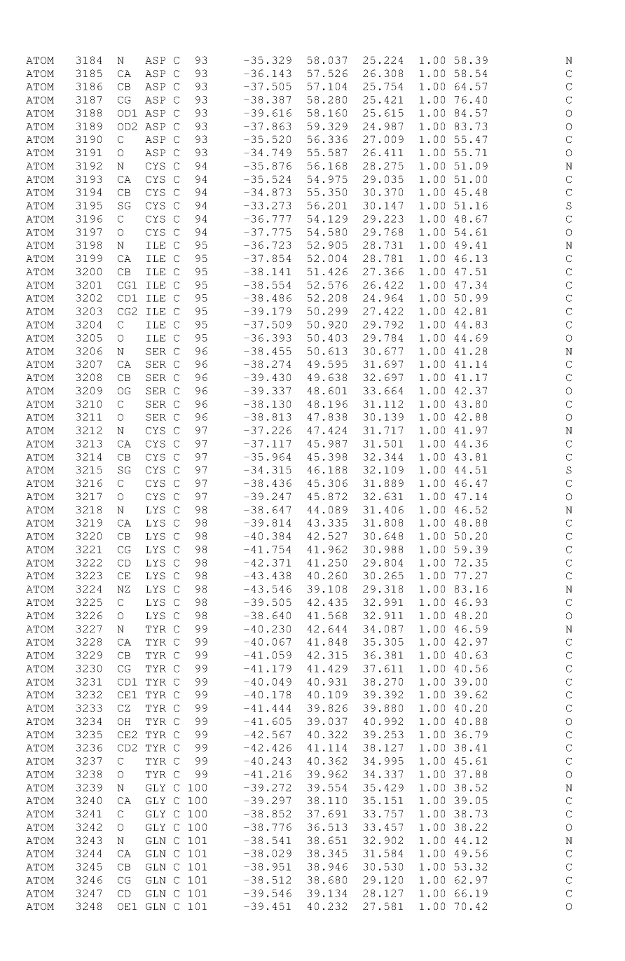

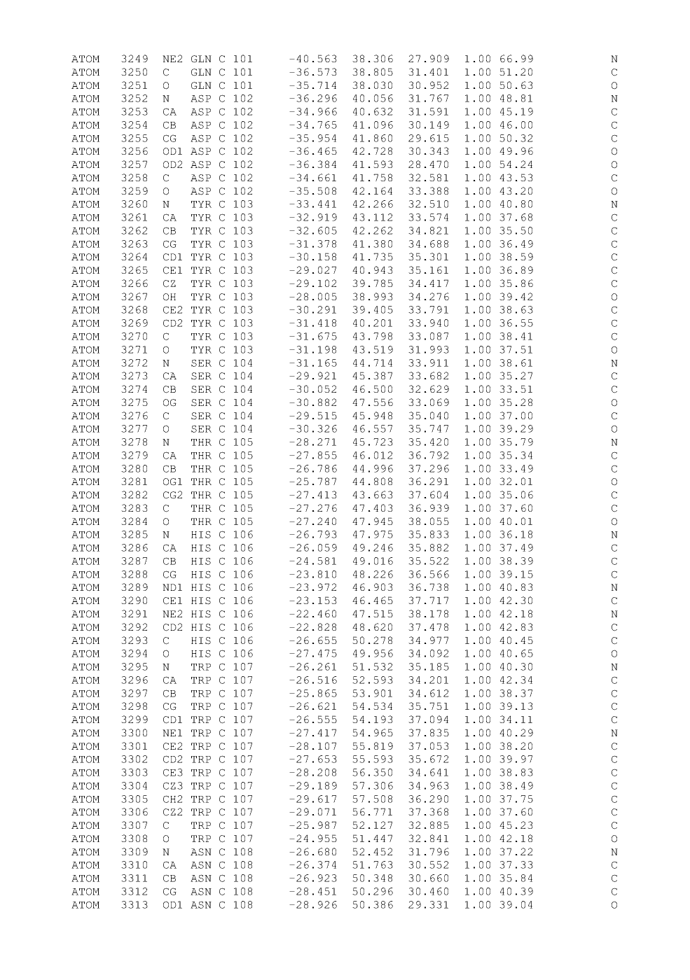

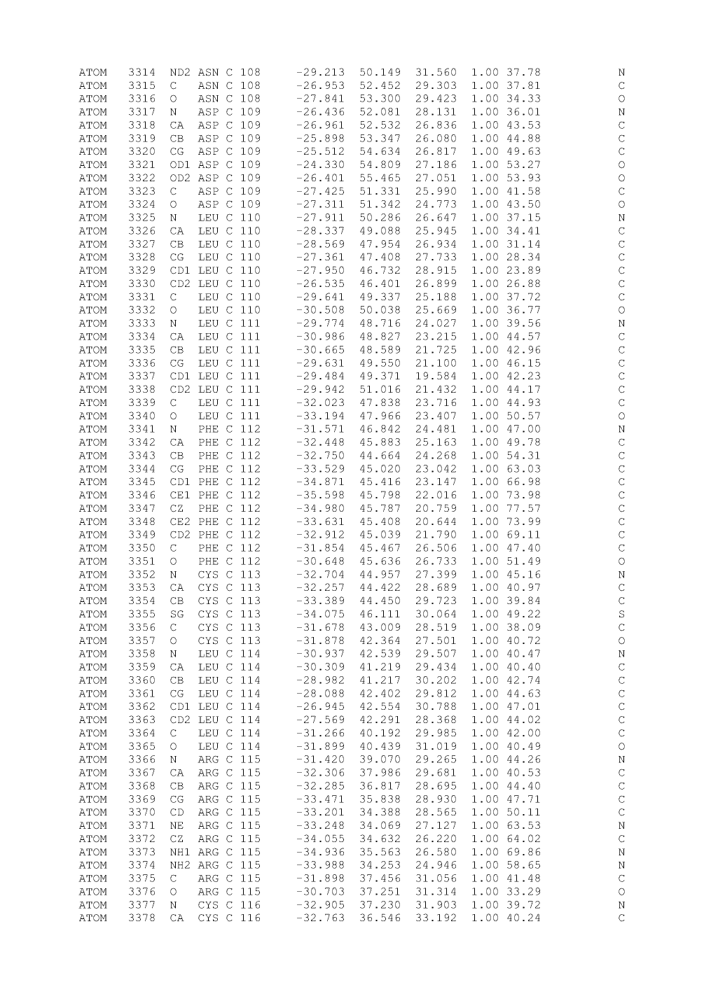

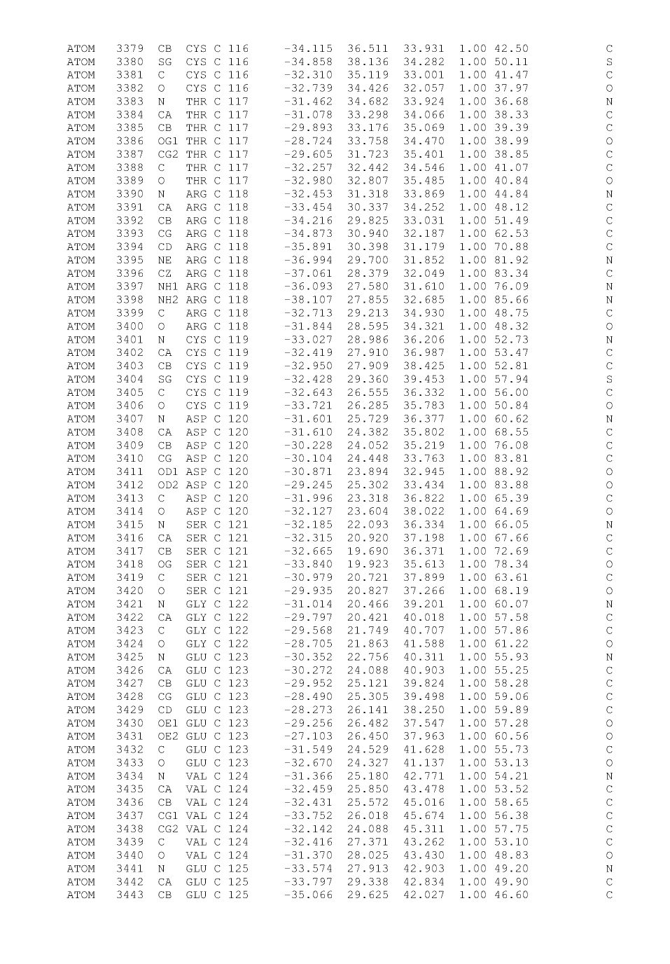

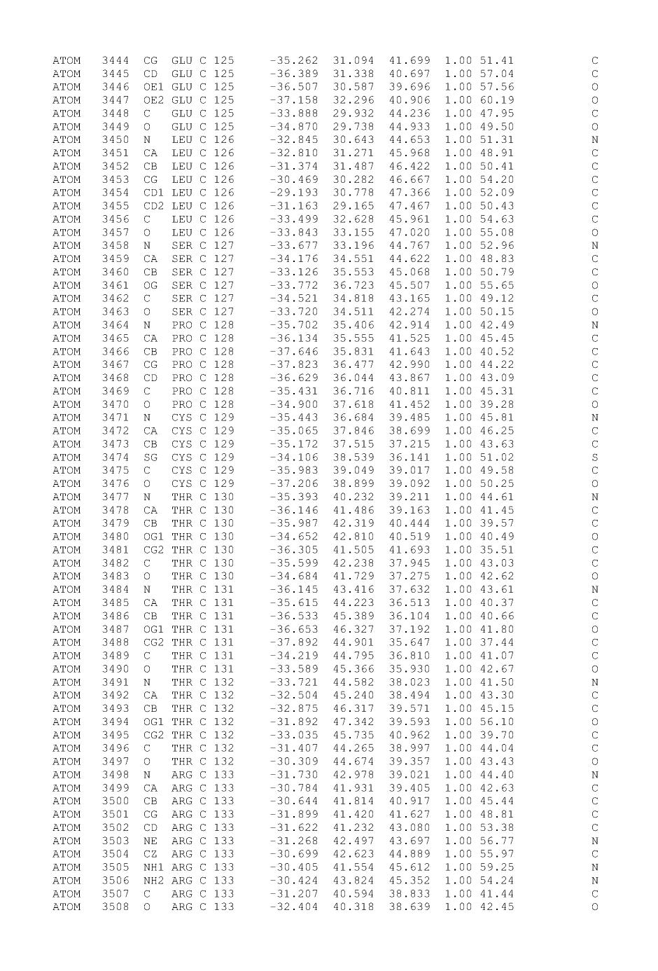

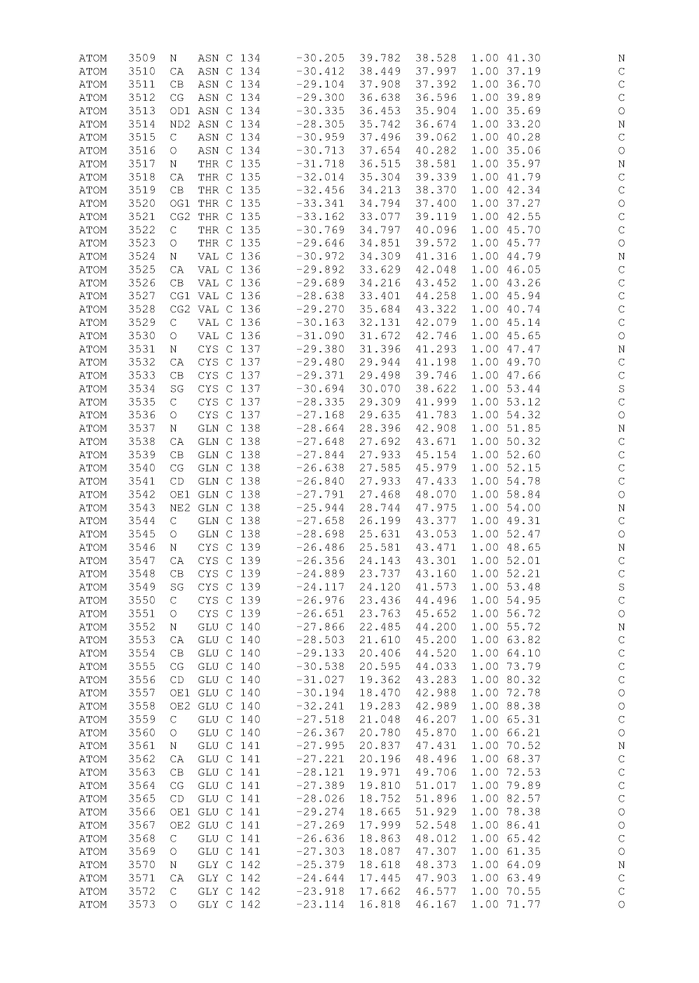

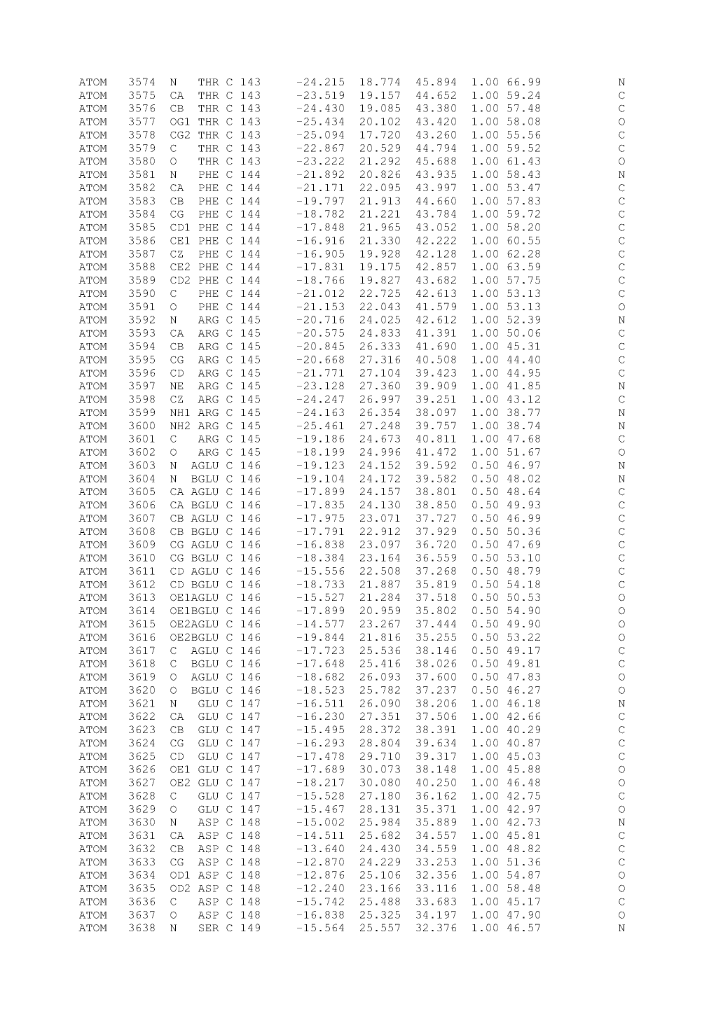

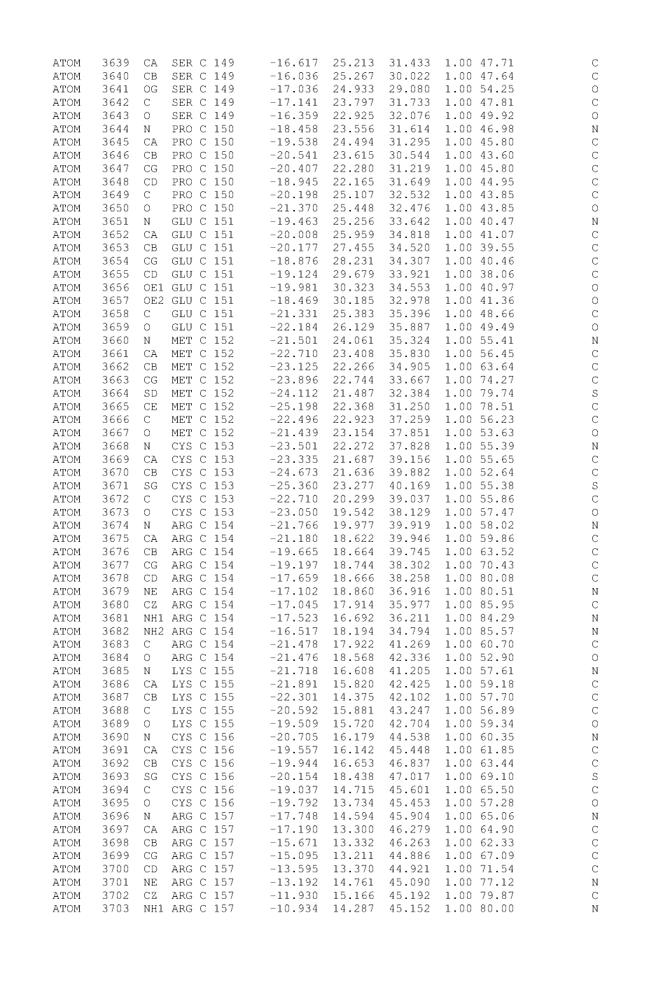

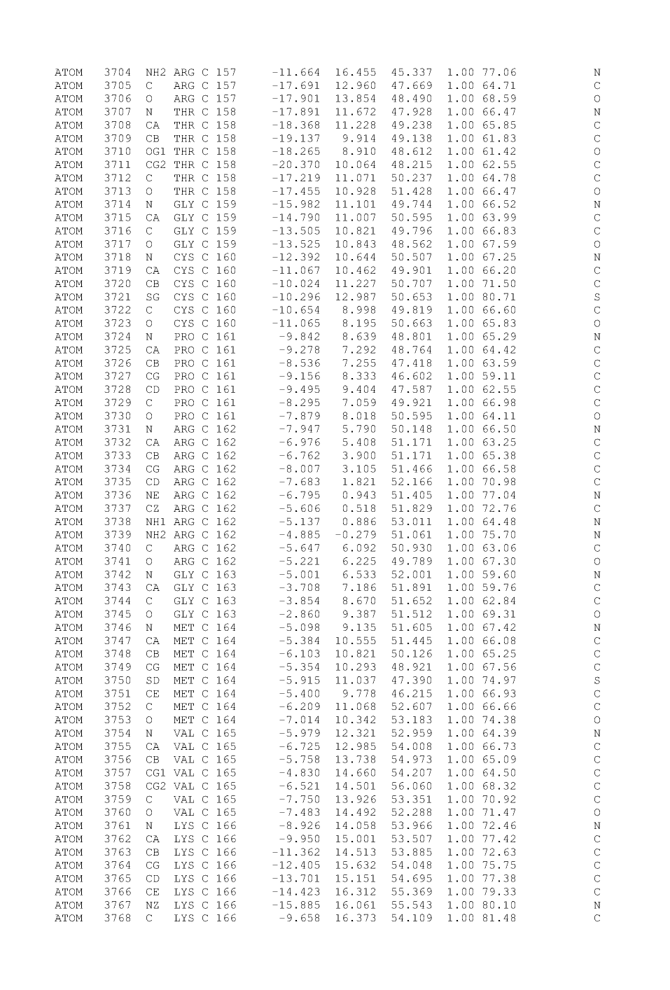

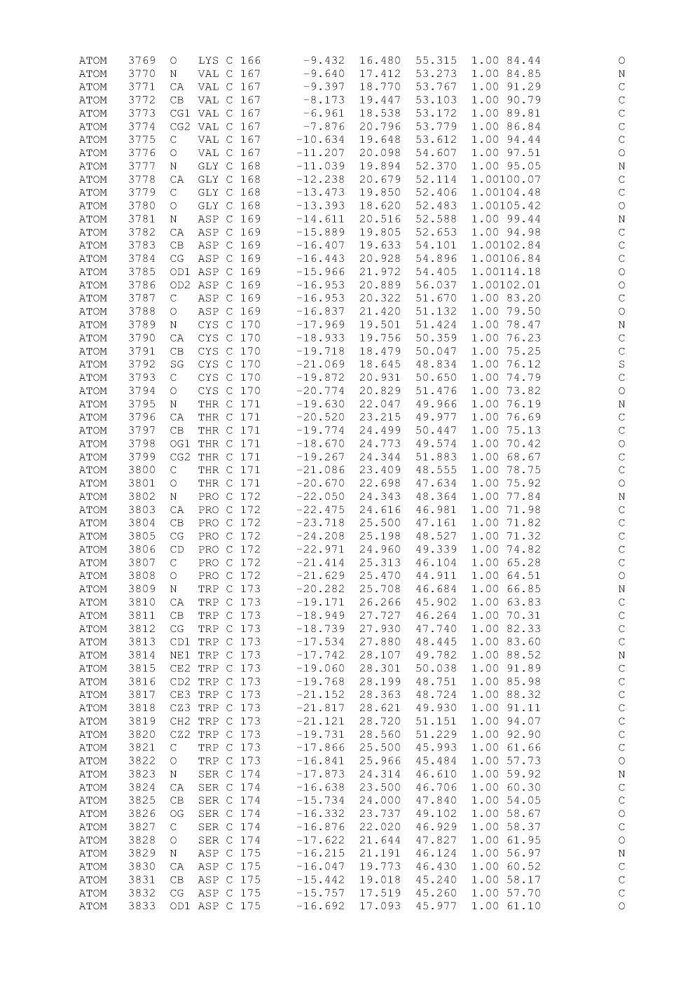

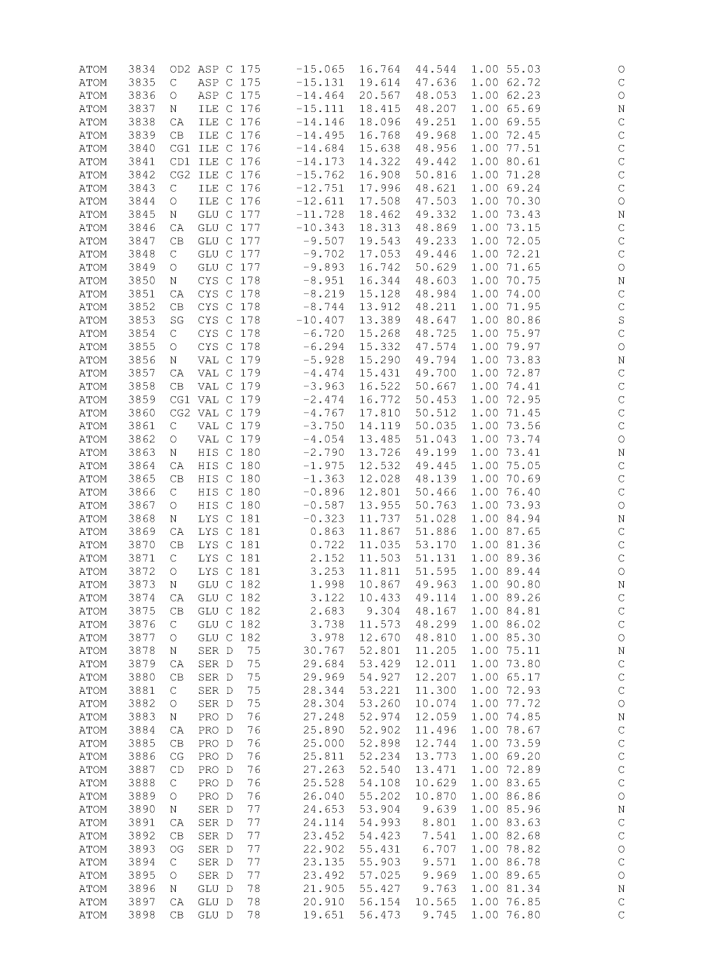

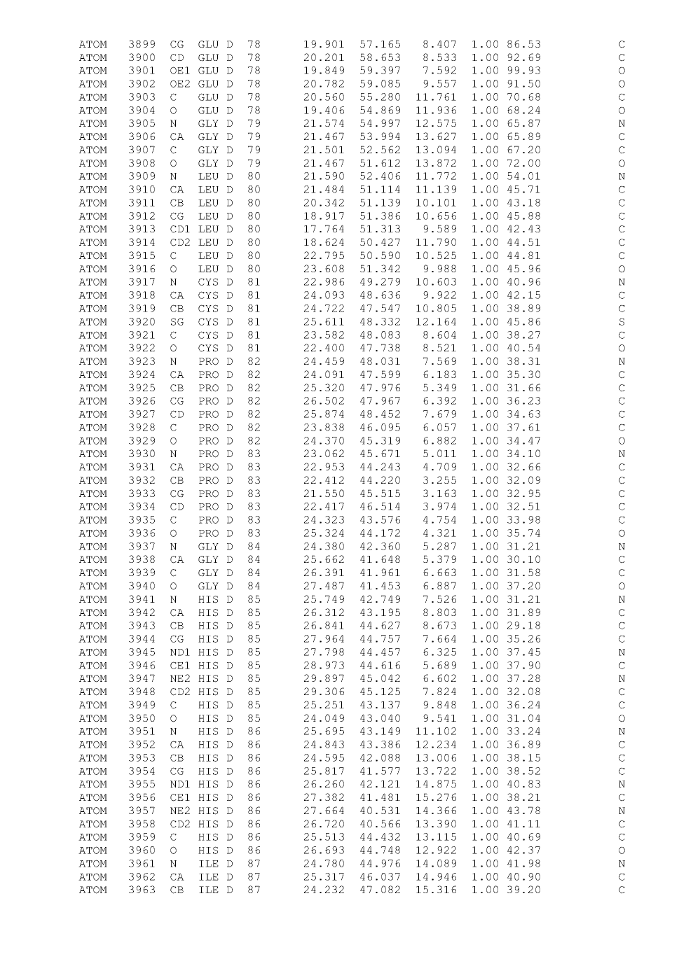

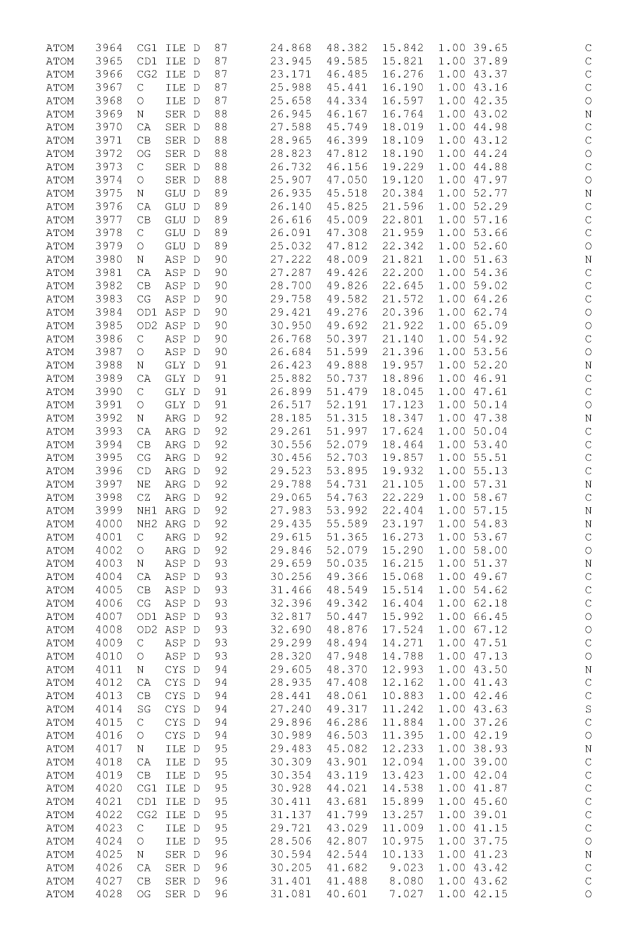

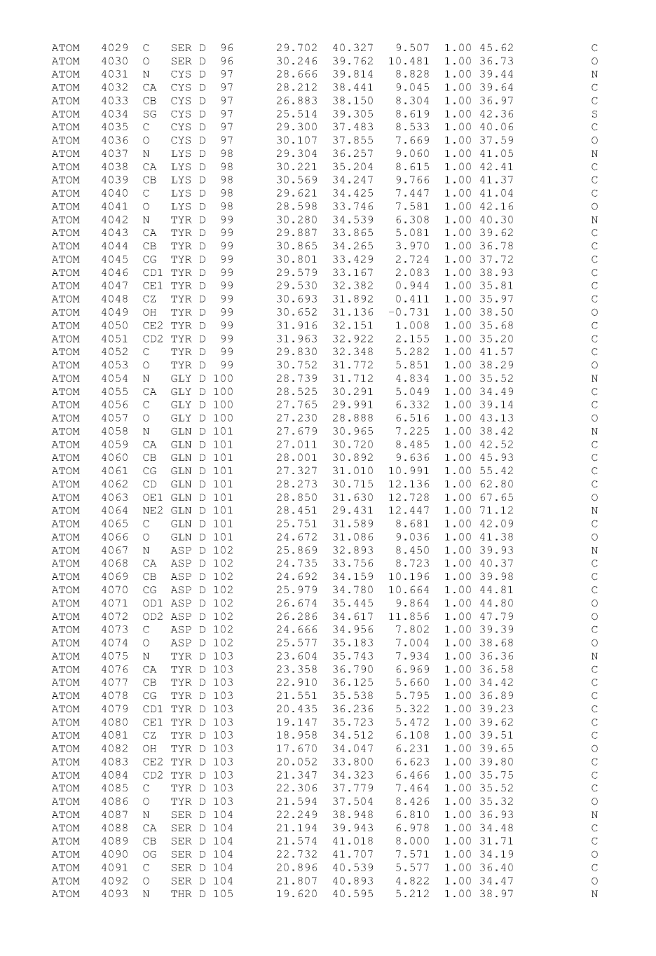

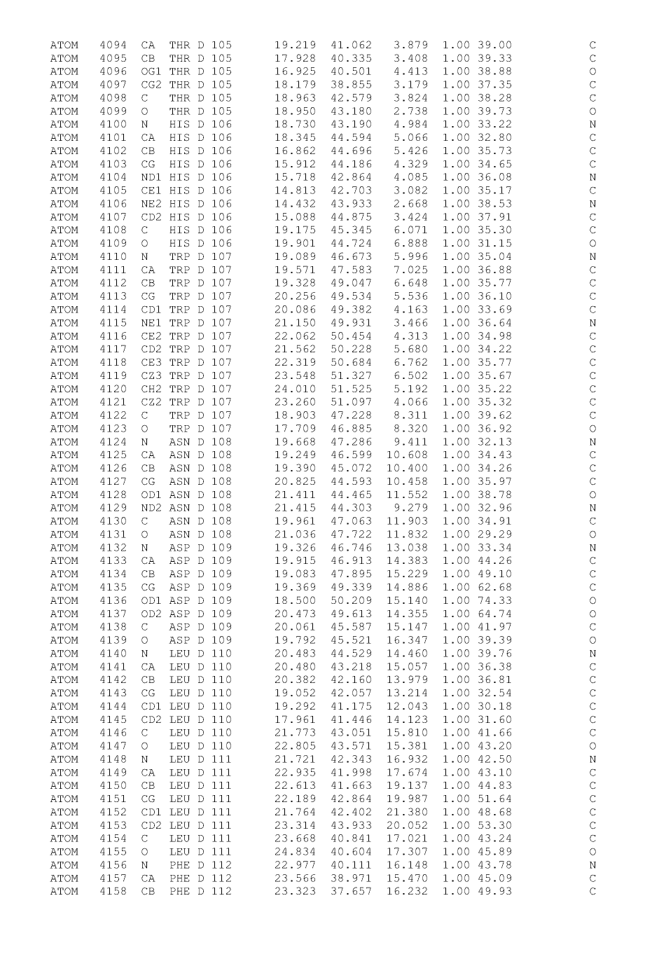

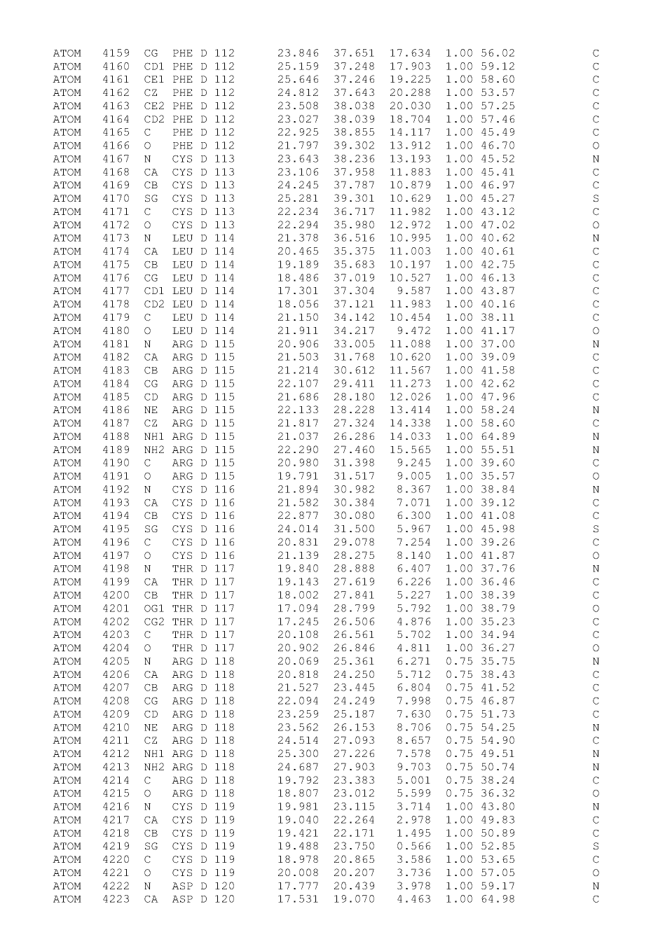

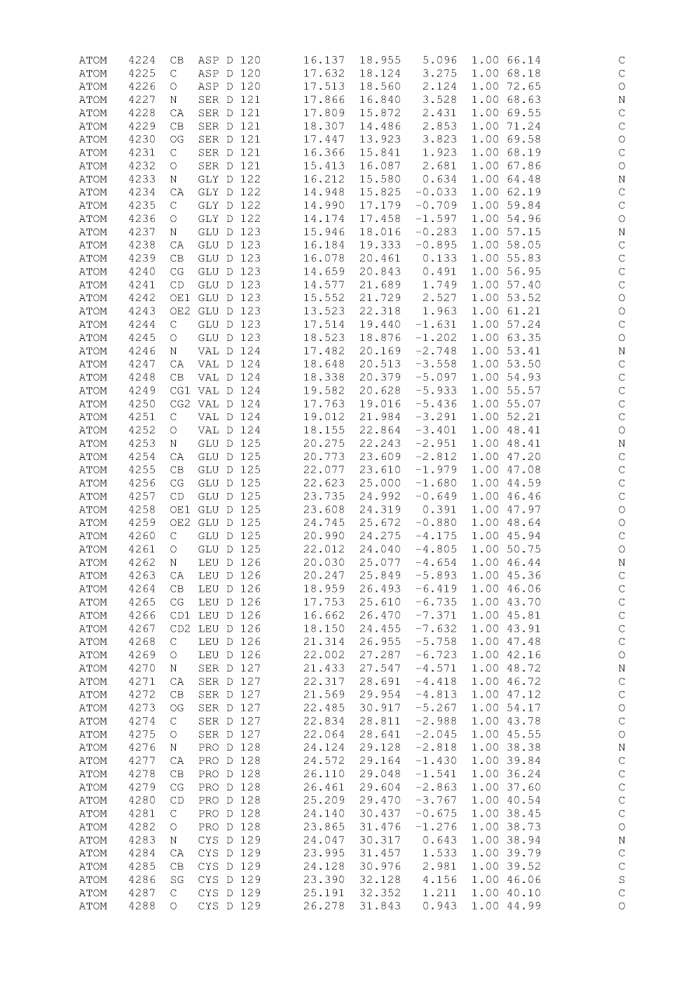

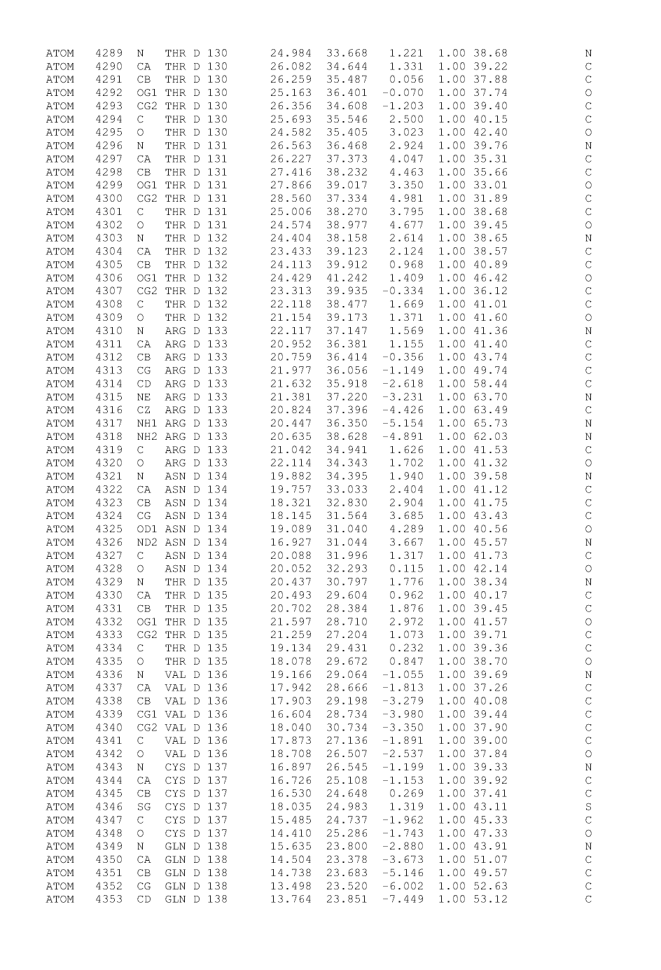

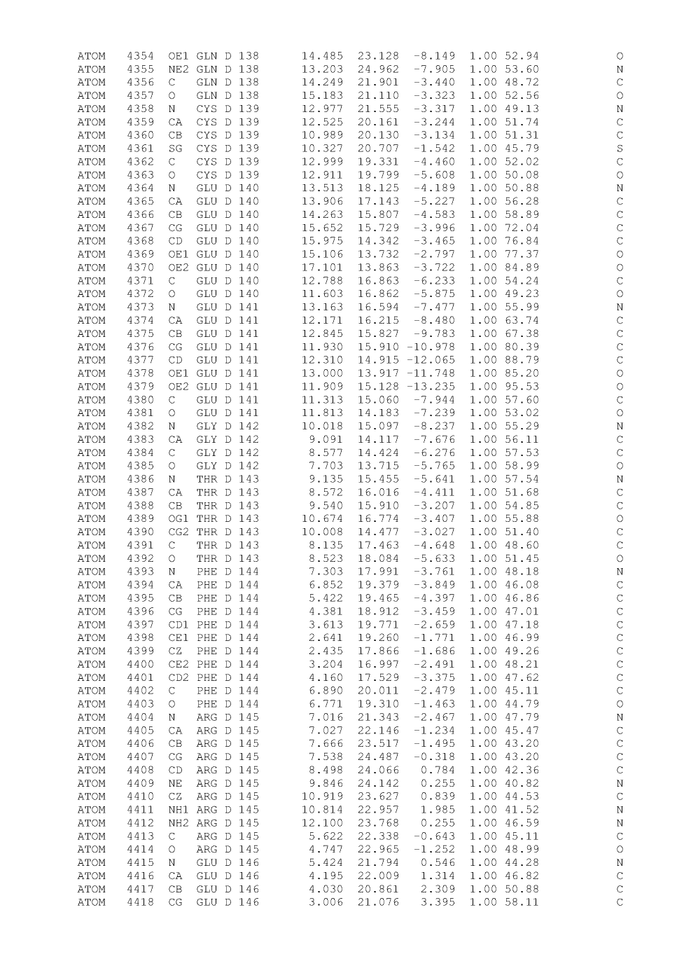

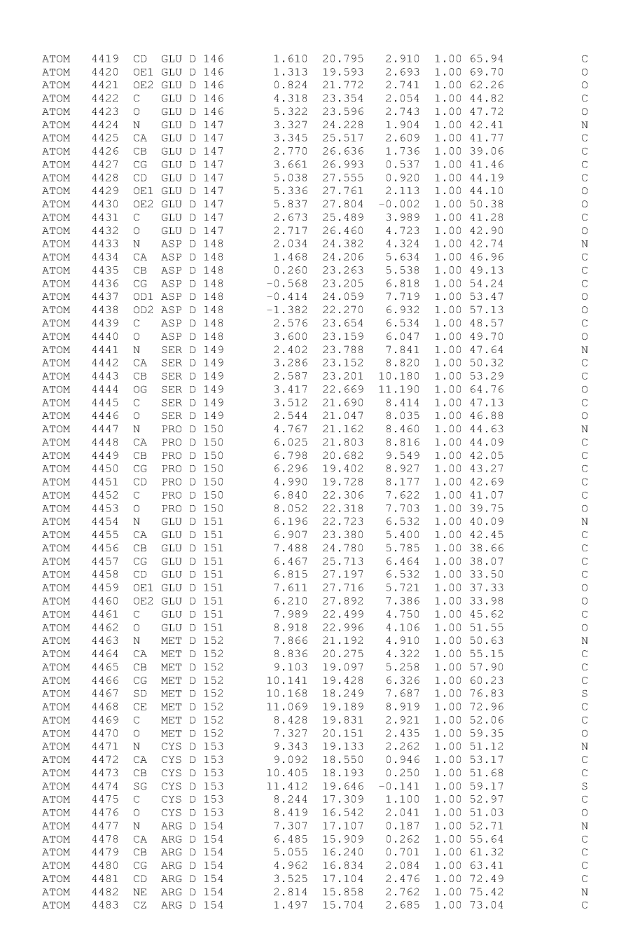

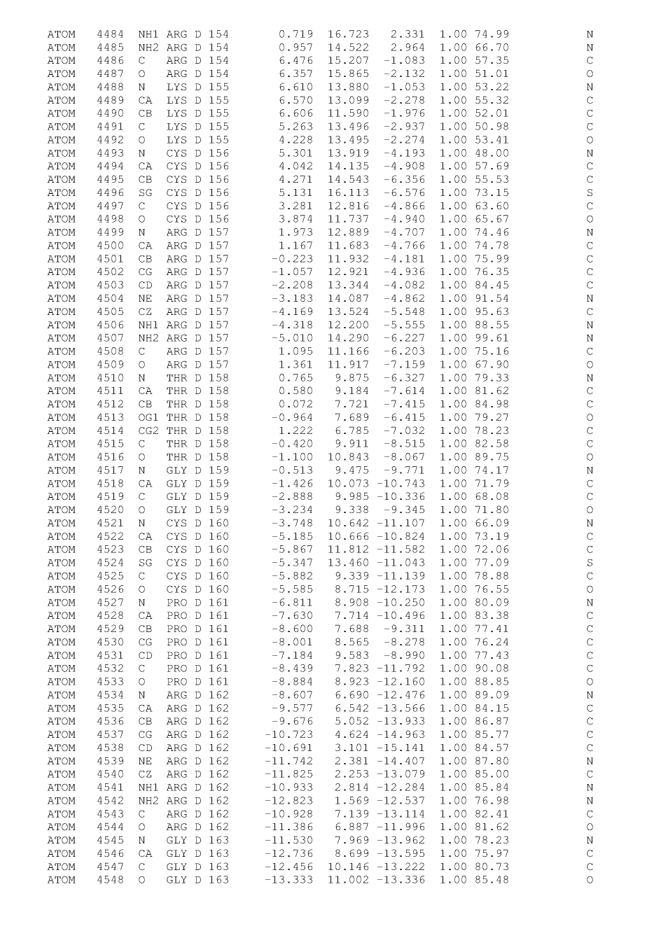

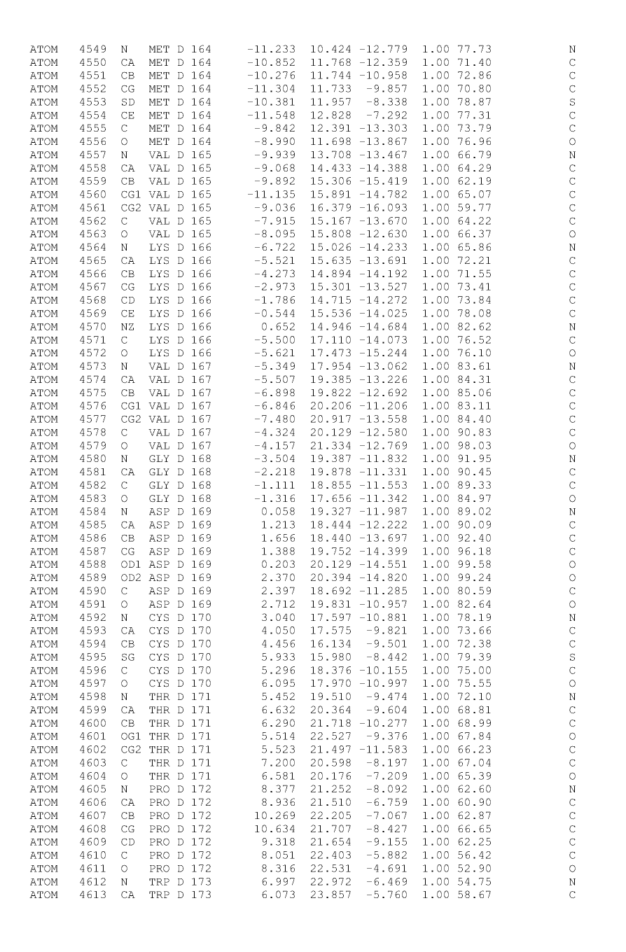

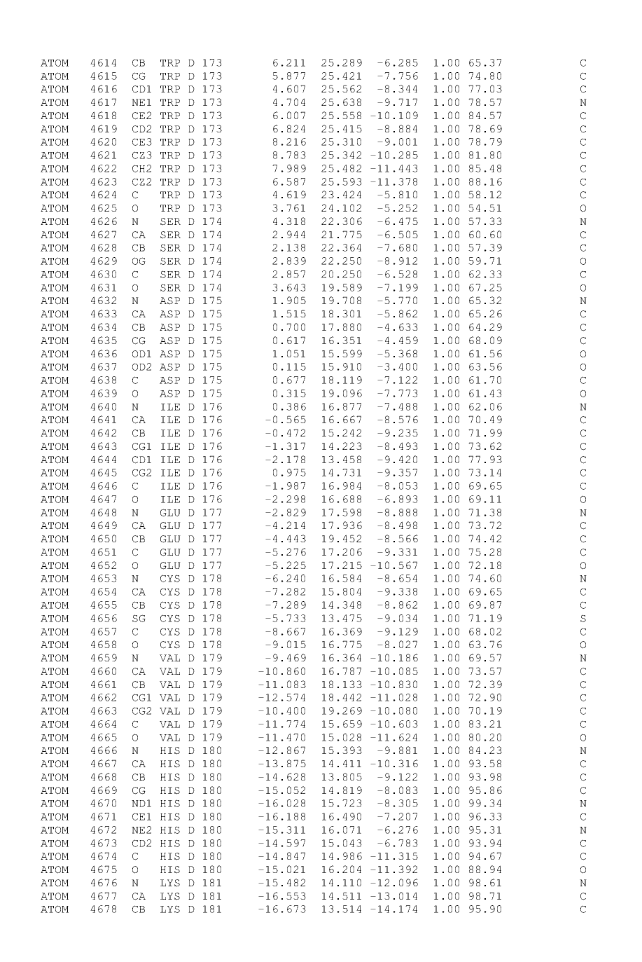

Lung epithelial cells expressing UL141 exhibited a marked reduction in cell surface expression of TRAIL-R2 and CD155, while intracellular levels of both molecules increased (FIGS. 22A and 22B). Previous studies revealed UL141 to be a potent inhibitor of NK cell killing via downregulation of the DNAM-1 activating ligands CD155 and CD112 (Tomasec et al., 2005), but were not designed to measure contributions of NK-mediated apoptosis regulated by DR signaling. TRAIL is poorly expressed in the majority of human NK cells isolated directly from peripheral blood, although, interestingly, it is present at high levels in the small percentage of CD56^{hi} NK. Consequently, ‘bulk’ NK cells were first activated with IFNα (FIG. 22C), a physiologically relevant inducer of TRAIL expression during viral infection in vivo (Sato et al., 2001; Takeda et al., 2001). Using these activated NK effectors, cellular targets transduced with control adenovirus vector were significantly more sensitive to NK-mediated apoptosis than those expressing UL141 (FIG. 22D). Anti-DNAM-1 blocking antibody reduced NK cell killing by ˜65%, and a similar reduction was seen in both control cells and those expressing UL141. Notably, the addition of soluble TRAIL-R2 in combination with anti-DNAM-1 reduced killing to an even greater extent, clearly demonstrating that NK cells utilize this DR to mediate their effector function. Importantly, the level of NK inhibition seen when blocking both DNAM-1 and TRAIL-R2 was higher in UL141 expressing targets than in control cells (˜11 fold vs. ˜4.5 fold), highlighting a selective importance of UL141 in promoting resistance to TRAIL. The observed sensitivity of UL141-expressing targets to NK killing via TRAIL and DNAM-1 is mediated by ‘residual’ levels of CD155 and TRAIL-R2 in these target cells (FIG. 22A), and is very likely relevant given that incomplete inhibition of their cell surface expression is also seen in HCMV infected cells (see FIGS. 16 and 17).

### Example 24: Discussion of Results

Herein is provided the first description of a herpesvirus gene that acts to inhibit TRAIL mediated apoptosis by specifically targeting expression of the TRAIL DRs. This study highlights the fundamental role that signaling by TNF-family cytokines plays in driving the evolution of host-attack and viral-retort that is critical for the success of persistent viral pathogens. The data is consistent with a model where gpUL141 binds directly to the ectodomain of the human TRAIL DRs in the lumen of the ER, sequestering them as a stable complex as both proteins accumulate. Consequently, transport through the Golgi apparatus and onward is impeded, and cells are desensitized to TRAIL killing. Notably, HCMV infection had previously been reported to sensitize cells to TRAIL and induce DR expression (Sedger et al., 1999), but this can now be explained by the use of the high-passage AD169 strain in those studies, which encodes a defective UL36 in combination with lacking the entire UL/b′ genomic region (Skaletskaya et al., 2001a) Cha et al., 1997). TRAIL expression is upregulated on the surface of HCMV infected dendritic cells (DC), promoting the death of virus-specific T cells that encounter them (Raftery et al., 2001). Perhaps the restriction of TRAIL DRs by UL141 is necessary to protect the infected DCs from TRAIL-mediated fratricide and/or suicide. TRAIL mRNA is also highly induced by HCMV in placental fibroblasts via the action of type I IFN (Andrews et al., 2007), suggesting a similar mechanism could be operable to thwart host immunity during congenital infection (Nigro and Adler, 2010). Consequently, HCMV may utilize the immune-suppressive activities of TRAIL to its advantage, while commensurately inhibiting its action in infected cells via the action of UL141. Intriguingly, and indicative of a multifaceted role for the TRAIL DR in CMV defense, dendritic cells from TRAIL-R2−/− mice produce increased levels of inflammatory cytokines when infected with mouse CMV (MCMV), promoting increased NK cell activation and enhanced control of viral replication in the spleen, but not the liver (Diehl et al., 2004). Although the mechanism(s) for this inhibitory role of TRAIL DR signalling in mice is not currently understood, it illustrates the importance of considering cell-type and tissue-specific roles for the TRAIL cytokine system in regulating antiviral immune defenses.

HCMV now joins a select set of viruses with predicted or demonstrated capacity to restrict TRAIL-mediated killing, with UL141 being the first herpesvirus gene shown to specifically target the TRAIL DRs. A complex encoded by the adenovirus type 2 E3 gene region (E3 6.7K/10.4K/14.5K) redirects TRAIL DR for lysosomal degradation (Benedict et al., 2001; Lichtenstein et al., 2004) and the HBV core protein inhibits TRAIL-R2 transcription (Du et al., 2009). A number of viruses preferentially target steps downstream of the TRAIL DR; indeed HCMV UL36 is an inhibitor of caspase-8. HIV infected macrophages and dendritic cells (DC) also show reduced sensitivity to TRAIL, commensurate with inhibition of TRAIL-R1 expression and increased levels of the prosurvival proteins FLIP and IAP-2 (Melki et al., 2010; Swingler et al., 2007). Cowpox and HPV-16 block TRAIL killing by altering formation of the death-inducing signalling complex (DISC) (Kabsch and Alonso, 2002; Marsters et al., 1996). In other herpesviruses, HHV-7 blocks TRAIL-R1 expression and TRAIL killing of CD4 T cells, albeit via an unknown mechanism (Secchiero et al., 2001). Kaposi's sarcoma associated herpesvirus (KSHV/HHV-8) encodes an orthologue of the cellular FLIP proteins (vFLIP) (Thome et al., 1997), competing for DISC assembly to inhibit TRAIL signaling. In latently infected B cells, Epstein Barr virus (EBV/HHV-4) restricts TRAIL killing by inducing higher NFκB-inducing via latent membrane protein-1 (Snow et al., 2006), perhaps helping to promote EBV-associated tumors (Li et al., 2011). Similar to HCMV UL36, the ribonucleotide reductase R1 subunits of herpes simplex virus types 1 and 2 binds caspase-8 to block its activation by TNFα and FasL (Dufour et al., 2011a; Dufour et al., 2011b), with TRAIL yet to be tested. Taken together, viral blockade of TRAIL-mediated apoptosis is likely to be of paramount importance given the wide swath of strategies that have evolved to inhibit it.

Although CD155 and CD112 share homology, as do TRAIL-R1 and -R2, these proteins show no primary sequence or predicted structural homology to each other. Consequently, whether UL141 utilizes a similar mechanism to bind to both the TRAIL DRs and CD155 remains an open question, and understanding the structural determinants for these interactions may shed light upon how these receptor/ligand systems function in the host. Until quite recently, interacting partners for the TNFRs were thought to be restricted to the trimeric TNF-family ligands. However, when HVEM/TNFRSF14 was found to bind the inhibitory cosignaling receptor BTLA this dogma was reassessed (Sedy et al., 2005). Interestingly, HCMV UL144 also targets this signaling system. UL144 is a partial functional-orthologue of HVEM that binds to BTLA, but not to LIGHT, and potently inhibits T cell proliferation (Cheung et al., 2005; Sedy et al., 2008). Our data highlight UL141 as the first non-TNF family protein that can interact with the ectodomain of the TRAIL DRs, providing further evidence for TNFR binding partners that extend outside of the canonical family. This interaction is highly specific, as high affinity binding of UL141 was not detected to any other member of the TNFR superfamily. It is intriguing that UL141 binds to TRAIL-R1 with a much lower affinity than to TRAIL-R2, as this exactly mimics what is seen for TRAIL binding (Truneh et al., 2000), and suggests that low affinity binding to TRAIL-R1 may have some biological significance that is currently underappreciated. Along these lines, TRAIL-R2 is normally expressed intracellularly at high abundance in uninfected cells, with UL141 greatly enhancing these levels. The UL141-mediated accumulation of TRAIL DR, as well as CD155, raises the exciting possibility that these host-cell proteins may have yet-to-be described roles as intracellular signalers, as well as potential regulators of viral persistence.

UL141 is now known to be required for restricting the cell surface expression of at least four cellular proteins, TRAIL-R1, -R2, CD155 and CD112. CD155 was the first identified target of UL141 (Tomasec et al., 2005), and the decreased sensitivity of cells expressing UL141 to NK killing is ascribed in part to its inhibition of NK activation via DNAM 1. DNAM 1 is an activating receptor that is a key initial component of NK activation/licensing to kill its target. Killing itself can then be mediated through cytotoxic granule release in conjunction with signaling by TNF-family ligands that bind to cognate death receptors. In order to assess whether UL141 restriction of TRAIL DR expression contributes to NK inhibition, a physiologically relevant assay was developed where IFNα-activated NK cells were used as effectors. Interestingly, although CD56^{hi}NK cells only compose a small percentage of circulating NK cells in peripheral blood (˜5%), these cells express high levels of TRAIL. Consequently, since many more NK cells present in human tissues are CD56hi (Poli et al., 2009), this suggests that our assays with NK isolated from blood may even underrepresent the contribution of TRAIL in NK control of HCMV. Also, it is important to distinguish the distinct roles played by DNAM-1 and TRAIL in NK killing. DNAM-1 is involved in the 1° ‘decision-making’ process, while TRAIL participates in executing that decision. This is highlighted by the fact that in our assays, antibody blockade of DNAM-1 would not affect the negative signal mediated by CD155 binding to its ‘paired’ NK inhibitory receptor, TIGIT (Yu et al., 2009). In total, UL141 imposes a multi-layered strategy to inhibit NK cells through dampening both their initial activation and downstream killing.

Finally, targeting of TRAIL DRs, TNFR-1 and HVEM by the UL138-144 UL/b′ locus now defines this gene cluster as being highly focused on modulating signaling by the TNFR superfamily. Additionally, UL141 and UL142 (Ashiru et al., 2009) stand out within this cluster as having proven NK-modulating functions. As NK cells also express BTLA, perhaps UL144 will soon join their ranks Notably, UL141 and UL144 are also conserved and immediately adjacent orfs in the rhesus CMV genome (Hansen et al., 2003), further emphasizing their likely importance in CMV-modulation of

TABLE 1
Binding kinetics measured by SPR.

| | Immobilized | In solution | | | | | kₒₙ | k_{off} | K_{Dave}^{b)} | |
|---|---|---|---|---|---|---|---|---|---|---|
| | (ligand) | (analyte) | K_{Deq} [M] | X | Rₘₐₓ | K_{D} [M] | [M⁻¹s⁻¹] | [s⁻¹] | [nM] | |
| A | UL141-Fc | TRAIL-R2 | 19.8 × 10⁻⁹ | 0.71 | 39.9 | 21.4 × 10⁻⁹ | 2.64 × 10⁵ | 5.64 × 10⁻³ | 20 | nM |
| B | TRAIL-R2-Fc | UL141 | n.d.^{a)} | 1.33 | 41.2 | 5.96 × 10⁻⁹ | 1.21 × 10⁴ | 7.21 × 10⁻⁵ | 6 | nM^{c)} |
| C | TRAIL-R1-Fc | UL141 | 2.27 × 10⁻⁹ | 0.88 | 40.2 | 2.33 × 10⁻⁶ | 6.02 × 10³ | 1.40 × 10⁻² | 2.3 | μM^{c)} |
| D | CD155-Fc | UL141 | n.d.^{a)} | 2.11 | 31.3 | 1.97 × 10⁻⁹ | 1.76 × 10⁵ | 3.46 × 10⁻⁴ | 2 | nM |
| E | CD155-Fc | UL141- | n.d.^{a)} | 1.98 | 87.1 | 2.19 × 10⁻⁹ | 1.52 × 10⁵ | 3.33 × 10⁻⁴ | 2 | nM |
| | | TRAIL-R2 | | | | | | | | |

^{a)}Samples with low analyte concentrations did not reach chemical equilibrium (plateau phase) during injection, which is required to perform a reliable steady-state analysis (K_{Deq}).
^{b)}Average equilibrium binding affinity (K_{Dave}) was derived from both K_{Deq} and K_{D}.
^{c)}values from ref. 12.

primate innate immunity.

TABLE 2
Data collection and refinement statistics.

| | | | UL141-TR2 | | UL141-TR2 | |
|---|---|---|---|---|---|---|
| | UL141-TR2 | | C6 crystal | | Multi-crystal | |
| | Native | | derivative | | derivative | |
| Data collection | | | | | | |
| statistics | | | | | | |
| Space group | P2₁2₁2₁ | | P2₁2₁2₁ | | P2₁2₁2₁ | |
| Cell dimension | | | | | | |
| a, b, c (Å) | 67.71, 97.65, 141.31 | | 67.91, 97.04, 141.42 | | 67.99, 97.04, 141.64 | |
| α, β, γ (°) | 90.0, 90.0, 90.0 | | 90.0, 90.0, 90.0 | | 90.0, 90.0, 90.0 | |
| Resolution range (Å) | 65.13-2.50 | | 51.78-2.30 | | 19.83-2.10 | |
| ^{a}[outer shell] | [2.82-2.50] | | [2.36-2.30] | | [2.21-2.10] | |
| Wavelength (Å) | 0.9698 | | 0.9795 | | 0.9795 | |
| No. reflections | 33185 | | 40153 | | 100305 | |
| R_{merge} (%) | 8.1 | [69.2] | 9.2 | [65.3] | 10.4 | [66.6] |
| Multiplicity | 5.3 | [5.4] | 7.4 | [7.5] | 7.5 | [10.3] |
| Average I/σ(I) | 13.2 | [3.1] | 8.9 | [2.5] | 8.4 | [2.1] |
| Completeness (%) | 100.0 | [100.0] | 99.99 | [100.0] | 99.42 | [97.5] |

| | UL141-TR2 |
|---|---|
| Refinement | |
| statistics | |
| No. Atoms | 4999 |
| Protein | 4685 |
| Carbohydrate | 70 |
| Waters | 239 |
| Other solvent | 5 |
| R_{work}/R_{free} | 0.223/0.274 |
| Ramachandran | |
| plot (%) | |
| Favored | 95.6 |
| Allowed | 99.6 |
| R.m.s. | |
| deviations | |
| Bonds (Å) | 0.013 |
| Angles (°) | 1.45 |
| B-factors | |
| (Å²) | |
| Protein | 51.0 |
| Carbohydrate | 52.8 |
| Waters | 54.5 |
| Other solvent | 43.9 |

*Values in parenthesis refer to highest resolution shell

TABLE 3
Determination of the binding contribution accessed by surface plasmon
resonance of a specific residue by alanine scanning on TRAIL-R2

| Patch | Alanine | Binding to | | K_{D} | Kₒₙ | K_{off} | | |
|---|---|---|---|---|---|---|---|---|
| No. | mutation | TRAIL | UL141 | [nM] | [M⁻¹s⁻¹] | [s⁻¹] | Rₘₐₓ | X² |
| | WT | — | 6 nM | 5.95 | 1.21 × | 7.21 × | 210 | 1.5 |
| | | | | | 10⁴ | 10⁻⁵ | | |
| | | 4 nM | — | 3.96 | 0.23 × | 9.11 × | 115 | 2.5 |
| | | | | | 10⁴ | 10⁻⁶ | | |
| 3 | P150 | — | YES | 8.32 | 4.70 × | 3.91 × | 101 | 1.5 |
| | | | | | 10⁴ | 10⁻⁴ | | |
| | | YES | — | 4.29 | 0.21 × | 9.02 × | 130 | 3.1 |
| | | | | | 10⁴ | 10⁻⁶ | | |
| | E151 | NO | NO | n.d.* | n.d.* | n.d.* | n.d.* | n.d.* |
| 3U | D148 | — | 10-fold | 55.32 | 2.35 × | 1.30 × | 100 | 2.7 |
| | | | lower | | 10⁴ | 10⁻³ | | |
| | | YES | — | 4.48 | 0.80 × | 3.58 × | 122 | 2.5 |
| | | | | | 10⁴ | 10⁻⁵ | | |
| 5 | L110/ | — | 10-fold | 42.67 | 1.50 × | 6.40 × | 68 | 1.2 |
| | L114 | | lower | | 10⁴ | 10⁻⁴ | | |
| | | NO | — | n.d.* | n.d.* | n.d.* | n.d.* | n.d.* |
| | F112 | — | 100- | 630.28 | 8.98 × | 5.66 × | 248 | 1.8 |
| | | | fold | | 10³ | 10⁻³ | | |
| | | | lower | | | | | |
| | | YES | — | 3.94 | 0.25 × | 9.86 × | 150 | 3.8 |
| | | | | | 10⁴ | 10⁻⁶ | | |
| 3T | M152 | — | YES | 7.49 | 5.10 × | 3.82 × | 400 | 1.3 |
| | | | | | 10⁴ | 10⁻⁴ | | |
| | | 50- | — | 202.38 | 3.35 × | 6.78 × | 118 | 2.2 |
| | | fold | | | 10³ | 10⁻⁴ | | |
| | | lower | | | | | | |
| | | — | YES | 6.26 | 3.88 × | 2.43 × | 320 | 1.9 |
| | | | | | 10⁴ | 10⁻⁴ | | |
| | R154 | 10- | — | 45.70 | 2.56 × | 1.17 × | 109 | 2.7 |
| | | fold | | | 10³ | 10⁻⁴ | | |
| | | lower | | | | | | |
| | | — | YES | 7.74 | 4.91 × | 3.80 × | 352 | 1.2 |
| | | | | | 10⁴ | 10⁻⁴ | | |
| | K155 | 10- | — | 39.66 | 3.48 × | 1.38 × | 120 | 2.9 |
| | | fold | | | 10³ | 10⁻⁴ | | |
| | | lower | | | | | | |
| | M152/ | — | YES | 7.39 | 3.65 × | 2.70 × | 69 | 1.1 |
| | R154/ | | | | 10⁴ | 10⁻⁴ | | |
| | K155 | NO | — | n.d.* | n.d.* | n.d.* | n.d.* | n.d.* |
| 4 | Y103/ | NO | NO | n.d.* | n.d.* | n.d.* | n.d.* | n.d.* |
| | R133 | | | | | | | |
| | Y103/ | NO | NO | n.d.* | n.d.* | n.d.* | n.d.* | n.d.* |
| | N134 | | | | | | | |
| 1 | V167 | — | 2.5- | 15.48 | 3.10 × | 4.80 × | 74 | 1.1 |
| | | | fold | | 10⁴ | 10⁻⁴ | | |
| | | | lower | | | | | |
| | | YES | — | 4.35 | 0.22 × | 9.57 × | 400 | 4.1 |
| | | | | | 10⁴ | 10⁻⁶ | | |
| 1-2 | V167/ | — | NO | n.d.* | n.d.* | n.d.* | n.d.* | n.d.* |
| | W173/ | YES | — | 4.15 | 0.26 × | 1.08 × | 35 | 2.8 |
| | V179 | | | | 10⁴ | 10⁻⁵ | | |
| 6 | E78/ | — | 10-fold | 60.4 | 2.5 × | 1.51 × | 35 | 1.9 |
| | D109 | | lower | | 10⁴ | 10⁻³ | | |
| | | YES | — | 3.92 | 0.23 × | 9.01 × | 442 | 2.9 |
| | | | | | 10⁴ | 10⁻⁶ | | |

*n.d.—Binding was not detected.

TABLE 4
PCR cloning primers for UL141 and
TR2 expression constructs

hcmvUL141/30for/BamHI
5′-CCGGGATCCCTCGTTCCCCTTCGCCACCG-3′
hcmvUL141/217rev/His/EcoRI (short)
5′-CCGGAATTCTCAGTGATGGTGATGG
TGATGGTCGGCGCGGCCGATATAG-3′
hcmvUL141/279rev/His/EcoRI (long-long)
5′-CCGGAATTCTCAGTGATGGTGATGG
TGATGTCCCCGAGTGGCCCAGGG-3′
huTR2-Fc/58for/EcoRI
5′-CCGGAATTCCAACAAGACCTAGCTCCCCA-3′
huTR2-Fc/184rev/PstI
5′-CCGCTGCAGGCCTGATTCTTTGTGGACACA-3′
hcmvUL141-Fc/37for/EcoRI
5′-CCGGAATTCGACATTGCCGAAAAGATGTGG-3′
hcmvUL141-Fc/247rev/PstI (middle)
5′-CCGCTGCAGGCAGTCGCCGGGGAGCC-3′
hcmvUL141-Fc/273rev/PstI (long)
5′-CCGCTGCAGAGACATTCCGGTGTCTATGTC-3′

TABLE 5
List of multi-site mutation primers
Single-stranded multi-site (3-4) mutation
primers for Quick Change II Multi-site Kit

R133A_N134A_TRAIL-R2-Fc
5′-GTCCCTGCACCACGACCGCAGCCACAGTGTGTCAGTGCG-3′
Y103_TRAIL-R2-Fc
5′-TCCTGCAAATATGGACAGGACGCTAGCACTCAGTGGAATGAC-3′
L110A_F112A_L114A_R115A_TRAIL-R2-Fc
5′-CTCACTGGAATGACGCCCTTGCCTGCGCGGCCTGCACCAGGTGT
G-3′
D109_TRAIL-R2-Fc
5′-CAGGACTATAGCACTCACTGGAATGCCCTCCTTTTCTGCTTG-3′
E147A_D148A_P150A_E151A_TRAIL-R2-Fc
5′-GCACCTTCCGGGAAGCAGCTTCTGCTGCGATGTGCCGGAAGTG-3′
M152A_R154A_K155A_TRAIL-R2-Fc
5′-AGAAGATTCTCCTGAGGCGTGCGCGGCGTGCCGCACAGGGTGT-3′
V167A_TRAIL-R2-Fc
5′-TGTCCCAGAGGGATGGTCAAGGCCGGTGATTGTACACCC-3′
W173A_I176A_V179A_TRAIL-R2-Fc
5′-GATTGTACACCCGCGAGTGACGCCGAATGTGCCCACAAAGAATC
A-3′
E78_G79_TRAIL-R2-Fc
5′-AGGTCCAGCCCCTCAGCGGCATTGTGTCCACCTGGACACCAT-3′

### REFERENCES

1. Sedy, J. R., Spear, P. G. & Ware, C. F. Cross-regulation between herpesviruses and the TNF superfamily members. Nat Rev Immunol 8, 861-73 (2008).
2. Baumgarth, N., Choi, Y. S., Rothaeusler, K., Yang, Y. & Herzenberg, L. A. B cell lineage contributions to antiviral host responses. Curr Top Microbiol Immunol 319, 41-61 (2008).
3. Schleiss, M. R. Prospects for development and potential impact of a vaccine against congenital cytomegalovirus (CMV) infection. J Pediatr 151, 564-70 (2007).
4. Cha, T. A. et al. Human cytomegalovirus clinical isolates carry at least 19 genes not found in laboratory strains. J Virol 70, 78-83 (1996).
5. Dolan, A. et al. Genetic content of wild-type human cytomegalovirus. J Gen Virol 85, 1301-12 (2004).
6. Loewendorf, A. & Benedict, C. A. Modulation of host innate and adaptive immune defenses by cytomegalovirus: timing is everything. Journal of internal medicine 267, 483-501 (2010).
7. Smith, C. A., Farrah, T. & Goodwin, R. G. The TNF receptor superfamily of cellular and viral proteins: activation, costimulation, and death. Cell 76, 959-62 (1994).
8. Cha, T. A. et al. Genotypic stability of cold-adapted influenza virus vaccine in an efficacy clinical trial. Journal of Clinical Microbiology 38, 839-45 (2000).
9. Ware, C. F. & Sedy, J. R. TNF Superfamily Networks: bidirectional and interference pathways of the herpesvirus entry mediator (TNFSF14). Current opinion in immunology 23, 627-31 (2011).
10. Benedict, C. A., Banks, T. A. & Ware, C. F. Death and survival: viral regulation of TNF signaling pathways. Curr Opin Immunol 15, 59-65 (2003).
11. Roy, C. R. & Mocarski, E. S. Pathogen subversion of cell-intrinsic innate immunity. NatImmunol 8, 1179-87 (2007).
12. Smith, W. et al. Human cytomegalovirus UL141 targets the TRAIL death receptors to inhibit host innate defenses. Cell Host & Microbe under review (2012).
13. Prod'homme, V. et al. Human cytomegalovirus UL141 promotes efficient downregulation of the natural killer cell activating ligand CD112. J Gen Virol 91, 2034-9 (2010).
14. Bottino, C. et al. Identification of PVR (CD155) and Nectin-2 (CD112) as cell surface ligands for the human DNAM-1 (CD226) activating molecule. The Journal of experimental medicine 198, 557-67 (2003).
15. Fuchs, A., Cella, M., Giurisato, E., Shaw, A. S. & Colonna, M. Cutting edge: CD96 (tactile) promotes NK cell-target cell adhesion by interacting with the poliovirus receptor (CD155). Journal of immunology 172, 3994-8 (2004).
16. Tomasec, P. et al. Downregulation of natural killer cell-activating ligand CD155 by human cytomegalovirus UL141. Nature immunology 6, 181-8 (2005).
17. Smyth, M. J. et al. Tumor necrosis factor-related apoptosis-inducing ligand (TRAIL) contributes to interferon gamma-dependent natural killer cell protection from tumor metastasis. The Journal of experimental medicine 193, 661-70 (2001).
18. Johnsen, M. G., Rasmussen, O. F., Albrechtsen, M. & Borkhardt, B. In vivo expression of the 29000 Mr protein from RNA-2 of pea early browning tobravirus. The Journal of general virology 72 (Pt 6), 1223-7 (1991).
19. Kayagaki, N. et al. Involvement of TNF-related apoptosis-inducing ligand in human CD4+ T cell-mediated cytotoxicity. Journal of immunology 162, 2639-47 (1999).
20. Cha, S. S. et al. Crystal structure of TRAIL-DR5 complex identifies a critical role of the unique frame insertion in conferring recognition specificity. The Journal of biological chemistry 275, 31171-7 (2000).
21. Mongkolsapaya, J. et al. Structure of the TRAIL-DR5 complex reveals mechanisms conferring specificity in apoptotic initiation. Nature structural biology 6, 1048-53 (1999).
22. Hymowitz, S. G. et al. Triggering cell death: the crystal structure of Apo2L/TRAIL in a complex with death receptor 5. Molecular cell 4, 563-71 (1999).
23. Banner, D. W. et al. Crystal structure of the soluble human 55 kd TNF receptor-human TNF beta complex: implications for TNF receptor activation. Cell 73, 431-45 (1993).
24. Naismith, J. H., Brandhuber, B. J., Devine, T. Q. & Sprang, S. R. Seeing double: crystal structures of the type I TNF receptor. Journal of molecular recognition: JMR 9, 113-7 (1996).
25. Li, Y. et al. Inducible resistance of tumor cells to tumor necrosis factor-related apoptosis inducing ligand receptor 2-mediated apoptosis by generation of a blockade at the death domain function. Cancer research 66, 8520-8 (2006).
26. Fellouse, F. A. et al. Molecular recognition by a binary code. Journal of molecular biology 348, 1153-62 (2005).
27. Lam, J., Nelson, C. A., Ross, F. P., Teitelbaum, S. L. & Fremont, D. H. Crystal structure of the TRANCE/RANKL cytokine reveals determinants of receptor-ligand specificity. The Journal of clinical investigation 108, 971-9 (2001).
28. Goh, C. R., Loh, C. S. & Porter, A. G. Aspartic acid 50 and tyrosine 108 are essential for receptor binding and cytotoxic activity of tumour necrosis factor beta (lymphotoxin). Protein engineering 4, 785-91 (1991).
29. Goh, C. R. & Porter, A. G. Structural and functional domains in human tumour necrosis factors. Protein engineering 4, 385-9 (1991).
30. Schneider, P. et al. TRAIL receptors 1 (DR4) and 2 (DR5) signal FADD-dependent apoptosis and activate NF-kappaB. Immunity 7, 831-6 (1997).
31. Yamagishi, J. et al. Mutational analysis of structure-activity relationships in human tumor necrosis factor-alpha. Protein engineering 3, 713-9 (1990).
32. Stengel, K. F. et al. Structure of TIGIT immunoreceptor bound to poliovirus receptor reveals a cell-cell adhesion and signaling mechanism that requires cis-trans receptor clustering. Proceedings of the National Academy of Sciences of the United States of America 109, 5399-404 (2012).
33. Compaan, D. M. et al. Attenuating lymphocyte activity: the crystal structure of the BTLAHVEM complex. J Biol Chem 280, 39553-61 (2005).
34. van der Sloot, A. M. et al. Designed tumor necrosis factor-related apoptosis-inducing ligand variants initiating apoptosis exclusively via the DR5 receptor. Proceedings of the National Academy of Sciences of the United States of America 103, 8634-9 (2006).
35. Sato, K. et al. Antiviral response by natural killer cells through TRAIL gene induction by IFN-alpha/beta. Eur J Immunol. 31, 3138-46 (2001).
36. Muller, S., Zocher, G., Steinle, A. & Stehle, T. Structure of the HCMV UL16-MICB complex elucidates select binding of a viral immunoevasin to diverse NKG2D ligands. PLoS pathogens 6, e1000723 (2010).
37. Stebbins, C. E. & Galan, J. E. Structural mimicry in bacterial virulence. Nature 412, 701-5 (2001).
38. Alcami, A. Viral mimicry of cytokines, chemokines and their receptors. Nature reviews. Immunology 3, 36-50 (2003).
39. Vales-Gomez, M., Browne, H. & Reyburn, H. T. Expression of the UL16 glycoprotein of Human Cytomegalovirus protects the virus-infected cell from attack by natural killer cells. BMC immunology 4, 4 (2003).
40. Alexander, J. M. et al. Structural basis of chemokine sequestration by a herpesvirus decoy receptor. Cell 111, 343-56 (2002).
41. Carfi, A., Smith, C. A., Smolak, P. J., McGrew, J. & Wiley, D. C. Structure of a soluble secreted chemokine inhibitor vCCI (p35) from cowpox virus. Proceedings of the National Academy of Sciences of the United States of America 96, 12379-83 (1999).
42. Cheung, T. C. et al. T cell intrinsic heterodimeric complexes between HVEM and BTLA determine receptivity to the surrounding microenvironment. J Immunol 183, 7286-96 (2009).
43. Cheung, T. C. et al. Evolutionarily divergent herpesviruses modulate T cell activation by targeting the herpesvirus entry mediator cosignaling pathway. Proc Natl Acad Sci U S A (2005).
44. Battye, T. G., Kontogiannis, L., Johnson, O., Powell, H. R. & Leslie, A. G. iMOSFLM: a new graphical interface for diffraction-image processing with MOSFLM. Acta crystallographica. Section D, Biological crystallography 67, 271-81 (2011).
45. Evans, P. Scaling and assessment of data quality. Acta crystallographica. Section D, Biological crystallography 62, 72-82 (2006).
46. Liu, Q., Zhang, Z. & Hendrickson, W. A. Multi-crystal anomalous diffraction for lowresolution macromolecular phasing. Acta crystallographica. Section D, Biological crystallography 67, 45-59 (2011).
47. Sheldrick, G. M. A short history of SHELX. Acta crystallographica. Section A, Foundations of crystallography 64, 112-22 (2008).
48. McCoy, A. J. et al. Phaser crystallographic software. Journal of applied crystallography 40, 658-674 (2007).
49. Cowtan, K. D. & Zhang, K. Y. Density modification for macromolecular phase improvement. Progress in biophysics and molecular biology 72, 245-70 (1999).
50. Panjikar, S., Parthasarathy, V., Lamzin, V. S., Weiss, M. S. & Tucker, P. A. Autorickshaw: an automated crystal structure determination platform as an efficient tool for the validation of an X-ray diffraction experiment. Acta crystallographica. Section D, Biological crystallography 61, 449-57 (2005).
51. Emsley, P. & Cowtan, K. Coot: model-building tools for molecular graphics. Acta crystallographica. Section D, Biological crystallography 60, 2126-32 (2004).
52. Murshudov, G. N., Vagin, A. A. & Dodson, E. J. Refinement of macromolecular structures by the maximum-likelihood method. Acta crystallographica. Section D, Biological crystallography 53, 240-55 (1997).
53. Lovell, S. C. et al. Structure validation by Calpha geometry: phi,psi and Cbeta deviation. Proteins 50, 437-50 (2003).
54. Loewendorf A, Benedict C A. Modulation of host innate and adaptive immune defenses by cytomegalovirus: Timing is everything. J Intern Med. 2010; 267:483-501.
55. Benedict C, Butrovich K, Lurain N, Corbeil J, Rooney I, Schenider P, et al. Cutting Edge: A novel viral TNF receptor superfamily member in virulent strains of human cytomegalovirus. J Immunol. 1999; 162:6967-70.
56. Cheung T C, Humphreys I R, Potter K G, Norris P S, Shumway H M, Tran B R, et al. Evolutionarily divergent herpesviruses modulate T cell activation by targeting the herpesvirus entry mediator cosignaling pathway. Proc Natl Acad Sci USA. 2005.
57. Sedy J R, Spear P G, Ware C F. Cross-regulation between herpesviruses and the TNF superfamily members. Nat Rev Immunol. 2008; 8(11):861-73. PMCID: 2665163.
58. Takeda K, Cretney E, Hayakawa Y, Ota T, Akiba H, Ogasawara K, et al. TRAIL identifies immature natural killer cells in newborn mice and adult mouse liver. Blood. 2005; 105(5):2082-9.
58. Andrews, J. I., Griffith, T. S., and Meier, J. L. (2007). Cytomegalovirus and the role of interferon in the expression of tumor necrosis factor-related apoptosis-inducing ligand in the placenta. American journal of obstetrics and gynecology 197, 608 e601-606.
59. Ashiru, O., Bennett, N. J., Boyle, L. H., Thomas, M., Trowsdale, J., and Wills, M. R. (2009). NKG2D ligand MICA is retained in the cis-Golgi apparatus by human cytomegalovirus protein UL142. J Viro 183, 12345-12354.
60. Ashkenazi, A., and Dixit, V. M. (1999). Apoptosis control by death and decoy receptors. Curr Opin Cell Biol 11, 255-260.
61. Baillie, J., Sahlender, D. A., and Sinclair, J. H. (2003). Human cytomegalovirus infection inhibits tumor necrosis factor alpha (TNF-alpha) signaling by targeting the 55-kilodalton TNF-alpha receptor. J Virol 77, 7007-7016.
62. Benedict, C., Butrovich, K., Lurain, N., Corbeil, J., Rooney, I., Schenider, P., Tschopp, J., and Ware, C. (1999). Cutting Edge: A novel viral TNF receptor superfamily member in virulent strains of human cytomegalovirus. J Immunol 162, 6967-6970.
63. Benedict, C. A. (2003). Viruses and the TNF-related cytokines, an evolving battle. Cytokine Growth Factor Rev 14, 349-357.
64. Benedict, C. A., Norris, P. S., Prigozy, T. I., Bodmer, J. L., Mahn, J. A., Garnett, C. T., Maranon, F., Tschopp, J., Gooding, L. R., and Ware, C. F. (2001). Three Adenovirus E3 Proteins Cooperate to Evade Apoptosis by Tumor Necrosis Factor-related Apoptosisinducing Ligand Receptor-1 and -2. J Biol Chem 276, 3270-3278.
65. Bodmer, J. L., Holler, N., Reynard, S., Vinciguerra, P., Schneider, P., Juo, P., Blenis, J., and Tschopp, J. (2000). TRAIL receptor-2 signals apoptosis through FADD and caspase-8. Nat Cell Biol 24, 241-243.
66. Bodmer, J. L., Schneider, P., and Tschopp, J. (2002). The molecular architecture of the TNF superfamily. Trends Biochem Sci 27, 19-26.
67. Bossen, C., Ingold, K., Tardivel, A., Bodmer, J. L., Gaide, O., Hertig, S., Ambrose, C., Tschopp, J., and Schneider, P. (2006). Interactions of tumor necrosis factor (TNF) and TNF receptor family members in the mouse and human. J Biol Chem 281, 13964-13971.
68. Cha, T. A., Tom, E., Kemble, G. W., Duke, G. M., Mocarski, E. S., and Spaete, R. R. (1996). Human cytomegalovirus clinical isolates carry at least 19 genes not found in laboratory strains. J Viro 170, 78-83.
69. Chalupny, N. J., Rein-Weston, A., Dosch, S., and Cosman, D. (2006). Downregulation of the NKG2D ligand MICA by the human cytomegalovirus glycoprotein UL142. Biochem Biophys Res Commun 346, 175-181.
70. Chen, L. H., Jiang, C. C., Kiejda, K. A., Wang, Y. F., Thorne, R. F., Zhang, X. D., and Hersey, P. (2007). Thapsigargin sensitizes human melanoma cells to TRAIL-induced apoptosis by up-regulation of TRAIL-R2 through the unfolded protein response. Carcinogenesis 28, 2328-2336.
71. Cheung, T. C., Humphreys, I. R., Potter, K. G., Norris, P. S., Shumway, H. M., Tran, B. R., Patterson, G., Jean-Jacques, R., Yoon, M., Spear, P. G., et al. (2005). Evolutionarily divergent herpesviruses modulate T cell activation by targeting the herpesvirus entry mediator cosignaling pathway. Proc Natl Acad Sci USA 102, 13218-13223.
72. Cobbs, C. S., Harkins, L., Samanta, M., Gillespie, G. Y., Bharara, S., King, P. H., Nabors, L. B., Cobbs, C. G., and Britt, W. J. (2002). Human cytomegalovirus infection and expression in human malignant glioma. Cancer Res 62, 3347-3350.
73. Cummins, N., and Badley, A. (2009). The TRAIL to viral pathogenesis: the good, the bad and the ugly. Curr Mol Med 9, 495-505.
74. Dargan, D. J., Douglas, E., Cunningham, C., Jamieson, F., Stanton, R. J., Baluchova, K., McSharry, B. P., Tomasec, P., Emery, V. C., Percivalle, E., et al. (2010). Sequential mutations associated with adaptation of human cytomegalovirus to growth in cell culture. J Gen Virol 91, 1535-1546.
75. Diehl, G. E., Yue, H. H., Hsieh, K., Kuang, A. A., Ho, M., Morici, L. A., Lenz, L. L., Cado, D., Riley, L. W., and Winoto, A. (2004). TRAIL-R as a negative regulator of innate immune cell responses. Immunity 21, 877-889.
76. Du, J., Liang, X., Liu, Y., Qu, Z., Gao, L., Han, L., Liu, S., Cui, M., Shi, Y., Zhang, Z., et al. (2009). Hepatitis B virus core protein inhibits TRAIL-induced apoptosis of hepatocytes by blocking DR5 expression. Cell Death Differ 16, 219-229.
77. Dufour, F., Sasseville, A. M., Chabaud, S., Massie, B., Siegel, R. M., and Langelier, Y. (2011). The ribonucleotide reductase R1 subunits of herpes simplex virus types 1 and 2 protect cells against TNFalpha- and FasL-induced apoptosis by interacting with caspase-8. Apoptosis 16, 256-271.
78. Gatherer, D., Seirafian, S., Cunningham, C., Holton, M., Dargan, D. J., Baluchova, K., Hector, R. D., Galbraith, J., Herzyk, P., Wilkinson, G. W., et al. (2011). High-resolution human cytomegalovirus transcriptome. Proc Natl Acad Sci USA 108, 19755-19760.
79. Hahn, G., Khan, H., Baldanti, F., Koszinowski, U. H., Revello, M. G., and Gerna, G. (2002). The human cytomegalovirus ribonucleotide reductase homolog UL45 is dispensable for growth in endothelial cells, as determined by a BAC-cloned clinical isolate of human cytomegalovirus with preserved wild-type characteristics. J Virol 76, 9551-9555.
80. Hahn, G., Revello, M. G., Patrone, M., Percivalle, E., Campanini, G., Sarasini, A., Wagner, M., Gallina, A., Milanesi, G., Koszinowski, U., et al. (2004). Human cytomegalovirus UL131-128 genes are indispensable for virus growth in endothelial cells and virus transfer to leukocytes. J Virol 78, 10023-10033.
81. Hansen, S. G., Strelow, L. I., Franchi, D. C., Anders, D. G., and Wong, S. W. (2003). Complete sequence and genomic analysis of rhesus cytomegalovirus. J Virol 77, 6620-6636.
82. Herbeuval, J. P., Boasso, A., Grivel, J. C., Hardy, A. W., Anderson, S. A., Dolan, M. J., Chougnet, C., Lifson, J. D., and Shearer, G. M. (2005a). TNF-related apoptosis-inducing ligand (TRAIL) in HIV-1-infected patients and its in vitro production by antigenpresenting cells. Blood 105, 2458-2464.
83. Herbeuval, J. P., Grivel, J. C., Boasso, A., Hardy, A. W., Chougnet, C., Dolan, M. J., Yagita, H., Lifson, J. D., and Shearer, G. M. (2005b). CD4+ T-cell death induced by infectious and noninfectious HIV-1: role of type 1 interferon-dependent, TRAIL/DR5-mediated apoptosis. Blood 106, 3524-3531.
84. Herold, S., Steinmueller, M., von Wulffen, W., Cakarova, L., Pinto, R., Pleschka, S., Mack, M., Kuziel, W. A., Corazza, N., Brunner, T., et al. (2008). Lung epithelial apoptosis in influenza virus pneumonia: the role of macrophage-expressed TNF-related apoptosis inducing ligand. J Exp Med 205, 3065-3077.
85. Ishikawa, E., Nakazawa, M., Yoshinari, M., and Minami, M. (2005). Role of tumor necrosis factor-related apoptosis-inducing ligand in immune response to influenza virus infection in mice. J Virol 79, 7658-7663.
86. Jarvis, M. A., Borton, J. A., Keech, A. M., Wong, J., Britt, W. J., Magun, B. E., and Nelson, J. A. (2006). Human cytomegalovirus attenuates interleukin-1beta and tumor necrosis factor alpha proinflammatory signaling by inhibition of NF-kappaB activation. J Virol 80, 5588-5598.
87. Kabsch, K., and Alonso, A. (2002). The human papillomavirus type 16 E5 protein impairs TRAIL- and FasL-mediated apoptosis in HaCaT cells by different mechanisms. J Virol 76, 12162-12172.
88. Kotelkin, A., Prikhod'ko, E. A., Cohen, J. I., Collins, P. L., and Bukreyev, A. (2003). Respiratory syncytial virus infection sensitizes cells to apoptosis mediated by tumor necrosis factor-related apoptosis-inducing ligand. J Virol 77, 9156-9172.
89. Le, V. T., Trilling, M., and Hengel, H. (2011). The cytomegaloviral protein pUL138 acts as potentiator of tumor necrosis factor (TNF) receptor 1 surface density to enhance ULb′-encoded modulation of TNF-alpha signaling. Journal of Virology 85, 13260-13270.
90. Leithner, K., Stacher, E., Wurm, R., Ploner, F., Quehenberger, F., Wohlkoenig, C., Balint, Z., Polachova, J., Olschewski, A., Samonigg, H., et al. (2009). Nuclear and cytoplasmic death receptor 5 as prognostic factors in patients with non-small cell lung cancer treated with chemotherapy. Lung Cancer 65, 98-104.
91. Li, S. S., Yang, S., Wang, S., Yang, X. M., Tang, Q. L., and Wang, S. H. (2011). Latent membrane protein 1 mediates the resistance of nasopharyngeal carcinoma cells to TRAIL-induced apoptosis by activation of the PI3K/Akt signaling pathway. Oncology Rpts 26, 1573-1579.
92. Lichtenstein, D. L., Doronin, K., Toth, K., Kuppuswamy, M., Wold, W. S., and Tollefson, A. E. (2004). Adenovirus E3-6.7K protein is required in conjunction with the E3-RID protein complex for the internalization and degradation of TRAIL receptor 2. J Virol 78, 12297-12307.
93. Loewendorf, A., and Benedict, C. A. (2010). Modulation of host innate and adaptive immune defenses by cytomegalovirus: Timing is everything. J Intern Med 267, 483-501.
94. McSharry, B. P., Jones, C. J., Skinner, J. W., Kipling, D., and Wilkinson, G. W. (2001). Human telomerase reverse transcriptase-immortalized MRC-5 and HCA2 human fibroblasts are fully permissive for human cytomegalovirus. J Gen Virol 82, 855-863.
95. Melki, M. T., Saidi, H., Dufour, A., Olivo-Marin, J. C., and Gougeon, M. L. (2010). Escape of HIV-1-infected dendritic cells from TRAIL-mediated NK cell cytotoxicity during NK-DC cross-talk—a pivotal role of HMGB1. PLoS pathogens 6, e1000862.
96. Mocarski, E. S., Upton, J. W., and Kaiser, W. J. (2012). Viral infection and the evolution of caspase 8-regulated apoptotic and necrotic death pathways. Nat Rev Immunol 12, 79-88.
97. Montag, C., Wagner, J. A., Gruska, I., Vetter, B., Wiebusch, L., and Hagemeier, C. (2011). The latency-associated UL138 gene product of human cytomegalovirus sensitizes cells to tumor necrosis factor alpha (TNF-alpha) signaling by upregulating TNF-alpha receptor 1 cell surface expression. J Virol 85, 11409-11421.
98. Murphy, E., Yu, D., Grimwood, J., Schmutz, J., Dickson, M., Jarvis, M. A., Hahn, G.,
99. Nelson, J. A., Myers, R. M., and Shenk, T. E. (2003). Coding potential of laboratory and clinical strains of human cytomegalovirus. Proc Natl Acad Sci USA 100, 14976-14981.
100. Nigro, G., and Adler, S. P. (2010). Cytomegalovirus infections during pregnancy. Curr Opin Obstet Gynecol. 23, 123-128.
101. Poli, A., Michel, T., Theresine, M., Andres, E., Hentges, F., and Zimmer, J. (2009). CD56bright natural killer (NK) cells: an important NK cell subset. Immunology 126, 458-465.
102. Prod'homme, V., Sugrue, D. M., Stanton, R. J., Nomoto, A., Davies, J., Rickards, C. R., Cochrane, D., Moore, M., Wilkinson, G. W., and Tomasec, P. (2010). Human cytomegalovirus UL141 promotes efficient downregulation of the natural killer cell activating ligand CD112. J Gen Virol 91, 2034-2039.
103. Prod'homme, V., Tomasec, P., Cunningham, C., Lemberg, M. K., Stanton, R. J., McSharry, B. P., Wang, E. C., Cuff, S., Martoglio, B., Davison, A. J., et al. (2012). Human Cytomegalovirus UL40 Signal Peptide Regulates Cell Surface Expression of the NK Cell Ligands HLA-E and gpUL18. J Immunol 188, 2794-2804.
104. Raftery, M. J., Schwab, M., Eibert, S. M., Samstag, Y., Walczak, H., and Schonrich, G. (2001). Targeting the function of mature dendritic cells by human cytomegalovirus: a multilayered viral defense strategy. Immunity 15, 997-1009.
105. Sato, K., Hida, S., Takayanagi, H., Yokochi, T., Kayagaki, N., Takeda, K., Yagita, H., Okumura, K., Tanaka, N., Taniguchi, T., et al. (2001). Antiviral response by natural killer cells through TRAIL gene induction by IFN-alpha/beta. Eur J Immunol 31, 3138-3146.
106. Schleiss, M. R. (2007). Prospects for development and potential impact of a vaccine against congenital cytomegalovirus (CMV) infection. J Pediatr 151, 564-570.
107. Schneider, P. (2000). Production of recombinant TRAIL and TRAIL receptor: Fc chimeric proteins. Methods Enzymol 322, 325-345.
108. Schneider, K., Loewendorf, A., De Trez, C., Fulton, J., Rhode, A., Shumway, H., Ha, S., Patterson, G., Pfeffer, K., Nedospasov, S. A., et al. (2008). Lymphotoxin-mediated crosstalk between B cells and splenic stroma promotes the initial type I interferon response to cytomegalovirus. Cell Host Microbe 3, 67-76.
109. Secchiero, P., Mirandola, P., Zella, D., Celeghini, C., Gonelli, A., Vitale, M., Capitani, S., and Zauli, G. (2001). Human herpesvirus 7 induces the functional up-regulation of tumor necrosis factor-related apoptosis-inducing ligand (TRAIL) coupled to TRAIL-R1 down modulation in CD4(+) T cells. Blood 98, 2474-2481.
110. Sedger, L. M., Shows, D. M., Blanton, R. A., Peschon, J. J., Goodwin, R. G., Cosman, D., and Wiley, S. R. (1999). IFN-gamma mediates a novel antiviral activity through dynamic modulation of TRAIL and TRAIL receptor expression. J Immunol 163, 920-926.
111. Sedy, J. R., Gavrieli, M., Potter, K. G., Hurchla, M. A., Lindsley, R. C., Hildner, K., Scheu, S., Pfeffer, K., Ware, C. F., Murphy, T. L., et al. (2005). B and T lymphocyte attenuator regulates T cell activation through interaction with herpesvirus entry mediator. Nat Immunol 6, 90-98.
112. Sedy, J. R., Spear, P. G., and Ware, C. F. (2008). Cross-regulation between herpesviruses and the TNF superfamily members. Nat Rev Immunol 8, 861-873.
113. Simanek, A. M., Dowd, J. B., Pawelec, G., Melzer, D., Dutta, A., and Aiello, A. E. (2011). Seropositivity to cytomegalovirus, inflammation, all-cause and cardiovascular diseaserelated mortality in the United States. PLoS One 6, e16103.
113. Skaletskaya, A., Bartle, L. M., Chittenden, T., McCormick, A. L., Mocarski, E. S., and Goldmacher, V. S. (2001). A cytomegalovirus-encoded inhibitor of apoptosis that suppresses caspase-8 activation. Proc Natl Acad Sci USA 98, 7829-7834.
114. Smyth, M. J., Cretney, E., Takeda, K., Wiltrout, R. H., Sedger, L. M., Kayagaki, N., Yagita, H., and Okumura, K. (2001). Tumor necrosis factor-related apoptosis-inducing ligand (TRAIL) contributes to interferon gamma-dependent natural killer cell protection from tumor metastasis. J Exp Med 193, 661-670.
115. Snow, A. L., Lambert, S. L., Natkunam, Y., Esquivel, C. O., Krams, S. M., and Martinez, O. M. (2006). EBV can protect latently infected B cell lymphomas from death receptor-induced apoptosis. J Immunol 177, 3283-3293.
116. Soderberg-Naucler, C. (2006). Does cytomegalovirus play a causative role in the development of various inflammatory diseases and cancer? J Intern Med 259, 219-246.
117. Stanton, R. J., McSharry, B. P., Armstrong, M., Tomasec, P., and Wilkinson, G. W. (2008). Re-engineering adenovirus vector systems to enable high-throughput analyses of gene function. Biotechniques 45, 659-662, 664-658.
118. Stanton, R. J., Baluchova, K., Dargan, D. J., Cunningham, C., Sheehy, O., Seirafian, S., McSharry, B. P., Neale, M. L., Davies, J. A., Tomasec, P., et al. (2010). Reconstruction of the complete human cytomegalovirus genome in a BAC reveals RL13 to be a potent inhibitor of replication. J Clin Invest 120, 3191-3208.
119. Swingler, S., Mann, A. M., Zhou, J., Swingler, C., and Stevenson, M. (2007). Apoptotic killing of HIV-1-infected macrophages is subverted by the viral envelope glycoprotein. PLoS pathogens 3, 1281-1290.
120. Thome, M., Schneider, P., Hofmann, K., Fickenscher, H., Meinl, E., Neipel, F., Mattmann, C., Burns, K., Bodmer, J. L., Schroter, M., et al. (1997). Viral FLICE-inhibitory proteins (FLIPs) prevent apoptosis induced by death receptors. Nature 386, 517-521.
121. Tomasec, P., Braud, V. M., Rickards, C., Powell, M. B., McSharry, B. P., Gadola, S., Cerundolo, V., Borysiewicz, L. K., McMichael, A. J., and Wilkinson, G. W. (2000). Surface expression of HLA-E, an inhibitor of natural killer cells, enhanced by human cytomegalovirus gpUL40. Science 287, 1031.
122. Tomasec, P., Wang, E. C., Davison, A. J., Vojtesek, B., Armstrong, M., Griffin, C., McSharry, B. P., Morris, R. J., Llewellyn-Lacey, S., Rickards, C., et al. (2005). Downregulation of natural killer cell-activating ligand CD155 by human cytomegalovirus UL141. Nat Immunol 6, 181-188.
123. Truneh, A., Sharma, S., Silverman, C., Khandekar, S., Reddy, M. P., Deen, K. C., McLaughlin, M. M., Srinivasula, S. M., Livi, G. P., Marshall, L. A., et al. (2000). Temperature-sensitive differential affinity of TRAIL for its receptors. DR5 is the highest affinity receptor. J Biol Chem 275, 23319-23325.
124. Wang, J., Li, Y., Kinjo, Y., Mac, T. T., Gibson, D., Painter, G. F., Kronenberg, M., and Zajonc, D. M. (2010). Lipid binding orientation within CD1d affects recognition of Borrelia burgorferi antigens by NKT cells. Proc Natl Acad Sci USA 107, 1535-1540.
125. Wenger, T., Mattern, J., Penzel, R., Gassler, N., Haas, T. L., Sprick, M. R., Walczak, H., Krammer, P. H., Debatin, K. M., and Herr, I. (2006). Specific resistance upon lentiviral TRAIL transfer by intracellular retention of TRAIL receptors. Cell Death and Differentiation 13, 1740-1751.
126. Wilkinson, G. W., Tomasec, P., Stanton, R. J., Armstrong, M., Prod'homme, V., Aicheler, R., McSharry, B. P., Rickards, C. R., Cochrane, D., Llewellyn-Lacey, S., et al. (2008). Modulation of natural killer cells by human cytomegalovirus. J Clin Virol 41, 206-212.
127. Yu, X., Harden, K., Gonzalez, L. C., Francesco, M., Chiang, E., Irving, B., Tom, I., Ivelja, S., Refino, C. J., Clark, H. et al. (2009). The surface protein TIGIT suppresses T cell activation by promoting the generation of mature immunoregulatory dendritic cells. Nat Immunol 10, 48-57.
128. Zhang, X. D., Franco, A. V., Nguyen, T., Gray, C. P., and Hersey, P. (2000). Differential localization and regulation of death and decoy receptors for TNF-related apoptosis-inducing ligand (TRAIL) in human melanoma cells. J Immunol 164, 3961-3970.
129. Zhou, J., Law, H. K., Cheung, C. Y., Ng, I. H., Peiris, J. S., and Lau, Y. L. (2006). Functional tumor necrosis factor-related apoptosis-inducing ligand production by avian influenza virus-infected macrophages. J Inf Dis 193, 945-953.

1. A composition comprising a TRAIL-R2 receptor or fragment thereof bound to a ligand in crystalline form.

2. The composition of claim 1 wherein the ligand is bound to an amino acid sequence of TRAIL-R2 receptor that comprises, consists of or consists essentially of amino acid residues 58-184 of TRAIL-R2 receptor or a subsequence, portion, homologue, variant or derivative thereof.

3. The composition of claim 1 wherein the ligand is bound to an amino acid sequence of TRAIL-R2 receptor that comprises, consists of or consists essentially of amino acid residues 58-212 of TRAIL-R2 receptor or a subsequence, portion, homologue, variant or derivative thereof.

4. The composition of claim 1 wherein the crystalline form has unit cell parameters of a=67.74 Å, b=97.01 Å and c=140.94 Å or a=67.71 Å, b=97.67 Å, c=141.31 Å.

5. The composition of claim 1 comprising the relative structural coordinates set forth in FIG. 23 wherein the resolution is 2.1 Angstrom.

6. The composition of claim 1 comprising a structure set forth in FIG. 1 or FIG. 3.

7. The composition of claim 1 wherein the TRAIL-R2 receptor has one or more binding patches in contact with the ligand, the binding patches comprising, consisting of or consisting essentially of:
i. amino acid residues E78 and D109 of a TRAIL-R2 receptor;
ii. amino acid residue D148 of a TRAIL-R2 receptor;
iii. amino acid residues V167, V179 and W173 of a TRAIL-R2 receptor;
iv. amino acid residues Y103, N134 and R133 of a TRAIL-R2 receptor;
v. amino acid residues L110, L114 and F112 of a TRAIL-R2 receptor; and
vi. amino acid residues E151 and E147 of a TRAIL-R2 receptor;
or a subsequence, portion, homologue, variant or derivative thereof.

8. The composition of claim 1 wherein the ligand is UL141.

9. An isolated or purified ligand binding site of a TRAIL-R2 receptor, or a subsequence, portion, homologue, variant or derivative thereof.

10. The isolated or purified ligand binding site or subsequence, portion, homologue, variant or derivative thereof of claim 9 comprising a structure set forth in any one of FIG. 1, 2, 3, 4 or 6.

11. The isolated or purified ligand binding site or subsequence, portion, homologue, variant or derivative thereof of claim 9 comprising, consisting of or consisting essentially of amino acid residues 58-184 of TRAIL-R2 receptor or a subsequence, portion, homologue, variant or derivative thereof.

12. The isolated or purified ligand binding site or subsequence, portion, homologue, variant or derivative thereof of claim 9 comprising, consisting of or consisting essentially of amino acid residues 58-212 of TRAIL-R2 receptor or a subsequence, portion, homologue, variant or derivative thereof.

13. The isolated or purified ligand binding site or subsequence, portion, homologue, variant or derivative thereof of claim 9 comprising, consisting of or consisting essentially of structural coordinates set forth in FIG. 23, or a subsequence, portion, homologue, variant or derivative thereof.

14. The isolated or purified ligand binding site or subsequence, portion, homologue, variant or derivative thereof of claim 9 comprising, consisting of or consisting essentially of one or more of:
i. amino acid residues E78 and D109 of a TRAIL-R2 receptor;
ii. amino acid residue D148 of a TRAIL-R2 receptor;
iii. amino acid residues V167, V179 and W173 of a TRAIL-R2 receptor;
iv. amino acid residues Y103, N134 and R133 of a TRAIL-R2 receptor;
v. amino acid residues L110, L114 and F112 of a TRAIL-R2 receptor; and
vi. amino acid residues E151 and E147 of a TRAIL-R2 receptor;
or a subsequence, portion, homologue, variant or derivative thereof.

15. A method of designing a compound, protein or peptide that interacts with a TRAIL-R2 receptor, the method comprising:
i. use of the composition of claim 5 to design the compound, protein or peptide, wherein the compound, protein or peptide interacts with a ligand binding site of the TRAIL-R2 receptor, the ligand binding site comprising, consisting of or consisting essentially of amino acid residues 58-184 of the TRAIL-R2 receptor or a subsequence, portion, homologue, variant or derivative thereof;
ii. producing or synthesizing the compound, protein or peptide;
iii. contacting the compound, protein or peptide with the TRAIL-R2 receptor; and
iv. detecting interaction of the compound, protein or peptide with the ligand binding site comprising, consisting of or consisting essentially of amino acid residues 58-184 of the TRAIL-R2 receptor or a subsequence, portion, homologue, variant or derivative thereof.

16. A method of identifying a binding agent that interacts with at least one amino acid of a TRAIL-R2 receptor ligand binding site, the method comprising:
i. providing a test agent;
ii. contacting the test agent with a protein or peptide comprising, consisting of or consisting essentially of amino acid residues 58-184 of the TRAIL-R2 receptor or subsequence, portion, homologue, variant or derivative thereof; and
iii. detecting interaction of the test agent with the protein or peptide comprising, consisting of or consisting essentially of amino acid residues 58-184 of TRAIL-R2 receptor or subsequence, portion, homologue, variant or derivative thereof.

17. The method of claim 16, the method comprising:
i. providing a test agent;
ii. contacting the test agent with a protein or peptide comprising, consisting of or consisting essentially of amino acid residues 58-212 of the TRAIL-R2 receptor or subsequence, portion, homologue, variant or derivative thereof; and
iii. detecting interaction of the test agent with the protein or peptide comprising, consisting of or consisting essentially of amino acid residues 58-212 of TRAIL-R2 receptor or subsequence, portion, homologue, variant or derivative thereof.

18. The method of claim 16, wherein the method comprises identifying a binding agent that interacts with one or more binding patches of the TRAIL-R2 receptor, the binding patches comprising, consisting of or consisting essentially of:
i. amino acid residues E78 and D109 of a TRAIL-R2 receptor;
ii. amino acid residue D148 of the TRAIL-R2 receptor;
iii. amino acid residues V167, V179 and W173 of a TRAIL-R2 receptor;
iv. amino acid residues Y103, N134 and R133 of aTRAIL-R2 receptor;
v. amino acid residues L110, L114 and F112 of a TRAIL-R2 receptor; and
vi. amino acid residues E151 and E147 of the TRAIL-R2 receptor; or a subsequence, portion, homologue, variant or derivative thereof; the method comprising:
i. providing a test agent;
ii. contacting the test agent with a protein or peptide comprising, consisting of or consisting essentially of one or more of the binding patches of the TRAIL receptor; and
iii. detecting interaction of the test agent with the protein or peptide comprising, consisting of or consisting essentially of one or more of the binding patches of the TRAIL receptor.

19. The method of claim 16 wherein the binding agent modulates binding of a TRAIL-R2 receptor to a ligand that interacts with at least one amino acid of a TRAIL receptor ligand binding site.

20. (canceled)

21. (canceled)

22. The method of claim 16 wherein the binding agent is an antibody, an inhibitory nucleic acid or a ligand mimetic.

23.-36. (canceled)


SEQUENCE LISTING (34 sequences)

SEQ ID NO: 1 - protein, 205 aa, Homo Sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 2 - protein, 281 aa, Homo Sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 3 - protein, 233 aa, Homo Sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 281 aa, Homo Sapiens [sequence omitted]

SEQ ID NO: 5 - protein, 317 aa, Homo Sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 6 - protein, 338 aa, Human herpesvirus 5
MCRRESLRTLPWLFWVLLSCPRLLEYSSSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGIAAFLGFWSIFTVCFLCYLCYLQCCGRWCPTPGRGRRGGEGYRRLPTYDSYPGVKKMKR

SEQ ID NO: 7 - protein, 109 aa, Homo Sapiens
PLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESG

SEQ ID NO: 8 - protein, 109 aa, Homo Sapiens
PSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESG

SEQ ID NO: 9 - protein, 107 aa, Homo Sapiens
FKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFG

SEQ ID NO: 10 - protein, 108 aa, Homo Sapiens
LKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESA

SEQ ID NO: 11 - protein, 125 aa, Homo Sapiens
EPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNC

SEQ ID NO: 12 - protein, 122 aa, Homo Sapiens
PPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNC

SEQ ID NO: 13 - protein, 126 aa, Homo Sapiens
HKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKS

SEQ ID NO: 14 - protein, 128 aa, Homo Sapiens
SPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQIC

SEQ ID NO: 15 - protein, 108 aa, Homo Sapiens
PPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSR

SEQ ID NO: 16 - DNA, 20 bp, Artificial Sequence
  Synthetic polynucleotide
ccggcgacgtggtctcataa

SEQ ID NO: 17 - DNA, 20 bp, Artificial Sequence
  Synthetic polynucleotide
atcgcggcatttttgggatt

SEQ ID NO: 18 - DNA, 29 bp, Artificial Sequence
  Synthetic polynucleotide
ccgggatccctcgttccccttcgccaccg

SEQ ID NO: 19 - DNA, 49 bp, Artificial Sequence
  Synthetic polynucleotide
ccggaattctcagtgatggtgatggtgatggtcggcgcggccgatatag

SEQ ID NO: 20 - DNA, 48 bp, Artificial Sequence
  Synthetic polynucleotide
ccggaattctcagtgatggtgatggtgatgtccccgagtggcccaggg

SEQ ID NO: 21 - DNA, 29 bp, Artificial Sequence
  Synthetic polynucleotide
ccggaattccaacaagacctagctcccca

SEQ ID NO: 22 - DNA, 30 bp, Artificial Sequence
  Synthetic polynucleotide
ccgctgcaggcctgattctttgtggacaca

SEQ ID NO: 23 - DNA, 30 bp, Artificial Sequence
  Synthetic polynucleotide
ccggaattcgacattgccgaaaagatgtgg

SEQ ID NO: 24 - DNA, 26 bp, Artificial Sequence
  Synthetic polynucleotide
ccgctgcaggcagtcgccggggagcc

SEQ ID NO: 25 - DNA, 30 bp, Artificial Sequence
  Synthetic polynucleotide
ccgctgcagagacattccggtgtctatgtc

SEQ ID NO: 26 - DNA, 39 bp, Artificial Sequence
  Synthetic polynucleotide
gtccctgcaccacgaccgcagccacagtgtgtcagtgcg

SEQ ID NO: 27 - DNA, 42 bp, Artificial Sequence
  Synthetic polynucleotide
tcctgcaaatatggacaggacgctagcactcagtggaatgac

SEQ ID NO: 28 - DNA, 45 bp, Artificial Sequence
  Synthetic polynucleotide
ctcactggaatgacgcccttgcctgcgcggcctgcaccaggtgtg

SEQ ID NO: 29 - DNA, 42 bp, Artificial Sequence
  Synthetic polynucleotide
caggactatagcactcactggaatgccctccttttctgcttg

SEQ ID NO: 30 - DNA, 43 bp, Artificial Sequence
  Synthetic polynucleotide
gcaccttccgggaagcagcttctgctgcgatgtgccggaagtg

SEQ ID NO: 31 - DNA, 43 bp, Artificial Sequence
  Synthetic polynucleotide
agaagattctcctgaggcgtgcgcggcgtgccgcacagggtgt

SEQ ID NO: 32 - DNA, 39 bp, Artificial Sequence
  Synthetic polynucleotide
tgtcccagagggatggtcaaggccggtgattgtacaccc

SEQ ID NO: 33 - DNA, 45 bp, Artificial Sequence
  Synthetic polynucleotide
gattgtacacccgcgagtgacgccgaatgtgcccacaaagaatca

SEQ ID NO: 34 - DNA, 42 bp, Artificial Sequence
  Synthetic polynucleotide
aggtccagcccctcagcggcattgtgtccacctggacaccat